SEQUENCE LISTING (284 sequences)

SEQ ID NO: 1 - protein, 5 aa, Artificial Sequence
  Description of Artificial Sequence Synthetic peptide
  VHH1 and VHH2 CDR1 (Kabat)
SYRMG

SEQ ID NO: 2 - protein, 5 aa, Artificial Sequence
  Description of Artificial Sequence Synthetic peptide
  VHH3 CDR1 (Kabat)
INVMG

SEQ ID NO: 3 - protein, 5 aa, Artificial Sequence
  Description of Artificial Sequence Synthetic peptide
  VHH4 CDR1 (Kabat)
SNAMG

SEQ ID NO: 4 - protein, 5 aa, Artificial Sequence
  Description of Artificial Sequence Synthetic peptide
  VHH5 CDR1 (Kabat)
SYAMG

SEQ ID NO: 5 - protein, 5 aa, Artificial Sequence
  Description of Artificial Sequence Synthetic peptide
  VHH6 CDR1 (Kabat)
SDAMG

SEQ ID NO: 6 - protein, 5 aa, Artificial Sequence
  Description of Artificial Sequence Synthetic peptide
  VHH7 CDR1 (Kabat)
INVMG

SEQ ID NO: 7 - protein, 5 aa, Artificial Sequence
  Description of Artificial Sequence Synthetic peptide
  VHH9 CDR1 (Kabat)
TYRMG

SEQ ID NO: 8 - protein, 5 aa, Artificial Sequence
  Description of Artificial Sequence Synthetic peptide
  VHH10 CDR1 (Kabat)
RYAMG

SEQ ID NO: 9 - protein, 7 aa, Artificial Sequence
  Description of Artificial Sequence Synthetic peptide
  VHH12 CDR1 (Kabat)
FNTYAMG

SEQ ID NO: 10 - protein, 7 aa, Artificial Sequence
  Description of Artificial Sequence Synthetic peptide
  VHH1 and VHH2 CDR1 (Chothia)
GLTFSSY

SEQ ID NO: 11 - protein, 7 aa, Artificial Sequence
  Description of Artificial Sequence Synthetic peptide
  VHH3 CDR1 (Chothia)
GSIFSIN

SEQ ID NO: 12 - protein, 7 aa, Artificial Sequence
  Description of Artificial Sequence Synthetic peptide
  VHH4 CDR1 (Chothia)
GTSVSSN

SEQ ID NO: 13 - protein, 7 aa, Artificial Sequence
  Description of Artificial Sequence Synthetic peptide
  VHH5 CDR1 (Chothia)
GRTFSSY

SEQ ID NO: 14 - protein, 7 aa, Artificial Sequence
  Description of Artificial Sequence Synthetic peptide
  VHH6 CDR1 (Chothia)
GSSVSSD

SEQ ID NO: 15 - protein, 7 aa, Artificial Sequence
  Description of Artificial Sequence Synthetic peptide
  VHH7 CDR1 (Chothia)
RSIGSIN

SEQ ID NO: 16 - protein, 7 aa, Artificial Sequence
  Description of Artificial Sequence Synthetic peptide
  VHH9 CDR1 (Chothia)
GRTFSTY

SEQ ID NO: 17 - protein, 7 aa, Artificial Sequence
  Description of Artificial Sequence Synthetic peptide
  VHH10 CDR1 (Chothia)
GFTFTRY

SEQ ID NO: 18 - protein, 7 aa, Artificial Sequence
  Description of Artificial Sequence Synthetic peptide
  VHH11 CDR1 (Chothia)
GRTFTTY

SEQ ID NO: 19 - protein, 9 aa, Artificial Sequence
  Description of Artificial Sequence Synthetic peptide
  VHH12 CDR1 (Chothia)
GRTLSFNTY

SEQ ID NO: 20 - protein, 8 aa, Artificial Sequence
  Description of Artificial Sequence Synthetic peptide
  VHH1 and VHH2 CDR1 (IMGT)
GLTFSSYR

SEQ ID NO: 21 - protein, 8 aa, Artificial Sequence
  Description of Artificial Sequence Synthetic peptide
  VHH3 CDR1 (IMGT)
GSIFSINV

SEQ ID NO: 22 - protein, 8 aa, Artificial Sequence
  Description of Artificial Sequence Synthetic peptide
  VHH4 CDR1 (IMGT)
GTSVSSNA

SEQ ID NO: 23 - protein, 8 aa, Artificial Sequence
  Description of Artificial Sequence Synthetic peptide
  VHH5 CDR1 (IMGT)
GRTFSSYA

SEQ ID NO: 24 - protein, 8 aa, Artificial Sequence
  Description of Artificial Sequence Synthetic peptide
  VHH6 CDR1 (IMGT)
GSSVSSDA

SEQ ID NO: 25 - protein, 8 aa, Artificial Sequence
  Description of Artificial Sequence Synthetic peptide
  VHH7 CDR1 (IMGT)
RSIGSINV

SEQ ID NO: 26 - protein, 8 aa, Artificial Sequence
  Description of Artificial Sequence Synthetic peptide
  VHH9 CDR1 (IMGT)
GRTFSTYR

SEQ ID NO: 27 - protein, 8 aa, Artificial Sequence
  Description of Artificial Sequence Synthetic peptide
  VHH10 CDR1 (IMGT)
GFTFTRYA

SEQ ID NO: 28 - protein, 8 aa, Artificial Sequence
  Description of Artificial Sequence Synthetic peptide
  VHH11 CDR1 (IMGT)
GRTFTTYR

SEQ ID NO: 29 - protein, 10 aa, Artificial Sequence
  Description of Artificial Sequence Synthetic peptide
  VHH12 CDR1 (IMGT)
GRTLSFNTYA

SEQ ID NO: 30 - protein, 20 aa, Artificial Sequence
  Description of Artificial Sequence Synthetic peptide
  VHH1 and VHH2 CDR2 (Kabat)
AIDWNGRGTYYRYYADSVKG

SEQ ID NO: 31 - protein, 16 aa, Artificial Sequence
  Description of Artificial Sequence Synthetic peptide
  VHH3 CDR2 (Kabat)
RINGGGITHYAESVKG

SEQ ID NO: 32 - protein, 16 aa, Artificial Sequence
  Description of Artificial Sequence Synthetic peptide
  VHH4 CDR2 (Kabat)
FIDRIATTTIATSVKG

SEQ ID NO: 33 - protein, 17 aa, Artificial Sequence
  Description of Artificial Sequence Synthetic peptide
  VHH5 CDR2 (Kabat)
AITWNGGTTYYADSVKG

SEQ ID NO: 34 - protein, 16 aa, Artificial Sequence
  Description of Artificial Sequence Synthetic peptide
  VHH6 CDR2 (Kabat)
FISGGGTTTYADSVKG

SEQ ID NO: 35 - protein, 16 aa, Artificial Sequence
  Description of Artificial Sequence Synthetic peptide
  VHH7 CDR2 (Kabat)
RITGGGSTHYAESVKG

SEQ ID NO: 36 - protein, 17 aa, Artificial Sequence
  Description of Artificial Sequence Synthetic peptide
  VHH9 CDR2 (Kabat)
AISWSGGSTTYADPVKG

SEQ ID NO: 37 - protein, 17 aa, Artificial Sequence
  Description of Artificial Sequence Synthetic peptide
  VHH10 CDR2 (Kabat)
AISWSGSSAGYGDSVKG

SEQ ID NO: 38 - protein, 17 aa, Artificial Sequence
  Description of Artificial Sequence Synthetic peptide
  VHH11 CDR2 (Kabat)
AIRWSGGRTLYADSVKG

SEQ ID NO: 39 - protein, 17 aa, Artificial Sequence
  Description of Artificial Sequence Synthetic peptide
  VHH12 CDR2 (Kabat)
SITWNGGSTSYADSVKG

SEQ ID NO: 40 - protein, 9 aa, Artificial Sequence
  Description of Artificial Sequence Synthetic peptide
  VHH1 and VHH2 CDR2 (Chothia)
DWNGRGTYY

SEQ ID NO: 41 - protein, 5 aa, Artificial Sequence
  Description of Artificial Sequence Synthetic peptide
  VHH3 CDR2 (Chothia)
NGGGI

SEQ ID NO: 42 - protein, 5 aa, Artificial Sequence
  Description of Artificial Sequence Synthetic peptide
  VHH4 CDR2 (Chothia)
DRIAT

SEQ ID NO: 43 - protein, 6 aa, Artificial Sequence
  Description of Artificial Sequence Synthetic peptide
  VHH5 CDR2 (Chothia)
TWNGGT

SEQ ID NO: 44 - protein, 5 aa, Artificial Sequence
  Description of Artificial Sequence Synthetic peptide
  VHH6 CDR2 (Chothia)
SGGGT

SEQ ID NO: 45 - protein, 5 aa, Artificial Sequence
  Description of Artificial Sequence Synthetic peptide
  VHH7 CDR2 (Chothia)
TGGGS

SEQ ID NO: 46 - protein, 6 aa, Artificial Sequence
  Description of Artificial Sequence Synthetic peptide
  VHH9 CDR2 (Chothia)
SWSGGS

SEQ ID NO: 47 - protein, 6 aa, Artificial Sequence
  Description of Artificial Sequence Synthetic peptide
  VHH10 CDR2 (Chothia)
SWSGSS

SEQ ID NO: 48 - protein, 6 aa, Artificial Sequence
  Description of Artificial Sequence Synthetic peptide
  VHH11 CDR2 (Chothia)
RWSGGR

SEQ ID NO: 49 - protein, 6 aa, Artificial Sequence
  Description of Artificial Sequence Synthetic peptide
  VHH12 CDR2 (Chothia)
TWNGGS

SEQ ID NO: 50 - protein, 10 aa, Artificial Sequence
  Description of Artificial Sequence Synthetic peptide
  VHH1 and VHH2 CDR2 (IMGT)
IDWNGRGTYY

SEQ ID NO: 51 - protein, 7 aa, Artificial Sequence
  Description of Artificial Sequence Synthetic peptide
  VHH3 CDR2 (IMGT)
INGGGIT

SEQ ID NO: 52 - protein, 7 aa, Artificial Sequence
  Description of Artificial Sequence Synthetic peptide
  VHH4 CDR2 (IMGT)
IDRIATT

SEQ ID NO: 53 - protein, 8 aa, Artificial Sequence
  Description of Artificial Sequence Synthetic peptide
  VHH5 CDR2 (IMGT)
ITWNGGTT

SEQ ID NO: 54 - protein, 7 aa, Artificial Sequence
  Description of Artificial Sequence Synthetic peptide
  VHH6 CDR2 (IMGT)
ISGGGTT

SEQ ID NO: 55 - protein, 7 aa, Artificial Sequence
  Description of Artificial Sequence Synthetic peptide
  VHH7 CDR2 (IMGT)
ITGGGST

SEQ ID NO: 56 - protein, 8 aa, Artificial Sequence
  Description of Artificial Sequence Synthetic peptide
  VHH9 CDR2 (IMGT)
ISWSGGST

SEQ ID NO: 57 - protein, 8 aa, Artificial Sequence
  Description of Artificial Sequence Synthetic peptide
  VHH10 CDR2 (IMGT)
ISWSGSSA

SEQ ID NO: 58 - protein, 8 aa, Artificial Sequence
  Description of Artificial Sequence Synthetic peptide
  VHH11 CDR2 (IMGT)
IRWSGGRT

SEQ ID NO: 59 - protein, 8 aa, Artificial Sequence
  Description of Artificial Sequence Synthetic peptide
  VHH12 CDR2 (IMGT)
ITWNGGST

SEQ ID NO: 60 - protein, 13 aa, Artificial Sequence
  Description of Artificial Sequence Synthetic peptide
  VHH1 CDR3 (Kabat)
GSIDLNWYGGMDY

SEQ ID NO: 61 - protein, 15 aa, Artificial Sequence
  Description of Artificial Sequence Synthetic peptide
  VHH2 CDR3 (Kabat)
TTVLTDPRVLNEYAT

SEQ ID NO: 62 - protein, 12 aa, Artificial Sequence
  Description of Artificial Sequence Synthetic peptide
  VHH3 CDR3 (Kabat)
DVFGSSGYVETY

SEQ ID NO: 63 - protein, 5 aa, Artificial Sequence
  Description of Artificial Sequence Synthetic peptide
  VHH4 CDR3 (Kabat)
PLTAR

SEQ ID NO: 64 - protein, 7 aa, Artificial Sequence
  Description of Artificial Sequence Synthetic peptide
  VHH5 CDR3 (Kabat)
DPFNQGY

SEQ ID NO: 65 - protein, 5 aa, Artificial Sequence
  Description of Artificial Sequence Synthetic peptide
  VHH6 CDR3 (Kabat)
PLTSR

SEQ ID NO: 66 - protein, 22 aa, Artificial Sequence
  Description of Artificial Sequence Synthetic peptide
  VHH7 CDR3 (Kabat)
MVNPIITAWGTIGVREIPDYDY

SEQ ID NO: 67 - protein, 5 aa, Artificial Sequence
  Description of Artificial Sequence Synthetic peptide
  VHH9 CDR3 (Kabat)
DQRGY

SEQ ID NO: 68 - protein, 7 aa, Artificial Sequence
  Description of Artificial Sequence Synthetic peptide
  VHH10 CDR3 (Kabat)
DPFNQGY

SEQ ID NO: 69 - protein, 21 aa, Artificial Sequence
  Description of Artificial Sequence Synthetic peptide
  VHH11 CDR3 (Kabat)
DLAEYSGTYSSPADSPAGYDY

SEQ ID NO: 70 - protein, 14 aa, Artificial Sequence
  Description of Artificial Sequence Synthetic peptide
  VHH12 CDR3 (Kabat)
ARYYVSGTYFPANY

SEQ ID NO: 71 - protein, 13 aa, Artificial Sequence
  Description of Artificial Sequence Synthetic peptide
  VHH1 CDR3 (Chothia)
GSIDLNWYGGMDY

SEQ ID NO: 72 - protein, 15 aa, Artificial Sequence
  Description of Artificial Sequence Synthetic peptide
  VHH2 CDR3 (Chothia)
TTVLTDPRVLNEYAT

SEQ ID NO: 73 - protein, 12 aa, Artificial Sequence
  Description of Artificial Sequence Synthetic peptide
  VHH3 CDR3 (Chothia)
DVFGSSGYVETY

SEQ ID NO: 74 - protein, 5 aa, Artificial Sequence
  Description of Artificial Sequence Synthetic peptide
  VHH4 CDR3 (Chothia)
PLTAR

SEQ ID NO: 75 - protein, 7 aa, Artificial Sequence
  Description of Artificial Sequence Synthetic peptide
  VHH5 CDR3 (Chothia)
DPFNQGY

SEQ ID NO: 76 - protein, 5 aa, Artificial Sequence
  Description of Artificial Sequence Synthetic peptide
  VHH6 CDR3 (Chothia)
PLTSR

SEQ ID NO: 77 - protein, 22 aa, Artificial Sequence
  Description of Artificial Sequence Synthetic peptide
  VHH7 CDR3 (Chothia)
MVNPIITAWGTIGVREIPDYDY

SEQ ID NO: 78 - protein, 5 aa, Artificial Sequence
  Description of Artificial Sequence Synthetic peptide
  VHH9 CDR3 (Chothia)
DQRGY

SEQ ID NO: 79 - protein, 7 aa, Artificial Sequence
  Description of Artificial Sequence Synthetic peptide
  VHH10 CDR3 (Chothia)
DPFNQGY

SEQ ID NO: 80 - protein, 21 aa, Artificial Sequence
  Description of Artificial Sequence Synthetic peptide
  VHH11 CDR3 (Chothia)
DLAEYSGTYSSPADSPAGYDY

SEQ ID NO: 81 - protein, 14 aa, Artificial Sequence
  Description of Artificial Sequence Synthetic peptide
  VHH12 CDR3 (Chothia)
ARYYVSGTYFPANY

SEQ ID NO: 82 - protein, 16 aa, Artificial Sequence
  Description of Artificial Sequence Synthetic peptide
  VHH1 CDR3 (IMGT)
CAAGSIDLNWYGGMDY

SEQ ID NO: 83 - protein, 18 aa, Artificial Sequence
  Description of Artificial Sequence Synthetic peptide
  VHH2 CDR3 (IMGT)
CAATTVLTDPRVLNEYAT

SEQ ID NO: 84 - protein, 14 aa, Artificial Sequence
  Description of Artificial Sequence Synthetic peptide
  VHH3 CDR3 (IMGT)
KADVFGSSGYVETY

SEQ ID NO: 85 - protein, 7 aa, Artificial Sequence
  Description of Artificial Sequence Synthetic peptide
  VHH4 CDR3 (IMGT)
NHPLTAR

SEQ ID NO: 86 - protein, 9 aa, Artificial Sequence
  Description of Artificial Sequence Synthetic peptide
  VHH5 CDR3 (IMGT)
AADPFNQGY

SEQ ID NO: 87 - protein, 7 aa, Artificial Sequence
  Description of Artificial Sequence Synthetic peptide
  VHH6 CDR3 (IMGT)
NHPLTSR

SEQ ID NO: 88 - protein, 24 aa, Artificial Sequence
  Description of Artificial Sequence Synthetic peptide
  VHH7 CDR3 (IMGT)
ASMVNPIITAWGTIGVREIPDYDY

SEQ ID NO: 89 - protein, 6 aa, Artificial Sequence
  Description of Artificial Sequence Synthetic peptide
  VHH9 CDR3 (IMGT)
NDQRGY

SEQ ID NO: 90 - protein, 9 aa, Artificial Sequence
  Description of Artificial Sequence Synthetic peptide
  VHH10 CDR3 (IMGT)
AADPFNQGY

SEQ ID NO: 91 - protein, 23 aa, Artificial Sequence
  Description of Artificial Sequence Synthetic peptide
  VHH11 CDR3 (IMGT)
AADLAEYSGTYSSPADSPAGYDY

SEQ ID NO: 92 - protein, 16 aa, Artificial Sequence
  Description of Artificial Sequence Synthetic peptide
  VHH12 CDR3 (IMGT)
AAARYYVSGTYFPANY

SEQ ID NO: 93 - protein, 125 aa, Artificial Sequence
  Description of Artificial Sequence Synthetic polypeptide
  VHH1 - VH amino acid sequence
QVQLVESGGGLVQAGGSLKLACAAPGLTFSSYRMGWFRQAPGQEREFVAAIDWNGRGTYYRYYADSVKGRSTISRDNAKNTMYLQMNSLKPEDTAVYYCAAGSIDLNWYGGMDYWGQGTQVTVSS

SEQ ID NO: 94 - protein, 127 aa, Artificial Sequence
  Description of Artificial Sequence Synthetic polypeptide
  VHH2 - VH amino acid sequence
EVQVVESGGGLVQAGGSLKLACAAPGLTFSSYRMGWFRQAPGQEREFVAAIDWNGRGTYYRYYADSVKGRSTISRDNAKNTVYLQMNSLKPEDTAVYYCAATTVLTDPRVLNEYATWGQGTQVTVSS

SEQ ID NO: 95 - protein, 120 aa, Artificial Sequence
  Description of Artificial Sequence Synthetic polypeptide
  VHH3 - VH amino acid sequence
QLQLVESGGGLVQPGGSLRLSCAASGSIFSINVMGWYRQAPGKQRELVARINGGGITHYAESVKGRFTISRDNAKNTVYLQMNSLKPEDTAAYYCKADVFGSSGYVETYWGQGTQVTVSS

SEQ ID NO: 96 - protein, 113 aa, Artificial Sequence
  Description of Artificial Sequence Synthetic polypeptide
  VHH4 - VH amino acid sequence
EVQVVESGGGLVQAGGSLRLSCAVSGTSVSSNAMGWYRQAPGKQREWVGFIDRIATTTIATSVKGRFAITRDNAKNTVYLQMSGLKPEDTAVYYCNHPLTARWGQGTQVTVSS

SEQ ID NO: 97 - protein, 116 aa, Artificial Sequence
  Description of Artificial Sequence Synthetic polypeptide
  VHH5 - VH amino acid sequence
QVQLVESGGGLVQAGGSLRLSCAASGRTFSSYAMGWFRQAPGKEREFVAAITWNGGTTYYADSVKGRFTISRDNAKNTVYLQMNSLKPEDTAVYYCAADPFNQGYWGQGTQVTVSS

SEQ ID NO: 98 - protein, 113 aa, Artificial Sequence
  Description of Artificial Sequence Synthetic polypeptide
  VHH6 - VH amino acid sequence
EVQLVESGGGLVQAGGSLRLSCAVSGSSVSSDAMGWYRQAPGNQRAWVAFISGGGTTTYADSVKGRFTISRDNTKNTVYLHMNSLKPEDTAVYYCNHPLTSRWGQGTQVTVSS

SEQ ID NO: 99 - protein, 130 aa, Artificial Sequence
  Description of Artificial Sequence Synthetic polypeptide
  VHH7 - VH amino acid sequence
EVQVVESGGGLVQAGGSLRLACVASRSIGSINVMGWYRQAPGKQRDLVARITGGGSTHYAESVKGRFTISRDNAKNTVYLQMNSLEPEDTAVYYCASMVNPIITAWGTIGVREIPDYDYWGQGTQVTVSS

SEQ ID NO: 100 - protein, 113 aa, Artificial Sequence
  Description of Artificial Sequence Synthetic polypeptide
  VHH9 - VH amino acid sequence
QVQLVESGGGLVQAGGSLRLSCAVSGRTFSTYRMGWFRQAPGKERSFVAAISWSGGSTTYADPVKGRFTISRDNAKNTVYLRMNSLKPEDTAVYYCNDQRGYWGQGTLVTVSS

SEQ ID NO: 101 - protein, 116 aa, Artificial Sequence
  Description of Artificial Sequence Synthetic polypeptide
  VHH10 - VH amino acid sequence
EVQVVESGGGLVQAGGSLRLSCAASGFTFTRYAMGWFRQAPGKERSFVAAISWSGSSAGYGDSVKGRFTISRDNAKNTLYLQMNSLKPEDTAVYYCAADPFNQGYWGQGTQVTVSS

SEQ ID NO: 102 - protein, 130 aa, Artificial Sequence
  Description of Artificial Sequence Synthetic polypeptide
  VHH11 - VH amino acid sequence
EVQVVESGGGLVQAGGSLRLSCAASGRTFTTYRMGWFRQAPGKEREFVAAIRWSGGRTLYADSVKGRFTISRDNAKNTAYLQMNNLRPEDTAVYYCAADLAEYSGTYSSPADSPAGYDYWGQGTQVTVSS

SEQ ID NO: 103 - protein, 125 aa, Artificial Sequence
  Description of Artificial Sequence Synthetic polypeptide
  VHH12 - VH amino acid sequence
QVQLVETGGGLVQAGDSLRLSCAASGRTLSFNTYAMGWFRQAPGKEREFVASITWNGGSTSYADSVKGRFTITRDNAKNTATLRMNSLQPDDTAVYYCAAARYYVSGTYFPANYWGQGTQVTVSS

SEQ ID NO: 104 - protein, 371 aa, Artificial Sequence
  Description of Artificial Sequence Synthetic polypeptide
  VHH1-linker- mono-Fc protein
QVQLVESGGGLVQAGGSLKLACAAPGLTFSSYRMGWFRQAPGQEREFVAAIDWNGRGTYYRYYADSVKGRSTISRDNAKNTMYLQMNSLKPEDTAVYYCAAGSIDLNWYGGMDYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 105 - protein, 373 aa, Artificial Sequence
  Description of Artificial Sequence Synthetic polypeptide
  VHH2-linker- mono-Fc protein
EVQVVESGGGLVQAGGSLKLACAAPGLTFSSYRMGWFRQAPGQEREFVAAIDWNGRGTYYRYYADSVKGRSTISRDNAKNTVYLQMNSLKPEDTAVYYCAATTVLTDPRVLNEYATWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 106 - protein, 366 aa, Artificial Sequence
  Description of Artificial Sequence Synthetic polypeptide
  VHH3-linker- mono-Fc protein
QLQLVESGGGLVQPGGSLRLSCAASGSIFSINVMGWYRQAPGKQRELVARINGGGITHYAESVKGRFTISRDNAKNTVYLQMNSLKPEDTAAYYCKADVFGSSGYVETYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 107 - protein, 359 aa, Artificial Sequence
  Description of Artificial Sequence Synthetic polypeptide
  VHH4-linker- mono-Fc protein
  MOD_RES (96)..(96): Any amino acid
  MOD_RES (99)..(99): Any amino acid
EVQVVESGGGLVQAGGSLRLSCAVSGTSVSSNAMGWYRQAPGKQREWVGFIDRIATTTIATSVKGRFAITRDNAKNTVYLQMSGLKPEDTAVYYCXHPXTARWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 108 - protein, 362 aa, Artificial Sequence
  Description of Artificial Sequence Synthetic polypeptide
  VHH5-linker- mono-Fc protein
QVQLVESGGGLVQAGGSLRLSCAASGRTFSSYAMGWFRQAPGKEREFVAAITWNGGTTYYADSVKGRFTISRDNAKNTVYLQMNSLKPEDTAVYYCAADPFNQGYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 109 - protein, 359 aa, Artificial Sequence
  Description of Artificial Sequence Synthetic polypeptide
  VHH6-linker- mono-Fc protein
EVQLVESGGGLVQAGGSLRLSCAVSGSSVSSDAMGWYRQAPGNQRAWVAFISGGGTTTYADSVKGRFTISRDNTKNTVYLHMNSLKPEDTAVYYCNHPLTSRWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 110 - protein, 376 aa, Artificial Sequence
  Description of Artificial Sequence Synthetic polypeptide
  VHH7-linker- mono-Fc protein
EVQVVESGGGLVQAGGSLRLACVASRSIGSINVMGWYRQAPGKQRDLVARITGGGSTHYAESVKGRFTISRDNAKNTVYLQMNSLEPEDTAVYYCASMVNPIITAWGTIGVREIPDYDYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 111 - protein, 359 aa, Artificial Sequence
  Description of Artificial Sequence Synthetic polypeptide
  VHH9-linker- mono-Fc protein
QVQLVESGGGLVQAGGSLRLSCAVSGRTFSTYRMGWFRQAPGKERSFVAAISWSGGSTTYADPVKGRFTISRDNAKNTVYLRMNSLKPEDTAVYYCNDQRGYWGQGTLVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 112 - protein, 362 aa, Artificial Sequence
  Description of Artificial Sequence Synthetic polypeptide
  VHH10-linker- mono-Fc protein
EVQVVESGGGLVQAGGSLRLSCAASGFTFTRYAMGWFRQAPGKERSFVAAISWSGSSAGYGDSVKGRFTISRDNAKNTLYLQMNSLKPEDTAVYYCAADPFNQGYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 113 - protein, 376 aa, Artificial Sequence
  Description of Artificial Sequence Synthetic polypeptide
  VHH11-linker- mono-Fc protein
EVQVVESGGGLVQAGGSLRLSCAASGRTFTTYRMGWFRQAPGKEREFVAAIRWSGGRTLYADSVKGRFTISRDNAKNTAYLQMNNLRPEDTAVYYCAADLAEYSGTYSSPADSPAGYDYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 114 - protein, 371 aa, Artificial Sequence
  Description of Artificial Sequence Synthetic polypeptide
  VHH12-linker- mono-Fc protein
QVQLVETGGGLVQAGDSLRLSCAASGRTLSFNTYAMGWFRQAPGKEREFVASITWNGGSTSYADSVKGRFTITRDNAKNTATLRMNSLQPDDTAVYYCAAARYYVSGTYFPANYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 115 - protein, 461 aa, Artificial Sequence
  Description of Artificial Sequence Synthetic polypeptide
  IgA Heavy Chain
QVQLVQSGAEVKKPGSSVKVSCKSSGGTSNNYAISWVRQAPGQGLDWMGGISPIFGSTAYAQKFQGRVTISADIFSNTAYMELNSLTSEDTAVYFCARHGNYYYYSGMDVWGQGTTVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 116 - protein, 216 aa, Artificial Sequence
  Description of Artificial Sequence Synthetic polypeptide
  IgA Light Chain
QSALTQPPAVSGTPGQRVTISCSGSDSNIGRRSVNWYQQFPGTAPKLLIYSNDQRPSVVPDRFSGSKSGTSASLAISGLQSEDEAEYYCAAWDDSLKGAVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 117 - protein, 153 aa, Artificial Sequence
  Description of Artificial Sequence Synthetic polypeptide
  IgA J Chain
SRDSSASASRVAGITAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 118 - protein, 626 aa, Homo sapiens
  human pIgR extracellular domain (ECD)
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRHHHHHH

SEQ ID NO: 119 - protein, 117 aa, Homo sapiens
  human pIgR extracellular domain 1 (D1)
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVGSHHHHHH

SEQ ID NO: 120 - protein, 123 aa, Homo sapiens
  human pIgR extracellular domain 2 (D2)
SQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPGSHHHHHH

SEQ ID NO: 121 - protein, 121 aa, Homo sapiens
  human pIgR extracellular domain 3 (D3)
KPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTGSHHHHHH

SEQ ID NO: 122 - protein, 114 aa, Homo sapiens
  human pIgR extracellular domain 5 (D5)
GEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 123 - protein, 230 aa, Homo sapiens
  human pIgR extracellular domain 1-domain 2 (D1-D2)
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPGSHHHHHH

SEQ ID NO: 124 - protein, 228 aa, Homo sapiens
  human pIgR extracellular domain 2-domain 3 (D2-D3)
SQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNGSHHHHHH

SEQ ID NO: 125 - protein, 224 aa, Homo sapiens
  human pIgR extracellular domain 4-domain 5 (D4-D5)
STIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 126 - protein, 7 aa, Oncorhynchus mykiss
  teleost fish pIgR CDR1 of D1
GPQYASY

SEQ ID NO: 127 - protein, 3 aa, Oncorhynchus mykiss
  teleost fish pIgR CDR2 of D1
DAP

SEQ ID NO: 128 - protein, 8 aa, Oncorhynchus mykiss
  teleost fish pIgR CDR3 of D1
VGGVWSAD

SEQ ID NO: 129 - protein, 633 aa, Mus sp.
  mouse pIgR extracellular domain (ECD)
KSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSDTHVYTKDIGRNVTIECPFKRENAPSKKSLCKKTNQSCELVIDSTEKVNPSYIGRAKLFMKGTDLTVFYVNISHLTHNDAGLYICQAGEGPSADKKNVDLQVLAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERTRGSSHVNPTDANARAKVALEEEVVDSSISEKENKAIPNPGPFANEREIQNVGDQAQENRASGDAGSADGQSRSSSSKHHHHHH

SEQ ID NO: 130 - protein, 27 aa, Artificial Sequence
  Description of Artificial Sequence Synthetic peptide
  hinge region (AA)
EPKTPKPQPQPQLQPQPNPTTESKSPK

SEQ ID NO: 131 - DNA, 82 bp, Artificial Sequence
  Description of Artificial Sequence Synthetic oligonucleotide
  hinge region (DNA)
gaacccaagacaccaaaaccacaaccacaaccacaactacaaccacaacccaatcctacaacagaatccaagagccccaaaa

SEQ ID NO: 132 - DNA, 657 bp, Homo sapiens
  Human IgG1 Mono-Fc DNA sequence
agcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccaagcccccatcccgggaggagatgaccaagaaccaggtcagcctgagctgcctggtcaaaggcttctatcccagcgacatcgccgtggagtgggagagcaatgggcagccggagaacaactacaagaccacggtgcccgtgctggactccgacggctccttcagactcgcaagctatctcaccgtggacaagagcagatggcagcaggggaacgtcttctcatgctccgtgatgcatgaggctctgcacaaccactacacgcagaagagcctctccctgtctccgggtaaa

SEQ ID NO: 133 - DNA, 375 bp, Artificial Sequence
  Description of Artificial Sequence Synthetic polynucleotide
  DNA sequence coding for VHH1
  modified_base (349)..(349): a, c, t, g, unknown or other
  modified_base (351)..(351): a, c, t, g, unknown or other
caggtgcagctggtggagtctgggggaggattggtgcaggctgggggctctctgaaactcgcctgtgcagcacctggacttaccttcagttcgtatcgcatgggctggttccgccaggctccagggcaggagcgtgagtttgtagcagctattgattggaatggtcgtggcacatattatcgatactatgcagactccgtgaagggccgatccaccatttccagagacaacgccaagaacacgatgtatctgcaaatgaacagcctgaaacctgaggacacggccgtttattactgtgcagcaggttcgatcgaccttaactggtacggcggcatggactactggggcnangggacccaggtcaccgtctcctca

SEQ ID NO: 134 - DNA, 381 bp, Artificial Sequence
  Description of Artificial Sequence Synthetic polynucleotide
  DNA sequence coding for VHH2
gaggtgcaggtggtggagtctgggggaggattggtgcaggctgggggctctctgaaactcgcctgtgcagcacctggacttaccttcagttcgtatcgcatgggctggttccgccaggctccagggcaggagcgtgagtttgtagcagctattgattggaatggtcgtggcacatattatcgatactatgcagactccgtgaagggccgatccaccatttccagagacaacgccaagaacacggtgtatctgcaaatgaacagcctgaaacctgaggacacggccgtttattactgtgcagctactacggtattaactgaccctagggttcttaatgagtatgccacatggggccaggggacccaggtcaccgtctcctca

SEQ ID NO: 135 - DNA, 360 bp, Artificial Sequence
  Description of Artificial Sequence Synthetic polynucleotide
  DNA sequence coding for VHH3
cagttgcagctcgtggagtctgggggaggcttggtgcagcctggggggtctctgagactctcctgtgcagcctctggaagcatcttcagtatcaatgttatgggctggtaccgccaggctccagggaagcagcgcgagttggtcgcacgtattaatggaggtggcattacacactatgcagagtccgtgaagggccgattcaccatctccagagacaacgccaagaacacggtgtatctgcaaatgaacagcctgaaacctgaggacacagccgcatattactgtaaggcagatgtgttcggtagtagcgggtacgtagaaacctactggggccaggggacccaggtcaccgtctcctca

SEQ ID NO: 136 - DNA, 339 bp, Artificial Sequence
  Description of Artificial Sequence Synthetic polynucleotide
  DNA sequence coding for VHH4
gaggtgcaggtggtggagtctgggggaggcttggtgcaggctgggggctctctgagactctcctgtgcagtctctggaacctccgtcagtagcaatgccatgggttggtaccgccaggctccagggaagcagcgcgagtgggtcggatttattgatcgtattgctaccacgacgattgcaacctccgtgaagggccgattcgccatcaccagagacaacgccaagaacacggtgtatctccaaatgagcggcctgaaacctgaggacacagccgtctattactgtaatcatccattgaccgctcggtggggccaggggacccaggtcaccgtctcctca

SEQ ID NO: 137 - DNA, 348 bp, Artificial Sequence
  Description of Artificial Sequence Synthetic polynucleotide
  DNA sequence coding for VHH5
caggtgcagctggtggagtctgggggaggcttggtgcaggctgggggctctctgagactctcctgtgcagcctctggacgcaccttcagtagctatgccatgggctggttccgccaggctccagggaaggagcgtgagtttgtagcagctattacctggaatggtggtaccacatactatgcagactccgtgaagggccgattcaccatctccagagacaacgccaagaacacggtgtatctgcaaatgaacagcctgaaacctgaggacacggccgtttattactgtgcagcagacccattcaaccaaggctactggggccaggggacccaggtcaccgtctcctca

SEQ ID NO: 138 - DNA, 339 bp, Artificial Sequence
  Description of Artificial Sequence Synthetic polynucleotide
  DNA sequence coding for VHH6
gaggtgcagctcgtggagtctggaggaggcttggtgcaggctggggggtctctgagactctcctgtgcagtctctggaagctccgtcagtagcgatgccatgggttggtaccgccaggctccagggaatcagcgcgcgtgggtcgcatttatttctggtggtggtaccacaacctatgcagactccgttaagggccgattcaccatctccagagacaacaccaagaacacggtgtatctccacatgaacagcctgaaacctgaagacacagccgtctattactgtaatcatccattgacgtctcggtggggccaggggacccaggtcaccgtctcctca

SEQ ID NO: 139 - DNA, 390 bp, Artificial Sequence
  Description of Artificial Sequence Synthetic polynucleotide
  DNA sequence coding for VHH7
gaggtgcaggtggtggagtctgggggaggattggtgcaggctggggggtctctgagactcgcctgtgtagcctctagaagcatcggcagtatcaatgttatgggctggtaccgccaggctccagggaagcagcgcgacttggtcgcacgtattactggaggtggcagtacacactacgcagagtccgtgaagggccgattcaccatctccagagacaacgccaagaacacggtgtatctgcaaatgaacagcctggaacctgaggacacggccgtttattactgtgcgtcaatggtaaaccctatcattacggcttggggtacgattggtgtgcgcgagattcccgactatgactactggggccaggggacccaggtcaccgtctcctca

SEQ ID NO: 140 - DNA, 348 bp, Artificial Sequence
  Description of Artificial Sequence Synthetic polynucleotide
  DNA sequence coding for VHH10
gaggtgcaggtggtggagtctgggggaggcttggtgcaggctggggggtctctgagactctcctgtgcagcctctggattcaccttcacccgctatgccatgggctggttccgccaggctccagggaaggagcgatcgtttgtagcagctattagctggagtggtagtagcgcaggctatggagactccgtgaagggccgattcaccatctccagagacaacgccaagaacacgctgtatctgcaaatgaacagtctaaaacctgaggacacggccgtttattactgtgcagcagacccattcaaccaaggctactggggccaggggacccaggtcaccgtctcctca

SEQ ID NO: 141 - DNA, 390 bp, Artificial Sequence
  Description of Artificial Sequence Synthetic polynucleotide
  DNA sequence coding for VHH11
gaggtgcaggtggtggagtctgggggaggattggtgcaggctgggggctctctgagactctcctgtgcagcctctggacgcaccttcactacctatcgcatgggctggttccgccaggctccagggaaggagcgagagtttgtagcagctattcgctggagtggtggtcgcacattgtatgcagactccgtgaagggccgattcaccatctccagagacaacgccaagaacacagcgtatctgcaaatgaacaacctgagacctgaggacacggccgtttattactgtgcagcagatctagccgagtatagtggtacttactccagccctgcggactcccccgctgggtatgactactggggccaggggacccaggtcaccgtctcctca

SEQ ID NO: 142 - DNA, 375 bp, Artificial Sequence
  Description of Artificial Sequence Synthetic polynucleotide
  DNA sequence coding for VHH12
caggtgcagctggtcgaaactgggggaggattggtgcaggctggggactctctgagactctcctgtgcagcctctggacgcaccctcagcttcaacacctatgccatgggctggttccgccaggctccagggaaggagcgtgaatttgtagcctctattacctggaatggtggaagcacaagctacgcagactccgtgaagggccgattcaccatcaccagagacaacgccaagaacacggctactctgcgaatgaatagcctgcagcccgacgacacggccgtgtattactgtgcagcagcccgatactatgtgagtggtacttacttccccgcgaattactggggccaggggacccaggtcaccgtctcctca

SEQ ID NO: 143 - protein, 31 aa, Homo sapiens
  Exemplary stalk sequence of human pIgR
EKAVADTRDQADGSRASVDSGSSEEQGGSSR

SEQ ID NO: 144 - protein, 33 aa, Mus sp.
  Exemplary stalk sequence of mouse pIgR
EREIQNVGDQAQENRASGDAGSADGQSRSSSSK

SEQ ID NO: 145 - protein, 33 aa, Mus sp.
  Exemplary stalk sequence of mouse pIgR
EREIQNVRDQAQENRASGDAGSADGQSRSSSSK

SEQ ID NO: 146 - protein, 219 aa, Homo sapiens
  Human IgG1 Mono-Fc AA sequence
SPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 147 - protein, 100 aa, Artificial Sequence
  Description of Artificial Sequence Synthetic polypeptide
  Exemplary flexible linker 1
  MISC_FEATURE (1)..(100): This sequence may encompass 1-20 “Glu Ala Ala Ala Lys” repeating units
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 148 - protein, 100 aa, Artificial Sequence
  Description of Artificial Sequence Synthetic polypeptide
  Exemplary flexible linker 2
  MISC_FEATURE (1)..(100): This sequence may encompass 1-20 “Gly Gly Gly Gly Ser” repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 149 - protein, 80 aa, Artificial Sequence
  Description of Artificial Sequence Synthetic polypeptide
  Exemplary flexible linker 3
  MISC_FEATURE (1)..(80): This sequence may encompass 1-20 “Gly Gly Gly Ser” repeating units
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 150 - protein, 15 aa, Artificial Sequence
  Description of Artificial Sequence Synthetic peptide
  Exemplary hinge region 1
EPKSCDKTHTCPPCP

SEQ ID NO: 151 - protein, 12 aa, Artificial Sequence
  Description of Artificial Sequence Synthetic peptide
  Exemplary hinge region 2
ERKCCVECPPCP

SEQ ID NO: 152 - protein, 62 aa, Artificial Sequence
  Description of Artificial Sequence Synthetic polypeptide
  Exemplary hinge region 3
ELKTPLGDTTHTCPRCPEPKSCDTPPPCPRCPEPKSCDTPPPCPRCPEPKSCDTPPPCPRCP

SEQ ID NO: 153 - protein, 12 aa, Artificial Sequence
  Description of Artificial Sequence Synthetic peptide
  Exemplary hinge region 4
ESKYGPPCPSCP

SEQ ID NO: 154 - protein, 10 aa, Artificial Sequence
  Description of Artificial Sequence Synthetic peptide
  VHH1 and VHH 2 CDR1 (Exemplary)
GLTFSSYRMG

SEQ ID NO: 155 - protein, 10 aa, Artificial Sequence
  Description of Artificial Sequence Synthetic peptide
  VHH3 CDR1 (Exemplary)
GSIFSINVMG

SEQ ID NO: 156 - protein, 10 aa, Artificial Sequence
  Description of Artificial Sequence Synthetic peptide
  VHH4 CDR1 (Exemplary)
GTSVSSNAMG

SEQ ID NO: 157 - protein, 10 aa, Artificial Sequence
  Description of Artificial Sequence Synthetic peptide
  VHH5 CDR1 (Exemplary)
GRTFSSYAMG

SEQ ID NO: 158 - protein, 10 aa, Artificial Sequence
  Description of Artificial Sequence Synthetic peptide
  VHH6 CDR1 (Exemplary)
GSSVSSDAMG

SEQ ID NO: 159 - protein, 10 aa, Artificial Sequence
  Description of Artificial Sequence Synthetic peptide
  VHH7 CDR1 (Exemplary)
RSIGSINVMG

SEQ ID NO: 160 - protein, 10 aa, Artificial Sequence
  Description of Artificial Sequence Synthetic peptide
  VHH9 CDR1 (Exemplary)
GRTFSTYRMG

SEQ ID NO: 161 - protein, 10 aa, Artificial Sequence
  Description of Artificial Sequence Synthetic peptide
  VHH10 CDR1 (Exemplary)
GFTFTRYAMG

SEQ ID NO: 162 - protein, 10 aa, Artificial Sequence
  Description of Artificial Sequence Synthetic peptide
  VHH11 CDR1 (Exemplary)
GRTFTTYRMG

SEQ ID NO: 163 - protein, 12 aa, Artificial Sequence
  Description of Artificial Sequence Synthetic peptide
  VHH12 CDR1 (Exemplary)
GRTLSFNTYAMG

SEQ ID NO: 164 - protein, 6 aa, Artificial Sequence
  Description of Artificial Sequence Synthetic peptide
  VHH1 and VHH 2 CDR1 (Contact)
SSYRMG

SEQ ID NO: 165 - protein, 6 aa, Artificial Sequence
  Description of Artificial Sequence Synthetic peptide
  VHH3 CDR1 (Contact)
SINVMG

SEQ ID NO: 166 - protein, 6 aa, Artificial Sequence
  Description of Artificial Sequence Synthetic peptide
  VHH4 CDR1 (Contact)
SSNAMG

SEQ ID NO: 167 - protein, 6 aa, Artificial Sequence
  Description of Artificial Sequence Synthetic peptide
  VHH5 CDR1 (Contact)
SSYAMG

SEQ ID NO: 168 - protein, 6 aa, Artificial Sequence
  Description of Artificial Sequence Synthetic peptide
  VHH6 CDR1 (Contact)
SSDAMG

SEQ ID NO: 169 - protein, 6 aa, Artificial Sequence
  Description of Artificial Sequence Synthetic peptide
  VHH7 CDR1 (Contact)
SINVMG

SEQ ID NO: 170 - protein, 6 aa, Artificial Sequence
  Description of Artificial Sequence Synthetic peptide
  VHH9 CDR1 (Contact)
STYRMG

SEQ ID NO: 171 - protein, 6 aa, Artificial Sequence
  Description of Artificial Sequence Synthetic peptide
  VHH10 CDR1 (Contact)
TRYAMG

SEQ ID NO: 172 - protein, 6 aa, Artificial Sequence
  Description of Artificial Sequence Synthetic peptide
  VHH11 CDR1 (Contact)
TTYRMG

SEQ ID NO: 173 - protein, 8 aa, Artificial Sequence
  Description of Artificial Sequence Synthetic peptide
  VHH12 CDR1 (Contact)
SFNTYAMG

SEQ ID NO: 174 - protein, 10 aa, Artificial Sequence
  Description of Artificial Sequence Synthetic peptide
  VHH1 and VHH 2 CDR1 (AbM)
GLTFSSYRMG

SEQ ID NO: 175 - protein, 10 aa, Artificial Sequence
  Description of Artificial Sequence Synthetic peptide
  VHH3 CDR1 (AbM)
GSIFSINVMG

SEQ ID NO: 176 - protein, 10 aa, Artificial Sequence
  Description of Artificial Sequence Synthetic peptide
  VHH4 CDR1 (AbM)
GTSVSSNAMG

SEQ ID NO: 177 - protein, 10 aa, Artificial Sequence
  Description of Artificial Sequence Synthetic peptide
  VHH5 CDR1 (AbM)
GRTFSSYAMG

SEQ ID NO: 178 - protein, 10 aa, Artificial Sequence
  Description of Artificial Sequence Synthetic peptide
  VHH6 CDR1 (AbM)
GSSVSSDAMG

SEQ ID NO: 179 - protein, 10 aa, Artificial Sequence
  Description of Artificial Sequence Synthetic peptide
  VHH7 CDR1 (AbM)
RSIGSINVMG

SEQ ID NO: 180 - protein, 10 aa, Artificial Sequence
  Description of Artificial Sequence Synthetic peptide
  VHH9 CDR1 (AbM)
GRTFSTYRMG

SEQ ID NO: 181 - protein, 10 aa, Artificial Sequence
  Description of Artificial Sequence Synthetic peptide
  VHH10 CDR1 (AbM)
GFTFTRYAMG

SEQ ID NO: 182 - protein, 10 aa, Artificial Sequence
  Description of Artificial Sequence Synthetic peptide
  VHH11 CDR1 (AbM)
GRTFTTYRMG

SEQ ID NO: 183 - protein, 12 aa, Artificial Sequence
  Description of Artificial Sequence Synthetic peptide
  VHH12 CDR1 (AbM)
GRTLSFNTYAMG

SEQ ID NO: 184 - protein, 20 aa, Artificial Sequence
  Description of Artificial Sequence Synthetic peptide
  VHH1 and VHH 2 CDR2 (Exemplary)
AIDWNGRGTYYRYYADSVKG

SEQ ID NO: 185 - protein, 16 aa, Artificial Sequence
  Description of Artificial Sequence Synthetic peptide
  VHH3 CDR2 (Exemplary)
RINGGGITHYAESVKG

SEQ ID NO: 186 - protein, 16 aa, Artificial Sequence
  Description of Artificial Sequence Synthetic peptide
  VHH4 CDR2 (Exemplary)
FIDRIATTTIATSVKG

SEQ ID NO: 187 - protein, 17 aa, Artificial Sequence
  Description of Artificial Sequence Synthetic peptide
  VHH5 CDR2 (Exemplary)
AITWNGGTTYYADSVKG

SEQ ID NO: 188 - protein, 16 aa, Artificial Sequence
  Description of Artificial Sequence Synthetic peptide
  VHH6 CDR2 (Exemplary)
FISGGGTTTYADSVKG

SEQ ID NO: 189 - protein, 16 aa, Artificial Sequence
  Description of Artificial Sequence Synthetic peptide
  VHH7 CDR2 (Exemplary)
RITGGGSTHYAESVKG

SEQ ID NO: 190 - protein, 17 aa, Artificial Sequence
  Description of Artificial Sequence Synthetic peptide
  VHH9 CDR2 (Exemplary)
AISWSGGSTTYADPVKG

SEQ ID NO: 191 - protein, 17 aa, Artificial Sequence
  Description of Artificial Sequence Synthetic peptide
  VHH10 CDR2 (Exemplary)
AISWSGSSAGYGDSVKG

SEQ ID NO: 192 - protein, 17 aa, Artificial Sequence
  Description of Artificial Sequence Synthetic peptide
  VHH11 CDR2 (Exemplary)
AIRWSGGRTLYADSVKG

SEQ ID NO: 193 - protein, 17 aa, Artificial Sequence
  Description of Artificial Sequence Synthetic peptide
  VHH12 CDR2 (Exemplary)
SITWNGGSTSYADSVKG

SEQ ID NO: 194 - protein, 16 aa, Artificial Sequence
  Description of Artificial Sequence Synthetic peptide
  VHH1 and VHH2 CDR2 (Contact)
FVAAIDWNGRGTYYRY

SEQ ID NO: 195 - protein, 12 aa, Artificial Sequence
  Description of Artificial Sequence Synthetic peptide
  VHH3 CDR2 (Contact)
LVARINGGGITH

SEQ ID NO: 196 - protein, 12 aa, Artificial Sequence
  Description of Artificial Sequence Synthetic peptide
  VHH4 CDR2 (Contact)
WVGFIDRIATTT

SEQ ID NO: 197 - protein, 13 aa, Artificial Sequence
  Description of Artificial Sequence Synthetic peptide
  VHH5 CDR2 (Contact)
FVAAITWNGGTTY

SEQ ID NO: 198 - protein, 12 aa, Artificial Sequence
  Description of Artificial Sequence Synthetic peptide
  VHH6 CDR2 (Contact)
WVAFISGGGTTT

SEQ ID NO: 199 - protein, 12 aa, Artificial Sequence
  Description of Artificial Sequence Synthetic peptide
  VHH7 CDR2 (Contact)
LVARITGGGSTH

SEQ ID NO: 200 - protein, 13 aa, Artificial Sequence
  Description of Artificial Sequence Synthetic peptide
  VHH9 CDR2 (Contact)
FVAAISWSGGSTT

SEQ ID NO: 201 - protein, 13 aa, Artificial Sequence
  Description of Artificial Sequence Synthetic peptide
  VHH10 CDR2 (Contact)
FVAAISWSGSSAG

SEQ ID NO: 202 - protein, 13 aa, Artificial Sequence
  Description of Artificial Sequence Synthetic peptide
  VHH11 CDR2 (Contact)
FVAAIRWSGGRTL

SEQ ID NO: 203 - protein, 13 aa, Artificial Sequence
  Description of Artificial Sequence Synthetic peptide
  VHH12 CDR2 (Contact)
FVASITWNGGSTS

SEQ ID NO: 204 - protein, 13 aa, Artificial Sequence
  Description of Artificial Sequence Synthetic peptide
  VHH1 and VHH 2CDR2 (AbM)
AIDWNGRGTYYRY

SEQ ID NO: 205 - protein, 9 aa, Artificial Sequence
  Description of Artificial Sequence Synthetic peptide
  VHH3 CDR2 (AbM)
RINGGGITH

SEQ ID NO: 206 - protein, 9 aa, Artificial Sequence
  Description of Artificial Sequence Synthetic peptide
  VHH4 CDR2 (AbM)
FIDRIATTT

SEQ ID NO: 207 - protein, 10 aa, Artificial Sequence
  Description of Artificial Sequence Synthetic peptide
  VHH5 CDR2 (AbM)
AITWNGGTTY

SEQ ID NO: 208 - protein, 9 aa, Artificial Sequence
  Description of Artificial Sequence Synthetic peptide
  VHH6 CDR2 (AbM)
FISGGGTTT

SEQ ID NO: 209 - protein, 9 aa, Artificial Sequence
  Description of Artificial Sequence Synthetic peptide
  VHH7 CDR2 (AbM)
RITGGGSTH

SEQ ID NO: 210 - protein, 10 aa, Artificial Sequence
  Description of Artificial Sequence Synthetic peptide
  VHH9 CDR2 (AbM)
AISWSGGSTT

SEQ ID NO: 211 - protein, 10 aa, Artificial Sequence
  Description of Artificial Sequence Synthetic peptide
  VHH10 CDR2 (AbM)
AISWSGSSAG

SEQ ID NO: 212 - protein, 10 aa, Artificial Sequence
  Description of Artificial Sequence Synthetic peptide
  VHH11 CDR2 (AbM)
AIRWSGGRTL

SEQ ID NO: 213 - protein, 10 aa, Artificial Sequence
  Description of Artificial Sequence Synthetic peptide
  VHH12 CDR2 (AbM)
SITWNGGSTS

SEQ ID NO: 214 - protein, 13 aa, Artificial Sequence
  Description of Artificial Sequence Synthetic peptide
  VHH1 CDR3 (Exemplary)
GSIDLNWYGGMDY

SEQ ID NO: 215 - protein, 15 aa, Artificial Sequence
  Description of Artificial Sequence Synthetic peptide
  VHH2 CDR3 (Exemplary)
TTVLTDPRVLNEYAT

SEQ ID NO: 216 - protein, 12 aa, Artificial Sequence
  Description of Artificial Sequence Synthetic peptide
  VHH3 CDR3 (Exemplary)
DVFGSSGYVETY

SEQ ID NO: 217 - protein, 5 aa, Artificial Sequence
  Description of Artificial Sequence Synthetic peptide
  VHH4 CDR3 (Exemplary)
PLTAR

SEQ ID NO: 218 - protein, 7 aa, Artificial Sequence
  Description of Artificial Sequence Synthetic peptide
  VHH5 CDR3 (Exemplary)
DPFNQGY

SEQ ID NO: 219 - protein, 5 aa, Artificial Sequence
  Description of Artificial Sequence Synthetic peptide
  VHH6 CDR3 (Exemplary)
PLTSR

SEQ ID NO: 220 - protein, 22 aa, Artificial Sequence
  Description of Artificial Sequence Synthetic peptide
  VHH7 CDR3 (Exemplary)
MVNPIITAWGTIGVREIPDYDY

SEQ ID NO: 221 - protein, 4 aa, Artificial Sequence
  Description of Artificial Sequence Synthetic peptide
  VHH9 CDR3 (Exemplary)
QRGY

SEQ ID NO: 222 - protein, 7 aa, Artificial Sequence
  Description of Artificial Sequence Synthetic peptide
  VHH10 CDR3 (Exemplary)
DPFNQGY

SEQ ID NO: 223 - protein, 21 aa, Artificial Sequence
  Description of Artificial Sequence Synthetic peptide
  VHH11 CDR3 (Exemplary)
DLAEYSGTYSSPADSPAGYDY

SEQ ID NO: 224 - protein, 14 aa, Artificial Sequence
  Description of Artificial Sequence Synthetic peptide
  VHH12 CDR3 (Exemplary)
ARYYVSGTYFPANY

SEQ ID NO: 225 - protein, 14 aa, Artificial Sequence
  Description of Artificial Sequence Synthetic peptide
  VHH1 CDR3 (Contact)
AAGSIDLNWYGGMD

SEQ ID NO: 226 - protein, 16 aa, Artificial Sequence
  Description of Artificial Sequence Synthetic peptide
  VHH2 CDR3 (Contact)
AATTVLTDPRVLNEYA

SEQ ID NO: 227 - protein, 13 aa, Artificial Sequence
  Description of Artificial Sequence Synthetic peptide
  VHH3 CDR3 (Contact)
KADVFGSSGYVET

SEQ ID NO: 228 - protein, 6 aa, Artificial Sequence
  Description of Artificial Sequence Synthetic peptide
  VHH4 CDR3 (Contact)
NHPLTA

SEQ ID NO: 229 - protein, 8 aa, Artificial Sequence
  Description of Artificial Sequence Synthetic peptide
  VHH5 CDR3 (Contact)
AADPFNQG

SEQ ID NO: 230 - protein, 6 aa, Artificial Sequence
  Description of Artificial Sequence Synthetic peptide
  VHH6 CDR3 (Contact)
NHPLTS

SEQ ID NO: 231 - protein, 23 aa, Artificial Sequence
  Description of Artificial Sequence Synthetic peptide
  VHH7 CDR3 (Contact)
ASMVNPIITAWGTIGVREIPDYD

SEQ ID NO: 232 - protein, 5 aa, Artificial Sequence
  Description of Artificial Sequence Synthetic peptide
  VHH9 CDR3 (Contact)
NDQRG

SEQ ID NO: 233 - protein, 8 aa, Artificial Sequence
  Description of Artificial Sequence Synthetic peptide
  VHH10 CDR3 (Contact)
AADPFNQG

SEQ ID NO: 234 - protein, 22 aa, Artificial Sequence
  Description of Artificial Sequence Synthetic peptide
  VHH11 CDR3 (Contact)
AADLAEYSGTYSSPADSPAGYD

SEQ ID NO: 235 - protein, 15 aa, Artificial Sequence
  Description of Artificial Sequence Synthetic peptide
  VHH12 CDR3 (Contact)
AAARYYVSGTYFPAN

SEQ ID NO: 236 - protein, 13 aa, Artificial Sequence
  Description of Artificial Sequence Synthetic peptide
  VHH1 CDR3 (AbM)
GSIDLNWYGGMDY

SEQ ID NO: 237 - protein, 15 aa, Artificial Sequence
  Description of Artificial Sequence Synthetic peptide
  VHH2 CDR3 (AbM)
TTVLTDPRVLNEYAT

SEQ ID NO: 238 - protein, 12 aa, Artificial Sequence
  Description of Artificial Sequence Synthetic peptide
  VHH3 CDR3 (AbM)
DVFGSSGYVETY

SEQ ID NO: 239 - protein, 5 aa, Artificial Sequence
  Description of Artificial Sequence Synthetic peptide
  VHH4 CDR3 (AbM)
PLTAR

SEQ ID NO: 240 - protein, 7 aa, Artificial Sequence
  Description of Artificial Sequence Synthetic peptide
  VHH5 CDR3 (AbM)
DPFNQGY

SEQ ID NO: 241 - protein, 5 aa, Artificial Sequence
  Description of Artificial Sequence Synthetic peptide
  VHH6 CDR3 (AbM)
PLTSR

SEQ ID NO: 242 - protein, 22 aa, Artificial Sequence
  Description of Artificial Sequence Synthetic peptide
  VHH7 CDR3 (AbM)
MVNPIITAWGTIGVREIPDYDY

SEQ ID NO: 243 - protein, 4 aa, Artificial Sequence
  Description of Artificial Sequence Synthetic peptide
  VHH9 CDR3 (AbM)
QRGY

SEQ ID NO: 244 - protein, 7 aa, Artificial Sequence
  Description of Artificial Sequence Synthetic peptide
  VHH10 CDR3 (AbM)
DPFNQGY

SEQ ID NO: 245 - protein, 21 aa, Artificial Sequence
  Description of Artificial Sequence Synthetic peptide
  VHH11 CDR3 (AbM)
DLAEYSGTYSSPADSPAGYDY

SEQ ID NO: 246 - protein, 14 aa, Artificial Sequence
  Description of Artificial Sequence Synthetic peptide
  VHH12 CDR3 (AbM)
ARYYVSGTYFPANY

SEQ ID NO: 247 - protein, 113 aa, Homo sapiens
  hpIgR_073
  MOD_RES (96)..(96): Any amino acid
  MOD_RES (99)..(99): Any amino acid
EVQVVESGGGLVQAGGSLRLSCAVSGTSVSSNAMGWYRQAPGKQREWVGFIDRIATTTIATSVKGRFAITRDNAKNTVYLQMSGLKPEDTAVYYCXHPXTARWGQGTQVTVSS

SEQ ID NO: 248 - protein, 130 aa, Homo sapiens
  hpIgR_201
QVQLVESGGGLVQPGGSLRLSCAASGRPNSKYAMAWFRRAPGKEREFQAAINWSGANTYYGDSVKGRFTISRDNAKNTVTLQMNNLNPEDTAVYYCAADNRAYTYHTFDYYQTDASYVYWGQGTQVTVSS

SEQ ID NO: 249 - protein, 127 aa, Mus sp.
  mpIgR_349
QLQLVESGGGLVQAGGSLRLSCAASGRTFSTSTMGWFRQAPGKEREFVAAIQWSSASASTYYYYADSVKGRFTISRDNARNTVSLQMNSLKPEDTAVYYCANLVFRVGALKERDDYWGQGTQVTVSS

SEQ ID NO: 250 - protein, 110 aa, Homo sapiens
  hpIgR_D1
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVS

SEQ ID NO: 251 - protein, 110 aa, Mus sp.
  mpIgR_D1
KSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVS

SEQ ID NO: 252 - protein, 110 aa, Artificial Sequence
  Description of Artificial Sequence Synthetic polypeptide
  Consensus
  MOD_RES (8)..(8): Glu or Gln
  MOD_RES (11)..(11): Asn or Ser
  MOD_RES (16)..(16): Asn or Asp
  MOD_RES (26)..(26): Pro or Asp
  MOD_RES (43)..(43): Arg or Ser
  MOD_RES (45)..(45): Gly or Met
  MOD_RES (47)..(47): Ile or Thr
  MOD_RES (53)..(53): Glu or Asn
  MOD_RES (58)..(58): Ser or Lys
  MOD_RES (59)..(59): Lys or Glu
  MOD_RES (67)..(67): Thr or Ile
  MOD_RES (73)..(73): Gly or Asn
  MOD_RES (80)..(80): Ala or Glu
  MOD_RES (89)..(89): Arg or Ser
  MOD_RES (96)..(96): Ile or Thr
  MOD_RES (97)..(97): Asn or Ser
  MOD_RES (98)..(98): Ser or Asn
KSPIFGPXEVXSIEGXSVSITCYYPXTSVNRHTRKYWCRQGAXGXCXTLISSXGYLSXXYAGRANLXNFPENXTFVINIXQLSQDDSGXYKCGLGXXXRGLSFDVSLEVS

SEQ ID NO: 253 - protein, 113 aa, Oncorhynchus mykiss
  tpIgR_D1
RVTTVGDLAVLEGRSVMIPCHYGPQYASYVKYWCRGSVKDLCTSLVRSDAPRGPAAAGEDKVVMFDDPVQQVFTVTMTELQKEDSGWYWCGVEVGGVWSADVTASLHINVIQG

SEQ ID NO: 254 - protein, 109 aa, Homo sapiens
  hpIgR_D1
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEV

SEQ ID NO: 255 - protein, 107 aa, Artificial Sequence
  Description of Artificial Sequence Synthetic polypeptide
  hD1_tCDR1
KSPIFGPEEVNSVEGNSVSITCYYGPQYASYRKYWCRQGARGGCITLISSEGYVSSKYAGRANLTNFPENGTFVVNIAQLSQDDSGRYKCGLGINSRGLSFDVSLEV

SEQ ID NO: 256 - protein, 109 aa, Artificial Sequence
  Description of Artificial Sequence Synthetic polypeptide
  hD1_tCDR2
KSPIFGPEEVNSVEGNSVSITCYYPPTSVNRHTRKYWCRQGARGGCITLISSDAPVSSKYAGRANLTNFPENGTFVVNIAQLSQDDSGRYKCGLGINSRGLSFDVSLEV

SEQ ID NO: 257 - protein, 112 aa, Artificial Sequence
  Description of Artificial Sequence Synthetic polypeptide
  hD1_tCDR3
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGVGGVWSADLSFDVSLEV

SEQ ID NO: 258 - protein, 7 aa, Artificial Sequence
  Description of Artificial Sequence Synthetic peptide
  VHH11 CDR1 (Kabat)
FTTYRMG

SEQ ID NO: 259 - protein, 5 aa, Artificial Sequence
  Description of Artificial Sequence Synthetic peptide
  VHH11 CDR1 (Kabat) - alternative
TYRMG

SEQ ID NO: 260 - protein, 7 aa, Artificial Sequence
  Description of Artificial Sequence Synthetic peptide
  VHH1 and VHH2 CDR2 (Chothia) - alternative
WNGRGTY

SEQ ID NO: 261 - protein, 3 aa, Artificial Sequence
  Description of Artificial Sequence Synthetic peptide
  VHH3 CDR2 (Chothia) - alternative
GGG

SEQ ID NO: 262 - protein, 3 aa, Artificial Sequence
  Description of Artificial Sequence Synthetic peptide
  VHH4 CDR2 (Chothia) - alternative
RIA

SEQ ID NO: 263 - protein, 4 aa, Artificial Sequence
  Description of Artificial Sequence Synthetic peptide
  VHH5 CDR2 (Chothia) - alternative
WNGG

SEQ ID NO: 264 - protein, 3 aa, Artificial Sequence
  Description of Artificial Sequence Synthetic peptide
  VHH6 CDR2 (Chothia) - alternative
GGG

SEQ ID NO: 265 - protein, 3 aa, Artificial Sequence
  Description of Artificial Sequence Synthetic peptide
  VHH7 CDR2 (Chothia) - alternative
GGG

SEQ ID NO: 266 - protein, 4 aa, Artificial Sequence
  Description of Artificial Sequence Synthetic peptide
  VHH9 CDR2 (Chothia) - alternative
WSGG

SEQ ID NO: 267 - protein, 4 aa, Artificial Sequence
  Description of Artificial Sequence Synthetic peptide
  VHH10 CDR2 (Chothia) - alternative
WSGS

SEQ ID NO: 268 - protein, 4 aa, Artificial Sequence
  Description of Artificial Sequence Synthetic peptide
  VHH11 CDR2 (Chothia) - alternative
WSGG

SEQ ID NO: 269 - protein, 4 aa, Artificial Sequence
  Description of Artificial Sequence Synthetic peptide
  VHH12 CDR2 (Chothia) - alternative
WNGG

SEQ ID NO: 270 - protein, 11 aa, Artificial Sequence
  Description of Artificial Sequence Synthetic peptide
  VHH1 and VHH2 CDR2 (IMGT) - alternative
IDWNGRGTYYR

SEQ ID NO: 271 - protein, 4 aa, Artificial Sequence
  Description of Artificial Sequence Synthetic peptide
  VHH9 CDR3 (Kabat) - alternative
QRGY

SEQ ID NO: 272 - protein, 11 aa, Artificial Sequence
  Description of Artificial Sequence Synthetic peptide
  VHH1 CDR3 (Chothia) - alternative
SIDLNWYGGMD

SEQ ID NO: 273 - protein, 13 aa, Artificial Sequence
  Description of Artificial Sequence Synthetic peptide
  VHH2 CDR3 (Chothia) - alternative
TVLTDPRVLNEYA

SEQ ID NO: 274 - protein, 10 aa, Artificial Sequence
  Description of Artificial Sequence Synthetic peptide
  VHH3 CDR3 (Chothia) - alternative
VFGSSGYVET

SEQ ID NO: 275 - protein, 3 aa, Artificial Sequence
  Description of Artificial Sequence Synthetic peptide
  VHH4 CDR3 (Chothia) - alternative
LTA

SEQ ID NO: 276 - protein, 5 aa, Artificial Sequence
  Description of Artificial Sequence Synthetic peptide
  VHH5 CDR3 (Chothia) - alternative
PFNQG

SEQ ID NO: 277 - protein, 3 aa, Artificial Sequence
  Description of Artificial Sequence Synthetic peptide
  VHH6 CDR3 (Chothia) - alternative
LTS

SEQ ID NO: 278 - protein, 20 aa, Artificial Sequence
  Description of Artificial Sequence Synthetic peptide
  VHH7 CDR3 (Chothia) - alternative
VNPIITAWGTIGVREIPDYD

SEQ ID NO: 279 - protein, 2 aa, Artificial Sequence
  Description of Artificial Sequence Synthetic peptide
  VHH9 CDR3 (Chothia) - alternative
RG

SEQ ID NO: 280 - protein, 5 aa, Artificial Sequence
  Description of Artificial Sequence Synthetic peptide
  VHH10 CDR3 (Chothia) - alternative
PFNQG

SEQ ID NO: 281 - protein, 19 aa, Artificial Sequence
  Description of Artificial Sequence Synthetic peptide
  VHH11 CDR3 (Chothia) - alternative
LAEYSGTYSSPADSPAGYD

SEQ ID NO: 282 - protein, 12 aa, Artificial Sequence
  Description of Artificial Sequence Synthetic peptide
  VHH12 CDR3 (Chothia) - alternative
RYYVSGTYFPAN

SEQ ID NO: 283 - protein, 15 aa, Artificial Sequence
  Description of Artificial Sequence Synthetic peptide
  VHH1 CDR3 (IMGT) - alternative
AAGSIDLNWYGGMDY

SEQ ID NO: 284 - protein, 17 aa, Artificial Sequence
  Description of Artificial Sequence Synthetic peptide
  VHH2 CDR3 (IMGT) - alternative
AATTVLTDPRVLNEYAT

(19) United States
(12) Patent Application Publication
(10) Pub. No.: US 2022/0324970 A1
(43) Pub. Date: Oct. 13, 2022

(54) MATERIALS AND METHODS FOR MULTIDIRECTIONAL BIOTRANSPORTATION

(71) Applicant: JANSSEN BIOTECH, INC., Horsham, PA (US)

(72) Inventors: Rajkumar GANESAN, Blue Bell, PA (US); Bharathikumar Vellalore MARUTHACHALAM, Downington, PA (US); Adam ZWOLAK, Bala Cynwyd, PA (US); Brian GEIST, West Chester, PA (US); Xiefan LIN-SCHMIDT, Ambler, PA (US); Sathyadevi VENKATARAMANI, Blue Bell, PA (US); Sanjaya SINGH, Blue Bell, PA (US)

(73) Assignee: JANSSEN BIOTECH, INC., Horsham, PA (US)

(21) Appl. No.: 17/630,718

(22) PCT Filed: Jul. 31, 2020
(86) PCT No.: PCT/US2020/044497

§ 371 Date: Jan. 27, 2022

Related U.S. Application Data
(60) Provisional application No. 62/940,228, filed on Nov. 25, 2019.
(60) Provisional application No. 62/940,232, filed on Nov. 25, 2019.
(60) Provisional application No. 62/940,220, filed on Nov. 25, 2019.
(60) Provisional application No. 62/940,208, filed on Nov. 25, 2019.
(60) Provisional application No. 62/940,206, filed on Nov. 25, 2019.
(60) Provisional application No. 62/940,200, filed on Nov. 25, 2019.
(60) Provisional application No. 62/940,196, filed on Nov. 25, 2019.
(60) Provisional application No. 62/882,387, filed on Aug. 2, 2019.
(60) Provisional application No. 62/882,361, filed on Aug. 2, 2019.
(60) Provisional application No. 62/882,346, filed on Aug. 2, 2019.
(60) Provisional application No. 62/882,291, filed on Aug. 2, 2019.

(51) Int. Cl. C07K 16/28; A61K 47/68; A61K 47/65

(52) U.S. Cl. CPC C07K 16/283; A61K 47/6803; A61K 47/65; A61K 47/6849; C07K 2317/569; C07K 2317/22; C07K 2317/92; C07K 2319/00

(57) ABSTRACT

A method is described for delivering a single domain antibody or a therapeutic molecule from an apical surface of a polymeric immunoglobulin receptor (pIgR)-expressing cell to a basolateral surface of the pIgR-expressing cell comprising contacting the pIgR-expressing cell with the single domain antibody or the therapeutic molecule, wherein the single domain antibody binds to pIgR, and the therapeutic molecule comprises an agent and the single domain antibody. A method is also described for transporting such a therapeutic molecule to systemic circulation or lamina propria or gastrointestinal tract of a subject comprising administering the therapeutic molecule to the subject via oral delivery, buccal delivery, nasal delivery or inhalation delivery.

### CROSS-REFERENCE TO RELATED APPLICATIONS

This application claims the benefit of U.S. Provisional Patent Application No. 62/940,232, filed Nov. 25, 2019, U.S. Provisional Patent Application No. 62/940,228, filed Nov. 25, 2019, U.S. Provisional Patent Application No. 62/940,220, filed Nov. 25, 2019, U.S. Provisional Patent Application No. 62/940,208, filed Nov. 25, 2019, U.S. Provisional Patent Application No. 62/940,206, filed Nov. 25, 2019, U.S. Provisional Patent Application No. 62/940,200, filed Nov. 25, 2019, U.S. Provisional Patent Application No. 62/940,196, filed Nov. 25, 2019, U.S. Provisional Patent Application No. 62/882,387, filed Aug. 2, 2019, U.S. Provisional Patent Application No. 62/882,361, filed Aug. 2, 2019, U.S. Provisional Patent Application No. 62/882,346, filed Aug. 2, 2019 and U.S. Provisional Patent Application No. 62/882,291, filed Aug. 2, 2019, each of which is incorporated by reference herein in its entirety.

### SEQUENCE LISTING

This application incorporates by reference a Sequence Listing submitted with this application as a text format, entitled “14620-204-228_SL.txt”, created on Jul. 28, 2020 having a size of 169,502 bytes.

### 1. FIELD

Provided herein are single domain antibodies (e.g., VHH domains) and uses thereof for delivering agents (e.g., therapeutic agents), including by transporting the agents from an apical surface of a polymeric immunoglobulin receptor (pIgR)-expressing cell to a basolateral surface of the pIgR-expressing cell.

### 2. BACKGROUND

Biologics have been the driving force in pharmaceutical space with increasing potential to address many diseases, disorders, and conditions, including chronic diseases and various unmet medical needs. Indeed, the number of biologics in development continues to increase exponentially, particularly in the therapeutic areas of cancer and cancer related conditions, rare diseases, neurologic disorders, and immunological or inflammatory diseases, disorders, and conditions, including autoimmune disorders.

However, delivery of biologics is challenging, partially due to their molecular weights and complexity. Whereas the molecular weight of synthesized small molecule drugs ranges in the few hundred to perhaps a few thousand Daltons (Da), the molecular weight of biologics can reach upward of 150,000 Da. Their relatively large size limits their transport across the epithelium, including transport through the mucosal epithelial barrier, and there are transport challenges for biologics to get to and through the mucosa. Consequently, the most prevalent mode of administration is invasive administration very often requiring the services of a health professional in a costly healthcare setting. Thus, there is need in art for effective drug administration methods particularly for biologics via less-invasive or non-invasive routes such as oral delivery, buccal delivery, nasal delivery or inhalation delivery.

### 3. SUMMARY

In one aspect, provided herein is a method for delivering from an apical surface of a polymeric immunoglobulin receptor (pIgR)-expressing cell to a basolateral surface of the pIgR-expressing cell comprising contacting the pIgR-expressing cell with (i) a single domain antibody that binds to pIgR, or (ii) a therapeutic molecule comprising an agent and the single domain antibody.

In another aspect, provided herein is a method for transporting a therapeutic molecule to a basolateral surface of the pIgR-expressing cell of a subject, comprising administering to the subject the therapeutic molecule comprising an agent and a single domain antibody. In some embodiments, the therapeutic molecule is administered to the subject via oral delivery, buccal delivery, nasal delivery or inhalation delivery. In some embodiments, the therapeutic agent is transported from an apical surface of a pIgR-expressing cell to a basolateral surface of the pIgR-expressing cell in the subject.

In another aspect, provided herein is a method for transporting a therapeutic molecule to systemic circulation of a subject, comprising administering to the subject the therapeutic molecule comprising an agent and a single domain antibody, wherein the therapeutic molecule is administered to the subject via oral delivery, buccal delivery, nasal delivery or inhalation delivery. In some embodiments, the therapeutic agent is transported from an apical surface of a pIgR-expressing cell to a basolateral surface of the pIgR-expressing cell in the subject.

In yet another aspect, provided herein is a method for transporting a therapeutic molecule to lamina propria or gastrointestinal tract of a subject, comprising administering to the subject the therapeutic molecule comprising an agent and a single domain antibody, wherein the therapeutic molecule is administered to the subject via oral delivery, buccal delivery, nasal delivery or inhalation delivery. In some embodiments, the therapeutic agent is transported from an apical surface of a pIgR-expressing cell to a basolateral surface of the pIgR-expressing cell in the subject.

In some embodiments, the single domain antibody or the therapeutic molecule comprising the agent and the single domain antibody is capable of being transported from the basolateral surface of the pIgR-expressing cell to the apical surface of the pIgR-expressing cell.

In some embodiments, the pIgR-expressing cell is an epithelial cell. In some embodiments, the epithelia cell is an intestinal lumen cell or an airway epithelial cell.

In some embodiments, the agent is a diabetes medication. In some embodiments, the diabetes medication is selected from a group consisting of insulin, glucagon-like-peptide-1, insulin-mimic peptides, and glucagon-like-peptide-1-mimic peptides.

In some embodiments, the agent is a peptide or an antibody or a fragment thereof. In some embodiments, the antibody or fragment thereof is selected from a group consisting of an anti-TNF-alpha antibody or a fragment thereof, an anti-IL23 antibody or a fragment thereof, and an antibody that binds to a receptor of IL23 or a fragment thereof.

In some embodiments, the agent is a vaccine. In some embodiments, the vaccine is for preventing an infection selected from a group consisting of Vibrio, Cholera, Typhoid, Rotavirus, Tuberculosis, HIV, Flu, Ebola, and Sendai.

In another aspect, provide herein is a process for providing a molecule to a subject, comprising administering to the subject the molecule comprising an agent and a single domain antibody that binds to polymeric immunoglobulin receptor (pIgR), wherein the molecule is administered to the subject via oral delivery, buccal delivery, nasal delivery or inhalation delivery.

In some embodiments, the molecule is capable of being provided to a basolateral surface of an pIgR-expressing cell from an apical surface of the pIgR-expressing cell in the subject.

In some embodiments, the molecule is capable of being provided to an apical surface of the pIgR-expressing cell from a basolateral surface of an pIgR-expressing cell in the subject.

In some embodiments, the pIgR-expressing cell is an epithelial cell. In some embodiments, the epithelia cell is an intestinal lumen cell or an airway epithelial cell.

In some embodiments, the agent is a diabetes medication. In some embodiments, the diabetes medication is selected from a group consisting of insulin, glucagon-like-peptide-1, insulin-mimic peptides, and glucagon-like-peptide-1-mimic peptides.

In some embodiments, the agent is a peptide or an antibody or a fragment thereof. In some embodiments, the antibody or fragment thereof is selected from a group consisting of an anti-TNF-alpha antibody or a fragment thereof, an anti-IL23 antibody or a fragment thereof, and an antibody that binds to a receptor of IL23 or a fragment thereof.

In some embodiments, the agent is a vaccine. In some embodiments, the vaccine is for preventing an infection selected from a group consisting of Vibrio, Cholera, Typhoid, Rotavirus, Tuberculosis, HIV, Flu, Ebola, and Sendai.

In another aspect, provided herein is a process comprising steps for providing a molecule to a subject.

In some embodiments, the molecule comprises an agent and a single domain antibody that binds to pIgR.

In some embodiments, the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an antibiotic, or an antibody-antibiotic conjugate.

In some embodiments, the agent is an antibody or fragment thereof, a peptide, or a vaccine.

In some embodiments, the single domain antibody is genetically fused or chemically conjugated to the agent.

In one aspect, provided herein is a system for providing a molecule to lamina propria or gastrointestinal tract of a subject, comprising a molecule suitable for administering to the subject, the molecule comprising an agent and a single domain antibody that binds to pIgR, wherein the molecule is administered to the subject via oral delivery, buccal delivery, nasal delivery or inhalation delivery, or a combination thereof.

In some embodiments, the agent is a diabetes medication. In some embodiments, the diabetes medication is selected from a group consisting of insulin, glucagon-like-peptide-1, insulin-mimic peptides, and glucagon-like-peptide-1-mimic peptides.

In some embodiments, the agent is a peptide or an antibody or a fragment thereof. In some embodiments, the antibody or fragment thereof is selected from a group consisting of an anti-TNF-alpha antibody or a fragment thereof, an anti-IL23 antibody or a fragment thereof, and an antibody that binds to a receptor of IL23 or a fragment thereof.

In some embodiments, the agent is a vaccine. In some embodiments, the vaccine is for preventing an infection selected from a group consisting of Vibrio, Cholera, Typhoid, Rotavirus, Tuberculosis, HIV, Flu, Ebola, and Sendai.

In another aspect, provided herein is a system comprising a means for providing a molecule to lamina propria or gastrointestinal tract of a subject.

In some embodiments, the molecule comprises an agent and a single domain antibody that binds to pIgR.

In some embodiments, the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an antibiotic, or an antibody-antibiotic conjugate.

In some embodiments, the agent is an antibody or fragment thereof, a peptide, or a vaccine.

In some embodiments, the single domain antibody is genetically fused or chemically conjugated to the agent.

In some embodiments, the single domain antibody binds to an extracellular domain 1, an extracellular domain 2, an extracellular domain 1-2, an extracellular domain 3, an extracellular domain 2-3, an extracellular domain 4-5, or an extracellular domain 5 of pIgR.

In some embodiments, the single domain antibody binds to an extracellular domain 1 of pIgR. In some embodiments, the single domain antibody binds to an extracellular domain 2 of pIgR. In some embodiments, the single domain antibody binds to an extracellular domain 1-2 of pIgR. In some embodiments, the single domain antibody binds to an extracellular domain 3 of pIgR. In some embodiments, the single domain antibody binds to an extracellular domain 2-3 of pIgR. In some embodiments, the single domain antibody binds to an extracellular domain 4-5 of pIgR. In some embodiments, the single domain antibody binds to an extracellular domain 5 of pIgR.

In some embodiments, the single domain antibody competes with IgA binding to the pIgR. In some embodiments, the single domain antibody promotes IgA binding to the pIgR.

In some embodiments, the K_{D} of the binding of the single domain antibody to pIgR is from about 4 to about 525 nM. In some embodiments, the K_{D} of the binding of the single domain antibody to pIgR is less than about 50 nM. In some embodiments, the K_{D} of the binding of the single domain antibody to pIgR is from about 4 to about 34 nM.

In some embodiments, the Tₘ of the single domain antibody is from about 53 to about 77° C. In other embodiments, the Tₘ of the single domain antibody is from 53.9 to 76.4° C.

In some embodiments, pIgR is human pIgR. In other embodiments, pIgR is mouse pIgR.

In some embodiments, the single domain antibody provided herein does not bind to a stalk sequence of human pIgR (e.g., SEQ ID NO:143 and/or a stalk sequence of mouse pIgR (e.g., SEQ ID NO:144 or SEQ ID NO:145).

In some embodiments, the single domain antibody comprises a CDR3 sequence of

(SEQ ID NO: 60)
GSIDLNWYGGMDY,
(SEQ ID NO: 61)
TTVLTDPRVLNEYAT,
(SEQ ID NO: 62)
DVFGSSGYVETY,
(SEQ ID NO: 63)
PLTAR,
(SEQ ID NO: 64)
DPFNQGY,
(SEQ ID NO: 65)
PLTSR,
(SEQ ID NO: 66)
MVNPIITAWGTIGVREIPDYDY,
(SEQ ID NO: 67)
DQRGY,
(SEQ ID NO: 271)
QRGY,
(SEQ ID NO: 68)
DPFNQGY,
(SEQ ID NO: 69)
DLAEYSGTYSSPADSPAGYDY,
(SEQ ID NO: 70)
ARYYVSGTYFPANY,
(SEQ ID NO: 71)
GSIDLNWYGGMDY,
(SEQ ID NO: 272)
SIDLNWYGGMD,
(SEQ ID NO: 72)
TTVLTDPRVLNEYAT,
(SEQ ID NO: 273)
TVLTDPRVLNEYA,
(SEQ ID NO: 73)
DVFGSSGYVETY,
(SEQ ID NO: 274)
VFGSSGYVET,
(SEQ ID NO: 74)
PLTAR,
(SEQ ID NO: 275)
LTA,
(SEQ ID NO: 75)
DPFNQGY,
(SEQ ID NO: 276)
PFNQG,
(SEQ ID NO: 76)
PLTSR,
(SEQ ID NO: 277)
LTS,
(SEQ ID NO: 77)
MVNPIITAWGTIGVREIPDYDY,
(SEQ ID NO: 278)
VNPIITAWGTIGVREIPDYD,
(SEQ ID NO: 78)
DQRGY,
(SEQ ID NO: 279)
RG,
(SEQ ID NO: 79)
DPFNQGY,
(SEQ ID NO: 280)
PFNQG,
(SEQ ID NO: 80)
DLAEYSGTYSSPADSPAGYDY,
(SEQ ID NO: 281)
LAEYSGTYSSPADSPAGYD,
(SEQ ID NO: 81)
ARYYVSGTYFPANY,
(SEQ ID NO: 282)
RYYVSGTYFPAN,
(SEQ ID NO: 82)
CAAGSIDLNWYGGMDY,
(SEQ ID NO: 283)
AAGSIDLNWYGGMDY,
(SEQ ID NO: 83)
CAATTVLTDPRVLNEYAT,
(SEQ ID NO: 284)
AATTVLTDPRVLNEYAT,
(SEQ ID NO: 84)
KADVFGSSGYVETY,
(SEQ ID NO: 85)
NHPLTAR,
(SEQ ID NO: 86)
AADPFNQGY,
(SEQ ID NO: 87)
NHPLTSR,
(SEQ ID NO: 88)
ASMVNPIITAWGTIGVREIPDYDY,
(SEQ ID NO: 89)
NDQRGY,
(SEQ ID NO: 90)
AADPFNQGY,
(SEQ ID NO: 91)
AADLAEYSGTYSSPADSPAGYDY,
(SEQ ID NO: 92)
AAARYYVSGTYFPANY,
(SEQ ID NO: 214)
GSIDLNWYGGMDY,
(SEQ ID NO: 215)
TTVLTDPRVLNEYAT,
(SEQ ID NO: 216)
DVFGSSGYVETY,
(SEQ ID NO: 217)
PLTAR,
(SEQ ID NO: 218)
DPFNQGY,
(SEQ ID NO: 219)
PLTSR,
(SEQ ID NO: 220)
MVNPIITAWGTIGVREIPDYDY,
(SEQ ID NO: 221)
QRGY,
(SEQ ID NO: 222)
DPFNQGY,
(SEQ ID NO: 223)
DLAEYSGTYSSPADSPAGYDY,
(SEQ ID NO: 224)
ARYYVSGTYFPANY,
(SEQ ID NO: 225)
AAGSIDLNWYGGMD,
(SEQ ID NO: 226)
AATTVLTDPRVLNEYA,
(SEQ ID NO: 227)
KADVFGSSGYVET,
(SEQ ID NO: 228)
NHPLTA,
(SEQ ID NO: 229)
AADPFNQG,
(SEQ ID NO: 230)
NHPLTS,
(SEQ ID NO: 231)
ASMVNPIITAWGTIGVREIPDYD,
(SEQ ID NO: 232)
NDQRG,
(SEQ ID NO: 233)
AADPFNQG,
(SEQ ID NO: 234)
AADLAEYSGTYSSPADSPAGYD,
(SEQ ID NO: 235)
AAARYYVSGTYFPAN,
(SEQ ID NO: 236)
GSIDLNWYGGMDY,
(SEQ ID NO: 237)
TTVLTDPRVLNEYAT,
(SEQ ID NO: 238)
DVFGSSGYVETY,
(SEQ ID NO: 239)
PLTAR,
(SEQ ID NO: 240)
DPFNQGY,
(SEQ ID NO: 241)
PLTSR,
(SEQ ID NO: 242)
MVNPIITAWGTIGVREIPDYDY,
(SEQ ID NO: 243)
QRGY,
(SEQ ID NO: 244)
DPFNQGY,
(SEQ ID NO: 245)
DLAEYSGTYSSPADSPAGYDY,
or
(SEQ ID NO: 246)
ARYYVSGTYFPANY.

In some embodiments, the single domain antibody comprises a CDR2 sequence of

| (SEQ ID NO: 30) | |
|---|---|
| | AIDWNGRGTYYRYYADSVKG, |
| (SEQ ID NO: 31) | |
| | RINGGGITHYAESVKG, |
| (SEQ ID NO: 32) | |
| | FIDRIATTTIATSVKG, |
| (SEQ ID NO: 33) | |
| | AITWNGGTTYYADSVKG, |
| (SEQ ID NO: 34) | |
| | FISGGGTTTYADSVKG, |
| (SEQ ID NO: 35) | |
| | RITGGGSTHYAESVKG, |
| (SEQ ID NO: 36) | |
| | AISWSGGSTTYADPVKG, |
| (SEQ ID NO: 37) | |
| | AISWSGSSAGYGDSVKG, |
| (SEQ ID NO: 38) | |
| | AIRWSGGRTLYADSVKG, |
| (SEQ ID NO: 39) | |
| | SITWNGGSTSYADSVKG, |
| (SEQ ID NO: 40) | |
| | DWNGRGTYY, |
| (SEQ ID NO: 260) | |
| | WNGRGTY, |
| (SEQ ID NO: 41) | |
| | NGGGI, |
| (SEQ ID NO: 261) | |
| | GGG, |
| (SEQ ID NO: 42) | |
| | DRIAT, |
| (SEQ ID NO: 262) | |
| | RIA, |
| (SEQ ID NO: 43) | |
| | TWNGGT, |
| (SEQ ID NO: 263) | |
| | WNGG, |
| (SEQ ID NO: 44) | |
| | SGGGT, |
| (SEQ ID NO: 264) | |
| | GGG, |
| (SEQ ID NO: 45) | |
| | TGGGS, |
| (SEQ ID NO: 265) | |
| | GGG, |
| (SEQ ID NO: 46) | |
| | SWSGGS, |
| (SEQ ID NO: 266) | |
| | WSGG, |
| (SEQ ID NO: 47) | |
| | SWSGSS, |
| (SEQ ID NO: 267) | |
| | WSGS, |
| (SEQ ID NO: 48) | |
| | RWSGGR, |
| (SEQ ID NO: 268) | |
| | WSGG, |
| (SEQ ID NO: 49) | |
| | TWNGGS, |
| (SEQ ID NO: 269) | |
| | WNGG, |
| (SEQ ID NO: 50) | |
| | IDWNGRGTYY, |
| (SEQ ID NO: 270) | |
| | IDWNGRGTYYR, |
| (SEQ ID NO: 51) | |
| | INGGGIT, |
| (SEQ ID NO: 52) | |
| | IDRIATT, |
| (SEQ ID NO: 53) | |
| | ITWNGGTT, |
| (SEQ ID NO: 54) | |
| | ISGGGTT, |
| (SEQ ID NO: 55) | |
| | ITGGGST, |
| (SEQ ID NO: 56) | |
| | ISWSGGST, |
| (SEQ ID NO: 57) | |
| | ISWSGSSA, |
| (SEQ ID NO: 58) | |
| | IRWSGGRT, |
| (SEQ ID NO: 59) | |
| | ITWNGGST, |
| (SEQ ID NO: 184) | |
| | AIDWNGRGTYYRYYADSVKG, |
| (SEQ ID NO: 185) | |
| | RINGGGITHYAESVKG, |
| (SEQ ID NO: 186) | |
| | FIDRIATTTIATSVKG, |
| (SEQ ID NO: 187) | |
| | AITWNGGTTYYADSVKG, |
| (SEQ ID NO: 188) | |
| | FISGGGTTTYADSVKG, |
| (SEQ ID NO: 189) | |
| | RITGGGSTHYAESVKG, |
| (SEQ ID NO: 190) | |
| | AISWSGGSTTYADPVKG, |
| (SEQ ID NO: 191) | |
| | AISWSGSSAGYGDSVKG, |
| (SEQ ID NO: 192) | |
| | AIRWSGGRTLYADSVKG, |
| (SEQ ID NO: 193) | |
| | SITWNGGSTSYADSVKG, |
| (SEQ ID NO: 194) | |
| | FVAAIDWNGRGTYYRY, |
| (SEQ ID NO: 195) | |
| | LVARINGGGITH, |
| (SEQ ID NO: 196) | |
| | WVGFIDRIATTT, |
| (SEQ ID NO: 197) | |
| | FVAAITWNGGTTY, |
| (SEQ ID NO: 198) | |
| | WVAFISGGGTTT, |
| (SEQ ID NO: 199) | |
| | LVARITGGGSTH, |
| (SEQ ID NO: 200) | |
| | FVAAISWSGGSTT, |
| (SEQ ID NO: 201) | |
| | FVAAISWSGSSAG, |
| (SEQ ID NO: 202) | |
| | FVAAIRWSGGRTL, |
| (SEQ ID NO: 203) | |
| | FVASITWNGGSTS, |
| (SEQ ID NO: 204) | |
| | AIDWNGRGTYYRY, |
| (SEQ ID NO: 205) | |
| | RINGGGITH, |
| (SEQ ID NO: 206) | |
| | FIDRIATTT, |
| (SEQ ID NO: 207) | |
| | AITWNGGTTY, |
| (SEQ ID NO: 208) | |
| | FISGGGTTT, |
| (SEQ ID NO: 209) | |
| | RITGGGSTH, |
| (SEQ ID NO: 210) | |
| | AISWSGGSTT, |
| (SEQ ID NO: 211) | |
| | AISWSGSSAG, |
| (SEQ ID NO: 212) | |
| | AIRWSGGRTL, |
| | or |
| (SEQ ID NO: 213) | |
| | SITWNGGSTS. |

In some embodiments, the single domain antibody comprises a CDR1 sequence of

| (SEQ ID NO: 1) | |
|---|---|
| | SYRMG, |
| (SEQ ID NO: 2) | |
| | INVMG, |
| (SEQ ID NO: 3) | |
| | SNAMG, |
| (SEQ ID NO: 4) | |
| | SYAMG, |
| (SEQ ID NO: 5) | |
| | SDAMG, |
| (SEQ ID NO: 6) | |
| | INVMG, |
| (SEQ ID NO: 7) | |
| | TYRMG, |
| (SEQ ID NO: 8) | |
| | RYAMG, |
| (SEQ ID NO: 258) | |
| | FTTYRMG, |
| (SEQ ID NO: 259) | |
| | TYRMG, |
| (SEQ ID NO: 9) | |
| | FNTYAMG, |
| (SEQ ID NO: 10) | |
| | GLTFSSY, |
| (SEQ ID NO: 11) | |
| | GSIFSIN, |
| (SEQ ID NO: 12) | |
| | GTSVSSN, |
| (SEQ ID NO: 13) | |
| | GRTFSSY, |
| (SEQ ID NO: 14) | |
| | GSSVSSD, |
| (SEQ ID NO: 15) | |
| | RSIGSIN, |
| (SEQ ID NO: 16) | |
| | GRTFSTY, |
| (SEQ ID NO: 17) | |
| | GFTFTRY, |
| (SEQ ID NO: 18) | |
| | GRTFTTY, |
| (SEQ ID NO: 19) | |
| | GRTLSFNTY, |
| (SEQ ID NO: 20) | |
| | GLTFSSYR, |
| (SEQ ID NO: 21) | |
| | GSIFSINV, |
| (SEQ ID NO: 22) | |
| | GTSVSSNA, |
| (SEQ ID NO: 23) | |
| | GRTFSSYA, |
| (SEQ ID NO: 24) | |
| | GSSVSSDA, |
| (SEQ ID NO: 25) | |
| | RSIGSINV, |
| (SEQ ID NO: 26) | |
| | GRTFSTYR, |
| (SEQ ID NO: 27) | |
| | GFTFTRYA, |
| (SEQ ID NO: 28) | |
| | GRTFTTYR, |
| (SEQ ID NO: 29) | |
| | GRTLSFNTYA, |
| (SEQ ID NO: 154) | |
| | GLTFSSYRMG, |
| (SEQ ID NO: 155) | |
| | GSIFSINVMG, |
| (SEQ ID NO: 156) | |
| | GTSVSSNAMG, |
| (SEQ ID NO: 157) | |
| | GRTFSSYAMG, |
| (SEQ ID NO: 158) | |
| | GSSVSSDAMG, |
| (SEQ ID NO: 159) | |
| | RSIGSINVMG, |
| (SEQ ID NO: 160) | |
| | GRTFSTYRMG, |
| (SEQ ID NO: 161) | |
| | GFTFTRYAMG, |
| (SEQ ID NO: 162) | |
| | GRTFTTYRMG, |
| (SEQ ID NO: 163) | |
| | GRTLSFNTYAMG, |
| (SEQ ID NO: 164) | |
| | SSYRMG, |
| (SEQ ID NO: 165) | |
| | SINVMG, |
| (SEQ ID NO: 166) | |
| | SSNAMG, |
| (SEQ ID NO: 167) | |
| | SSYAMG, |
| (SEQ ID NO: 168) | |
| | SSDAMG, |
| (SEQ ID NO: 169) | |
| | SINVMG, |
| (SEQ ID NO: 170) | |
| | STYRMG, |
| (SEQ ID NO: 171) | |
| | TRYAMG, |
| (SEQ ID NO: 172) | |
| | TTYRMG, |
| (SEQ ID NO: 173) | |
| | SFNTYAMG, |
| (SEQ ID NO: 174) | |
| | GLTFSSYRMG, |
| (SEQ ID NO: 175) | |
| | GSIFSINVMG, |
| (SEQ ID NO: 176) | |
| | GTSVSSNAMG, |
| (SEQ ID NO: 177) | |
| | GRTFSSYAMG, |
| (SEQ ID NO: 178) | |
| | GSSVSSDAMG, |
| (SEQ ID NO: 179) | |
| | RSIGSINVMG, |
| (SEQ ID NO: 180) | |
| | GRTFSTYRMG, |
| (SEQ ID NO: 181) | |
| | GFTFTRYAMG, |
| (SEQ ID NO: 182) | |
| | GRTFTTYRMG, |
| | or |
| (SEQ ID NO: 183) | |
| | GRTLSFNTYAMG. |

In some embodiments, the single domain antibody provided herein comprises a CDR1 sequence, a CDR2 sequence, and a CDR3 sequence of the single domain antibody selected from the group consisting of:
a) VHH1:

i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);

ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);

iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);

iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);

v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or

vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:

i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);

ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);

iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);

iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);

v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or

vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:

i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);

ii) the CDR1 sequence of GSIF SIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);

iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);

iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);

v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or

vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:

i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);

ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);

iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);

iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);

v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or

vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:

i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);

ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);

iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);

iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);

v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or

vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:

i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);

ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);

iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);

iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);

v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or

vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:

i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);

ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);

iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);

iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);

v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or

vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:

i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);

ii) the CDR1 sequence of GRTF STY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);

iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);

iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);

v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or

vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:

i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);

ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);

iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);

iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);

v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or

vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:

i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);

ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);

iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);

iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);

v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or

vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:

i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);

ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);

iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);

iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);

v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or

vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

In some embodiments, the single domain antibody comprises a framework derived from the framework of any of the single domain antibodys comprising the sequences of

(SEQ ID NO: 93)
QVQLVESGGGLVQAGGSLKLACAAPGLTFSSYRMGWFRQAPGQEREFVAA
IDWNGRGTYYRYYADSVKGRSTISRDNAKNTMYLQMNSLKPEDTAVYYCA
AGSIDLNWYGGMDYWGQGTQVTVSS,
(SEQ ID NO: 94)
EVQVVESGGGLVQAGGSLKLACAAPGLTFSSYRMGWFRQAPGQEREFVAA
IDWNGRGTYYRYYADSVKGRSTISRDNAKNTVYLQMNSLKPEDTAVYYCA
ATTVLTDPRVLNEYATWGQGTQVTVSS,
(SEQ ID NO: 95)
QLQLVESGGGLVQPGGSLRLSCAASGSIFSINVMGWYRQAPGKQRELVAR
INGGGITHYAESVKGRFTISRDNAKNTVYLQMNSLKPEDTAAYYCKADVF
GSSGYVETYWGQGTQVTVSS,
(SEQ ID NO: 96)
EVQVVESGGGLVQAGGSLRLSCAVSGTSVSSNAMGWYRQAPGKQREWVGF
IDRIATTTIATSVKGRFAITRDNAKNTVYLQMSGLKPEDTAVYYCNHPLT
ARWGQGTQVTVSS,
(SEQ ID NO: 97)
QVQLVESGGGLVQAGGSLRLSCAASGRTFSSYAMGWFRQAPGKEREFVAA
ITWNGGTTYYADSVKGRFTISRDNAKNTVYLQMNSLKPEDTAVYYCAADP
FNQGYWGQGTQVTVSS,
(SEQ ID NO: 98)
EVQLVESGGGLVQAGGSLRLSCAVSGSSVSSDAMGWYRQAPGNQRAWVAF
ISGGGTTTYADSVKGRFTISRDNTKNTVYLHMNSLKPEDTAVYYCNHPLT
SRWGQGTQVTVSS,
(SEQ ID NO: 99)
EVQVVESGGGLVQAGGSLRLACVASRSIGSINVMGWYRQAPGKQRDLVAR
ITGGGSTHYAESVKGRFTISRDNAKNTVYLQMNSLEPEDTAVYYCASMVN
PIITAWGTIGVREIPDYDYWGQGTQVTVSS,
(SEQ ID NO: 100)
QVQLVESGGGLVQAGGSLRLSCAVSGRTFSTYRMGWFRQAPGKERSFVAA
ISWSGGSTTYADPVKGRFTISRDNAKNTVYLRMNSLKPEDTAVYYCNDQR
GYWGQGTLVTVSS,
(SEQ ID NO: 101)
EVQVVESGGGLVQAGGSLRLSCAASGFTFTRYAMGWFRQAPGKERSFVAA
ISWSGSSAGYGDSVKGRFTISRDNAKNTLYLQMNSLKPEDTAVYYCAADP
FNQGYWGQGTQVTVSS,
(SEQ ID NO: 102)
EVQVVESGGGLVQAGGSLRLSCAASGRTFTTYRMGWFRQAPGKEREFVAA
IRWSGGRTLYADSVKGRFTISRDNAKNTAYLQMNNLRPEDTAVYYCAADL
AEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFV
ASITWNGGSTSYADSVKGRFTITRDNAKNTATLRMNSLQPDDTAVYYCAA
ARYYVSGTYFPANYWGQGTQVTVSS.

In some embodiments, the single domain antibody comprises a framework comprising sequence having at least 75%, 80%, 85%, 90%, 95%, 96%, 97%, 98%, 99%, or 100% sequence identity with the sequence of

(SEQ ID NO: 93)
QVQLVESGGGLVQAGGSLKLACAAPGLTFSSYRMGWFRQAPGQEREFVAA
IDWNGRGTYYRYYADSVKGRSTISRDNAKNTMYLQMNSLKPEDTAVYYCA
AGSIDLNWYGGMDYWGQGTQVTVSS,
(SEQ ID NO: 94)
EVQVVESGGGLVQAGGSLKLACAAPGLTFSSYRMGWFRQAPGQEREFVAA
IDWNGRGTYYRYYADSVKGRSTISRDNAKNTVYLQMNSLKPEDTAVYYCA
ATTVLTDPRVLNEYATWGQGTQVTVSS,
(SEQ ID NO: 95)
QLQLVESGGGLVQPGGSLRLSCAASGSIFSINVMGWYRQAPGKQRELVAR
INGGGITHYAESVKGRFTISRDNAKNTVYLQMNSLKPEDTAAYYCKADVF
GSSGYVETYWGQGTQVTVSS,
(SEQ ID NO: 96)
EVQVVESGGGLVQAGGSLRLSCAVSGTSVSSNAMGWYRQAPGKQREWVGF
IDRIATTTIATSVKGRFAITRDNAKNTVYLQMSGLKPEDTAVYYCNHPLT
ARWGQGTQVTVSS,
(SEQ ID NO: 97)
QVQLVESGGGLVQAGGSLRLSCAASGRTFSSYAMGWFRQAPGKEREFVAA
ITWNGGTTYYADSVKGRFTISRDNAKNTVYLQMNSLKPEDTAVYYCAADP
FNQGYWGQGTQVTVSS,
(SEQ ID NO: 98)
EVQLVESGGGLVQAGGSLRLSCAVSGSSVSSDAMGWYRQAPGNQRAWVAF
ISGGGTTTYADSVKGRFTISRDNTKNTVYLHMNSLKPEDTAVYYCNHPLT
SRWGQGTQVTVSS,
(SEQ ID NO: 99)
EVQVVESGGGLVQAGGSLRLACVASRSIGSINVMGWYRQAPGKQRDLVAR
ITGGGSTHYAESVKGRFTISRDNAKNTVYLQMNSLEPEDTAVYYCASMVN
PIITAWGTIGVREIPDYDYWGQGTQVTVSS,
(SEQ ID NO: 100)
QVQLVESGGGLVQAGGSLRLSCAVSGRTFSTYRMGWFRQAPGKERSFVAA
ISWSGGSTTYADPVKGRFTISRDNAKNTVYLRMNSLKPEDTAVYYCNDQR
GYWGQGTLVTVSS,
(SEQ ID NO: 101)
EVQVVESGGGLVQAGGSLRLSCAASGFTFTRYAMGWFRQAPGKERSFVAA
ISWSGSSAGYGDSVKGRFTISRDNAKNTLYLQMNSLKPEDTAVYYCAADP
FNQGYWGQGTQVTVSS,
(SEQ ID NO: 102)
EVQVVESGGGLVQAGGSLRLSCAASGRTFTTYRMGWFRQAPGKEREFVAA
IRWSGGRTLYADSVKGRFTISRDNAKNTAYLQMNNLRPEDTAVYYCAADL
AEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFV
ASITWNGGSTSYADSVKGRFTITRDNAKNTATLRMNSLQPDDTAVYYCAA
ARYYVSGTYFPANYWGQGTQVTVSS.

In some embodiments, the single domain antibody is comprised of a sequence having at least 75%, 80%, 85%, 90%, 95%, 96%, 97%, 98%, 99%, or 100% sequence identity with the sequence of

(SEQ ID NO: 93)
QVQLVESGGGLVQAGGSLKLACAAPGLTFSSYRMGWFRQAPGQEREFVAA
IDWNGRGTYYRYYADSVKGRSTISRDNAKNTMYLQMNSLKPEDTAVYYCA
AGSIDLNWYGGMDYWGQGTQVTVSS,
(SEQ ID NO: 94)
EVQVVESGGGLVQAGGSLKLACAAPGLTFSSYRMGWFRQAPGQEREFVAA
IDWNGRGTYYRYYADSVKGRSTISRDNAKNTVYLQMNSLKPEDTAVYYCA
ATTVLTDPRVLNEYATWGQGTQVTVSS,
(SEQ ID NO: 95)
QLQLVESGGGLVQPGGSLRLSCAASGSIFSINVMGWYRQAPGKQRELVAR
INGGGITHYAESVKGRFTISRDNAKNTVYLQMNSLKPEDTAAYYCKADVF
GSSGYVETYWGQGTQVTVSS,
(SEQ ID NO: 96)
EVQVVESGGGLVQAGGSLRLSCAVSGTSVSSNAMGWYRQAPGKQREWVGF
IDRIATTTIATSVKGRFAITRDNAKNTVYLQMSGLKPEDTAVYYCNHPLT
ARWGQGTQVTVSS,
(SEQ ID NO: 97)
QVQLVESGGGLVQAGGSLRLSCAASGRTFSSYAMGWFRQAPGKEREFVAA
ITWNGGTTYYADSVKGRFTISRDNAKNTVYLQMNSLKPEDTAVYYCAADP
FNQGYWGQGTQVTVSS,
(SEQ ID NO: 98)
EVQLVESGGGLVQAGGSLRLSCAVSGSSVSSDAMGWYRQAPGNQRAWVAF
ISGGGTTTYADSVKGRFTISRDNTKNTVYLHMNSLKPEDTAVYYCNHPLT
SRWGQGTQVTVSS,
(SEQ ID NO: 99)
EVQVVESGGGLVQAGGSLRLACVASRSIGSINVMGWYRQAPGKQRDLVAR
ITGGGSTHYAESVKGRFTISRDNAKNTVYLQMNSLEPEDTAVYYCASMVN
PIITAWGTIGVREIPDYDYWGQGTQVTVSS,
(SEQ ID NO: 100)
QVQLVESGGGLVQAGGSLRLSCAVSGRTFSTYRMGWFRQAPGKERSFVAA
ISWSGGSTTYADPVKGRFTISRDNAKNTVYLRMNSLKPEDTAVYYCNDQR
GYWGQGTLVTVSS,
(SEQ ID NO: 101)
EVQVVESGGGLVQAGGSLRLSCAASGFTFTRYAMGWFRQAPGKERSFVAA
ISWSGSSAGYGDSVKGRFTISRDNAKNTLYLQMNSLKPEDTAVYYCAADP
FNQGYWGQGTQVTVSS,
(SEQ ID NO: 102)
EVQVVESGGGLVQAGGSLRLSCAASGRTFTTYRMGWFRQAPGKEREFVAA
IRWSGGRTLYADSVKGRFTISRDNAKNTAYLQMNNLRPEDTAVYYCAADL
AEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFV
ASITWNGGSTSYADSVKGRFTITRDNAKNTATLRMNSLQPDDTAVYYCAA
ARYYVSGTYFPANYWGQGTQVTVSS.

In some embodiments, the single domain antibody is genetically fused or chemically conjugated to the agent.

In some embodiments, the single domain antibody provided herein further comprises a linker between the single domain antibody and the agent. In some embodiments, the linker is a polypeptide. In some embodiments, the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

In some embodiments, the single domain antibody is chemically-conjugated to the agent. In other embodiments, the single domain antibody is non-covalently bound to the agent.

In some embodiments, the method provided herein does not inhibit pIgR-mediated transcytosis of IgA.

In some embodiments, the single domain antibody comprises a CDR1 sequence of

| (SEQ ID NO: 3) | |
|---|---|
| | SNAMG, |
| (SEQ ID NO: 6) | |
| | INVMG, |
| (SEQ ID NO: 7) | |
| | TYRMG, |
| (SEQ ID NO: 8) | |
| | RYAMG, |
| (SEQ ID NO: 258) | |
| | FTTYRMG, |
| (SEQ ID NO: 259) | |
| | TYRMG, |
| (SEQ ID NO: 9) | |
| | FNTYAMG, |
| (SEQ ID NO: 12) | |
| | GTSVSSN, |
| (SEQ ID NO: 13) | |
| | GRTFSSY, |
| (SEQ ID NO: 15) | |
| | RSIGSIN, |
| (SEQ ID NO: 16) | |
| | GRTFSTY, |
| (SEQ ID NO: 17) | |
| | GFTFTRY, |
| (SEQ ID NO: 18) | |
| | GRTFTTY, |
| (SEQ ID NO: 19) | |
| | GRTLSFNTY, |
| (SEQ ID NO: 22) | |
| | GTSVSSNA, |
| (SEQ ID NO: 25) | |
| | RSIGSINV, |
| (SEQ ID NO: 26) | |
| | GRTFSTYR, |
| (SEQ ID NO: 27) | |
| | GFTFTRYA, |
| (SEQ ID NO: 28) | |
| | GRTFTTYR, |
| (SEQ ID NO: 29) | |
| | GRTLSFNTYA, |
| (SEQ ID NO: 156) | |
| | GTSVSSNAMG, |
| (SEQ ID NO: 159) | |
| | RSIGSINVMG, |
| (SEQ ID NO: 160) | |
| | GRTFSTYRMG, |
| (SEQ ID NO: 161) | |
| | GFTFTRYAMG, |
| (SEQ ID NO: 162) | |
| | GRTFTTYRMG, |
| (SEQ ID NO: 163) | |
| | GRTLSFNTYAMG, |
| (SEQ ID NO: 166) | |
| | SSNAMG, |
| (SEQ ID NO: 169) | |
| | SINVMG, |
| (SEQ ID NO: 170) | |
| | STYRMG, |
| (SEQ ID NO: 171) | |
| | TRYAMG, |
| (SEQ ID NO: 172) | |
| | TTYRMG, |
| (SEQ ID NO: 173) | |
| | SFNTYAMG, |
| (SEQ ID NO: 176) | |
| | GTSVSSNAMG, |
| (SEQ ID NO: 179) | |
| | RSIGSINVMG, |
| (SEQ ID NO: 180) | |
| | GRTFSTYRMG, |
| (SEQ ID NO: 181) | |
| | GFTFTRYAMG, |
| (SEQ ID NO: 182) | |
| | GRTFTTYRMG, |
| | or |
| (SEQ ID NO: 183) | |
| | GRTLSFNTYAMG. |

In some embodiments, the single domain antibody comprises a CDR2 sequence of

| (SEQ ID NO: 32) | |
|---|---|
| | FIDRIATTTIATSVKG, |
| (SEQ ID NO: 35) | |
| | RITGGGSTHYAESVKG, |
| (SEQ ID NO: 36) | |
| | AISWSGGSTTYADPVKG, |
| (SEQ ID NO: 37) | |
| | AISWSGSSAGYGDSVKG, |
| (SEQ ID NO: 38) | |
| | AIRWSGGRTLYADSVKG, |
| (SEQ ID NO: 39) | |
| | SITWNGGSTSYADSVKG, |
| (SEQ ID NO: 42) | |
| | DRIAT, |
| (SEQ ID NO: 262) | |
| | RIA, |
| (SEQ ID NO: 45) | |
| | TGGGS, |
| (SEQ ID NO: 265) | |
| | GGG, |
| (SEQ ID NO: 46) | |
| | SWSGGS, |
| (SEQ ID NO: 266) | |
| | WSGG, |
| (SEQ ID NO: 47) | |
| | SWSGSS, |
| (SEQ ID NO: 267) | |
| | WSGS, |
| (SEQ ID NO: 48) | |
| | RWSGGR, |
| (SEQ ID NO: 268) | |
| | WSGG, |
| (SEQ ID NO: 49) | |
| | TWNGGS, |
| (SEQ ID NO: 269) | |
| | WNGG, |
| (SEQ ID NO: 52) | |
| | IDRIATT, |
| (SEQ ID NO: 55) | |
| | ITGGGST, |
| (SEQ ID NO: 56) | |
| | ISWSGGST, |
| (SEQ ID NO: 57) | |
| | ISWSGSSA, |
| (SEQ ID NO: 58) | |
| | IRWSGGRT, |
| (SEQ ID NO: 59) | |
| | ITWNGGST, |
| (SEQ ID NO: 186) | |
| | FIDRIATTTIATSVKG, |
| (SEQ ID NO: 189) | |
| | RITGGGSTHYAESVKG, |
| (SEQ ID NO: 190) | |
| | AISWSGGSTTYADPVKG, |
| (SEQ ID NO: 191) | |
| | AISWSGSSAGYGDSVKG, |
| (SEQ ID NO: 192) | |
| | AIRWSGGRTLYADSVKG, |
| (SEQ ID NO: 193) | |
| | SITWNGGSTSYADSVKG, |
| (SEQ ID NO: 196) | |
| | WVGFIDRIATTT, |
| (SEQ ID NO: 199) | |
| | LVARITGGGSTH, |
| (SEQ ID NO: 200) | |
| | FVAAISWSGGSTT, |
| (SEQ ID NO: 201) | |
| | FVAAISWSGSSAG, |
| (SEQ ID NO: 202) | |
| | FVAAIRWSGGRTL, |
| (SEQ ID NO: 203) | |
| | FVASITWNGGSTS, |
| (SEQ ID NO: 206) | |
| | FIDRIATTT, |
| (SEQ ID NO: 209) | |
| | RITGGGSTH, |
| (SEQ ID NO: 210) | |
| | AISWSGGSTT, |
| (SEQ ID NO: 211) | |
| | AISWSGSSAG, |
| (SEQ ID NO: 212) | |
| | AIRWSGGRTL, |
| | or |
| (SEQ ID NO: 213) | |
| | SITWNGGSTS. |

In some embodiments, the single domain antibody comprises a CDR3 sequence of

| (SEQ ID NO: 63) | |
|---|---|
| | PLTAR, |
| (SEQ ID NO: 66) | |
| | MVNPIITAWGTIGVREIPDYDY, |
| (SEQ ID NO: 67) | |
| | DQRGY, |
| (SEQ ID NO: 271) | |
| | QRGY, |
| (SEQ ID NO: 68) | |
| | DPFNQGY, |
| (SEQ ID NO: 69) | |
| | DLAEYSGTYSSPADSPAGYDY, |
| (SEQ ID NO: 70) | |
| | ARYYVSGTYFPANY, |
| (SEQ ID NO: 74) | |
| | PLTAR, |
| (SEQ ID NO: 275) | |
| | LTA, |
| (SEQ ID NO: 77) | |
| | MVNPIITAWGTIGVREIPDYDY, |
| (SEQ ID NO: 278) | |
| | VNPIITAWGTIGVREIPDYD, |
| (SEQ ID NO: 78) | |
| | DQRGY, |
| (SEQ ID NO: 279) | |
| | RG, |
| (SEQ ID NO: 79) | |
| | DPFNQGY, |
| (SEQ ID NO: 280) | |
| | PFNQG, |
| (SEQ ID NO: 80) | |
| | DLAEYSGTYSSPADSPAGYDY, |
| (SEQ ID NO: 281) | |
| | LAEYSGTYSSPADSPAGYD, |
| (SEQ ID NO: 81) | |
| | ARYYVSGTYFPANY, |
| (SEQ ID NO: 282) | |
| | RYYVSGTYFPAN, |
| (SEQ ID NO: 85) | |
| | NHPLTAR, |
| (SEQ ID NO: 88) | |
| | ASMVNPIITAWGTIGVREIPDYDY, |
| (SEQ ID NO: 89) | |
| | NDQRGY, |
| (SEQ ID NO: 90) | |
| | AADPFNQGY, |
| (SEQ ID NO: 91) | |
| | AADLAEYSGTYSSPADSPAGYDY, |
| (SEQ ID NO: 92) | |
| | AAARYYVSGTYFPANY, |
| (SEQ ID NO: 217) | |
| | PLTAR, |
| (SEQ ID NO: 220) | |
| | MVNPIITAWGTIGVREIPDYDY, |
| (SEQ ID NO: 221) | |
| | QRGY, |
| (SEQ ID NO: 222) | |
| | DPFNQGY, |
| (SEQ ID NO: 223) | |
| | DLAEYSGTYSSPADSPAGYDY, |
| (SEQ ID NO: 224) | |
| | ARYYVSGTYFPANY, |
| (SEQ ID NO: 228) | |
| | NHPLTA, |
| (SEQ ID NO: 231) | |
| | ASMVNPIITAWGTIGVREIPDYD, |
| (SEQ ID NO: 232) | |
| | NDQRG, |
| (SEQ ID NO: 233) | |
| | AADPFNQG, |
| (SEQ ID NO: 234) | |
| | AADLAEYSGTYSSPADSPAGYD, |
| (SEQ ID NO: 235) | |
| | AAARYYVSGTYFPAN, |
| (SEQ ID NO: 239) | |
| | PLTAR, |
| (SEQ ID NO: 242) | |
| | MVNPIITAWGTIGVREIPDYDY, |
| (SEQ ID NO: 243) | |
| | QRGY, |
| (SEQ ID NO: 244) | |
| | DPFNQGY, |
| (SEQ ID NO: 245) | |
| | DLAEYSGTYSSPADSPAGYDY, |
| | or |
| (SEQ ID NO: 246) | |
| | ARYYVSGTYFPANY. |

### 4. BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A and 1B are schematics showing the pathway of pIgR-mediated bidirectional transcytosis. FIG. 1A shows that molecules binding to the secretory component (domains 1-5) of the pIgR ectodomain, such as dimeric IgA (natural ligand) or VHH (artificial pIgR ligand), can transcytose the epithelial cell from the basolateral to the apical direction and reach the mucosal lumen from blood. This secretory component-mediated forward transport can be used for delivering molecules to the mucosal lumen from systemic circulation. Described herein are VHH molecules that bind to the secretory component and transcytose from the basolateral to the apical side of the epithelium. FIG. 1B shows that molecules binding to the stalk region of the pIgR ectodomain (any artificial ligand) can transcytose the epithelial cell from the apical to the basolateral direction and reach the blood from mucosal lumen. This stalk-mediated reverse transport can be used for delivering molecules to systemic circulation following oral consumption.

FIG. 2 illustrates data on epitope mapping of pIgR binders. Nine HIS-tagged pIgR constructs (D1, D2, D3, D4, D5, D1-D2, D2-D3, D3-D4 and D4-D5 were expressed and purified from HEK293 cells using immobilized metal ion affinity chromatography. Because the expression and purification yield were very low for two constructs (D4 and D3-D4), these were not used for binding studies. The heat map of FIG. 2 shows the binding of VHH-mono-Fc molecules to immobilized pIgR constructs in electrochemiluminescence units. K_{D} values for all positive interactions were measured by bio-layer interferometry. The heat map of FIG. 2 indicates that the epitopes of VHH2 and VHH3 are primarily contained within hpIgR domain-1, the epitopes of VHH4 and VHH6 are primarily contained within hpIgR domain-2, and the epitopes of other six VHHs are primarily contained within hpIgR domains 4-5.

FIGS. 3A-3B illustrate data on the effect of VHH on IgA binding to hpIgR-ECD. FIG. 3A shows K_{D} values for full-length hpIgR ECD binding to immobilized VHH-mono-Fc in the absence (blue) and presence (red) of dIgA2. FIG. 3B shows the K_{D} values for immobilized dIgA2 binding to hpIgR ectodomain with and without the presence of VHH-mono-Fc molecules. dIgA2 was immobilized using amine-reactive biosensors, and the binding of pIgR and pIgR-VHH complexes were measured by bio-layer interferometry. Three molecules (VHH2, VHH3 and VHH5) had a negative effect on IgA binding to pIgR. Other VHH molecules display a small positive effect on IgA binding to pIgR.

FIG. 4 depicts the results of assays on the transcytosis activity of VHH-mono-Fc molecules. The top panel is a schematic of the EpiAirway primary human lung tissue model used for assaying VHH transcytosis. The meso-scale discovery (MSD) assay was developed to quantify the amount of VHH present in the basolateral and apical chambers before and after transcytosis. A biotinylated anti-VHH antibody was used to capture VHH-mono-Fc molecules on streptavidin plates and a ruthenylated anti-human-Fc antibody was used as a detection antibody. The bottom panel is a graph showing the amount of VHH present in the apical mucus 24 hours post VHH treatment. Five VHH molecules (VHH2, VHH6, VHH9, VHH11 and VHH12) showed greater than 20-fold increase in their mucosal amount relative to control VHH molecules (VHH1, VHH13, and VHH14).

FIG. 5 illustrates data showing tracking pIgR and VHH across the primary human lung tissue model. The left panel of FIG. 5 is a heatmap showing the amount of pIgR retained on the EpiAirway primary human lung tissue model following transcytosis. The right panel of FIG. 5 is a heatmap showing the amount of VHH retained on the EpiAirway primary human lung tissue model following transcytosis. Following 48 hours post-treatment, tissue samples were fixed, permeabilized and stained for hpIgR and VHH. The amount of pIgR and VHH retained across the tissue model was quantified by indirect immunofluorescence using Opera Phenix confocal laser microscopy. FIG. 5 shows that VHHs displayed distinct profiles of pIgR and VHH distribution across the tissue depth dimension. FIG. 5 also shows that Among the five VHHs that showed potent transcytosis, VHH2, VHH9 and VHH12-treated tissue models showed higher VHH staining near the apical surface than the other VHHs. VHH6-treated model showed the lowest staining for both VHH and pIgR across the tissue thickness. Imaging studies corroborated transcytosis results and showed colocalization of hpIgR and VHH, especially closer to the apical epithelium.

FIG. 6A is a schematic showing the structure of pIgR.

FIG. 6B is a schematic showing a mechanism of pIgR-mediated transport. Figure adapted from Kaetzel, Curr. Biol., 2001, 11(1):R35-38.

FIG. 7 shows the expression of pIgR in various organs.

FIG. 8 shows selection criteria used to assess VHH molecules that were generated from mpIgR antigen.

FIG. 9 shows selection criteria used to assess VHH molecules that were generated from hpIgR antigen.

FIG. 10 shows the results of an assay for ability of VHH molecules to bind to MDCK cells expressing pIgR.

FIG. 11 shows the expression of hpIgR on MDCK cells. Staining shows hpIgR located on the surface and interior of the monolayer of MDCK cells. The distribution of hpIgR staining within the monolayer is not uniform. Initial experiments show hpIgR receptor density at about 6000 on the surface per cell. The blue color indicates Hoechst stain for nucleus, the green color indicates antibody staining, and the red indicates anti-Rab5 staining.

FIGS. 12A-12B show the results of a VHH transcytosis assay using MDCK-hpIgR cells, as described in Example 3. Apical VHH amounts at 0, 24, and 48 hours are shown in FIG. 12B, left panel. Fold increase in apical VHH amounts at 24 hours relative to a control VHH is shown in FIG. 12B, right panel.

FIG. 12C shows transcytosis activity of VHH-mono-Fc molecules across MDCK-hpIgR monolayers from the basolateral to the apical chamber. Fold increase in apical VHH amounts at 24 hours relative to control VHH is shown.

FIG. 13 shows sequence characteristics of a set of VHH molecules, with regions of highly conserved sequence similarity are shown (SEQ ID NOS.: 93-95, 97-103 and 247-249).

FIG. 14 is a chart summarizing the purification of VHH molecules.

FIG. 15 shows the results for A-SEC purification of VHH molecules.

FIG. 16 shows the results for SEC-MALS analysis of VHH molecules.

FIG. 17 shows the results of a thermal stability assay of VHH molecules by differential scanning fluorimetry (DSF).

FIG. 18 depicts the EpiAirway human tissue model.

FIG. 19 shows the results of a VHH transcytosis assay using the EpiAirway model. The left panel shows a heat map of the amount of each tested VHH in the apical mucus at 0, 24 and 48 hours. Electrochemiluminescence (ECLU) unites obtained from the MSD assay was plotted as a heat map. The top right panel shows the amount of VHH in the apical mucus at 24 hours, and bottom right panel shows the fold increase of VHH over control in the apical mucus. The top right panel shows that five VHHs (VHH2, VHH6, VHH9, VHH11 and VHH12) showed >20-fold increase in their mucosal amount relative to control VHH molecules, and also that VHH12 showed 38-fold increase in mucus relative to control VHH and displayed the highest transcytosis activity.

FIG. 20 shows the results of IgA transcytosis assay using the EpiAirway model. FIG. 20 shows that VHH2 and VHH12-treated tissue samples stained strongly for VHH and colocalized with pIgR relative to VHH3 and VHH14 (negative control).

FIG. 21 shows colocalization of hpIgR and VHH.

FIG. 22 shows 3D reconstruction shows localization of hpIgR and VHH to the apical surface of the EpiAirway model.

FIG. 23 shows that the EpiAirway tissue model is on a slanted membrane.

FIG. 24 illustrates a strategy for Opera Phenix imaging and analysis to overcome slanted tissue issues with EpiAirway tissue model.

FIG. 25 shows the crystal structure of unliganded hpIgR in an inactive conformation. The figure is adapted from Stadtmueller et al., Elife, Mar. 4, 2016, e10640.

FIG. 26 shows structure of pIgR:IgA complex by constrained scattering modeling. The figure is adapted from Bonner et al., J. Biol. Chem., 2009, 284(8):5077-87.

FIG. 27A shows a structural model for IgA transcytosis. The figure is adapted from Stadtmueller et al., Elife, Mar. 4, 2016, e10640.

FIG. 27B shows a schematic of pIgR-mediated dimeric IgA transport across the mucosal epithelial barrier. (1) IgA production by plasma cells and IgA dimerization; (2) Binding of dimeric IgA (dIgA) to pIgR ECD on the basolateral side of the epithelium (pIgR-dIgA interactions are mediated by domains 1 and 5 of pIgR and Fc and J chains of dIgA); (3) pIgR-mediated transcytosis of dimeric IgA (clathrin-mediated endocytosis drives the basolateral to apical transport, and upon reaching the apical side, pIgR ECD is proteolytically cleaved and released into mucus along with IgA. Mucosal IgA in complex with secreted pIgR ECD (secretory component) is termed as secretory IgA (sIgA)); and (4) Neutralization of mucosal antigens by sIgA.

FIGS. 28A-28D show the effect of IgA on VHH binding to hpIgR.

FIG. 29 shows the results of domain-level epitope mapping of pIgR binders VHH1, VHH2, VHH3, VHH4, VHH5, VHH6, VHH7, VHH9, VHH10, VHH11 and VHH12. The top panel cartoon is adapted from Stadtmueller et al., Elife, Mar. 4, 2016, e10640.

FIG. 30A shows binding kinetics for hpIgR D2 binders.

FIG. 30B shows binding kinetics for hpIgR D4-D5 binders.

FIG. 31 shows properties of VHH2 and VHH3 (SEQ ID NOS.: 93-95).

FIG. 32A illustrates structure of domains and sequences of hpIgR and shows that D1 is necessary for IgA binding to hpIgR. The figure is adapted from Stadtmueller et al., Elife, Mar. 4, 2016, e10640 (SEQ ID NOS.: 250-252).

FIG. 32B shows the structure of secretory IgA1 (sIgA1), the complex between dimeric IgA and secretory component, obtained by constrained modelling of solution scattering and AUC information (created from PDB ID 3CHN). Heavy chain is shown in orange, light chain is shown in green, J chain is shown in pink and secretory component is shown in teal. The figure is adapted from Bonner et al., Mucosal Immunol., 2:74-84 (2009).

FIGS. 33A-33D show the results of VHH/IgA competition studies of Example 6. The crystal structures in FIG. 33A is adapted from Stadtmueller et al., Elife, Mar. 4, 2016, e10640 (SEQ ID NOS.: 250 and 253-257). FIG. 33B shows a cartoon representation of hpIgR domain-1 created from PDB ID 5D4K. CDR1, CDR2 and CDR3 of hpIgR domain-1 are shown in orange, pink and light red, respectively, wherein hpIgR domain-1 CDRs were swapped with corresponding teleost fish CDRs to test the influence of hpIgR domain-1 CDRs on VHH binding. FIG. 33C shows IgA binding to immobilized pIgR constructions, including KD values (KD, Kon, or Koff). FIG. 33D shows kinetic parameters for VHH2 and VHH3 binding to sensor immobilized HIS-tagged pIgR protein constructs. The KD, Kon, or Koff are shown in the lower left, upper left and upper right panels, respectively. FIG. 33D shows that the hD1_tCDR2 construct did not show binding to both VHH2 and VHH3. Binding kinetic parameters were obtained by bio-layer interferometry, and the fold change in K_{D} values for VHH2 and VHH3 binding to pIgR domain constructs relative to full-length hpIgR ECD is shown in shown in the lower right panel.

FIG. 34 shows data describing how VHH2 and VHH3 compete with one another for binding to pIgR.

FIG. 35 illustrates that four molecules (VHH3, VHH4, VHH5 and VHH6) recognize buried epitopes on pIgR.

FIGS. 36A-36B shows that VHH3 recognizes a complex epitope on the hpIgR domain-1 interface.

FIGS. 37A-37B show results of VHH-mono-Fc molecules in forward and reverse transcytosis assays using MDCK-hpIgR monolayers, as described in Example 7. These results demonstrate bidirectional transport. FIG. 37A shows the results for the forward transcytosis (basolateral to apical direction), wherein 20 μg of test or control VHH-mono-Fc molecules were added to basolateral chamber and fold increase in apical [VHH] over control is shown at 24 hours (light gray) and 48 hours (dark gray) post treatment. For forward transcytosis, five VHH-mono-Fc molecules comprising a VHH2, VHH6, VHH9, VHH11 or VHH12 domain showed >20-fold increase in their apical concentration relative to control VHH-mono-Fc molecules at 48 hours, whereas VHH-mono-Fc molecules comprising a VHH4 domain showed a 15-fold increase in its apical concentration. FIG. 37B shows the results of reverse transcytosis (apical to basolateral direction), wherein 20 μg of test or control VHH-mono-Fc molecules were added to apical chamber and fold increase in basolateral [VHH] over control is shown at 24 hours (light gray) and 48 hours (dark gray) post treatment. VHH-mono-Fc molecules comprising a VHH6, VHH11, or VHH12 domain showed >10-fold increase in their basolateral concentration relative to control VHH-mono-Fc molecules at 48 hours. For reverse transcytosis, VHH-mono-Fc molecules comprising a VHH2, VHH4 or VHH9 domain showed >5-fold increase in their basolateral concentration relative to control VHH-mono-Fc molecules at 48 hours. Results for FIGS. 37A and 37B were obtained from three independent experiments, each containing two technical replicates.

FIGS. 38A-38B show results of VHH-mono-Fc molecules in forward and reverse transcytosis assays using MDCK-hpIgR monolayers, as described in Example 7. These resulted demonstrate bidirectional transport. To test forward transcytosis activity, 20 μg of test or control VHH-mono-Fc molecules were added to basolateral chamber and the amount of apical VHH-mono-Fc at 24 and 48 hours post treatment was quantified (B to A assay). To test reverse transcytosis activity, 20 μg of test or control VHH-mono-Fc molecules were added to apical chamber and the amount of basolateral VHH at 24 and 48 hours post treatment was quantified (A to B assay). Apical VHH (m) in B to A assay is shown in light gray and basolateral VHH (m) in A to B assay is shown in dark gray. FIG. 38A shows the comparison of forward and reverse transport of VHH-mono-Fc molecules at 24 hours post VHH treatment. FIG. 38B shows the comparison of forward and reverse transport of VHH-mono-Fc molecules at 48 hours post VHH treatment.

FIGS. 39A-39B show results for forward and reverse transcytosis kinetics of VHH-mono-Fc molecules across MDCK-hpIgR monolayers, as described in Example 7. FIG. 39A shows the results of forward transcytosis kinetics (basolateral to apical direction), wherein 20 μg of test or control VHH-mono-Fc molecules were added to the basolateral chamber. The amount of VHH present in the apical chamber (μg) was quantified and shown at different time points (0, 4, 8, 12, 24, 36 and 48 hours) post VHH treatment. The concentration of VHH-mono-Fc molecules increased over time in the apical chamber. For eight VHH-mono-Fc molecules, >10% of the basolateral VHH input (2 μg) was transported to the apical chamber (except VHH-mono-Fc molecules comprising a VHH3 or VHH7 domain). FIG. 39B shows the results of reverse transcytosis kinetics (apical to basolateral direction), wherein 20 μg of test or control VHH-mono-Fc molecules were added to the apical chamber. The amount of VHH present in the basolateral chamber (μg) was quantified and shown at different time points (0, 4, 8, 12, 24, 36 and 48 hours) post VHH treatment. The concentration of VHH-mono-Fc molecules increased over time in the basolateral chamber. For six VHH-mono-Fc molecules, >10% of the apical VHH input (2 μg) was transported to the basolateral chamber (VHH-mono-Fc molecules comprising a VHH2, VHH4, VHH6, VHH9, VHH11 or VHH12 domain).

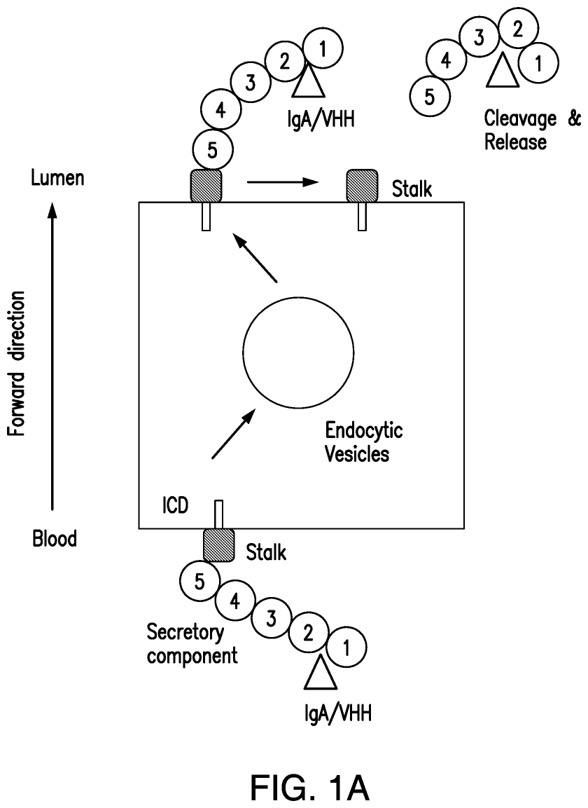

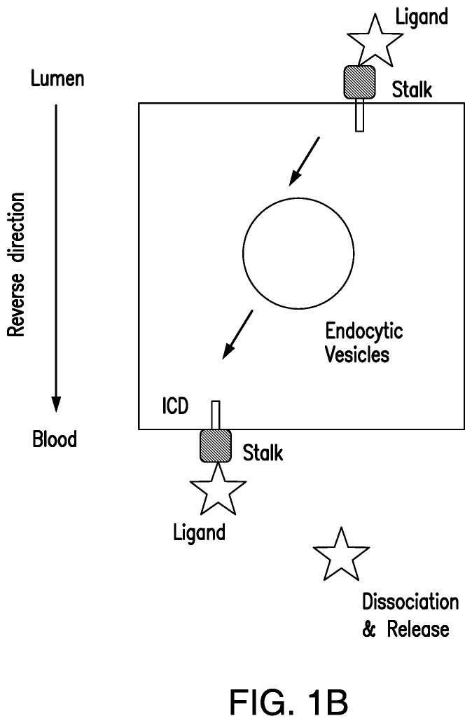

### 5. DETAILED DESCRIPTION

The present disclosure is based in part on the surprising finding that single domain antibodies (e.g., VHH domains) that bind to pIgR as provided herein are capable of transporting or facilitating to transport agents from an apical surface of a polymeric immunoglobulin receptor (pIgR)-expressing cell to a basolateral surface of the pIgR-expressing cell, and thus provide an effective method for administering therapeutic molecules (including diagnostic molecules), e.g., to systemic circulation or lamina propria or gastrointestinal tract of a subject, via, e.g., oral delivery, buccal delivery, nasal delivery or inhalation delivery.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)2 fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; PlUckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “antigen-binding fragment,” “antigen-binding domain,” “antigen-binding region,” and similar terms refer to that portion of a binding molecule, which comprises the amino acid residues that interact with an antigen and confer on the binding agent its specificity and affinity for the antigen (e.g., the CDRs). “Antigen-binding fragment” as used herein include “antibody fragment,” which comprise a portion of an intact antibody, such as the antigen-binding or variable region of the intact antibody. Examples of antibody fragments include, without limitation, Fab, Fab′, F(ab′)₂, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single variable domain antibodies (sdAbs) (see, e.g., Woolven et al., 1999, Immunogenetics 50: 98-101; and Streltsov et al., 2004, Proc Natl Acad Sci USA. 101:12444-49); and multispecific antibodies formed from antibody fragments.

“Single domain antibody” or “sdAb” as used herein refers to a single monomeric variable antibody domain and which is capable of antigen binding (e.g., single domain antibodies that bind to pIgR). Single domain antibodies include VHH domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, dromedary, alpaca and guanaco, as described herein. Other species besides Camelidae may produce heavy chain antibodies naturally devoid of light chain; VHHs derived from such other species are within the scope of the disclosure. In some embodiments, the single domain antibody (e.g., VHH) provided herein has a structure of FR1-CDR1-FR2-CDR2-FR3-CDR3-FR4. Single domain antibodies may be genetically fused or chemically conjugated to another molecule (e.g., an agent) as described herein.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen may be cross-reactive with related antigens. In certain embodiments, a binding molecule or antigen binding domain that binds to or specifically binds to an antigen does not cross-react with other antigens. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassays (MA) and enzyme linked immunosorbent assays (ELISAs). Typically a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis or RIA. With regard terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. A binding molecule or antigen binding domain that binds to an antigen includes one that is capable of binding the antigen with sufficient affinity such that the binding molecule is useful, for example, as a diagnostic agent in targeting the antigen. In certain embodiments, a binding molecule or antigen binding domain that binds to an antigen has a dissociation constant (K_{D}) of less than or equal to 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species (e.g., between human and cyno species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a MA, for example, performed with the Fab version of an antibody of interest and its antigen (Chen et al., 1999, J. Mol Biol 293:865-81). The K_{D} or K_{D} value may also be measured by using biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kon” may also be determined with the same biolayer interferometry (BLI) or surface plasmon resonance (SPR) techniques described above using, for example, the Octet®Red96, the Biacore®TM-2000, or the Biacore®TM-3000 system.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise a single domain antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1: 755-68). Also available for the preparation of human monoclonal antibodies are methods described in Cole et al., Monoclonal Antibodies and Cancer Therapy 77 (1985); Boerner et al., 1991, J. Immunol. 147(1):86-95; and van Dijk and van de Winkel, 2001, Curr. Opin. Pharmacol. 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6(5):561-66; Bruggemann and Taussing, 1997, Curr. Opin. Biotechnol. 8(4):455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid iomerizatio or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: a, 6, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309: 657-70. Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are noted below.

| Loop | Kabat | AbM | Chothia | Contact | IMGT |
|---|---|---|---|---|---|
| CDR L1 | L24 - - - L34 | L24 - - - L34 | L24 - - - L34 | L30 - - - L36 | L27 - - - L38 |
| CDR L2 | L50 - - - L56 | L50 - - - L56 | L50 - - - L56 | L46 - - - L55 | L56 - - - L65 |
| CDR L3 | L89 - - - L97 | L89 - - - L97 | L89 - - - L97 | L89 - - - L96 | L105 - - - L117 |
| CDR H1 | H31 - - - H35B | H26 - - - H35B | H26 - - - H32..34 | H30 - - - H35B | H27 - - - H38 |
| | (Kabat | | | | |
| | Numbering) | | | | |
| CDR H1 | H31 - - - H35 | H26 - - - H35 | H26 - - - H32 | H30 - - - H35 | |
| | (Chothia | | | | |
| | Numbering) | | | | |
| CDR H2 | H50 - - - H65 | H50 - - - H58 | H52 - - - H56 | H47 - - - H58 | H56 - - - H65 |
| CDR H3 | H95 - - - H102 | H95 - - - H102 | H95 - - - H102 | H93 - - - H101 | H105 - - - H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CHL CH2, and CH3 regions of the heavy chain and the CL region of the light chain.

The term “framework” or “FR” refers to those variable region residues flanking the CDRs. FR residues are present, for example, in chimeric, humanized, human, domain antibodies (e.g., single domain antibodies), diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody comprising a single domain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include but is not limited to an increase in forward or reverse transcytosis, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding a single domain antibody or an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a binding molecule (e.g., an antibody), for example, in isolated or purified form, together with a suitable amount of excipients.

The term “effective amount” or “therapeutically effective amount” as used herein refers to the amount of a single domain antibody or a therapeutic molecule comprising an agent and the single domain antibody or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., diabetes or a cancer).

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2. Single Domain Antibodies

5.2.1 Single Domain Antibodies Targeting pIgR

Provided herein are single domain antibodies (e.g., VHH domains) capable of binding to polymeric immunoglobulin receptor (pIgR), that can act as a delivery domain for therapeutic agents.

In various embodiments, the single domain antibodies (e.g., VHH domains) provided herein bind to human pIgR (Genbank ID: CR749533) (see Turula, H. & Wobus, C. E. The Role of the Polymeric Immunoglobulin Receptor and Secretory Immunoglobulins during Mucosal Infection and Immunity. Viruses 10 (2018)). In other embodiments, the single domain antibodies (e.g., VHH domains) provided herein bind to mouse pIgR.

Human pIgR (hpIgR) is an 82 kDa, single-pass transmembrane receptor containing a 620-residue extracellular domain (ECD), a 23-residue transmembrane domain and a 103-residue intracellular domain.

pIgR transports soluble polymeric forms of IgA and IgM into apical mucosal tissues from the basolateral side of the epithelium. The process of transporting polymeric immunoglobulins from the basolateral to apical side is transcytosis. Following transcytosis, the pIgR ECD that contains five domains (secretory component) is proteolytically cleaved and released into mucus with or without IgA. In addition to transcytosis, pIgR has several different functions that include, but are not limited to, conferring stability to IgA, immune exclusion, anti-inflammatory properties and homeostasis of commensals in the mucosal immune system.

Approximately 75% of total daily antibody production is directed to IgA molecules. In humans, there are two Ca genes encoding IgA subclass: IgA1 and IgA2 (IgA2m(1) and (2) allotypes). IgA1 has elongated hinge region lacking in IgA2, that contains several 0-glycan sites and is susceptible to proteolytic cleavage. Endogenous IgA is present in various forms in a compartment-dependent manner. Monomeric IgA (mIgA) is the predominant form in serum (at a concentration of 1-3 mg/mL), primarily as IgA1 (about 90%) produced in bone marrow. Dimeric IgA (dIgA) is formed via S-S bridging of the C-terminal Fc tailpiece with J chain. dIgA is produced locally at target site of action and transported across mucosal surface into secretions of respiratory, GI and genitourinary tracts. Secretory IgA (S-IgA) is formed via dIgA complex with extracellular domain of polymeric Ig receptor (pIgR). Cleavage of secretory component (SC) at the mucosal surface of epithelial cells releases S-IgA.

The polymeric immunoglobulin receptor (pIgR) binds to soluble dimeric IgA via Fc and J-chain mediated interactions. pIgR does not bind or transport IgG molecules across mucosal epithelium. Though IgG molecules lack a lumen-targeted active transport mechanism, conferring pIgR-binding abilities to IgG can mediate selective transport of IgG antibodies into the mucosal lumen.

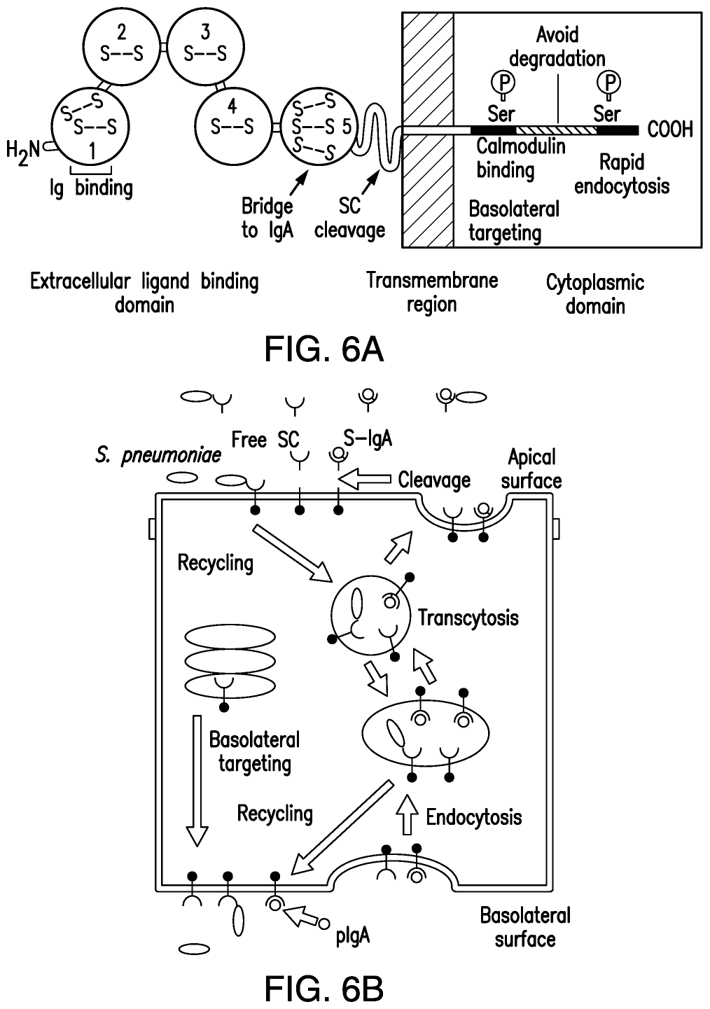

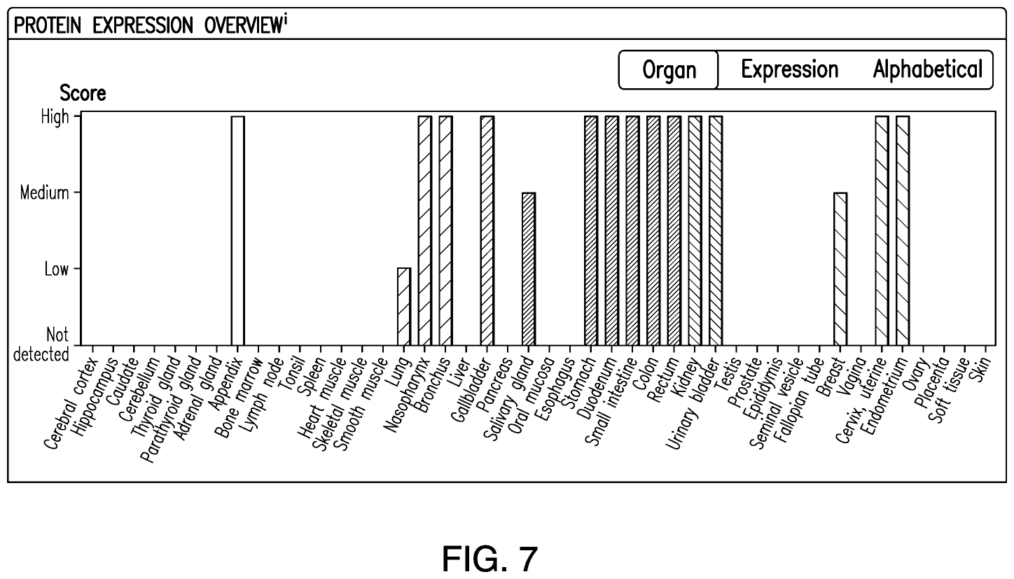

The structure of pIgR is summarized in FIG. 6A. A mechanism of pIgR-mediated transport is summarized in FIG. 6B. The expression of pIgR in various organs is shown in FIG. 7.

It is a surprising finding by the present disclosure that the single domain antibodies provided herein transport from an apical surface to a basolateral surface (reverse transcytosis) as well as from the basolateral to apical side (transcytosis).

In some embodiments, the single domain antibody (e.g., VHH domain) provided herein competes with IgA binding to the pIgR. In some embodiments, the single domain antibody (e.g., VHH domain) provided herein promotes IgA binding to the pIgR. In some embodiments, the K_{D} of the binding of the single domain antibody (e.g., VHH domain) provided herein to pIgR is from 4 to 525 nM. In some embodiments, the K_{D} of the binding of the single domain antibody (e.g., VHH domain) provided herein to pIgR is less than 525 nM. In some embodiments, the K_{D} of the binding of the single domain antibody (e.g., VHH domain) provided herein to pIgR is less than 400 nM. In some embodiments, the K_{D} of the binding of the single domain antibody (e.g., VHH domain) provided herein to pIgR is less than 350 nM. In some embodiments, the K_{D} of the binding of the single domain antibody (e.g., VHH domain) provided herein to pIgR is less than 300 nM. In some embodiments, the K_{D} of the binding of the single domain antibody (e.g., VHH domain) provided herein to pIgR is less than 250 nM. In some embodiments, the K_{D} of the binding of the single domain antibody (e.g., VHH domain) provided herein to pIgR is less than 200 nM. In some embodiments, the K_{D} of the binding of the single domain antibody (e.g., VHH domain) provided herein to pIgR is less than 150 nM. In some embodiments, the K_{D} of the binding of the single domain antibody (e.g., VHH domain) provided herein to pIgR is less than 100 nM. In some embodiments, the K_{D} of the binding of the single domain antibody (e.g., VHH domain) provided herein to pIgR is less than 50 nM. In some embodiments, the K_{D} of the binding of the single domain antibody (e.g., VHH domain) provided herein to pIgR is from 4 to 525 nm. In some embodiments, the K_{D} of the binding of the single domain antibody (e.g., VHH domain) provided herein to pIgR is from 4 to 34 nm. Bio-layer interferometry experiments described herein show 8 VHH domain binders having K_{D} values of <50 nM for binding to the human pIgR ectodomain (see Table 1).

In some embodiments, the Tₘ of the single domain antibody (e.g., VHH domain) is from 53 to 77° C. In some embodiments, the Tₘ of the single domain antibody (e.g., VHH domain) is from 53.9 to 76.4° C. In some embodiments, the Tₘ of the single domain antibody (e.g., VHH domain) is from 61 to 77° C. In some embodiments, the Tₘ of the single domain antibody (e.g., VHH domain) is from 61 to 71° C.

In some embodiments, the EC50 value for single domain antibody (e.g., VHH domain) binding to an MDCK-hpIgR cell is less than 10 nM. Six such binders comprising a VHH domain are described in Table 1.

In one aspect, provided herein is a VHH domain that binds to domain 1 of pIgR, wherein the VHH domain comprises the CDR1, CDR2 and/or CDR3 sequence of VHH2 or VHH3 described herein. Accordingly, in some embodiments, the VHH domain that bind to domain 1 of pIgR comprises the CDR1, CDR2 and CDR3 sequence of:

VHH2:
i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237); or

VHH3:
i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238).

In one aspect, provided herein is a VHH domain that binds to domain 2 of pIgR, wherein the VHH domain comprises the CDR1, CDR2 and/or CDR3 sequence of VHH4 or VHH6 described herein. Accordingly, in some embodiments, the VHH domain that bind to domain 2 of pIgR comprises the CDR1, CDR2 and CDR3 sequence of:

VHH4:
i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
ii) the CDR1 sequence of GTSVSSN (SEQ ID NO: 12), the CDR2 sequence of DRIAT (SEQ ID NO: 42) or MA (SEQ ID NO: 262), and the CDR3 sequence of PLTAR (SEQ ID NO: 74) or LTA (SEQ ID NO: 275);
iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239); or

VHH6:
i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277); or
iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241).

In one aspect, provided herein is a VHH domain that binds to domain 4-5 of pIgR, wherein the VHH domain comprises the CDR1, CDR2 and/or CDR3 sequence of VHH5, VHH7, VHH9, VHH10 or VHH11 described herein. Accordingly, in some embodiments, the VHH domain that bind to domain 4-5 of pIgR comprises the CDR1, CDR2 and CDR3 sequence of:

VHH5:
i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240); VHH7:

i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);

VHH9:
i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);

VHH10:
i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244); or

VHH11:
i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245).

In one aspect, provided herein is a VHH domain that binds to domain 5 of pIgR, wherein the VHH domain comprises the CDR1, CDR2 and/or CDR3 sequence of VHH12 described herein. Accordingly, in some embodiments, the VHH domain that bind to domain 5 of pIgR comprises the CDR1, CDR2 and CDR3 sequence of:

VHH12:
i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

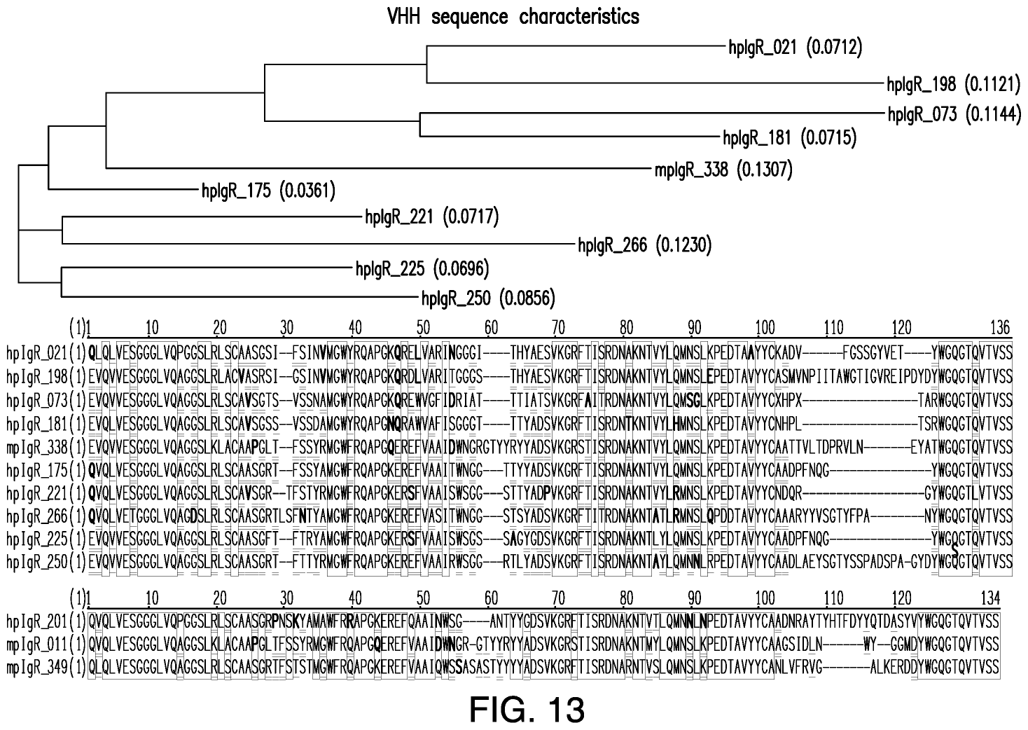

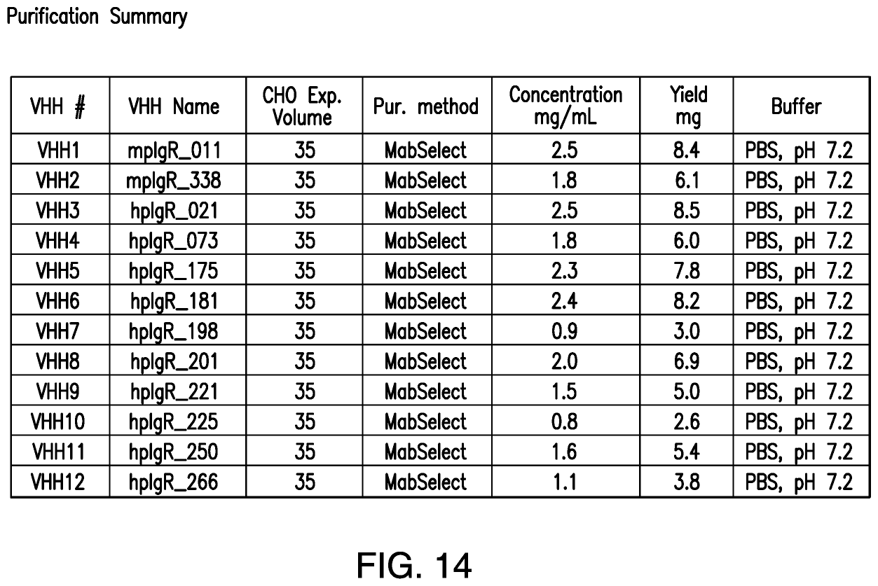

In some embodiments, the single domain antibodies provide herein are VHH domains. Exemplary VHH domains are generated as described below, and these VHH domains (referred to as mpIgR_011, hpIgR_021, hpIgR_073, hpIgR_175, hpIgR_181, hpIgR_198, hpIgR_201, hpIgR_221, hpIgR_225, hpIgR_250, hpIgR_266, mpIgR_338, and hpIgR_349) share some sequence characteristics, as shown in FIG. 13. Regions of highly conserved sequence similarity are shown in yellow. As indicated in FIG. 14, mpIgR_011 is VHH1, hpIgR_021 is VHH3, hpIgR_073 is VHH4, hpIgR_175 is VHH5, hpIgR_181 is VHH6, hpIgR_198 is VHH7, hpIgR_201 is VHH8, hpIgR_221 is VHH9, hpIgR_225 is VHH10, hpIgR_250 is VHH11, hpIgR_266 is VHH12, and mpIgR_338 is VHH2.

In some embodiments, the single domain antibody provided herein comprises one or more CDR sequences of any one of VHH1, VHH2, VHH3, VHH4, VHH5, VHH6, VHH7, VHH9, VHH10, VHH11, and VHH12.

Thus, in some embodiments, provided herein is a single domain antibody that binds to pIgR comprising the following structure: FR1-CDR1-FR2-CDR2-FR3-CDR3-FR4, wherein the CDR sequences are selected for those in VHH1, VHH2, VHH3, VHH4, VHH5, VHH6, VHH7, VHH9, VHH10, VHH11, and VHH12.

More specifically, provided herein is a single domain antibody that binds to pIgR comprising the following structure: FR1-CDR1-FR2-CDR2-FR3-CDR3-FR4, wherein
(i) CDR1 has an amino acid sequence selected from a group consisting of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), and GRTLSFNTYAMG (SEQ ID NO: 183);
(ii) CDR2 has an amino acid sequence selected from a group consisting of

(SEQ ID NO: 30)
AIDWNGRGTYYRYYADSVKG,
(SEQ ID NO: 31)
RINGGGITHYAESVKG,
(SEQ ID NO: 32)
FIDRIATTTIATSVKG,
(SEQ ID NO: 33)
AITWNGGTTYYADSVKG,
(SEQ ID NO: 34)
FISGGGTTTYADSVKG,
(SEQ ID NO: 35)
RITGGGSTHYAESVKG,
(SEQ ID NO: 36)
AISWSGGSTTYADPVKG,
(SEQ ID NO: 37)
AISWSGSSAGYGDSVKG,
(SEQ ID NO: 38)
AIRWSGGRTLYADSVKG,
(SEQ ID NO: 39)
SITWNGGSTSYADSVKG,
(SEQ ID NO: 40)
DWNGRGTYY,
(SEQ ID NO: 260)
WNGRGTY,
(SEQ ID NO: 41)
NGGGI,
(SEQ ID NO: 261)
GGG,
(SEQ ID NO: 42)
DRIAT,
(SEQ ID NO: 43)
TWNGGT,
(SEQ ID NO: 263)
WNGG,
(SEQ ID NO: 44)
SGGGT,
(SEQ ID NO: 264)
GGG,
(SEQ ID NO: 45)
TGGGS,
(SEQ ID NO: 265)
GGG,
(SEQ ID NO: 46)
SWSGGS,
(SEQ ID NO: 266)
WSGG,
(SEQ ID NO: 47)
SWSGSS,
(SEQ ID NO: 267)
WSGS,
(SEQ ID NO: 48)
RWSGGR,
(SEQ ID NO: 268)
WSGG,
(SEQ ID NO: 49)
TWNGGS,
(SEQ ID NO: 269)
WNGG,
(SEQ ID NO: 50)
IDWNGRGTYY,
(SEQ ID NO: 270)
IDWNGRGTYYR,
(SEQ ID NO: 51)
INGGGIT,
(SEQ ID NO: 52)
IDRIATT,
(SEQ ID NO: 53)
ITWNGGTT,
(SEQ ID NO: 54)
ISGGGTT,
(SEQ ID NO: 55)
ITGGGST,
(SEQ ID NO: 56)
ISWSGGST,
(SEQ ID NO: 57)
ISWSGSSA,
(SEQ ID NO: 58)
IRWSGGRT,
(SEQ ID NO: 59)
ITWNGGST,
(SEQ ID NO: 184)
AIDWNGRGTYYRYYADSVKG,
(SEQ ID NO: 185)
RINGGGITHYAESVKG,
(SEQ ID NO: 186)
FIDRIATTTIATSVKG,
(SEQ ID NO: 187)
AITWNGGTTYYADSVKG,
(SEQ ID NO: 188)
FISGGGTTTYADSVKG,
(SEQ ID NO: 189)
RITGGGSTHYAESVKG,
(SEQ ID NO: 190)
AISWSGGSTTYADPVKG,
(SEQ ID NO: 191)
AISWSGSSAGYGDSVKG,
(SEQ ID NO: 192)
AIRWSGGRTLYADSVKG,
(SEQ ID NO: 193)
SITWNGGSTSYADSVKG,
(SEQ ID NO: 194)
FVAAIDWNGRGTYYRY,
(SEQ ID NO: 195)
LVARINGGGITH,
(SEQ ID NO: 196)
WVGFIDRIATTT,
(SEQ ID NO: 197)
FVAAITWNGGTTY,
(SEQ ID NO: 198)
WVAFISGGGTTT,
(SEQ ID NO: 199)
LVARITGGGSTH,
(SEQ ID NO: 200)
FVAAISWSGGSTT,
(SEQ ID NO: 201)
FVAAISWSGSSAG,
(SEQ ID NO: 202)
FVAAIRWSGGRTL,
(SEQ ID NO: 203)
FVASITWNGGSTS,
(SEQ ID NO: 204)
AIDWNGRGTYYRY,
(SEQ ID NO: 205)
RINGGGITH,
(SEQ ID NO: 206)
FIDRIATTT,
(SEQ ID NO: 207)
AITWNGGTTY,
(SEQ ID NO: 208)
FISGGGTTT,
(SEQ ID NO: 209)
RITGGGSTH,
(SEQ ID NO: 210)
AISWSGGSTT,
(SEQ ID NO: 211)
AISWSGSSAG,
(SEQ ID NO: 212)
AIRWSGGRTL,
and
(SEQ ID NO: 213)
SITWNGGSTS;

and
(iii) CDR3 has an amino acid sequence selected from a group consisting of

(SEQ ID NO: 60)
GSIDLNWYGGMDY,
(SEQ ID NO: 61)
TTVLTDPRVLNEYAT,
(SEQ ID NO: 62)
DVFGSSGYVETY,
(SEQ ID NO: 63)
PLTAR,
(SEQ ID NO: 64)
DPFNQGY,
(SEQ ID NO: 65)
PLTSR,
(SEQ ID NO: 66)
MVNPIITAWGTIGVREIPDYDY,
(SEQ ID NO: 67)
DQRGY,
(SEQ ID NO: 271)
QRGY,
(SEQ ID NO: 68)
DPFNQGY,
(SEQ ID NO: 69)
DLAEYSGTYSSPADSPAGYDY,
(SEQ ID NO: 70)
ARYYVSGTYFPANY,
(SEQ ID NO: 71)
GSIDLNWYGGMDY,
(SEQ ID NO: 272)
SIDLNWYGGMD,
(SEQ ID NO: 72)
TTVLTDPRVLNEYAT,
(SEQ ID NO: 273)
TVLTDPRVLNEYA,
(SEQ ID NO: 73)
DVFGSSGYVETY,
(SEQ ID NO: 274)
VFGSSGYVET,
(SEQ ID NO: 74)
PLTAR,
(SEQ ID NO: 275)
LTA,
(SEQ ID NO: 75)
DPFNQGY,
(SEQ ID NO: 276)
PFNQG,
(SEQ ID NO: 76)
PLTSR,
(SEQ ID NO: 277)
LTS,
(SEQ ID NO: 77)
MVNPIITAWGTIGVREIPDYDY,
(SEQ ID NO: 278)
VNPIITAWGTIGVREIPDYD,
(SEQ ID NO: 78)
DQRGY,
(SEQ ID NO: 279)
RG,
(SEQ ID NO: 79)
DPFNQGY,
(SEQ ID NO: 280)
PFNQG,
(SEQ ID NO: 80)
DLAEYSGTYSSPADSPAGYDY,
(SEQ ID NO: 281)
LAEYSGTYSSPADSPAGYD,
(SEQ ID NO: 81)
ARYYVSGTYFPANY,
(SEQ ID NO: 282)
RYYVSGTYFPAN,
(SEQ ID NO: 82)
CAAGSIDLNWYGGMDY,
(SEQ ID NO: 283)
AAGSIDLNWYGGMDY,
(SEQ ID NO: 83)
CAATTVLTDPRVLNEYAT,
(SEQ ID NO: 284)
AATTVLTDPRVLNEYAT,
(SEQ ID NO: 84)
KADVFGSSGYVETY,
(SEQ ID NO: 85)
NHPLTAR,
(SEQ ID NO: 86)
AADPFNQGY,
(SEQ ID NO: 87)
NHPLTSR,
(SEQ ID NO: 88)
ASMVNPIITAWGTIGVREIPDYDY,
(SEQ ID NO: 89)
NDQRGY,
(SEQ ID NO: 90)
AADPFNQGY,
(SEQ ID NO: 91)
AADLAEYSGTYSSPADSPAGYDY,
(SEQ ID NO: 92)
AAARYYVSGTYFPANY,
(SEQ ID NO: 214)
GSIDLNWYGGMDY,
(SEQ ID NO: 215)
TTVLTDPRVLNEYAT,
(SEQ ID NO: 216)
DVFGSSGYVETY,
(SEQ ID NO: 217)
PLTAR,
(SEQ ID NO: 218)
DPFNQGY,
(SEQ ID NO: 219)
PLTSR,
(SEQ ID NO: 220)
MVNPIITAWGTIGVREIPDYDY,
(SEQ ID NO: 221)
QRGY,
(SEQ ID NO: 222)
DPFNQGY,
(SEQ ID NO: 223)
DLAEYSGTYSSPADSPAGYDY,
(SEQ ID NO: 224)
ARYYVSGTYFPANY,
(SEQ ID NO: 225)
AAGSIDLNWYGGMD,
(SEQ ID NO: 226)
AATTVLTDPRVLNEYA,
(SEQ ID NO: 227)
KADVFGSSGYVET,
(SEQ ID NO: 228)
NHPLTA,
(SEQ ID NO: 229)
AADPFNQG,
(SEQ ID NO: 230)
NHPLTS,
(SEQ ID NO: 231)
ASMVNPIITAWGTIGVREIPDYD,
(SEQ ID NO: 232)
NDQRG,
(SEQ ID NO: 233)
AADPFNQG,
(SEQ ID NO: 234)
AADLAEYSGTYSSPADSPAGYD,
(SEQ ID NO: 235)
AAARYYVSGTYFPAN,
(SEQ ID NO: 236)
GSIDLNWYGGMDY,
(SEQ ID NO: 237)
TTVLTDPRVLNEYAT,
(SEQ ID NO: 238)
DVFGSSGYVETY,
(SEQ ID NO: 239)
PLTAR,
(SEQ ID NO: 240)
DPFNQGY,
(SEQ ID NO: 241)
PLTSR,
(SEQ ID NO: 242)
MVNPIITAWGTIGVREIPDYDY,
(SEQ ID NO: 243)
QRGY,
(SEQ ID NO: 244)
DPFNQGY,
(SEQ ID NO: 245)
DLAEYSGTYSSPADSPAGYDY,
and
(SEQ ID NO: 246)
ARYYVSGTYFPANY.

In some embodiments, provided herein is a single domain antibody that binds to pIgR comprising a variable region (e.g., VH) comprising CDR1, CDR2, and CDR3 of any one of antibodies as set forth in Table 1.

In various embodiments of the aspects described herein the single domain antibody comprises a CDR1 sequence present in VHH1, e.g., the CDR1 sequence of SYRMG (SEQ ID NO: 1). In various embodiments of the aspects described herein the single domain antibody comprises a CDR1 sequence present in VHH2, e.g., the CDR1 sequence of SYRMG (SEQ ID NO: 1). In various embodiments of the aspects described herein the single domain antibody comprises a CDR1 sequence present in VHH3, e.g., the CDR1 sequence of INVMG (SEQ ID NO: 2). In various embodiments of the aspects described herein the single domain antibody comprises a CDR1 sequence present in VHH4, e.g., the CDR1 sequence of SNAMG (SEQ ID NO: 3). In various embodiments of the aspects described herein the single domain antibody comprises a CDR1 sequence present in VHH5, e.g., the CDR1 sequence of SYAMG (SEQ ID NO: 4). In various embodiments of the aspects described herein the single domain antibody comprises a CDR1 sequence present in VHH6, e.g., the CDR1 sequence of SDAMG (SEQ ID NO: 5). In various embodiments of the aspects described herein the single domain antibody comprises a CDR1 sequence present in VHH7, e.g., the CDR1 sequence of INVMG (SEQ ID NO: 6). In various embodiments of the aspects described herein the single domain antibody comprises a CDR1 sequence present in VHH9, e.g., the CDR1 sequence of TYRMG (SEQ ID NO: 7). In various embodiments of the aspects described herein the single domain antibody comprises a CDR1 sequence present in VHH10, e.g., the CDR1 sequence of RYAMG (SEQ ID NO: 8). In various embodiments of the aspects described herein the single domain antibody comprises a CDR1 sequence present in VHH11, e.g., the CDR1 sequence of TYRMG (SEQ ID NO: 259). In various embodiments of the aspects described herein the single domain antibody comprises a CDR1 sequence present in VHH12, e.g., the CDR1 sequence of FNTYAMG (SEQ ID NO: 9).

In various embodiments of the aspects described herein the single domain antibody comprises a CDR1 sequence present in VHH1, e.g., the CDR1 sequence of GLTFSSY (SEQ ID NO: 10). In various embodiments of the aspects described herein the single domain antibody comprises a CDR1 sequence present in VHH2, e.g., the CDR1 sequence of GLTFSSY (SEQ ID NO: 10). In various embodiments of the aspects described herein the single domain antibody comprises a CDR1 sequence present in VHH3, e.g., the CDR1 sequence of GSIF SIN (SEQ ID NO: 11). In various embodiments of the aspects described herein the single domain antibody comprises a CDR1 sequence present in VHH4, e.g., the CDR1 sequence of GTSVSSN (SEQ ID NO: 12). In various embodiments of the aspects described herein the single domain antibody comprises a CDR1 sequence present in VHH5, e.g., the CDR1 sequence of GRTFSSY (SEQ ID NO: 13). In various embodiments of the aspects described herein the single domain antibody comprises a CDR1 sequence present in VHH6, e.g., the CDR1 sequence of GSSVSSD (SEQ ID NO: 14). In various embodiments of the aspects described herein the single domain antibody comprises a CDR1 sequence present in VHH7, e.g., the CDR1 sequence of RSIGSIN (SEQ ID NO: 15). In various embodiments of the aspects described herein the single domain antibody comprises a CDR1 sequence present in VHH9, e.g., the CDR1 sequence of GRTFSTY (SEQ ID NO: 16). In various embodiments of the aspects described herein the single domain antibody comprises a CDR1 sequence present in VHH10, e.g., the CDR1 sequence of GFTFTRY (SEQ ID NO: 17). In various embodiments of the aspects described herein the single domain antibody comprises a CDR1 sequence present in VHH11, e.g., the CDR1 sequence of GRTFTTY (SEQ ID NO: 18). In various embodiments of the aspects described herein the single domain antibody comprises a CDR1 sequence present in VHH12, e.g., the CDR1 sequence of GRTLSFNTY (SEQ ID NO: 19).

In various embodiments of the aspects described herein the single domain antibody comprises a CDR1 sequence present in VHH1, e.g., the CDR1 sequence of GLTFSSYR (SEQ ID NO: 20). In various embodiments of the aspects described herein the single domain antibody comprises a CDR1 sequence present in VHH2, e.g., the CDR1 sequence of GLTFSSYR (SEQ ID NO: 20). In various embodiments of the aspects described herein the single domain antibody comprises a CDR1 sequence present in VHH3, e.g., the CDR1 sequence of GSIFSINV (SEQ ID NO: 21). In various embodiments of the aspects described herein the single domain antibody comprises a CDR1 sequence present in VHH4, e.g., the CDR1 sequence of GTSVSSNA (SEQ ID NO: 22). In various embodiments of the aspects described herein the single domain antibody comprises a CDR1 sequence present in VHH5, e.g., the CDR1 sequence of GRTFSSYA (SEQ ID NO: 23). In various embodiments of the aspects described herein the single domain antibody comprises a CDR1 sequence present in VHH6, e.g., the CDR1 sequence of GSSVSSDA (SEQ ID NO: 24). In various embodiments of the aspects described herein the single domain antibody comprises a CDR1 sequence present in VHH7, e.g., the CDR1 sequence of RSIGSINV (SEQ ID NO: 25). In various embodiments of the aspects described herein the single domain antibody comprises a CDR1 sequence present in VHH9, e.g., the CDR1 sequence of GRTFSTYR (SEQ ID NO: 26). In various embodiments of the aspects described herein the single domain antibody comprises a CDR1 sequence present in VHH10, e.g., the CDR1 sequence of GFTFTRYA (SEQ ID NO: 27). In various embodiments of the aspects described herein the single domain antibody comprises a CDR1 sequence present in VHH11, e.g., the CDR1 sequence of GRTFTTYR (SEQ ID NO: 28). In various embodiments of the aspects described herein the single domain antibody comprises a CDR1 sequence present in VHH12, e.g., the CDR1 sequence of GRTLSFNTYA (SEQ ID NO: 29).

In various embodiments of the aspects described herein the single domain antibody comprises a CDR1 sequence present in VHH1, e.g., the CDR1 sequence of GLTFSSYRMG (SEQ ID NO: 154). In various embodiments of the aspects described herein the single domain antibody comprises a CDR1 sequence present in VHH2, e.g., the CDR1 sequence of GLTFSSYRMG (SEQ ID NO: 154). In various embodiments of the aspects described herein the single domain antibody comprises a CDR1 sequence present in VHH3, e.g., the CDR1 sequence of GSIFSINVMG (SEQ ID NO: 155). In various embodiments of the aspects described herein the single domain antibody comprises a CDR1 sequence present in VHH4, e.g., the CDR1 sequence of GTSVSSNAMG (SEQ ID NO: 156). In various embodiments of the aspects described herein the single domain antibody comprises a CDR1 sequence present in VHH5, e.g., the CDR1 sequence of GRTFSSYAMG (SEQ ID NO: 157). In various embodiments of the aspects described herein the single domain antibody comprises a CDR1 sequence present in VHH6, e.g., the CDR1 sequence of GSSVSSDAMG (SEQ ID NO: 158). In various embodiments of the aspects described herein the single domain antibody comprises a CDR1 sequence present in VHH7, e.g., the CDR1 sequence of RSIGSINVMG (SEQ ID NO: 159). In various embodiments of the aspects described herein the single domain antibody comprises a CDR1 sequence present in VHH9, e.g., the CDR1 sequence of GRTFSTYRMG (SEQ ID NO: 160). In various embodiments of the aspects described herein the single domain antibody comprises a CDR1 sequence present in VHH10, e.g., the CDR1 sequence of RYAMG GFTFTRYAMG (SEQ ID NO: 161). In various embodiments of the aspects described herein the single domain antibody comprises a CDR1 sequence present in VHH11, e.g., the CDR1 sequence of GRTFTTYRMG (SEQ ID NO: 162). In various embodiments of the aspects described herein the single domain antibody comprises a CDR1 sequence present in VHH12, e.g., the CDR1 sequence of GRTLSFNTYAMG (SEQ ID NO: 163).

In various embodiments of the aspects described herein the single domain antibody comprises a CDR1 sequence present in VHH1, e.g., the CDR1 sequence of GLTFSSY SSYRMG (SEQ ID NO: 164). In various embodiments of the aspects described herein the single domain antibody comprises a CDR1 sequence present in VHH2, e.g., the CDR1 sequence of SSYRMG (SEQ ID NO: 164). In various embodiments of the aspects described herein the single domain antibody comprises a CDR1 sequence present in VHH3, e.g., the CDR1 sequence of SINVMG (SEQ ID NO: 165). In various embodiments of the aspects described herein the single domain antibody comprises a CDR1 sequence present in VHH4, e.g., the CDR1 sequence of SSNAMG (SEQ ID NO: 166). In various embodiments of the aspects described herein the single domain antibody comprises a CDR1 sequence present in VHH5, e.g., the CDR1 sequence of SSYAMG (SEQ ID NO: 167). In various embodiments of the aspects described herein the single domain antibody comprises a CDR1 sequence present in VHH6, e.g., the CDR1 sequence of SSDAMG (SEQ ID NO: 168). In various embodiments of the aspects described herein the single domain antibody comprises a CDR1 sequence present in VHH7, e.g., the CDR1 sequence of SINVMG (SEQ ID NO: 169). In various embodiments of the aspects described herein the single domain antibody comprises a CDR1 sequence present in VHH9, e.g., the CDR1 sequence of STYRMG (SEQ ID NO: 170). In various embodiments of the aspects described herein the single domain antibody comprises a CDR1 sequence present in VHH10, e.g., the CDR1 sequence of TRYAMG (SEQ ID NO: 171). In various embodiments of the aspects described herein the single domain antibody comprises a CDR1 sequence present in VHH11, e.g., the CDR1 sequence of TTYRMG (SEQ ID NO: 172). In various embodiments of the aspects described herein the single domain antibody comprises a CDR1 sequence present in VHH12, e.g., the CDR1 sequence of SFNTYAMG (SEQ ID NO: 173).

In various embodiments of the aspects described herein the single domain antibody comprises a CDR1 sequence present in VHH1, e.g., the CDR1 sequence of GLTFSSYRMG (SEQ ID NO: 174). In various embodiments of the aspects described herein the single domain antibody comprises a CDR1 sequence present in VHH2, e.g., the CDR1 sequence of GLTFSSYRMG (SEQ ID NO: 174). In various embodiments of the aspects described herein the single domain antibody comprises a CDR1 sequence present in VHH3, e.g., the CDR1 sequence of GSIFSINVMG (SEQ ID NO: 175). In various embodiments of the aspects described herein the single domain antibody comprises a CDR1 sequence present in VHH4, e.g., the CDR1 sequence of GTSVSSNAMG (SEQ ID NO: 176). In various embodiments of the aspects described herein the single domain antibody comprises a CDR1 sequence present in VHH5, e.g., the CDR1 sequence of GRTFSSYAMG (SEQ ID NO: 177). In various embodiments of the aspects described herein the single domain antibody comprises a CDR1 sequence present in VHH6, e.g., the CDR1 sequence of GSSVSSDAMG (SEQ ID NO: 178). In various embodiments of the aspects described herein the single domain antibody comprises a CDR1 sequence present in VHH7, e.g., the CDR1 sequence of RSIGSINVMG (SEQ ID NO: 179). In various embodiments of the aspects described herein the single domain antibody comprises a CDR1 sequence present in VHH9, e.g., the CDR1 sequence of GRTFSTYRMG (SEQ ID NO: 180). In various embodiments of the aspects described herein the single domain antibody comprises a CDR1 sequence present in VHH10, e.g., the CDR1 sequence of GFTFTRYAMG (SEQ ID NO: 181). In various embodiments of the aspects described herein the single domain antibody comprises a CDR1 sequence present in VHH11, e.g., the CDR1 sequence of GRTFTTYRMG (SEQ ID NO: 182). In various embodiments of the aspects described herein the single domain antibody comprises a CDR1 sequence present in VHH12, e.g., the CDR1 sequence of GRTLSFNTYAMG (SEQ ID NO: 183).

In various embodiments of the aspects described herein the single domain antibody comprises a CDR2 sequence present in VHH1, e.g., the CDR2 sequence of AIDWNGRGTYYRYYADSVKG (SEQ ID NO: 30). In various embodiments of the aspects described herein the single domain antibody comprises a CDR2 sequence present in VHH2, e.g., the CDR2 sequence of AIDWNGRGTYYRYYADSVKG (SEQ ID NO: 30). In various embodiments of the aspects described herein the single domain antibody comprises a CDR2 sequence present in VHH3, e.g., the CDR2 sequence of RINGGGITHYAESVKG (SEQ ID NO: 31). In various embodiments of the aspects described herein the single domain antibody comprises a CDR2 sequence present in VHH4, e.g., the CDR2 sequence of FIDRIATTTIATSVKG (SEQ ID NO: 32). In various embodiments of the aspects described herein the single domain antibody comprises a CDR2 sequence present in VHH5, e.g., the CDR2 sequence of AITWNGGTTYYADSVKG (SEQ ID NO: 33). In various embodiments of the aspects described herein the single domain antibody comprises a CDR2 sequence present in VHH6, e.g., the CDR2 sequence of FISGGGTTTYADSVKG (SEQ ID NO: 34). In various embodiments of the aspects described herein the single domain antibody comprises a CDR2 sequence present in VHH7, e.g., the CDR2 sequence of RITGGGSTHYAESVKG (SEQ ID NO: 35). In various embodiments of the aspects described herein the single domain antibody comprises a CDR2 sequence present in VHH9, e.g., the CDR2 sequence of AISWSGGSTTYADPVKG (SEQ ID NO: 36). In various embodiments of the aspects described herein the single domain antibody comprises a CDR2 sequence present in VHH10, e.g., the CDR2 sequence of AISWSGSSAGYGDSVKG (SEQ ID NO: 37). In various embodiments of the aspects described herein the single domain antibody comprises a CDR2 sequence present in VHH11, e.g., the CDR2 sequence of AIRWSGGRTLYADSVKG (SEQ ID NO: 38). In various embodiments of the aspects described herein the single domain antibody comprises a CDR2 sequence present in VHH12, e.g., the CDR2 sequence of SITWNGGSTSYADSVKG (SEQ ID NO: 39).

In various embodiments of the aspects described herein the single domain antibody comprises a CDR2 sequence present in VHH1, e.g., the CDR2 sequence of DWNGRGTYY (SEQ ID NO: 40) or WNGRGTY (SEQ ID NO: 260). In various embodiments of the aspects described herein the single domain antibody comprises a CDR2 sequence present in VHH2, e.g., the CDR2 sequence of DWNGRGTYY (SEQ ID NO: 40) or WNGRGTY (SEQ ID NO: 260). In various embodiments of the aspects described herein the single domain antibody comprises a CDR2 sequence present in VHH3, e.g., the CDR2 sequence of NGGGI (SEQ ID NO: 41) or GGG (SEQ ID NO: 261). In various embodiments of the aspects described herein the single domain antibody comprises a CDR2 sequence present in VHH4, e.g., the CDR2 sequence of DRIAT (SEQ ID NO: 42) or RIA (SEQ ID NO: 262). In various embodiments of the aspects described herein the single domain antibody comprises a CDR2 sequence present in VHH5, e.g., the CDR2 sequence of TWNGGT (SEQ ID NO: 43) or WNGG (SEQ ID NO: 263). In various embodiments of the aspects described herein the single domain antibody comprises a CDR2 sequence present in VHH6, e.g., the CDR2 sequence of SGGGT (SEQ ID NO: 44) or GGG (SEQ ID NO: 264). In various embodiments of the aspects described herein the single domain antibody comprises a CDR2 sequence present in VHH7, e.g., the CDR2 sequence of TGGGS (SEQ ID NO: 45) or GGG (SEQ ID NO: 265). In various embodiments of the aspects described herein the single domain antibody comprises a CDR2 sequence present in VHH9, e.g., the CDR2 sequence of SWSGGS (SEQ ID NO: 46) or WSGG (SEQ ID NO: 266). In various embodiments of the aspects described herein the single domain antibody comprises a CDR2 sequence present in VHH10, e.g., the CDR2 sequence of SWSGGS (SEQ ID NO: 47) or WSGS (SEQ ID NO: 267). In various embodiments of the aspects described herein the single domain antibody comprises a CDR2 sequence present in VHH11, e.g., the CDR2 sequence of RWSGGR (SEQ ID NO: 48) or WSGG (SEQ ID NO: 268). In various embodiments of the aspects described herein the single domain antibody comprises a CDR2 sequence present in VHH12, e.g., the CDR2 sequence of TWNGGS (SEQ ID NO: 49) or WNGG (SEQ ID NO: 269).

In various embodiments of the aspects described herein the single domain antibody comprises a CDR2 sequence present in VHH1, e.g., the CDR2 sequence of IDWNGRGTYY (SEQ ID NO: 50) or IDWNGRGTYYR (SEQ ID NO: 270). In various embodiments of the aspects described herein the single domain antibody comprises a CDR2 sequence present in VHH2, e.g., the CDR2 sequence of IDWNGRGTYY (SEQ ID NO: 50) or IDWNGRGTYYR (SEQ ID NO: 270). In various embodiments of the aspects described herein the single domain antibody comprises a CDR2 sequence present in VHH3, e.g., the CDR2 sequence of INGGGIT (SEQ ID NO: 51). In various embodiments of the aspects described herein the single domain antibody comprises a CDR2 sequence present in VHH4, e.g., the CDR2 sequence of IDRIATT (SEQ ID NO: 52). In various embodiments of the aspects described herein the single domain antibody comprises a CDR2 sequence present in VHH5, e.g., the CDR2 sequence of ITWNGGTT (SEQ ID NO: 53). In various embodiments of the aspects described herein the single domain antibody comprises a CDR2 sequence present in VHH6, e.g., the CDR2 sequence of ISGGGTT (SEQ ID NO: 54). In various embodiments of the aspects described herein the single domain antibody comprises a CDR2 sequence present in VHH7, e.g., the CDR2 sequence of ITGGGST (SEQ ID NO: 55). In various embodiments of the aspects described herein the single domain antibody comprises a CDR2 sequence present in VHH9, e.g., the CDR2 sequence of ISWSGGST (SEQ ID NO: 56). In various embodiments of the aspects described herein the single domain antibody comprises a CDR2 sequence present in VHH10, e.g., the CDR2 sequence of ISWSGSSA (SEQ ID NO: 57). In various embodiments of the aspects described herein the single domain antibody comprises a CDR2 sequence present in VHH11, e.g., the CDR2 sequence of IRWSGGRT (SEQ ID NO: 58). In various embodiments of the aspects described herein the single domain antibody comprises a CDR2 sequence present in VHH12, e.g., the CDR2 sequence of ITWNGGST (SEQ ID NO: 59).

In various embodiments of the aspects described herein the single domain antibody comprises a CDR2 sequence present in VHH1, e.g., the CDR2 sequence of AIDWNGRGTYYRYYADSVKG (SEQ ID NO: 184). In various embodiments of the aspects described herein the single domain antibody comprises a CDR2 sequence present in VHH2, e.g., the CDR2 sequence of AIDWNGRGTYYRYYADSVKG (SEQ ID NO: 184). In various embodiments of the aspects described herein the single domain antibody comprises a CDR2 sequence present in VHH3, e.g., the CDR2 sequence of RINGGGITHYAESVKG (SEQ ID NO: 185). In various embodiments of the aspects described herein the single domain antibody comprises a CDR2 sequence present in VHH4, e.g., the CDR2 sequence of FIDRIATTTIATSVKG (SEQ ID NO: 186). In various embodiments of the aspects described herein the single domain antibody comprises a CDR2 sequence present in VHH5, e.g., the CDR2 sequence of AITWNGGTTYYADSVKG (SEQ ID NO: 187). In various embodiments of the aspects described herein the single domain antibody comprises a CDR2 sequence present in VHH6, e.g., the CDR2 sequence of FISGGGTTTYADSVKG (SEQ ID NO: 188). In various embodiments of the aspects described herein the single domain antibody comprises a CDR2 sequence present in VHH7, e.g., the CDR2 sequence of RITGGGSTHYAESVKG (SEQ ID NO: 189). In various embodiments of the aspects described herein the single domain antibody comprises a CDR2 sequence present in VHH9, e.g., the CDR2 sequence of AISWSGGSTTYADPVKG (SEQ ID NO: 190). In various embodiments of the aspects described herein the single domain antibody comprises a CDR2 sequence present in VHH10, e.g., the CDR2 sequence of AISWSGSSAGYGDSVKG (SEQ ID NO: 191). In various embodiments of the aspects described herein the single domain antibody comprises a CDR2 sequence present in VHH11, e.g., the CDR2 sequence of AIRWSGGRTLYADSVKG (SEQ ID NO: 192). In various embodiments of the aspects described herein the single domain antibody comprises a CDR2 sequence present in VHH12, e.g., the CDR2 sequence of SITWNGGSTSYADSVKG (SEQ ID NO: 193).

In various embodiments of the aspects described herein the single domain antibody comprises a CDR2 sequence present in VHH1, e.g., the CDR2 sequence of FVAAIDWNGRGTYYRY (SEQ ID NO: 194). In various embodiments of the aspects described herein the single domain antibody comprises a CDR2 sequence present in VHH2, e.g., the CDR2 sequence of FVAAIDWNGRGTYYRY (SEQ ID NO: 194). In various embodiments of the aspects described herein the single domain antibody comprises a CDR2 sequence present in VHH3, e.g., the CDR2 sequence of LVARINGGGITH (SEQ ID NO: 195). In various embodiments of the aspects described herein the single domain antibody comprises a CDR2 sequence present in VHH4, e.g., the CDR2 sequence of WVGFIDRIATTT (SEQ ID NO: 196). In various embodiments of the aspects described herein the single domain antibody comprises a CDR2 sequence present in VHH5, e.g., the CDR2 sequence of FVAAITWNGGTTY (SEQ ID NO: 197). In various embodiments of the aspects described herein the single domain antibody comprises a CDR2 sequence present in VHH6, e.g., the CDR2 sequence of WVAFISGGGTTT (SEQ ID NO: 198). In various embodiments of the aspects described herein the single domain antibody comprises a CDR2 sequence present in VHH7, e.g., the CDR2 sequence of LVARITGGGSTH (SEQ ID NO: 199). In various embodiments of the aspects described herein the single domain antibody comprises a CDR2 sequence present in VHH9, e.g., the CDR2 sequence of FVAAISWSGGSTT (SEQ ID NO: 200). In various embodiments of the aspects described herein the single domain antibody comprises a CDR2 sequence present in VHH10, e.g., the CDR2 sequence of FVAAISWSGSSAG (SEQ ID NO: 201). In various embodiments of the aspects described herein the single domain antibody comprises a CDR2 sequence present in VHH11, e.g., the CDR2 sequence of FVAAIRWSGGRTL (SEQ ID NO: 202). In various embodiments of the aspects described herein the single domain antibody comprises a CDR2 sequence present in VHH12, e.g., the CDR2 sequence of FVASITWNGGSTS (SEQ ID NO: 203).

In various embodiments of the aspects described herein the single domain antibody comprises a CDR2 sequence present in VHH1, e.g., the CDR2 sequence of AIDWNGRGTYYRY (SEQ ID NO: 204). In various embodiments of the aspects described herein the single domain antibody comprises a CDR2 sequence present in VHH2, e.g., the CDR2 sequence of AIDWNGRGTYYRY (SEQ ID NO: 204). In various embodiments of the aspects described herein the single domain antibody comprises a CDR2 sequence present in VHH3, e.g., the CDR2 sequence of RINGGGITH (SEQ ID NO: 205). In various embodiments of the aspects described herein the single domain antibody comprises a CDR2 sequence present in VHH4, e.g., the CDR2 sequence of FIDRIATTT (SEQ ID NO: 206). In various embodiments of the aspects described herein the single domain antibody comprises a CDR2 sequence present in VHH5, e.g., the CDR2 sequence of AITWNGGTTY (SEQ ID NO: 207). In various embodiments of the aspects described herein the single domain antibody comprises a CDR2 sequence present in VHH6, e.g., the CDR2 sequence of FISGGGTTT (SEQ ID NO: 208). In various embodiments of the aspects described herein the single domain antibody comprises a CDR2 sequence present in VHH7, e.g., the CDR2 sequence of RITGGGSTH (SEQ ID NO: 209). In various embodiments of the aspects described herein the single domain antibody comprises a CDR2 sequence present in VHH9, e.g., the CDR2 sequence of AISWSGGSTT (SEQ ID NO: 210). In various embodiments of the aspects described herein the single domain antibody comprises a CDR2 sequence present in VHH10, e.g., the CDR2 sequence of AISWSGSSAG (SEQ ID NO: 211). In various embodiments of the aspects described herein the single domain antibody comprises a CDR2 sequence present in VHH11, e.g., the CDR2 sequence of AIRWSGGRTL (SEQ ID NO: 212). In various embodiments of the aspects described herein the single domain antibody comprises a CDR2 sequence present in VHH12, e.g., the CDR2 sequence of SITWNGGSTS (SEQ ID NO: 213).

In various embodiments of the aspects described herein the single domain antibody comprises a CDR3 sequence present in VHH1, e.g., the CDR3 sequence of GSIDLNWYGGMDY (SEQ ID NO: 60). In various embodiments of the aspects described herein the single domain antibody comprises a CDR3 sequence present in VHH2, e.g., the CDR3 sequence of TTVLTDPRVLNEYAT (SEQ ID NO: 61). In various embodiments of the aspects described herein the single domain antibody comprises a CDR3 sequence present in VHH3, e.g., the CDR3 sequence of DVFGSSGYVETY (SEQ ID NO: 62). In various embodiments of the aspects described herein the single domain antibody comprises a CDR3 sequence present in VHH4, e.g., the CDR3 sequence of PLTAR (SEQ ID NO: 63). In various embodiments of the aspects described herein the single domain antibody comprises a CDR3 sequence present in VHH5, e.g., the CDR3 sequence of DPFNQGY (SEQ ID NO: 64). In various embodiments of the aspects described herein the single domain antibody comprises a CDR3 sequence present in VHH6, e.g., the CDR3 sequence of PLTSR (SEQ ID NO: 65). In various embodiments of the aspects described herein the single domain antibody comprises a CDR3 sequence present in VHH7, e.g., the CDR3 sequence of MVNPIITAWGTIGVREIPDYDY (SEQ ID NO: 66). In various embodiments of the aspects described herein the single domain antibody comprises a CDR3 sequence present in VHH9, e.g., the CDR3 sequence of DQRGY (SEQ ID NO: 67) or QRGY (SEQ ID NO: 271). In various embodiments of the aspects described herein the single domain antibody comprises a CDR3 sequence present in VHH10, e.g., the CDR3 sequence of DPFNQGY (SEQ ID NO: 68). In various embodiments of the aspects described herein the single domain antibody comprises a CDR3 sequence present in VHH11, e.g., the CDR3 sequence of DLAEYSGTYSSPADSPAGYDY (SEQ ID NO: 69). In various embodiments of the aspects described herein the single domain antibody comprises a CDR3 sequence present in VHH12, e.g., the CDR3 sequence of ARYYVSGTYFPANY (SEQ ID NO: 70).

In various embodiments of the aspects described herein the single domain antibody comprises a CDR3 sequence present in VHH1, e.g., the CDR3 sequence of GSIDLNWYGGMDY (SEQ ID NO: 71) or SIDLNWYGGMD (SEQ ID NO: 272). In various embodiments of the aspects described herein the single domain antibody comprises a CDR3 sequence present in VHH2, e.g., the CDR3 sequence of TTVLTDPRVLNEYAT (SEQ ID NO: 72) or TVLTDPRVLNEYA (SEQ ID NO: 273). In various embodiments of the aspects described herein the single domain antibody comprises a CDR3 sequence present in VHH3, e.g., the CDR3 sequence of DVFGSSGYVETY (SEQ ID NO: 73) or VFGSSGYVET (SEQ ID NO: 274). In various embodiments of the aspects described herein the single domain antibody comprises a CDR3 sequence present in VHH4, e.g., the CDR3 sequence of PLTAR (SEQ ID NO: 74) or LTA (SEQ ID NO: 275). In various embodiments of the aspects described herein the single domain antibody comprises a CDR3 sequence present in VHH5, e.g., the CDR3 sequence of DPFNQGY (SEQ ID NO: 75) or PFNQG (SEQ ID NO: 276). In various embodiments of the aspects described herein the single domain antibody comprises a CDR3 sequence present in VHH6, e.g., the CDR3 sequence of PLTSR (SEQ ID NO: 76) or LTS (SEQ ID NO: 277). In various embodiments of the aspects described herein the single domain antibody comprises a CDR3 sequence present in VHH7, e.g., the CDR3 sequence of MVNPIITAWGTIGVREIPDYDY (SEQ ID NO: 77) or VNPIITAWGTIGVREIPDYD (SEQ ID NO: 278). In various embodiments of the aspects described herein the single domain antibody comprises a CDR3 sequence present in VHH9, e.g., the CDR3 sequence of DQRGY (SEQ ID NO: 78) or RG (SEQ ID NO: 279). In various embodiments of the aspects described herein the single domain antibody comprises a CDR3 sequence present in VHH10, e.g., the CDR3 sequence of DPFNQGY (SEQ ID NO: 79) or PFNQG (SEQ ID NO: 280). In various embodiments of the aspects described herein the single domain antibody comprises a CDR3 sequence present in VHH11, e.g., the CDR3 sequence of DLAEYSGTYSSPADSPAGYDY (SEQ ID NO: 80) or LAEYSGTYSSPADSPAGYD (SEQ ID NO: 281). In various embodiments of the aspects described herein the single domain antibody comprises a CDR3 sequence present in VHH12, e.g., the CDR3 sequence of ARYYVSGTYFPANY (SEQ ID NO: 81) or RYYVSGTYFPAN (SEQ ID NO: 282).

In various embodiments of the aspects described herein, the single domain antibody comprises a CDR3 sequence present in VHH1, e.g., the CDR3 sequence of CAAGSIDLNWYGGMDY (SEQ ID NO: 82) or AAGSIDLNWYGGMDY (SEQ ID NO: 283). In various embodiments of the aspects described herein the single domain antibody comprises a CDR3 sequence present in VHH2, e.g., the CDR3 sequence of CAATTVLTDPRVLNEYAT (SEQ ID NO: 83) or AATTVLTDPRVLNEYAT (SEQ ID NO: 284). In various embodiments of the aspects described herein the single domain antibody comprises a CDR3 sequence present in VHH3, e.g., the CDR3 sequence of KADVFGSSGYVETY (SEQ ID NO: 84). In various embodiments of the aspects described herein the single domain antibody comprises a CDR3 sequence present in VHH4, e.g., the CDR3 sequence of NHPLTAR (SEQ ID NO: 85). In various embodiments of the aspects described herein the single domain antibody comprises a CDR3 sequence present in VHH5, e.g., the CDR3 sequence of AADPFNQGY (SEQ ID NO: 86). In various embodiments of the aspects described herein the single domain antibody comprises a CDR3 sequence present in VHH6, e.g., the CDR3 sequence of NHPLTSR (SEQ ID NO: 87). In various embodiments of the aspects described herein the single domain antibody comprises a CDR3 sequence present in VHH7, e.g., the CDR3 sequence of ASMVNPIITAWGTIGVREIPDYDY (SEQ ID NO: 88). In various embodiments of the aspects described herein the single domain antibody comprises a CDR3 sequence present in VHH9, e.g., the CDR3 sequence of NDQRGY (SEQ ID NO: 89). In various embodiments of the aspects described herein the single domain antibody comprises a CDR3 sequence present in VHH10, e.g., the CDR3 sequence of AADPFNQGY (SEQ ID NO: 90). In various embodiments of the aspects described herein the single domain antibody comprises a CDR3 sequence present in VHH11, e.g., the CDR3 sequence of AADLAEYSGTYSSPADSPAGYDY (SEQ ID NO: 91). In various embodiments of the aspects described herein the single domain antibody comprises a CDR3 sequence present in VHH12, e.g., the CDR3 sequence of AAARYYVSGTYFPANY (SEQ ID NO: 92).

In various embodiments of the aspects described herein the single domain antibody comprises a CDR3 sequence present in VHH1, e.g., the CDR3 sequence of GSIDLNWYGGMDY (SEQ ID NO: 214). In various embodiments of the aspects described herein the single domain antibody comprises a CDR3 sequence present in VHH2, e.g., the CDR3 sequence of TTVLTDPRVLNEYAT (SEQ ID NO: 215). In various embodiments of the aspects described herein the single domain antibody comprises a CDR3 sequence present in VHH3, e.g., the CDR3 sequence of DVFGSSGYVETY (SEQ ID NO: 216). In various embodiments of the aspects described herein the single domain antibody comprises a CDR3 sequence present in VHH4, e.g., the CDR3 sequence of PLTAR (SEQ ID NO: 217). In various embodiments of the aspects described herein the single domain antibody comprises a CDR3 sequence present in VHH5, e.g., the CDR3 sequence of DPFNQGY (SEQ ID NO: 218). In various embodiments of the aspects described herein the single domain antibody comprises a CDR3 sequence present in VHH6, e.g., the CDR3 sequence of PLTSR (SEQ ID NO: 219). In various embodiments of the aspects described herein the single domain antibody comprises a CDR3 sequence present in VHH7, e.g., the CDR3 sequence of MVNPIITAWGTIGVREIPDYDY (SEQ ID NO: 220). In various embodiments of the aspects described herein the single domain antibody comprises a CDR3 sequence present in VHH9, e.g., the CDR3 sequence of QRGY (SEQ ID NO: 221). In various embodiments of the aspects described herein the single domain antibody comprises a CDR3 sequence present in VHH10, e.g., the CDR3 sequence of DPFNQGY (SEQ ID NO: 222). In various embodiments of the aspects described herein the single domain antibody comprises a CDR3 sequence present in VHH11, e.g., the CDR3 sequence of DLAEYSGTYSSPADSPAGYDY (SEQ ID NO: 223). In various embodiments of the aspects described herein the single domain antibody comprises a CDR3 sequence present in VHH12, e.g., the CDR3 sequence of ARYYVSGTYFPANY (SEQ ID NO: 224).

In various embodiments of the aspects described herein the single domain antibody comprises a CDR3 sequence present in VHH1, e.g., the CDR3 sequence of AAGSIDLNWYGGMD (SEQ ID NO: 225). In various embodiments of the aspects described herein the single domain antibody comprises a CDR3 sequence present in VHH2, e.g., the CDR3 sequence of AATTVLTDPRVLNEYA (SEQ ID NO: 226). In various embodiments of the aspects described herein the single domain antibody comprises a CDR3 sequence present in VHH3, e.g., the CDR3 sequence of KADVFGSSGYVET (SEQ ID NO: 227). In various embodiments of the aspects described herein the single domain antibody comprises a CDR3 sequence present in VHH4, e.g., the CDR3 sequence of NHPLTA (SEQ ID NO: 228). In various embodiments of the aspects described herein the single domain antibody comprises a CDR3 sequence present in VHH5, e.g., the CDR3 sequence of AADPFNQG (SEQ ID NO: 229). In various embodiments of the aspects described herein the single domain antibody comprises a CDR3 sequence present in VHH6, e.g., the CDR3 sequence of NHPLTS (SEQ ID NO: 230). In various embodiments of the aspects described herein the single domain antibody comprises a CDR3 sequence present in VHH7, e.g., the CDR3 sequence of ASMVNPIITAWGTIGVREIPDYD (SEQ ID NO: 231). In various embodiments of the aspects described herein the single domain antibody comprises a CDR3 sequence present in VHH9, e.g., the CDR3 sequence of NDQRG (SEQ ID NO: 232). In various embodiments of the aspects described herein the single domain antibody comprises a CDR3 sequence present in VHH10, e.g., the CDR3 sequence of AADPFNQG (SEQ ID NO: 233). In various embodiments of the aspects described herein the single domain antibody comprises a CDR3 sequence present in VHH11, e.g., the CDR3 sequence of AADLAEYSGTYSSPADSPAGYD (SEQ ID NO: 234). In various embodiments of the aspects described herein the single domain antibody comprises a CDR3 sequence present in VHH12, e.g., the CDR3 sequence of AAARYYVSGTYFPAN (SEQ ID NO: 235).

In various embodiments of the aspects described herein, the single domain antibody comprises a CDR3 sequence present in VHH1, e.g., the CDR3 sequence of GSIDLNWYGGMDY (SEQ ID NO: 236). In various embodiments of the aspects described herein the single domain antibody comprises a CDR3 sequence present in VHH2, e.g., the CDR3 sequence of TTVLTDPRVLNEYAT (SEQ ID NO: 237). In various embodiments of the aspects described herein the single domain antibody comprises a CDR3 sequence present in VHH3, e.g., the CDR3 sequence of DVFGSSGYVETY (SEQ ID NO: 238). In various embodiments of the aspects described herein the single domain antibody comprises a CDR3 sequence present in VHH4, e.g., the CDR3 sequence of PLTAR (SEQ ID NO: 239). In various embodiments of the aspects described herein the single domain antibody comprises a CDR3 sequence present in VHH5, e.g., the CDR3 sequence of DPFNQGY (SEQ ID NO: 240). In various embodiments of the aspects described herein the single domain antibody comprises a CDR3 sequence present in VHH6, e.g., the CDR3 sequence of PLTSR (SEQ ID NO: 241). In various embodiments of the aspects described herein the single domain antibody comprises a CDR3 sequence present in VHH7, e.g., the CDR3 sequence of MVNPIITAWGTIGVREIPDYDY (SEQ ID NO: 242). In various embodiments of the aspects described herein the single domain antibody comprises a CDR3 sequence present in VHH9, e.g., the CDR3 sequence of QRGY (SEQ ID NO: 243). In various embodiments of the aspects described herein the single domain antibody comprises a CDR3 sequence present in VHH10, e.g., the CDR3 sequence of DPFNQGY (SEQ ID NO: 244). In various embodiments of the aspects described herein the single domain antibody comprises a CDR3 sequence present in VHH11, e.g., the CDR3 sequence of DLAEYSGTYSSPADSPAGYDY (SEQ ID NO: 245). In various embodiments of the aspects described herein the single domain antibody comprises a CDR3 sequence present in VHH12, e.g., the CDR3 sequence of ARYYVSGTYFPANY (SEQ ID NO: 246).

In some embodiments, the single domain antibody provided herein comprises a CDR1 sequence, a CDR2 sequence, and a CDR3 sequence of the single domain antibody selected from the group consisting of:
a) VHH2:

i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);

ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);

iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);

iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);

v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or

vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
b) VHH3:

i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);

ii) the CDR1 sequence of GSIF SIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);

iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);

iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);

v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or

vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
c) VHH4:

i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);

ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);

iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);

iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);

v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or

vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
d) VHH5:

i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);

ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);

iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);

iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);

v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or

vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
e) VHH6:

i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);

ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);

iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);

iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);

v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or

vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
f) VHH7:

i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);

ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);

iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);

iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);

v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or

vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
g) VHH9:

i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);

ii) the CDR1 sequence of GRTF STY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);

iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);

iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);

v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or

vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
h) VHH10:

i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);

ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);

iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);

iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);

v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or

vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
i) VHH11:

i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);

ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);

iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);

iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);

v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or

vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
j) VHH12:

i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);

ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);

iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);

iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);

v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or

vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

In some embodiments, the single domain antibody provided herein comprises a CDR1 sequence, a CDR2 sequence, and a CDR3 sequence of the single domain antibody selected from the group consisting of: i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60); ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272); iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283); iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214); v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); and vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236).

In some embodiments, the single domain antibody provided herein comprises a CDR1 sequence, a CDR2 sequence, and a CDR3 sequence of the single domain antibody selected from the group consisting of: i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61); ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273); iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284); iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215); v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); and vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237).

In some embodiments, the single domain antibody provided herein comprises a CDR1 sequence, a CDR2 sequence, and a CDR3 sequence of the single domain antibody selected from the group consisting of: i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62); ii) the CDR1 sequence of GSIF SIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274); iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84); iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216); v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); and vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238).

In some embodiments, the single domain antibody provided herein comprises a CDR1 sequence, a CDR2 sequence, and a CDR3 sequence of the single domain antibody selected from the group consisting of: i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63); ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275); iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85); iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217); v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); and vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239).

In some embodiments, the single domain antibody provided herein comprises a CDR1 sequence, a CDR2 sequence, and a CDR3 sequence of the single domain antibody selected from the group consisting of: i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64); ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276); iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86); iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218); v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); and vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240).

In some embodiments, the single domain antibody provided herein comprises a CDR1 sequence, a CDR2 sequence, and a CDR3 sequence of the single domain antibody selected from the group consisting of: i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65); ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277); iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87); iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219); v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); and vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241).

In some embodiments, the single domain antibody provided herein comprises a CDR1 sequence, a CDR2 sequence, and a CDR3 sequence of the single domain antibody selected from the group consisting of: i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66); ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278); iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88); iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220); v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); and vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242).

In some embodiments, the single domain antibody provided herein comprises a CDR1 sequence, a CDR2 sequence, and a CDR3 sequence of the single domain antibody selected from the group consisting of: i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271); ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279); iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89); iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221); v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); and vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243).

In some embodiments, the single domain antibody provided herein comprises a CDR1 sequence, a CDR2 sequence, and a CDR3 sequence of the single domain antibody selected from the group consisting of: i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68); ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280); iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90); iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222); v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); and vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244).

In some embodiments, the single domain antibody provided herein comprises a CDR1 sequence, a CDR2 sequence, and a CDR3 sequence of the single domain antibody selected from the group consisting of: i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69); ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281); iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91); iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223); v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); and vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245).

In some embodiments, the single domain antibody provided herein comprises a CDR1 sequence, a CDR2 sequence, and a CDR3 sequence of the single domain antibody selected from the group consisting of: i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70); ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282); iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92); iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224); v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); and vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

In some embodiments, the single domain antibody provided herein has one or more CDR regions from VHH1.

In some embodiments, the single domain antibody has a CDR1 having an amino acid sequence of the CDR1 as set forth in SEQ ID NO: 93. In some embodiments, the single domain antibody has a CDR2 having an amino acid sequence of the CDR2 as set forth in SEQ ID NO: 93. In other embodiments, the single domain antibody has a CDR3 having an amino acid sequence of the CDR3 as set forth in SEQ ID NO: 93. In some embodiments, the single domain antibody has a CDR1 and a CDR2 having amino acid sequences of the CDR1 and the CDR2 as set forth in SEQ ID NO: 93. In some embodiments, the single domain antibody has a CDR1 and a CDR3 having amino acid sequences of the CDR1 and the CDR3 as set forth in SEQ ID NO: 93. In some embodiments, the single domain antibody has a CDR2 and a CDR3 having amino acid sequences of the CDR2 and the CDR3 as set forth in SEQ ID NO: 93. In some embodiments, the single domain antibody has a CDR1, a CDR2, and a CDR3 having amino acid sequences of the CDR1, the CDR2, and the CDR3 as set forth in SEQ ID NO: 93. CDR sequences can be determined according to well-known numbering systmes. As described above, CDR regions are well known to those skilled in the art and have been defined by well-known numbering systems. The residues from each of these hypervariable regions or CDRs are noted in Table 1 above. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the single domain antibody provided herein has one or more CDR regions from VHH2.

In some embodiments, the single domain antibody has a CDR1 having an amino acid sequence of the CDR1 as set forth in SEQ ID NO: 94. In some embodiments, the single domain antibody has a CDR2 having an amino acid sequence of the CDR2 as set forth in SEQ ID NO: 94. In other embodiments, the single domain antibody has a CDR3 having an amino acid sequence of the CDR3 as set forth in SEQ ID NO: 94. In some embodiments, the single domain antibody has a CDR1 and a CDR2 having amino acid sequences of the CDR1 and the CDR2 as set forth in SEQ ID NO: 94. In some embodiments, the single domain antibody has a CDR1 and a CDR3 having amino acid sequences of the CDR1 and the CDR3 as set forth in SEQ ID NO: 94. In some embodiments, the single domain antibody has a CDR2 and a CDR3 having amino acid sequences of the CDR2 and the CDR3 as set forth in SEQ ID NO: 94. In some embodiments, the single domain antibody has a CDR1, a CDR2, and a CDR3 having amino acid sequences of the CDR1, the CDR2, and the CDR3 as set forth in SEQ ID NO: 94. CDR sequences can be determined according to well-known numbering systems. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the single domain antibody provided herein has one or more CDR regions from VHH3.

In some embodiments, the single domain antibody has a CDR1 having an amino acid sequence of the CDR1 as set forth in SEQ ID NO: 95. In some embodiments, the single domain antibody has a CDR2 having an amino acid sequence of the CDR2 as set forth in SEQ ID NO: 95. In other embodiments, the single domain antibody has a CDR3 having an amino acid sequence of the CDR3 as set forth in SEQ ID NO: 95. In some embodiments, the single domain antibody has a CDR1 and a CDR2 having amino acid sequences of the CDR1 and the CDR2 as set forth in SEQ ID NO: 95. In some embodiments, the single domain antibody has a CDR1 and a CDR3 having amino acid sequences of the CDR1 and the CDR3 as set forth in SEQ ID NO: 95. In some embodiments, the single domain antibody has a CDR2 and a CDR3 having amino acid sequences of the CDR2 and the CDR3 as set forth in SEQ ID NO: 95. In some embodiments, the single domain antibody has a CDR1, a CDR2, and a CDR3 having amino acid sequences of the CDR1, the CDR2, and the CDR3 as set forth in SEQ ID NO: 95. CDR sequences can be determined according to well-known numbering systems. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the single domain antibody provided herein has one or more CDR regions from VHH4.

In some embodiments, the single domain antibody has a CDR1 having an amino acid sequence of the CDR1 as set forth in SEQ ID NO: 96. In some embodiments, the single domain antibody has a CDR2 having an amino acid sequence of the CDR2 as set forth in SEQ ID NO: 96. In other embodiments, the single domain antibody has a CDR3 having an amino acid sequence of the CDR3 as set forth in SEQ ID NO: 96. In some embodiments, the single domain antibody has a CDR1 and a CDR2 having amino acid sequences of the CDR1 and the CDR2 as set forth in SEQ ID NO: 96. In some embodiments, the single domain antibody has a CDR1 and a CDR3 having amino acid sequences of the CDR1 and the CDR3 as set forth in SEQ ID NO: 96. In some embodiments, the single domain antibody has a CDR2 and a CDR3 having amino acid sequences of the CDR2 and the CDR3 as set forth in SEQ ID NO: 96. In some embodiments, the single domain antibody has a CDR1, a CDR2, and a CDR3 having amino acid sequences of the CDR1, the CDR2, and the CDR3 as set forth in SEQ ID NO: 96. CDR sequences can be determined according to well-known numbering systems. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the single domain antibody provided herein has one or more CDR regions from VHH5.

In some embodiments, the single domain antibody has a CDR1 having an amino acid sequence of the CDR1 as set forth in SEQ ID NO: 97. In some embodiments, the single domain antibody has a CDR2 having an amino acid sequence of the CDR2 as set forth in SEQ ID NO: 97. In other embodiments, the single domain antibody has a CDR3 having an amino acid sequence of the CDR3 as set forth in SEQ ID NO: 97. In some embodiments, the single domain antibody has a CDR1 and a CDR2 having amino acid sequences of the CDR1 and the CDR2 as set forth in SEQ ID NO: 97. In some embodiments, the single domain antibody has a CDR1 and a CDR3 having amino acid sequences of the CDR1 and the CDR3 as set forth in SEQ ID NO: 97. In some embodiments, the single domain antibody has a CDR2 and a CDR3 having amino acid sequences of the CDR2 and the CDR3 as set forth in SEQ ID NO: 97. In some embodiments, the single domain antibody has a CDR1, a CDR2, and a CDR3 having amino acid sequences of the CDR1, the CDR2, and the CDR3 as set forth in SEQ ID NO: 97. CDR sequences can be determined according to well-known numbering systems. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the single domain antibody provided herein has one or more CDR regions from VHH6.

In some embodiments, the single domain antibody has a CDR1 having an amino acid sequence of the CDR1 as set forth in SEQ ID NO: 98. In some embodiments, the single domain antibody has a CDR2 having an amino acid sequence of the CDR2 as set forth in SEQ ID NO: 98. In other embodiments, the single domain antibody has a CDR3 having an amino acid sequence of the CDR3 as set forth in SEQ ID NO: 98. In some embodiments, the single domain antibody has a CDR1 and a CDR2 having amino acid sequences of the CDR1 and the CDR2 as set forth in SEQ ID NO: 98. In some embodiments, the single domain antibody has a CDR1 and a CDR3 having amino acid sequences of the CDR1 and the CDR3 as set forth in SEQ ID NO: 98. In some embodiments, the single domain antibody has a CDR2 and a CDR3 having amino acid sequences of the CDR2 and the CDR3 as set forth in SEQ ID NO: 98. In some embodiments, the single domain antibody has a CDR1, a CDR2, and a CDR3 having amino acid sequences of the CDR1, the CDR2, and the CDR3 as set forth in SEQ ID NO: 98. CDR sequences can be determined according to well-known numbering systems. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the single domain antibody provided herein has one or more CDR regions from VHH7.

In some embodiments, the single domain antibody has a CDR1 having an amino acid sequence of the CDR1 as set forth in SEQ ID NO: 99. In some embodiments, the single domain antibody has a CDR2 having an amino acid sequence of the CDR2 as set forth in SEQ ID NO: 99. In other embodiments, the single domain antibody has a CDR3 having an amino acid sequence of the CDR3 as set forth in SEQ ID NO: 99. In some embodiments, the single domain antibody has a CDR1 and a CDR2 having amino acid sequences of the CDR1 and the CDR2 as set forth in SEQ ID NO: 99. In some embodiments, the single domain antibody has a CDR1 and a CDR3 having amino acid sequences of the CDR1 and the CDR3 as set forth in SEQ ID NO: 99. In some embodiments, the single domain antibody has a CDR2 and a CDR3 having amino acid sequences of the CDR2 and the CDR3 as set forth in SEQ ID NO: 99. In some embodiments, the single domain antibody has a CDR1, a CDR2, and a CDR3 having amino acid sequences of the CDR1, the CDR2, and the CDR3 as set forth in SEQ ID NO: 99. CDR sequences can be determined according to well-known numbering systems. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the single domain antibody provided herein has one or more CDR regions from VHH9.

In some embodiments, the single domain antibody has a CDR1 having an amino acid sequence of the CDR1 as set forth in SEQ ID NO: 100. In some embodiments, the single domain antibody has a CDR2 having an amino acid sequence of the CDR2 as set forth in SEQ ID NO: 100. In other embodiments, the single domain antibody has a CDR3 having an amino acid sequence of the CDR3 as set forth in SEQ ID NO: 100. In some embodiments, the single domain antibody has a CDR1 and a CDR2 having amino acid sequences of the CDR1 and the CDR2 as set forth in SEQ ID NO: 100. In some embodiments, the single domain antibody has a CDR1 and a CDR3 having amino acid sequences of the CDR1 and the CDR3 as set forth in SEQ ID NO: 100. In some embodiments, the single domain antibody has a CDR2 and a CDR3 having amino acid sequences of the CDR2 and the CDR3 as set forth in SEQ ID NO: 100. In some embodiments, the single domain antibody has a CDR1, a CDR2, and a CDR3 having amino acid sequences of the CDR1, the CDR2, and the CDR3 as set forth in SEQ ID NO: 100. CDR sequences can be determined according to well-known numbering systems. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the single domain antibody provided herein has one or more CDR regions from VHH10.

In some embodiments, the single domain antibody has a CDR1 having an amino acid sequence of the CDR1 as set forth in SEQ ID NO: 101. In some embodiments, the single domain antibody has a CDR2 having an amino acid sequence of the CDR2 as set forth in SEQ ID NO: 101. In other embodiments, the single domain antibody has a CDR3 having an amino acid sequence of the CDR3 as set forth in SEQ ID NO: 101. In some embodiments, the single domain antibody has a CDR1 and a CDR2 having amino acid sequences of the CDR1 and the CDR2 as set forth in SEQ ID NO: 101. In some embodiments, the single domain antibody has a CDR1 and a CDR3 having amino acid sequences of the CDR1 and the CDR3 as set forth in SEQ ID NO: 101. In some embodiments, the single domain antibody has a CDR2 and a CDR3 having amino acid sequences of the CDR2 and the CDR3 as set forth in SEQ ID NO: 101. In some embodiments, the single domain antibody has a CDR1, a CDR2, and a CDR3 having amino acid sequences of the CDR1, the CDR2, and the CDR3 as set forth in SEQ ID NO: 101. CDR sequences can be determined according to well-known numbering systems. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the single domain antibody that binds to pIgR provided herein has one or more CDR regions from VHH11.

In some embodiments, the single domain antibody has a CDR1 having an amino acid sequence of the CDR1 as set forth in SEQ ID NO: 102. In some embodiments, the single domain antibody has a CDR2 having an amino acid sequence of the CDR2 as set forth in SEQ ID NO: 102. In other embodiments, the single domain antibody has a CDR3 having an amino acid sequence of the CDR3 as set forth in SEQ ID NO: 102. In some embodiments, the single domain antibody has a CDR1 and a CDR2 having amino acid sequences of the CDR1 and the CDR2 as set forth in SEQ ID NO: 102. In some embodiments, the single domain antibody has a CDR1 and a CDR3 having amino acid sequences of the CDR1 and the CDR3 as set forth in SEQ ID NO: 102. In some embodiments, the single domain antibody has a CDR2 and a CDR3 having amino acid sequences of the CDR2 and the CDR3 as set forth in SEQ ID NO: 102. In some embodiments, the single domain antibody has a CDR1, a CDR2, and a CDR3 having amino acid sequences of the CDR1, the CDR2, and the CDR3 as set forth in SEQ ID NO: 102. CDR sequences can be determined according to well-known numbering systems. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the single domain antibody that binds to pIgR provided herein has one or more CDR regions from VHH12.

In some embodiments, the single domain antibody has a CDR1 having an amino acid sequence of the CDR1 as set forth in SEQ ID NO: 103. In some embodiments, the single domain antibody has a CDR2 having an amino acid sequence of the CDR2 as set forth in SEQ ID NO: 103. In other embodiments, the single domain antibody has a CDR3 having an amino acid sequence of the CDR3 as set forth in SEQ ID NO: 103. In some embodiments, the single domain antibody has a CDR1 and a CDR2 having amino acid sequences of the CDR1 and the CDR2 as set forth in SEQ ID NO: 103. In some embodiments, the single domain antibody has a CDR1 and a CDR3 having amino acid sequences of the CDR1 and the CDR3 as set forth in SEQ ID NO: 103. In some embodiments, the single domain antibody has a CDR2 and a CDR3 having amino acid sequences of the CDR2 and the CDR3 as set forth in SEQ ID NO: 103. In some embodiments, the single domain antibody has a CDR1, a CDR2, and a CDR3 having amino acid sequences of the CDR1, the CDR2, and the CDR3 as set forth in SEQ ID NO: 103. CDR sequences can be determined according to well-known numbering systems. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the single domain antibody further comprises one or more framework regions of VHH1, VHH2, VHH3, VHH4, VHH5, VHH6, VHH7, VHH9, VHH10, VHH11 and VHH12.

In various embodiments of the aspects described herein, the single domain antibody comprises a framework derived from a VHH domain comprising the sequence of

(SEQ ID NO: 93)
QVQLVESGGGLVQAGGSLKLACAAPGLTFSSYRMGWFRQAPGQEREFVAA
IDWNGRGTYYRYYADSVKGRSTISRDNAKNTMYLQMNSLKPEDTAVYYCA
AGSIDLNWYGGMDYWGQGTQVTVSS.

In various embodiments of the aspects described herein, the single domain antibody comprises a framework derived from a VHH domain comprising the sequence of

(SEQ ID NO: 94)
EVQVVESGGGLVQAGGSLKLACAAPGLTFSSYRMGWFRQAPGQEREFVAA
IDWNGRGTYYRYYADSVKGRSTISRDNAKNTVYLQMNSLKPEDTAVYYCA
ATTVLTDPRVLNEYATWGQGTQVTVSS.

In various embodiments of the aspects described herein, the single domain antibody comprises a framework derived from a VHH domain comprising the sequence of

(SEQ ID NO: 95)
QLQLVESGGGLVQPGGSLRLSCAASGSIFSINVMGWYRQAPGKQRELVAR
INGGGITHYAESVKGRFTISRDNAKNTVYLQMNSLKPEDTAAYYCKADVF
GSSGYVETYWGQGTQVTVSS.

In various embodiments of the aspects described herein, the single domain antibody comprises a framework derived from a VHH domain comprising the sequence of

(SEQ ID NO: 96)
EVQVVESGGGLVQAGGSLRLSCAVSGTSVSSNAMGWYRQAPGKQREWVGF
IDRIATTTIATSVKGRFAITRDNAKNTVYLQMSGLKPEDTAVYYCNHPLT
ARWGQGTQVTVSS.

In various embodiments of the aspects described herein, the single domain antibody comprises a framework derived from a VHH domain comprising the sequence of

(SEQ ID NO: 97)
QVQLVESGGGLVQAGGSLRLSCAASGRTFSSYAMGWFRQAPGKEREFVAA
ITWNGGTTYYADSVKGRFTISRDNAKNTVYLQMNSLKPEDTAVYYCAADP
FNQGYWGQGTQVTVSS.

In various embodiments of the aspects described herein, the single domain antibody comprises a framework derived from a VHH domain comprising the sequence of

(SEQ ID NO: 98)
EVQLVESGGGLVQAGGSLRLSCAVSGSSVSSDAMGWYRQAPGNQRAWVAF
ISGGGTTTYADSVKGRFTISRDNTKNTVYLHMNSLKPEDTAVYYCNHPLT
SRWGQGTQVTVSS.

In various embodiments of the aspects described herein, the single domain antibody comprises a framework derived from a VHH domain comprising the sequence of

(SEQ ID NO: 99)
EVQVVESGGGLVQAGGSLRLACVASRSIGSINVMGWYRQAPGKQRDLVAR
ITGGGSTHYAESVKGRFTISRDNAKNTVYLQMNSLEPEDTAVYYCASMVN
PIITAWGTIGVREIPDYDYWGQGTQVTVSS.

In various embodiments of the aspects described herein, the single domain antibody comprises a framework derived from a VHH domain comprising the sequence of

(SEQ ID NO: 100)
QVQLVESGGGLVQAGGSLRLSCAVSGRTFSTYRMGWFRQAPGKERSFVAA
ISWSGGSTTYADPVKGRFTISRDNAKNTVYLRMNSLKPEDTAVYYCNDQR
GYWGQGTLVTVSS.

In various embodiments of the aspects described herein, the single domain antibody comprises a framework derived from a VHH domain comprising the sequence of

(SEQ ID NO: 101)
EVQVVESGGGLVQAGGSLRLSCAASGFTFTRYAMGWFRQAPGKERSFVAA
ISWSGSSAGYGDSVKGRFTISRDNAKNTLYLQMNSLKPEDTAVYYCAADP
FNQGYWGQGTQVTVSS.

In various embodiments of the aspects described herein, the single domain antibody comprises a framework derived from a VHH domain comprising the sequence of

(SEQ ID NO: 102)
EVQVVESGGGLVQAGGSLRLSCAASGRTFTTYRMGWFRQAPGKEREFVAA
IRWSGGRTLYADSVKGRFTISRDNAKNTAYLQMNNLRPEDTAVYYCAADL
AEYSGTYSSPADSPAGYDYWGQGTQVTVSS.

In various embodiments of the aspects described herein, the single domain antibody comprises a framework derived from a VHH domain comprising the sequence of

(SEQ ID NO: 103)
QVQLVETGGGLVQAGDSLRLSCAASGRTLSFNTYAMGWFRQAPGKEREFV
ASITWNGGSTSYADSVKGRFTITRDNAKNTATLRMNSLQPDDTAVYYCAA
ARYYVSGTYFPANYWGQGTQVTVSS.

In some embodiments, the single domain antibody provided herein comprises a FR1 having an amino acid sequence of the FR1 as set forth in SEQ ID NO: 93. In some embodiments, the single domain antibody provided herein comprises a FR2 having an amino acid sequence of the FR2 as set forth in SEQ ID NO: 93. In some embodiments, the single domain antibody provided herein comprises a FR3 having an amino acid sequence of the FR3 as set forth in SEQ ID NO: 93. In some embodiments, the single domain antibody provided herein comprises a FR4 having an amino acid sequence of the FR4 as set forth in SEQ ID NO: 93. In some embodiments, the single domain antibody provided herein comprises a FR1 and a FR2 having amino acid sequences of the FR1 and the FR2 as set forth in SEQ ID NO: 93. In some embodiments, the single domain antibody provided herein comprises a FR1 and a FR3 having amino acid sequences of the FR1 and the FR3 as set forth in SEQ ID NO: 93. In some embodiments, the single domain antibody provided herein comprises a FR1 and a FR4 having amino acid sequences of the FR1 and the FR4 as set forth in SEQ ID NO: 93. In some embodiments, the single domain antibody provided herein comprises a FR2 and a FR3 having amino acid sequences of the FR2 and the FR3 as set forth in SEQ ID NO: 93. In some embodiments, the single domain antibody provided herein comprises a FR2 and a FR4 having amino acid sequences of the FR2 and the FR4 as set forth in SEQ ID NO: 93. In some embodiments, the single domain antibody provided herein comprises a FR3 and a FR4 having amino acid sequences of the FR3 and the FR24 as set forth in SEQ ID NO: 93. In some embodiments, the single domain antibody provided herein comprises a FR1, a FR2, and a FR3 having amino acid sequences of the FR1, the FR2, and the FR3 as set forth in SEQ ID NO: 93. In some embodiments, the single domain antibody provided herein comprises a FR1, a FR2, and a FR4 having amino acid sequences of the FR1, the FR2, and the FR4 as set forth in SEQ ID NO: 93. In some embodiments, the single domain antibody provided herein comprises a FR1, a FR3, and a FR4 having amino acid sequences of the FR1, the FR3, and the FR4 as set forth in SEQ ID NO: 93. In some embodiments, the single domain antibody provided herein comprises a FR2, a FR3, and a FR4 having amino acid sequences of the FR2, the FR3, and the FR4 as set forth in SEQ ID NO: 93. In a specific embodiment, the single domain antibody provided herein comprises a FR1, a FR2, a FR3, and a FR4 having amino acid sequences of the FR1, the FR2, the FR3, and the FR4 as set forth in SEQ ID NO: 93.

In some embodiments, the single domain antibody provided herein comprises a FR1 having an amino acid sequence of the FR1 as set forth in SEQ ID NO: 94. In some embodiments, the single domain antibody provided herein comprises a FR2 having an amino acid sequence of the FR2 as set forth in SEQ ID NO: 94. In some embodiments, the single domain antibody provided herein comprises a FR3 having an amino acid sequence of the FR3 as set forth in SEQ ID NO: 94. In some embodiments, the single domain antibody provided herein comprises a FR4 having an amino acid sequence of the FR4 as set forth in SEQ ID NO: 94. In some embodiments, the single domain antibody provided herein comprises a FR1 and a FR2 having amino acid sequences of the FR1 and the FR2 as set forth in SEQ ID NO: 94. In some embodiments, the single domain antibody provided herein comprises a FR1 and a FR3 having amino acid sequences of the FR1 and the FR3 as set forth in SEQ ID NO: 94. In some embodiments, the single domain antibody provided herein comprises a FR1 and a FR4 having amino acid sequences of the FR1 and the FR4 as set forth in SEQ ID NO: 94. In some embodiments, the single domain antibody provided herein comprises a FR2 and a FR3 having amino acid sequences of the FR2 and the FR3 as set forth in SEQ ID NO: 94. In some embodiments, the single domain antibody provided herein comprises a FR2 and a FR4 having amino acid sequences of the FR2 and the FR4 as set forth in SEQ ID NO: 94. In some embodiments, the single domain antibody provided herein comprises a FR3 and a FR4 having amino acid sequences of the FR3 and the FR24 as set forth in SEQ ID NO: 94. In some embodiments, the single domain antibody provided herein comprises a FR1, a FR2, and a FR3 having amino acid sequences of the FR1, the FR2, and the FR3 as set forth in SEQ ID NO: 94. In some embodiments, the single domain antibody provided herein comprises a FR1, a FR2, and a FR4 having amino acid sequences of the FR1, the FR2, and the FR4 as set forth in SEQ ID NO: 94. In some embodiments, the single domain antibody provided herein comprises a FR1, a FR3, and a FR4 having amino acid sequences of the FR1, the FR3, and the FR4 as set forth in SEQ ID NO: 94. In some embodiments, the single domain antibody provided herein comprises a FR2, a FR3, and a FR4 having amino acid sequences of the FR2, the FR3, and the FR4 as set forth in SEQ ID NO: 94. In a specific embodiment, the single domain antibody provided herein comprises a FR1, a FR2, a FR3, and a FR4 having amino acid sequences of the FR1, the FR2, the FR3, and the FR4 as set forth in SEQ ID NO: 94.

In some embodiments, the single domain antibody provided herein comprises a FR1 having an amino acid sequence of the FR1 as set forth in SEQ ID NO: 95. In some embodiments, the single domain antibody provided herein comprises a FR2 having an amino acid sequence of the FR2 as set forth in SEQ ID NO: 95. In some embodiments, the single domain antibody provided herein comprises a FR3 having an amino acid sequence of the FR3 as set forth in SEQ ID NO: 95. In some embodiments, the single domain antibody provided herein comprises a FR4 having an amino acid sequence of the FR4 as set forth in SEQ ID NO: 95. In some embodiments, the single domain antibody provided herein comprises a FR1 and a FR2 having amino acid sequences of the FR1 and the FR2 as set forth in SEQ ID NO: 95. In some embodiments, the single domain antibody provided herein comprises a FR1 and a FR3 having amino acid sequences of the FR1 and the FR3 as set forth in SEQ ID NO: 95. In some embodiments, the single domain antibody provided herein comprises a FR1 and a FR4 having amino acid sequences of the FR1 and the FR4 as set forth in SEQ ID NO: 95. In some embodiments, the single domain antibody provided herein comprises a FR2 and a FR3 having amino acid sequences of the FR2 and the FR3 as set forth in SEQ ID NO: 95. In some embodiments, the single domain antibody provided herein comprises a FR2 and a FR4 having amino acid sequences of the FR2 and the FR4 as set forth in SEQ ID NO: 95. In some embodiments, the single domain antibody provided herein comprises a FR3 and a FR4 having amino acid sequences of the FR3 and the FR24 as set forth in SEQ ID NO: 95. In some embodiments, the single domain antibody provided herein comprises a FR1, a FR2, and a FR3 having amino acid sequences of the FR1, the FR2, and the FR3 as set forth in SEQ ID NO: 95. In some embodiments, the single domain antibody provided herein comprises a FR1, a FR2, and a FR4 having amino acid sequences of the FR1, the FR2, and the FR4 as set forth in SEQ ID NO: 95. In some embodiments, the single domain antibody provided herein comprises a FR1, a FR3, and a FR4 having amino acid sequences of the FR1, the FR3, and the FR4 as set forth in SEQ ID NO: 95. In some embodiments, the single domain antibody provided herein comprises a FR2, a FR3, and a FR4 having amino acid sequences of the FR2, the FR3, and the FR4 as set forth in SEQ ID NO: 95. In a specific embodiment, the single domain antibody provided herein comprises a FR1, a FR2, a FR3, and a FR4 having amino acid sequences of the FR1, the FR2, the FR3, and the FR4 as set forth in SEQ ID NO: 95.

In some embodiments, the single domain antibody provided herein comprises a FR1 having an amino acid sequence of the FR1 as set forth in SEQ ID NO: 96. In some embodiments, the single domain antibody provided herein comprises a FR2 having an amino acid sequence of the FR2 as set forth in SEQ ID NO: 96. In some embodiments, the single domain antibody provided herein comprises a FR3 having an amino acid sequence of the FR3 as set forth in SEQ ID NO: 96. In some embodiments, the single domain antibody provided herein comprises a FR4 having an amino acid sequence of the FR4 as set forth in SEQ ID NO: 96. In some embodiments, the single domain antibody provided herein comprises a FR1 and a FR2 having amino acid sequences of the FR1 and the FR2 as set forth in SEQ ID NO: 96. In some embodiments, the single domain antibody provided herein comprises a FR1 and a FR3 having amino acid sequences of the FR1 and the FR3 as set forth in SEQ ID NO: 96. In some embodiments, the single domain antibody provided herein comprises a FR1 and a FR4 having amino acid sequences of the FR1 and the FR4 as set forth in SEQ ID NO: 96. In some embodiments, the single domain antibody provided herein comprises a FR2 and a FR3 having amino acid sequences of the FR2 and the FR3 as set forth in SEQ ID NO: 96. In some embodiments, the single domain antibody provided herein comprises a FR2 and a FR4 having amino acid sequences of the FR2 and the FR4 as set forth in SEQ ID NO: 96. In some embodiments, the single domain antibody provided herein comprises a FR3 and a FR4 having amino acid sequences of the FR3 and the FR24 as set forth in SEQ ID NO: 96. In some embodiments, the single domain antibody provided herein comprises a FR1, a FR2, and a FR3 having amino acid sequences of the FR1, the FR2, and the FR3 as set forth in SEQ ID NO: 96. In some embodiments, the single domain antibody provided herein comprises a FR1, a FR2, and a FR4 having amino acid sequences of the FR1, the FR2, and the FR4 as set forth in SEQ ID NO: 96. In some embodiments, the single domain antibody provided herein comprises a FR1, a FR3, and a FR4 having amino acid sequences of the FR1, the FR3, and the FR4 as set forth in SEQ ID NO: 96. In some embodiments, the single domain antibody provided herein comprises a FR2, a FR3, and a FR4 having amino acid sequences of the FR2, the FR3, and the FR4 as set forth in SEQ ID NO: 96. In a specific embodiment, the single domain antibody provided herein comprises a FR1, a FR2, a FR3, and a FR4 having amino acid sequences of the FR1, the FR2, the FR3, and the FR4 as set forth in SEQ ID NO: 96.

In some embodiments, the single domain antibody provided herein comprises a FR1 having an amino acid sequence of the FR1 as set forth in SEQ ID NO: 97. In some embodiments, the single domain antibody provided herein comprises a FR2 having an amino acid sequence of the FR2 as set forth in SEQ ID NO: 97. In some embodiments, the single domain antibody provided herein comprises a FR3 having an amino acid sequence of the FR3 as set forth in SEQ ID NO: 97. In some embodiments, the single domain antibody provided herein comprises a FR4 having an amino acid sequence of the FR4 as set forth in SEQ ID NO: 97. In some embodiments, the single domain antibody provided herein comprises a FR1 and a FR2 having amino acid sequences of the FR1 and the FR2 as set forth in SEQ ID NO: 97. In some embodiments, the single domain antibody provided herein comprises a FR1 and a FR3 having amino acid sequences of the FR1 and the FR3 as set forth in SEQ ID NO: 97. In some embodiments, the single domain antibody provided herein comprises a FR1 and a FR4 having amino acid sequences of the FR1 and the FR4 as set forth in SEQ ID NO: 97. In some embodiments, the single domain antibody provided herein comprises a FR2 and a FR3 having amino acid sequences of the FR2 and the FR3 as set forth in SEQ ID NO: 97. In some embodiments, the single domain antibody provided herein comprises a FR2 and a FR4 having amino acid sequences of the FR2 and the FR4 as set forth in SEQ ID NO: 97. In some embodiments, the single domain antibody provided herein comprises a FR3 and a FR4 having amino acid sequences of the FR3 and the FR24 as set forth in SEQ ID NO: 97. In some embodiments, the single domain antibody provided herein comprises a FR1, a FR2, and a FR3 having amino acid sequences of the FR1, the FR2, and the FR3 as set forth in SEQ ID NO: 97. In some embodiments, the single domain antibody provided herein comprises a FR1, a FR2, and a FR4 having amino acid sequences of the FR1, the FR2, and the FR4 as set forth in SEQ ID NO: 97. In some embodiments, the single domain antibody provided herein comprises a FR1, a FR3, and a FR4 having amino acid sequences of the FR1, the FR3, and the FR4 as set forth in SEQ ID NO: 97. In some embodiments, the single domain antibody provided herein comprises a FR2, a FR3, and a FR4 having amino acid sequences of the FR2, the FR3, and the FR4 as set forth in SEQ ID NO: 97. In a specific embodiment, the single domain antibody provided herein comprises a FR1, a FR2, a FR3, and a FR4 having amino acid sequences of the FR1, the FR2, the FR3, and the FR4 as set forth in SEQ ID NO: 97.

In some embodiments, the single domain antibody provided herein comprises a FR1 having an amino acid sequence of the FR1 as set forth in SEQ ID NO: 98. In some embodiments, the single domain antibody provided herein comprises a FR2 having an amino acid sequence of the FR2 as set forth in SEQ ID NO: 98. In some embodiments, the single domain antibody provided herein comprises a FR3 having an amino acid sequence of the FR3 as set forth in SEQ ID NO: 98. In some embodiments, the single domain antibody provided herein comprises a FR4 having an amino acid sequence of the FR4 as set forth in SEQ ID NO: 98. In some embodiments, the single domain antibody provided herein comprises a FR1 and a FR2 having amino acid sequences of the FR1 and the FR2 as set forth in SEQ ID NO: 98. In some embodiments, the single domain antibody provided herein comprises a FR1 and a FR3 having amino acid sequences of the FR1 and the FR3 as set forth in SEQ ID NO: 98. In some embodiments, the single domain antibody provided herein comprises a FR1 and a FR4 having amino acid sequences of the FR1 and the FR4 as set forth in SEQ ID NO: 98. In some embodiments, the single domain antibody provided herein comprises a FR2 and a FR3 having amino acid sequences of the FR2 and the FR3 as set forth in SEQ ID NO: 98. In some embodiments, the single domain antibody provided herein comprises a FR2 and a FR4 having amino acid sequences of the FR2 and the FR4 as set forth in SEQ ID NO: 98. In some embodiments, the single domain antibody provided herein comprises a FR3 and a FR4 having amino acid sequences of the FR3 and the FR24 as set forth in SEQ ID NO: 98. In some embodiments, the single domain antibody provided herein comprises a FR1, a FR2, and a FR3 having amino acid sequences of the FR1, the FR2, and the FR3 as set forth in SEQ ID NO: 98. In some embodiments, the single domain antibody provided herein comprises a FR1, a FR2, and a FR4 having amino acid sequences of the FR1, the FR2, and the FR4 as set forth in SEQ ID NO: 98. In some embodiments, the single domain antibody provided herein comprises a FR1, a FR3, and a FR4 having amino acid sequences of the FR1, the FR3, and the FR4 as set forth in SEQ ID NO: 98. In some embodiments, the single domain antibody provided herein comprises a FR2, a FR3, and a FR4 having amino acid sequences of the FR2, the FR3, and the FR4 as set forth in SEQ ID NO: 98. In a specific embodiment, the single domain antibody provided herein comprises a FR1, a FR2, a FR3, and a FR4 having amino acid sequences of the FR1, the FR2, the FR3, and the FR4 as set forth in SEQ ID NO: 98.

In some embodiments, the single domain antibody provided herein comprises a FR1 having an amino acid sequence of the FR1 as set forth in SEQ ID NO: 99. In some embodiments, the single domain antibody provided herein comprises a FR2 having an amino acid sequence of the FR2 as set forth in SEQ ID NO: 99. In some embodiments, the single domain antibody provided herein comprises a FR3 having an amino acid sequence of the FR3 as set forth in SEQ ID NO: 99. In some embodiments, the single domain antibody provided herein comprises a FR4 having an amino acid sequence of the FR4 as set forth in SEQ ID NO: 99. In some embodiments, the single domain antibody provided herein comprises a FR1 and a FR2 having amino acid sequences of the FR1 and the FR2 as set forth in SEQ ID NO: 99. In some embodiments, the single domain antibody provided herein comprises a FR1 and a FR3 having amino acid sequences of the FR1 and the FR3 as set forth in SEQ ID NO: 99. In some embodiments, the single domain antibody provided herein comprises a FR1 and a FR4 having amino acid sequences of the FR1 and the FR4 as set forth in SEQ ID NO: 99. In some embodiments, the single domain antibody provided herein comprises a FR2 and a FR3 having amino acid sequences of the FR2 and the FR3 as set forth in SEQ ID NO: 99. In some embodiments, the single domain antibody provided herein comprises a FR2 and a FR4 having amino acid sequences of the FR2 and the FR4 as set forth in SEQ ID NO: 99. In some embodiments, the single domain antibody provided herein comprises a FR3 and a FR4 having amino acid sequences of the FR3 and the FR24 as set forth in SEQ ID NO: 99. In some embodiments, the single domain antibody provided herein comprises a FR1, a FR2, and a FR3 having amino acid sequences of the FR1, the FR2, and the FR3 as set forth in SEQ ID NO: 99. In some embodiments, the single domain antibody provided herein comprises a FR1, a FR2, and a FR4 having amino acid sequences of the FR1, the FR2, and the FR4 as set forth in SEQ ID NO: 99. In some embodiments, the single domain antibody provided herein comprises a FR1, a FR3, and a FR4 having amino acid sequences of the FR1, the FR3, and the FR4 as set forth in SEQ ID NO: 99. In some embodiments, the single domain antibody provided herein comprises a FR2, a FR3, and a FR4 having amino acid sequences of the FR2, the FR3, and the FR4 as set forth in SEQ ID NO: 99. In a specific embodiment, the single domain antibody provided herein comprises a FR1, a FR2, a FR3, and a FR4 having amino acid sequences of the FR1, the FR2, the FR3, and the FR4 as set forth in SEQ ID NO: 99.

In some embodiments, the single domain antibody provided herein comprises a FR1 having an amino acid sequence of the FR1 as set forth in SEQ ID NO: 100. In some embodiments, the single domain antibody provided herein comprises a FR2 having an amino acid sequence of the FR2 as set forth in SEQ ID NO: 100. In some embodiments, the single domain antibody provided herein comprises a FR3 having an amino acid sequence of the FR3 as set forth in SEQ ID NO: 100. In some embodiments, the single domain antibody provided herein comprises a FR4 having an amino acid sequence of the FR4 as set forth in SEQ ID NO: 100. In some embodiments, the single domain antibody provided herein comprises a FR1 and a FR2 having amino acid sequences of the FR1 and the FR2 as set forth in SEQ ID NO: 100. In some embodiments, the single domain antibody provided herein comprises a FR1 and a FR3 having amino acid sequences of the FR1 and the FR3 as set forth in SEQ ID NO: 100. In some embodiments, the single domain antibody provided herein comprises a FR1 and a FR4 having amino acid sequences of the FR1 and the FR4 as set forth in SEQ ID NO: 100. In some embodiments, the single domain antibody provided herein comprises a FR2 and a FR3 having amino acid sequences of the FR2 and the FR3 as set forth in SEQ ID NO: 100. In some embodiments, the single domain antibody provided herein comprises a FR2 and a FR4 having amino acid sequences of the FR2 and the FR4 as set forth in SEQ ID NO: 100. In some embodiments, the single domain antibody provided herein comprises a FR3 and a FR4 having amino acid sequences of the FR3 and the FR24 as set forth in SEQ ID NO: 100. In some embodiments, the single domain antibody provided herein comprises a FR1, a FR2, and a FR3 having amino acid sequences of the FR1, the FR2, and the FR3 as set forth in SEQ ID NO: 100. In some embodiments, the single domain antibody provided herein comprises a FR1, a FR2, and a FR4 having amino acid sequences of the FR1, the FR2, and the FR4 as set forth in SEQ ID NO: 100. In some embodiments, the single domain antibody provided herein comprises a FR1, a FR3, and a FR4 having amino acid sequences of the FR1, the FR3, and the FR4 as set forth in SEQ ID NO: 100. In some embodiments, the single domain antibody provided herein comprises a FR2, a FR3, and a FR4 having amino acid sequences of the FR2, the FR3, and the FR4 as set forth in SEQ ID NO: 100. In a specific embodiment, the single domain antibody provided herein comprises a FR1, a FR2, a FR3, and a FR4 having amino acid sequences of the FR1, the FR2, the FR3, and the FR4 as set forth in SEQ ID NO: 100.

In some embodiments, the single domain antibody provided herein comprises a FR1 having an amino acid sequence of the FR1 as set forth in SEQ ID NO: 101. In some embodiments, the single domain antibody provided herein comprises a FR2 having an amino acid sequence of the FR2 as set forth in SEQ ID NO: 101. In some embodiments, the single domain antibody provided herein comprises a FR3 having an amino acid sequence of the FR3 as set forth in SEQ ID NO: 101. In some embodiments, the single domain antibody provided herein comprises a FR4 having an amino acid sequence of the FR4 as set forth in SEQ ID NO: 101. In some embodiments, the single domain antibody provided herein comprises a FR1 and a FR2 having amino acid sequences of the FR1 and the FR2 as set forth in SEQ ID NO: 101. In some embodiments, the single domain antibody provided herein comprises a FR1 and a FR3 having amino acid sequences of the FR1 and the FR3 as set forth in SEQ ID NO: 101. In some embodiments, the single domain antibody provided herein comprises a FR1 and a FR4 having amino acid sequences of the FR1 and the FR4 as set forth in SEQ ID NO: 101. In some embodiments, the single domain antibody provided herein comprises a FR2 and a FR3 having amino acid sequences of the FR2 and the FR3 as set forth in SEQ ID NO: 101. In some embodiments, the single domain antibody provided herein comprises a FR2 and a FR4 having amino acid sequences of the FR2 and the FR4 as set forth in SEQ ID NO: 101. In some embodiments, the single domain antibody provided herein comprises a FR3 and a FR4 having amino acid sequences of the FR3 and the FR24 as set forth in SEQ ID NO: 101. In some embodiments, the single domain antibody provided herein comprises a FR1, a FR2, and a FR3 having amino acid sequences of the FR1, the FR2, and the FR3 as set forth in SEQ ID NO: 101. In some embodiments, the single domain antibody provided herein comprises a FR1, a FR2, and a FR4 having amino acid sequences of the FR1, the FR2, and the FR4 as set forth in SEQ ID NO: 101. In some embodiments, the single domain antibody provided herein comprises a FR1, a FR3, and a FR4 having amino acid sequences of the FR1, the FR3, and the FR4 as set forth in SEQ ID NO: 101. In some embodiments, the single domain antibody provided herein comprises a FR2, a FR3, and a FR4 having amino acid sequences of the FR2, the FR3, and the FR4 as set forth in SEQ ID NO: 101. In a specific embodiment, the single domain antibody provided herein comprises a FR1, a FR2, a FR3, and a FR4 having amino acid sequences of the FR1, the FR2, the FR3, and the FR4 as set forth in SEQ ID NO: 101.

In some embodiments, the single domain antibody provided herein comprises a FR1 having an amino acid sequence of the FR1 as set forth in SEQ ID NO: 102. In some embodiments, the single domain antibody provided herein comprises a FR2 having an amino acid sequence of the FR2 as set forth in SEQ ID NO: 102. In some embodiments, the single domain antibody provided herein comprises a FR3 having an amino acid sequence of the FR3 as set forth in SEQ ID NO: 102. In some embodiments, the single domain antibody provided herein comprises a FR4 having an amino acid sequence of the FR4 as set forth in SEQ ID NO: 102. In some embodiments, the single domain antibody provided herein comprises a FR1 and a FR2 having amino acid sequences of the FR1 and the FR2 as set forth in SEQ ID NO: 102. In some embodiments, the single domain antibody provided herein comprises a FR1 and a FR3 having amino acid sequences of the FR1 and the FR3 as set forth in SEQ ID NO: 102. In some embodiments, the single domain antibody provided herein comprises a FR1 and a FR4 having amino acid sequences of the FR1 and the FR4 as set forth in SEQ ID NO: 102. In some embodiments, the single domain antibody provided herein comprises a FR2 and a FR3 having amino acid sequences of the FR2 and the FR3 as set forth in SEQ ID NO: 102. In some embodiments, the single domain antibody provided herein comprises a FR2 and a FR4 having amino acid sequences of the FR2 and the FR4 as set forth in SEQ ID NO: 102. In some embodiments, the single domain antibody provided herein comprises a FR3 and a FR4 having amino acid sequences of the FR3 and the FR24 as set forth in SEQ ID NO: 102. In some embodiments, the single domain antibody provided herein comprises a FR1, a FR2, and a FR3 having amino acid sequences of the FR1, the FR2, and the FR3 as set forth in SEQ ID NO: 102. In some embodiments, the single domain antibody provided herein comprises a FR1, a FR2, and a FR4 having amino acid sequences of the FR1, the FR2, and the FR4 as set forth in SEQ ID NO: 102. In some embodiments, the single domain antibody provided herein comprises a FR1, a FR3, and a FR4 having amino acid sequences of the FR1, the FR3, and the FR4 as set forth in SEQ ID NO: 102. In some embodiments, the single domain antibody provided herein comprises a FR2, a FR3, and a FR4 having amino acid sequences of the FR2, the FR3, and the FR4 as set forth in SEQ ID NO: 102. In a specific embodiment, the single domain antibody provided herein comprises a FR1, a FR2, a FR3, and a FR4 having amino acid sequences of the FR1, the FR2, the FR3, and the FR4 as set forth in SEQ ID NO: 102.

In some embodiments, the single domain antibody provided herein comprises a FR1 having an amino acid sequence of the FR1 as set forth in SEQ ID NO: 103. In some embodiments, the single domain antibody provided herein comprises a FR2 having an amino acid sequence of the FR2 as set forth in SEQ ID NO: 103. In some embodiments, the single domain antibody provided herein comprises a FR3 having an amino acid sequence of the FR3 as set forth in SEQ ID NO: 103. In some embodiments, the single domain antibody provided herein comprises a FR4 having an amino acid sequence of the FR4 as set forth in SEQ ID NO: 103. In some embodiments, the single domain antibody provided herein comprises a FR1 and a FR2 having amino acid sequences of the FR1 and the FR2 as set forth in SEQ ID NO: 103. In some embodiments, the single domain antibody provided herein comprises a FR1 and a FR3 having amino acid sequences of the FR1 and the FR3 as set forth in SEQ ID NO: 103. In some embodiments, the single domain antibody provided herein comprises a FR1 and a FR4 having amino acid sequences of the FR1 and the FR4 as set forth in SEQ ID NO: 103. In some embodiments, the single domain antibody provided herein comprises a FR2 and a FR3 having amino acid sequences of the FR2 and the FR3 as set forth in SEQ ID NO: 103. In some embodiments, the single domain antibody provided herein comprises a FR2 and a FR4 having amino acid sequences of the FR2 and the FR4 as set forth in SEQ ID NO: 103. In some embodiments, the single domain antibody provided herein comprises a FR3 and a FR4 having amino acid sequences of the FR3 and the FR24 as set forth in SEQ ID NO: 103. In some embodiments, the single domain antibody provided herein comprises a FR1, a FR2, and a FR3 having amino acid sequences of the FR1, the FR2, and the FR3 as set forth in SEQ ID NO: 103. In some embodiments, the single domain antibody provided herein comprises a FR1, a FR2, and a FR4 having amino acid sequences of the FR1, the FR2, and the FR4 as set forth in SEQ ID NO: 103. In some embodiments, the single domain antibody provided herein comprises a FR1, a FR3, and a FR4 having amino acid sequences of the FR1, the FR3, and the FR4 as set forth in SEQ ID NO: 103. In some embodiments, the single domain antibody provided herein comprises a FR2, a FR3, and a FR4 having amino acid sequences of the FR2, the FR3, and the FR4 as set forth in SEQ ID NO: 103. In a specific embodiment, the single domain antibody provided herein comprises a FR1, a FR2, a FR3, and a FR4 having amino acid sequences of the FR1, the FR2, the FR3, and the FR4 as set forth in SEQ ID NO: 103.

In some embodiments, the single domain antibody provided herein is a humanized single domain antibody.

Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some specific embodiments, the single domain antibody comprises a sequence of

(SEQ ID NO: 93)
QVQLVESGGGLVQAGGSLKLACAAPGLTFSSYRMGWFRQAPGQEREFVAA
IDWNGRGTYYRYYADSVKGRSTISRDNAKNTMYLQMNSLKPEDTAVYYCA
AGSIDLNWYGGMDYWGQGTQVTVSS.

In some specific embodiments, the single domain antibody comprises a sequence of

(SEQ ID NO: 94)
EVQVVESGGGLVQAGGSLKLACAAPGLTFSSYRMGWFRQAPGQEREFVAA
IDWNGRGTYYRYYADSVKGRSTISRDNAKNTVYLQMNSLKPEDTAVYYCA
ATTVLTDPRVLNEYATWGQGTQVTVSS.

In some specific embodiments, the single domain antibody comprises a sequence of

(SEQ ID NO: 95)
QLQLVESGGGLVQPGGSLRLSCAASGSIFSINVMGWYRQAPGKQRELVAR
INGGGITHYAESVKGRFTISRDNAKNTVYLQMNSLKPEDTAAYYCKADVF
GSSGYVETYWGQGTQVTVSS.

In some specific embodiments, the single domain antibody comprises a sequence of

(SEQ ID NO: 96)
EVQVVESGGGLVQAGGSLRLSCAVSGTSVSSNAMGWYRQAPGKQREWVGF
IDRIATTTIATSVKGRFAITRDNAKNTVYLQMSGLKPEDTAVYYCNHPLT
ARWGQGTQVTVSS.

In some specific embodiments, the single domain antibody comprises a sequence of

(SEQ ID NO: 97)
QVQLVESGGGLVQAGGSLRLSCAASGRTFSSYAMGWFRQAPGKEREFVAA
ITWNGGTTYYADSVKGRFTISRDNAKNTVYLQMNSLKPEDTAVYYCAADP
FNQGYWGQGTQVTVSS.

In some specific embodiments, the single domain antibody comprises a sequence of

(SEQ ID NO: 98)
EVQLVESGGGLVQAGGSLRLSCAVSGSSVSSDAMGWYRQAPGNQRAWVAF
ISGGGTTTYADSVKGRFTISRDNTKNTVYLHMNSLKPEDTAVYYCNHPLT
SRWGQGTQVTVSS.

In some specific embodiments, the single domain antibody comprises a sequence of

(SEQ ID NO: 99)
EVQVVESGGGLVQAGGSLRLACVASRSIGSINVMGWYRQAPGKQRDLVAR
ITGGGSTHYAESVKGRFTISRDNAKNTVYLQMNSLEPEDTAVYYCASMVN
PIITAWGTIGVREIPDYDYWGQGTQVTVSS.

In some specific embodiments, the single domain antibody comprises a sequence of

(SEQ ID NO: 100)
QVQLVESGGGLVQAGGSLRLSCAVSGRTFSTYRMGWFRQAPGKERSFVAA
ISWSGGSTTYADPVKGRFTISRDNAKNTVYLRMNSLKPEDTAVYYCNDQR
GYWGQGTLVTVSS.

In some specific embodiments, the single domain antibody comprises a sequence of

(SEQ ID NO: 101)
EVQVVESGGGLVQAGGSLRLSCAASGFTFTRYAMGWFRQAPGKERSFVAA
ISWSGSSAGYGDSVKGRFTISRDNAKNTLYLQMNSLKPEDTAVYYCAADP
FNQGYWGQGTQVTVSS.

In some specific embodiments, the single domain antibody comprises a sequence of

(SEQ ID NO: 102)
EVQVVESGGGLVQAGGSLRLSCAASGRTFTTYRMGWFRQAPGKEREFVAA
IRWSGGRTLYADSVKGRFTISRDNAKNTAYLQMNNLRPEDTAVYYCAADL
AEYSGTYSSPADSPAGYDYWGQGTQVTVSS.

In some specific embodiments, the single domain antibody comprises a sequence of

(SEQ ID NO: 103)
QVQLVETGGGLVQAGDSLRLSCAASGRTLSFNTYAMGWFRQAPGKEREFV
ASITWNGGSTSYADSVKGRFTITRDNAKNTATLRMNSLQPDDTAVYYCAA
ARYYVSGTYFPANYWGQGTQVTVSS.

In certain embodiments, an antibody described herein or an antigen-binding fragment thereof comprises amino acid sequences with certain percent identity relative to any one of antibodies VHH1, VHH2, VHH3, VHH4, VHH5, VHH6, VHH7, VHH9, VHH10, VHH11 and VHH12.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and)(BLAST programs of Altschul et al., 1990, J. Mol. Biol. 215:403. BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the)(BLAST program parameters set, e.g., to score 50, word length=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of)(BLAST and NBLAST) can be used (see, e.g., National Center for Biotechnology Information (NCBI) on the worldwide web, ncbi.nlm.nih.gov). Another preferred, non limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In certain embodiments, the single domain antibody described herein comprises a VHH domain having at least 80%, at least 85%, at least 90%, at least 95%, at least 96%, at least 97%, at least 98%, or at least 99% sequence identity to the amino acid sequence of SEQ ID NO: 93, wherein the single domain antibody binds to pIgR.

In certain embodiments, the single domain antibody described herein comprises a VHH domain having at least 80%, at least 85%, at least 90%, at least 95%, at least 96%, at least 97%, at least 98%, or at least 99% sequence identity to the amino acid sequence of SEQ ID NO: 94, wherein the single domain antibody binds to pIgR.

In certain embodiments, the single domain antibody described herein comprises a VHH domain having at least 80%, at least 85%, at least 90%, at least 95%, at least 96%, at least 97%, at least 98%, or at least 99% sequence identity to the amino acid sequence of SEQ ID NO: 95, wherein the single domain antibody binds to pIgR.

In certain embodiments, the single domain antibody described herein comprises a VHH domain having at least 80%, at least 85%, at least 90%, at least 95%, at least 96%, at least 97%, at least 98%, or at least 99% sequence identity to the amino acid sequence of SEQ ID NO: 96, wherein the single domain antibody binds to pIgR.

In certain embodiments, the single domain antibody described herein comprises a VHH domain having at least 80%, at least 85%, at least 90%, at least 95%, at least 96%, at least 97%, at least 98%, or at least 99% sequence identity to the amino acid sequence of SEQ ID NO: 97, wherein the single domain antibody binds to pIgR.

In certain embodiments, the single domain antibody described herein comprises a VHH domain having at least 80%, at least 85%, at least 90%, at least 95%, at least 96%, at least 97%, at least 98%, or at least 99% sequence identity to the amino acid sequence of SEQ ID NO: 98, wherein the single domain antibody binds to pIgR.

In certain embodiments, the single domain antibody described herein comprises a VHH domain having at least 80%, at least 85%, at least 90%, at least 95%, at least 96%, at least 97%, at least 98%, or at least 99% sequence identity to the amino acid sequence of SEQ ID NO: 99, wherein the single domain antibody binds to pIgR.

In certain embodiments, the single domain antibody described herein comprises a VHH domain having at least 80%, at least 85%, at least 90%, at least 95%, at least 96%, at least 97%, at least 98%, or at least 99% sequence identity to the amino acid sequence of SEQ ID NO: 100, wherein the single domain antibody binds to pIgR.

In certain embodiments, the single domain antibody described herein comprises a VHH domain having at least 80%, at least 85%, at least 90%, at least 95%, at least 96%, at least 97%, at least 98%, or at least 99% sequence identity to the amino acid sequence of SEQ ID NO: 101, wherein the single domain antibody binds to pIgR.

In certain embodiments, the single domain antibody described herein comprises a VHH domain having at least 80%, at least 85%, at least 90%, at least 95%, at least 96%, at least 97%, at least 98%, or at least 99% sequence identity to the amino acid sequence of SEQ ID NO: 102, wherein the single domain antibody binds to pIgR.

In certain embodiments, the single domain antibody described herein comprises a VHH domain having at least 80%, at least 85%, at least 90%, at least 95%, at least 96%, at least 97%, at least 98%, or at least 99% sequence identity to the amino acid sequence of SEQ ID NO: 103, wherein the single domain antibody binds to pIgR.

In certain embodiments, the single domain antibody provided herein comprises a framework having at least 80%, at least 85%, at least 90%, at least 95%, at least 96%, at least 97%, at least 98%, or at least 99% sequence identity to the amino acid sequence of SEQ ID NO: 93, wherein the single domain antibody binds to pIgR.

In certain embodiments, the single domain antibody provided herein comprises a framework having at least 80%, at least 85%, at least 90%, at least 95%, at least 96%, at least 97%, at least 98%, or at least 99% sequence identity to the amino acid sequence of SEQ ID NO: 94, wherein the single domain antibody binds to pIgR.

In certain embodiments, the single domain antibody provided herein comprises a framework having at least 80%, at least 85%, at least 90%, at least 95%, at least 96%, at least 97%, at least 98%, or at least 99% sequence identity to the amino acid sequence of SEQ ID NO: 95, wherein the single domain antibody binds to pIgR.

In certain embodiments, the single domain antibody provided herein comprises a framework having at least 80%, at least 85%, at least 90%, at least 95%, at least 96%, at least 97%, at least 98%, or at least 99% sequence identity to the amino acid sequence of SEQ ID NO: 96, wherein the single domain antibody immunospecifically binds to pIgR.

In certain embodiments, the single domain antibody provided herein comprises a framework having at least 80%, at least 85%, at least 90%, at least 95%, at least 96%, at least 97%, at least 98%, or at least 99% sequence identity to the amino acid sequence of SEQ ID NO: 97, wherein the single domain antibody binds to pIgR.

In certain embodiments, the single domain antibody provided herein comprises a framework having at least 80%, at least 85%, at least 90%, at least 95%, at least 96%, at least 97%, at least 98%, or at least 99% sequence identity to the amino acid sequence of SEQ ID NO: 98, wherein the single domain antibody binds to pIgR.

In certain embodiments, the single domain antibody provided herein comprises a framework having at least 80%, at least 85%, at least 90%, at least 95%, at least 96%, at least 97%, at least 98%, or at least 99% sequence identity to the amino acid sequence of SEQ ID NO: 99, wherein the single domain antibody binds to pIgR.

In certain embodiments, the single domain antibody provided herein comprises a framework having at least 80%, at least 85%, at least 90%, at least 95%, at least 96%, at least 97%, at least 98%, or at least 99% sequence identity to the amino acid sequence of SEQ ID NO: 100, wherein the single domain antibody binds to pIgR.

In certain embodiments, the single domain antibody provided herein comprises a framework having at least 80%, at least 85%, at least 90%, at least 95%, at least 96%, at least 97%, at least 98%, or at least 99% sequence identity to the amino acid sequence of SEQ ID NO: 101, wherein the single domain antibody binds to pIgR.

In certain embodiments, the single domain antibody provided herein comprises a framework having at least 80%, at least 85%, at least 90%, at least 95%, at least 96%, at least 97%, at least 98%, or at least 99% sequence identity to the amino acid sequence of SEQ ID NO: 102, wherein the single domain antibody binds to pIgR.

In certain embodiments, the single domain antibody provided herein comprises a framework having at least 80%, at least 85%, at least 90%, at least 95%, at least 96%, at least 97%, at least 98%, or at least 99% sequence identity to the amino acid sequence of SEQ ID NO: 103, wherein the single domain antibody binds to pIgR.

5.2.2. Humanized Single Domain Antibodies

The single domain antibodies described herein include humanized single domain antibodies. General strategies to humanize single domain antibodies from Camelidae species have been described (see, e.g., Vincke et al., J. Biol. Chem., 2009, 284(5):3273-3284) and are useful for producing humanized VHH domains as disclosed herein. The design of humanized single domain antibodies from Camelidae species may include the hallmark residues in the VHH, such as residues 11, 37, 44, 45 and 47 (residue numbering according to Kabat) (Muyldermans, Reviews Mol Biotech 74:277-302 (2001).

Humanized antibodies, such as the humanized single domain antibodies disclosed herein can also be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 9317105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

In some embodiments, single domain antibodies provided herein can be humanized single domain antibodies that bind to pIgR, including human pIgR. For example, humanized single chain antibodies of the present disclosure may comprise one or more CDRs of VHH1, VHH2, VHH3, VHH4, VHH5, VHH6, VHH7, VHH9, VHH10, VHH11 and/or VHH12. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the CDRs of the parent non-human antibody are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains to be used in making the humanized antibodies can be important to reduce antigenicity. For example, according to the so-called “best-fit” method, the sequence of the variable domain of a non-human antibody is screened against the entire library of known human variable-domain sequences. The human sequence that is closest to that of the non-human antibody may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, VL6 subgroup I (VL6I) and VH subgroup III (VHIII). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). A one-step FR shuffling process may be used. Such a process has been shown to be efficient, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. This methodology typically results in epitope retention and identification of antibodies from multiple subclasses with distinct human V-segment CDRs.

The “human engineering” method involves altering a non-human antibody or antibody fragment by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody.

A deimmunized antibody is an antibody in which T-cell epitopes have been removed. Methods for making deimmunized antibodies have been described. See, e.g., Jones et al., Methods Mol Biol. 2009; 525:405-23, xiv, and De Groot et al., Cell. Immunol. 244:148-153(2006)). Deimmunized antibodies comprise T-cell epitope-depleted variable regions and human constant regions. Briefly, variable regions of an antibody are cloned and T-cell epitopes are subsequently identified by testing overlapping peptides derived from the variable regions of the antibody in a T cell proliferation assay. T cell epitopes are identified via in silico methods to identify peptide binding to human MHC class II. Mutations are introduced in the variable regions to abrogate binding to human MHC class II. Mutated variable regions are then utilized to generate the deimmunized antibody.

5.2.3. Single Domain Antibody Variants

In some embodiments, amino acid sequence modification(s) of the single domain antibodies that bind to pIgR described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the single domain antibodies that bind to pIgR described herein, it is contemplated that variants of the single domain antibodies that bind to pIgR described herein can be prepared. For example, single domain antibody variants can be prepared by introducing appropriate nucleotide changes into the encoding DNA, and/or by synthesis of the desired antibody or polypeptide. Those skilled in the art who appreciate that amino acid changes may alter post-translational processes of the single domain antibody.

In some embodiments, the single domain antibodies provided herein are chemically modified, for example, by the covalent attachment of any type of molecule to the single domain antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, or conjugation to one or more immunoglobulin domains (e.g., Fc or a portion of an Fc). Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may be a substitution, deletion, or insertion of one or more codons encoding the single domain antibody or polypeptide that results in a change in the amino acid sequence as compared with the original antibody or polypeptide. Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Single domain antibodies generated by conservative amino acid substitutions are included in the present disclosure. In a conservative amino acid substitution, an amino acid residue is replaced with an amino acid residue having a side chain with a similar charge. As described above, families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. conservative (e.g., within an amino acid group with similar properties and/or side chains) substitutions may be made, so as to maintain or not significantly change the properties.

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, any cysteine residue not involved in maintaining the proper conformation of the single domain antibody also may be substituted, for example, with another amino acid, such as alanine or serine, to improve the oxidative stability of the molecule and to prevent aberrant crosslinking.

The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the single domain antibody variant DNA.

5.2.4. In Vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, 2002, Methods. Mol. Biol. 178:1-37; and Bradbury and Marks, 2004, J. Immunol. Methods 290:29-49.

In a yeast display system (see, e.g., Boder et al., 1997, Nat. Biotech. 15:553-57; and Chao et al., 2006, Nat. Protocols 1:755-68), the antibody may be fused to the adhesion subunit of the yeast agglutinin protein Aga2p, which attaches to the yeast cell wall through disulfide bonds to Agalp. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the single chain antibody (e.g., scFv). Expression has been shown to correlate with the stability of the displayed protein, and thus antibodies can be selected for improved stability as well as affinity (see, e.g., Shusta et al., 1999, J. Mol. Biol. 292:949-56). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (see, e.g., U.S. Pat. Publication 2003/0186374; and Blaise et al., 2004, Gene 342:211-18).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., 2006, Nucleic Acids Res. 34:e127). In mRNA display, a covalent bond between antibody and mRNA is established using puromycin as an adaptor molecule (Wilson et al., 2001, Proc. Natl. Acad. Sci. USA 98:3750-55).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In some embodiments, mammalian display systems may be used.

Diversity may also be introduced into the CDRs of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., 2005, J. Biol. Chem. 280:607-17) or residues suspected of affecting affinity on experimental basis or structural reasons. Diversity may also be introduced by replacement of regions that are naturally diverse via DNA shuffling or similar techniques (see, e.g., Lu et al., 2003, J. Biol. Chem. 278:43496-507; U.S. Pat. Nos. 5,565,332 and 6,989,250). Alternative techniques target hypervariable loops extending into framework-region residues (see, e.g., Bond et al., 2005, J. Mol. Biol. 348:699-709) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, single domain antibodies can be immobilized onto solid supports, columns, pins, or cellulose/poly(vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, 2005, Nature Biotechnology 23:1105-16; Quiroz and Sinclair, 2010, Revista Ingeneria Biomedia 4:39-51; and references therein.

5.2.5. Modifications of Single Domain Antibodies

Covalent modifications of single domain antibodies are included within the scope of the present disclosure. Covalent modifications include reacting targeted amino acid residues of a single domain antibody with an organic derivatizing agent that is capable of reacting with selected side chains or the N- or C-terminal residues of the single domain antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the single domain antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337. The single domain antibody that binds to pIgR of the disclosure may also be genetically fused or conjugated to one or more immunoglobulin constant regions or portions thereof (e.g., Fc) to extend half-life and/or to impart known Fc-mediated effector functions.

The single chain antibody that binds to pIgR of the present disclosure may also be modified to form chimeric molecules comprising the single chain antibody that binds to pIgR fused to another, heterologous polypeptide or amino acid sequence, for example, an epitope tag (see, e.g., Terpe, 2003, Appl. Microbiol. Biotechnol. 60:523-33) or the Fc region of an IgG molecule (see, e.g., Aruffo, Antibody Fusion Proteins 221-42 (Chamow and Ashkenazi eds., 1999)). The single chain antibody that binds to pIgR may also be used to generate pIgR binding chimeric antigen receptor (CAR).

Also provided herein are fusion proteins comprising the single chain antibody that binds to pIgR of the disclosure and a heterologous polypeptide. In some embodiments, the heterologous polypeptide to which the antibody is genetically fused or chemically conjugated is useful for targeting the antibody to cells having cell surface-expressed pIgR.

Also provided herein are panels of antibodies that bind to an pIgR antigen. In specific embodiments, the panels of antibodies have different association rates, different dissociation rates, different affinities for an pIgR antigen, and/or different specificities for an pIgR antigen. In some embodiments, the panels comprise or consist of about 10, about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96-well or 384-well plates, for assays such as ELISAs.

5.2.6. Preparation of Single Domain Antibodies

Single domain antibodies provided herein may be produced by culturing cells transformed or transfected with a vector containing a single domain antibody-encoding nucleic acids. Polynucleotide sequences encoding polypeptide components of the antibody of the present disclosure can be obtained using standard recombinant techniques. Desired polynucleotide sequences may be isolated and sequenced from antibody producing cells such as hybridomas cells or B cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells suitable for expressing antibodies of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. Antibodies produced by the host cells are purified using standard protein purification methods as known in the art.

Methods for antibody production including vector construction, expression, and purification are further described in Plückthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherichia coli, in Antibody Expression and Production (Al-Rubeai ed., 2011); and Therapeutic Monoclonal Antibodies: From Bench to Clinic (An ed., 2009).

It is, of course, contemplated that alternative methods, which are well known in the art, may be employed to prepare anti-pIgR antibodies. For instance, the appropriate amino acid sequence, or portions thereof, may be produced by direct peptide synthesis using solid-phase techniques (see, e.g., Stewart et al., Solid-Phase Peptide Synthesis (1969); and Merrifield, 1963, J. Am. Chem. Soc. 85:2149-54). In vitro protein synthesis may be performed using manual techniques or by automation. Various portions of the anti-pIgR antibody may be chemically synthesized separately and combined using chemical or enzymatic methods to produce the desired anti-pIgR antibody. Alternatively, antibodies may be purified from cells or bodily fluids, such as milk, of a transgenic animal engineered to express the antibody, as disclosed, for example, in U.S. Pat. Nos. 5,545,807 and 5,827,690.

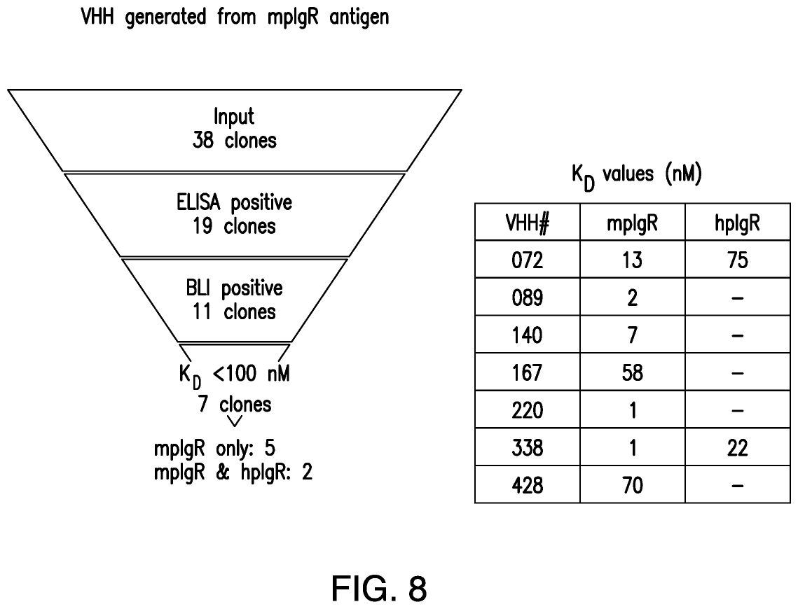

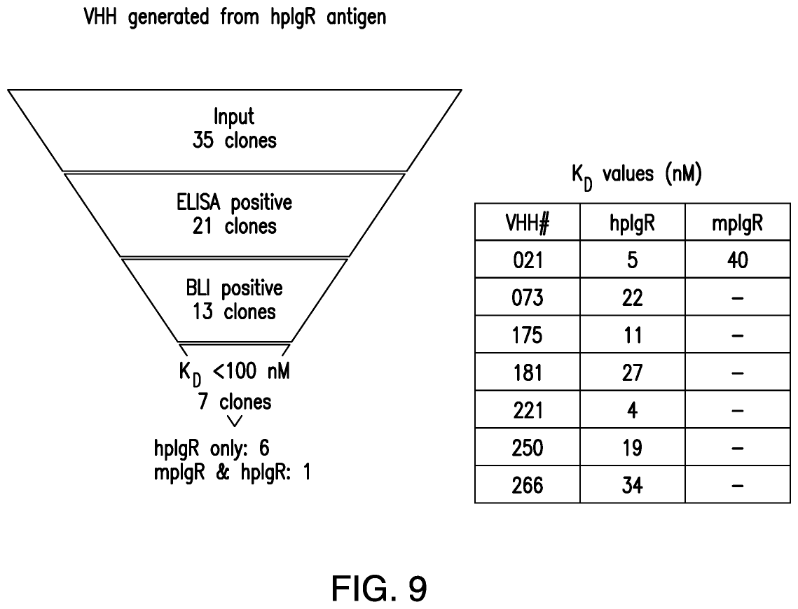

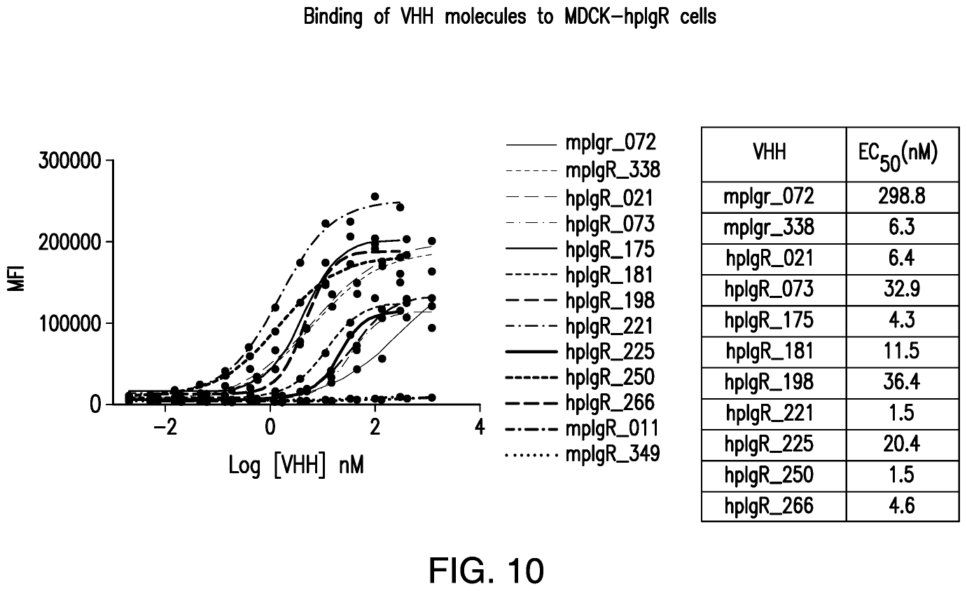

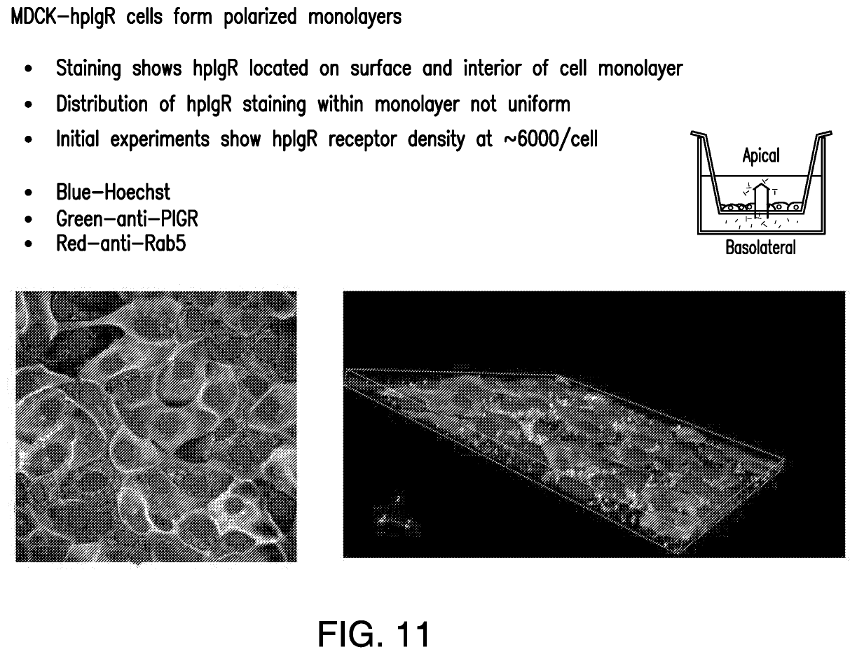

Specifically, the single domain antibodies, or other pIgR binders, can be generated by immunizing llamas using mpIgR and hpIgR extracellular domain (ECD), performing single B-cell sorting, undertaking V-gene extraction, cloning the pIgR binders, such as VHH-Fc fusions, and then performing small scale expression and purification. Additional screening of the single domain antibodies and other molecules that bind to pIgR can be performed, including one or more of selecting for ELISA-positive, BLI-positive, and K_{D} less than 100 nM. These selection criteria can be combined as shown in FIG. 8 (VHH generated from mpIgR antigen) and FIG. 9 (VHH generated from hpIgR antigen). Additionally, individual VHH binders (and other molecules that bind to pIgR) can be assayed for their ability to bind to MDCK cells expressing pIgR, e.g., hpIgR. Such assay can be performed using FACS analysis with MDCK cells expressing hpIgR, and measuring the mean fluorescence intensity (MFI) of fluorescently-labeled VHH molecules. The results of such experiment are shown in FIG. 10. The staining of hpIgR on a monolayer of MDCK cells is shown in FIG. 11.

### 5.3. Therapeutic Molecules Comprising the Single Domain Antibodies

In one aspect, provided herein is a therapeutic molecule comprising a single domain antibody (e.g., a VHH domain) provided herein and a therapeutic agent.

In various embodiments, the single domain antibody provided herein can be genetically fused or chemically conjugated to any agents for delivery of these agents, for example, protein-based entities. The single domain antibody may be chemically-conjugated to the agent, or otherwise non-covalently conjugated to the agent.

The single domain antibodies provided herein are useful for delivering agents that can be used to treat subjects, such as biologics (including protein based therapeutics such as peptides and antibodies), and nucleotide based therapeutics such as viral gene therapeutics or RNA therapeutics). For example, the agent can be a diabetes medication, optionally selected from a group consisting of insulin, glucagon-like-peptide-1, insulin-mimic peptides, and glucagon-like-peptide-1-mimic peptides. The agent can be a peptide or antibody (or a fragment thereof), optionally selected from a group consisting of an anti-TNF-alpha antibody or a fragment thereof, an anti-IL23 antibody or a fragment thereof, an antibody that binds to a receptor of IL23 or a fragment thereof, an IL23 receptor inhibitor, and an immune checkpoint antibody such as an anti-PD-1 antibody. The agent can also be a vaccine, such as a vaccine for preventing an infection selected from a group consisting of Vibrio, Cholera, Typhoid, Rotavirus, Tuberculosis, HIV, Flu, Ebola, and Sendai.

Thus, provided herein are single domain antibodies (e.g., VHH domains) that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, about 100, about 150, about 200, about 250, about 300, about 350, about 400, about 450 or about 500 amino acids, or over 500 amino acids) to generate fusion proteins, as well as uses thereof. In particular, provided herein are fusion proteins comprising an antigen-binding fragment of the single domain antibody provided herein (e.g., CDR1, CDR2, and/or CDR3) and a heterologous protein, polypeptide, or peptide. For example, an antibody that binds to a cell surface receptor expressed by a particular cell type may be fused or conjugated to a modified antibody provided herein.

Moreover, antibodies provided herein can be fused to marker or “tag” sequences, such as a peptide, to facilitate purification. In specific embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for fusing or conjugating moieties (including polypeptides) to antibodies are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., 1982, Immunol. Rev. 62:119-58; U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., 1991, Proc. Natl. Acad. Sci. USA, 88: 10535-39; Traunecker et al., 1988, Nature, 331:84-86; Zheng et al., 1995, J. Immunol. 154:5590-600; and Vil et al., 1992, Proc. Natl. Acad. Sci. USA 89:11337-41).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the single domain antibodies as provided herein, including, for example, antibodies with higher affinities and lower dissociation rates (see, e.g., U.S. Pat. Nos. 5,605,793; 5,811,238; 5,830,721; 5,834,252; and 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

In some embodiments, a single domain antibody provided herein (e.g., VHH domain) is conjugated to a second antibody to form an antibody heteroconjugate as described, for example, in U.S. Pat. No. 4,676,980.

Antibodies that bind to pIgR as provided herein may also be attached to solid supports, which are particularly useful for immunoassays or purification of the target antigen. Such solid supports include, but are not limited to, glass, cellulose, polyacrylamide, nylon, polystyrene, polyvinyl chloride, or polypropylene.

Other exemplary agents include, but are not limited to, a small molecule, a polynucleotide, a radioisotope, a toxin, an enzyme, an anticoagulant, a hormone, a cytokine, an anti-inflammatory molecule, an RNAi, a mRNA, a self-replicating RNA, an antibiotic, or an antibody-antibiotic conjugate. In one embodiment, the agent is an antibiotic. Exemplary antibiotics include, but are not limited to, macrolide antibiotic, a fluoroquinolone, a tetracycline, amoxicillin, ceftriaxone, penicillin G, linezolid, moxifloxacin, and azithromycin. Exemplary radioisotopes include, but are not limited to from ¹⁸F, ⁹⁹Tc, ¹¹¹In, ¹²³I, ²⁰¹Tl, ¹³³Xe, ¹¹C, ¹³N, ¹⁵O, ¹⁸F, ⁶²Cu, ⁶⁴Cu, ¹²⁴I, ⁷⁶Br, ⁸²Rb, ⁸⁹Zr and ⁶⁸Ga.

In other embodiments, antibodies provided herein are conjugated or recombinantly fused, e.g., to a diagnostic molecule.

Such diagnosis and detection can be accomplished, for example, by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, 225Acγ-emitting, Auger-emitting, β-emitting, an alpha-emitting or positron-emitting radioactive isotope. Exemplary radioactive isotopes include 3H, 11C, 13C, 15N, 18F, 19F, 55Co, 57Co, 60Co, 61Cu, 62Cu, 64Cu, 67Cu, 68Ga, 72As, 75Br, 86Y, 89Zr, 90Sr, 94mTc, 99mTc, 1151n, 1231, 1241, 1251, 1311, 211At, 212Bi, 213Bi, 223Ra, 226Ra, 225Ac and 227Ac.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers (see, e.g., Chari et al., 1992, Cancer Res. 52:127-31; and U.S. Pat. No. 5,208,020), thioether linkers, or hydrophilic linkers designed to evade multidrug transporter-mediated resistance (see, e.g., Kovtun et al., 2010, Cancer Res. 70:2528-37).

Conjugates of the antibody and agent may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MB S, sulfo-SIAB, sulfo-SMCC, sulfo-SMPB, and SVSB (succinimidyl-(4-vinylsulfone)benzoate). The present disclosure further contemplates that conjugates of antibodies and agents may be prepared using any suitable methods as disclosed in the art (see, e.g., Bioconjugate Techniques (Hermanson ed., 2d ed. 2008)).

Conventional conjugation strategies for antibodies and agents have been based on random conjugation chemistries involving the ε-amino group of Lys residues or the thiol group of Cys residues, which results in heterogenous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332: 41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50):12047-57).

5.3.1. Methods of Making a Genetically Fused Protein

In various embodiments, the single domain antibody is genetically fused to the agent. Genetic fusion may be accomplished by placing a linker (e.g., a polypeptide) between the single domain antibody and the agent. The linker may be a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

In various embodiments, the single domain antibody is genetically conjugated to a therapeutic molecule, with a hinge region linking the single domain antibody to the therapeutic molecule. The hinge region may be a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20. In some embodiments, the hinge region comprises the sequence EPKTPKPQPQPQLQPQPNPTTESKSPK (SEQ ID NO: 130), or comprises an amino acid sequence having at least 50, at least 55, at least 60, at least 65, at least 70, at least 75, at least 80, at least 85, at least 90, at least 95, at least 98 or at least 99%, sequence identity with EPKTPKPQPQPQLQPQPNPTTESKSPK (SEQ ID NO: 130). In some embodiments, the hinge region comprises the sequence EPKSCDKTHTCPPCP (SEQ ID NO: 150), or comprises an amino acid sequence having at least 50, at least 55, at least 60, at least 65, at least 70, at least 75, at least 80, at least 85, at least 90, at least 95, at least 98 or at least 99%, sequence identity with EPKSCDKTHTCPPCP (SEQ ID NO: 150). In some embodiments, the hinge region comprises the sequence ERKCCVECPPCP (SEQ ID NO: 151), or comprises an amino acid sequence having at least 50, at least 55, at least 60, at least 65, at least 70, at least 75, at least 80, at least 85, at least 90, at least 95, at least 98 or at least 99%, sequence identity with ERKCCVECPPCP (SEQ ID NO: 151). In some embodiments, the hinge region comprises the sequence ELKTPLGDTTHTCPRCP(EPKSCDTPPPCPRCP)₃ (SEQ ID NO: 152), or comprises an amino acid sequence having at least 50, at least 55, at least 60, at least 65, at least 70, at least 75, at least 80, at least 85, at least 90, at least 95, at least 98 or at least 99%, sequence identity with ELKTPLGDTTHTCPRCP(EPKSCDTPPPCPRCP)₃ (SEQ ID NO: 152). In some embodiments, the hinge region comprises the sequence ESKYGPPCPSCP (SEQ ID NO: 153), or comprises an amino acid sequence having at least 50, at least 55, at least 60, at least 65, at least 70, at least 75, at least 80, at least 85, at least 90, at least 95, at least 98 or at least 99%, sequence identity with ESKYGPPCPSCP (SEQ ID NO: 153).

Also provided herein are methods for making the various fusion proteins provided herein. In a specific embodiment, the fusion protein provided herein is recombinantly expressed.

Recombinant expression of a fusion protein provided herein may require construction of an expression vector containing a polynucleotide that encodes the protein or a fragment thereof. Once a polynucleotide encoding a protein provided herein or a fragment thereof has been obtained, the vector for the production of the molecule may be produced by recombinant DNA technology using techniques well-known in the art. Thus, methods for preparing a protein by expressing a polynucleotide containing an encoding nucleotide sequence are described herein. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding a fusion protein provided herein, or a fragment thereof, or a CDR, operably linked to a promoter.

The expression vector can be transferred to a host cell by conventional techniques and the transfected cells are then cultured by conventional techniques to produce a fusion protein provided herein. Thus, also provided herein are host cells containing a polynucleotide encoding a fusion protein provided herein or fragments thereof operably linked to a heterologous promoter.

A variety of host-expression vector systems may be utilized to express the fusion protein provided herein (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express a fusion protein provided herein in situ. These include but are not limited to microorganisms such as bacteria (e.g., E. coli and B. subtilis) transformed with recombinant bacteriophage DNA, plasmid DNA or cosmid DNA expression vectors containing coding sequences; yeast (e.g., Saccharomyces Pichia) transformed with recombinant yeast expression vectors containing coding sequences; insect cell systems infected with recombinant virus expression vectors (e.g., baculovirus) containing coding sequences; plant cell systems infected with recombinant virus expression vectors (e.g., cauliflower mosaic virus, CaMV, tobacco mosaic virus, TMV) or transformed with recombinant plasmid expression vectors (e.g., Ti plasmid) containing coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or, eukaryotic cells, especially for the expression of whole recombinant antibody molecule, can be used for the expression of a recombinant fusion protein. For example, mammalian cells such as Chinese hamster ovary cells (CHO), in conjunction with a vector such as the major intermediate early gene promoter element from human cytomegalovirus is an effective expression system for antibodies or variants thereof (Foecking et al., 1986, Gene 45:101; and Cockett et al., 1990, Bio/Technology 8:2). In some embodiments, fusion proteins provided herein are produced in CHO cells. In a specific embodiment, the expression of nucleotide sequences encoding the fusion proteins provided herein is regulated by a constitutive promoter, inducible promoter or tissue specific promoter.

In bacterial systems, a number of expression vectors may be advantageously selected depending upon the use intended for the fusion protein being expressed. For example, when a large quantity of such a fusion protein is to be produced, for the generation of pharmaceutical compositions of a fusion protein, vectors which direct the expression of high levels of fusion protein products that are readily purified may be desirable. Such vectors include, but are not limited to, the E. coli expression vector pUR278 (Ruther et al., 1983, EMBO 12:1791), in which the coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the fusion protein in infected hosts (e.g., see Logan & Shenk, 1984, Proc. Natl. Acad. Sci. USA 8 1:355-359). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NSO (a murine myeloma cell line that does not endogenously produce any immunoglobulin chains), CRL7O3O and HsS78Bst cells.

For long-term, high-yield production of recombinant proteins, stable expression can be utilized. For example, cell lines which stably express the fusion proteins may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the fusion protein. Such engineered cell lines may be particularly useful in screening and evaluation of compositions that interact directly or indirectly with the binding molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, N Y (1993); Kriegler, Gene Transfer and Expression, A Laboratory Manual, Stockton Press, N Y (1990); and in Chapters 12 and 13, Dracopoli et al. (eds.), Current Protocols in Human Genetics, John Wiley & Sons, N Y (1994); Colberre-Garapin et al., 1981, J. Mol. Biol. 150:1, which are incorporated by reference herein in their entireties.

The expression level of a fusion protein can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing a fusion protein is amplifiable, increase in the level of inhibitor present in culture of host cell will increase the number of copies of the marker gene. Since the amplified region is associated with the fusion protein gene, production of the fusion protein will also increase (Crouse et al., 1983, Mol. Cell. Biol. 3:257).

The host cell may be co-transfected with multiple expression vectors provided herein. The vectors may contain identical selectable markers which enable equal expression of respective encoding polypeptides. Alternatively, a single vector may be used which encodes, and is capable of expressing multiple polypeptides. The coding sequences may comprise cDNA or genomic DNA.

Once a fusion protein provided herein has been produced by recombinant expression, it may be purified by any method known in the art for purification of a polypeptide (e.g., an immunoglobulin molecule), for example, by chromatography (e.g., ion exchange, affinity, particularly by affinity for the specific antigen after Protein A, sizing column chromatography, and Kappa select affinity chromatography), centrifugation, differential solubility, or by any other standard technique for the purification of proteins. Further, the fusion protein molecules provided herein can be fused to heterologous polypeptide sequences described herein or otherwise known in the art to facilitate purification.

### 5.4. Polynucleotides

In certain embodiments, the disclosure provides polynucleotides that encode the single domain antibodies that bind to pIgR and fusion proteins comprising the single domain antibodies that bind of pIgR described herein. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

In exemplary embodiments, the nucleic acid molecule provided herein comprises a sequence that encodes the single domain antibody having the sequence of:

(SEQ ID NO: 93)
QVQLVESGGGLVQAGGSLKLACAAPGLTFSSYRMGWFRQAPGQEREFVAAI
DWNGRGTYYRYYADSVKGRSTISRDNAKNTMYLQMNSLKPEDTAVYYCAAG
SIDLNWYGGMDYWGQGTQVTVSS,
(SEQ ID NO: 94)
EVQVVESGGGLVQAGGSLKLACAAPGLTFSSYRMGWFRQAPGQEREFVAAI
DWNGRGTYYRYYADSVKGRSTISRDNAKNTVYLQMNSLKPEDTAVYYCAAT
TVLTDPRVLNEYATWGQGTQVTVSS,
(SEQ ID NO: 95)
QLQLVESGGGLVQPGGSLRLSCAASGSIFSINVMGWYRQAPGKQRELVARI
NGGGITHYAESVKGRFTISRDNAKNTVYLQMNSLKPEDTAAYYCKADVFGS
SGYVETYWGQGTQVTVSS,
(SEQ ID NO: 96)
EVQVVESGGGLVQAGGSLRLSCAVSGTSVSSNAMGWYRQAPGKQREWVGFI
DRIATTTIATSVKGRFAITRDNAKNTVYLQMSGLKPEDTAVYYCNHPLTAR
WGQGTQVTVSS,
(SEQ ID NO: 97)
QVQLVESGGGLVQAGGSLRLSCAASGRTFSSYAMGWFRQAPGKEREFVAAI
TWNGGTTYYADSVKGRFTISRDNAKNTVYLQMNSLKPEDTAVYYCAADPFN
QGYWGQGTQVTVSS,
(SEQ ID NO: 98)
EVQLVESGGGLVQAGGSLRLSCAVSGSSVSSDAMGWYRQAPGNQRAWVAFI
SGGGTTTYADSVKGRFTISRDNTKNTVYLHMNSLKPEDTAVYYCNHPLTSR
WGQGTQVTVSS,
(SEQ ID NO: 99)
EVQVVESGGGLVQAGGSLRLACVASRSIGSINVMGWYRQAPGKQRDLVARI
TGGGSTHYAESVKGRFTISRDNAKNTVYLQMNSLEPEDTAVYYCASMVNPI
ITAWGTIGVREIPDYDYWGQGTQVTVSS,
(SEQ ID NO: 100)
QVQLVESGGGLVQAGGSLRLSCAVSGRTFSTYRMGWFRQAPGKERSFVAAI
SWSGGSTTYADPVKGRFTISRDNAKNTVYLRMNSLKPEDTAVYYCNDQRGY
WGQGTLVTVSS,
(SEQ ID NO: 101)
EVQVVESGGGLVQAGGSLRLSCAASGFTFTRYAMGWFRQAPGKERSFVAAI
SWSGSSAGYGDSVKGRFTISRDNAKNTLYLQMNSLKPEDTAVYYCAADPFN
QGYWGQGTQVTVSS,
(SEQ ID NO: 102)
EVQVVESGGGLVQAGGSLRLSCAASGRTFTTYRMGWFRQAPGKEREFVAAI
RWSGGRTLYADSVKGRFTISRDNAKNTAYLQMNNLRPEDTAVYYCAADLAE
YSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFVA
SITWNGGSTSYADSVKGRFTITRDNAKNTATLRMNSLQPDDTAVYYCAAAR
YYVSGTYFPANYWGQGTQVTVSS.

In exemplary embodiments, the nucleic acid molecule comprises the sequence of:

(SEQ ID NO: 133)
CAGGTGCAGCTGGTGGAGTCTGGGGGAGGATTGGTGCAGGCTGGGGGCTCT
CTGAAACTCGCCTGTGCAGCACCTGGACTTACCTTCAGTTCGTATCGCATG
GGCTGGTTCCGCCAGGCTCCAGGGCAGGAGCGTGAGTTTGTAGCAGCTATT
GATTGGAATGGTCGTGGCACATATTATCGATACTATGCAGACTCCGTGAAG
GGCCGATCCACCATTTCCAGAGACAACGCCAAGAACACGATGTATCTGCAA
ATGAACAGCCTGAAACCTGAGGACACGGCCGTTTATTACTGTGCAGCAGGT
TCGATCGACCTTAACTGGTACGGCGGCATGGACTACTGGGGCNANGGGACC
CAGGTCACCGTCTCCTCA,
(SEQ ID NO: 134)
GAGGTGCAGGTGGTGGAGTCTGGGGGAGGATTGGTGCAGGCTGGGGGCTCT
CTGAAACTCGCCTGTGCAGCACCTGGACTTACCTTCAGTTCGTATCGCATG
GGCTGGTTCCGCCAGGCTCCAGGGCAGGAGCGTGAGTTTGTAGCAGCTATT
GATTGGAATGGTCGTGGCACATATTATCGATACTATGCAGACTCCGTGAAG
GGCCGATCCACCATTTCCAGAGACAACGCCAAGAACACGGTGTATCTGCAA
ATGAACAGCCTGAAACCTGAGGACACGGCCGTTTATTACTGTGCAGCTACT
ACGGTATTAACTGACCCTAGGGTTCTTAATGAGTATGCCACATGGGGCCAG
GGGACCCAGGTCACCGTCTCCTCA,
(SEQ ID NO: 135)
CAGTTGCAGCTCGTGGAGTCTGGGGGAGGCTTGGTGCAGCCTGGGGGGTCT
CTGAGACTCTCCTGTGCAGCCTCTGGAAGCATCTTCAGTATCAATGTTATG
GGCTGGTACCGCCAGGCTCCAGGGAAGCAGCGCGAGTTGGTCGCACGTATT
AATGGAGGTGGCATTACACACTATGCAGAGTCCGTGAAGGGCCGATTCACC
ATCTCCAGAGACAACGCCAAGAACACGGTGTATCTGCAAATGAACAGCCTG
AAACCTGAGGACACAGCCGCATATTACTGTAAGGCAGATGTGTTCGGTAGT
AGCGGGTACGTAGAAACCTACTGGGGCCAGGGGACCCAGGTCACCGTCTCC
TCA,
(SEQ ID NO: 136)
GAGGTGCAGGTGGTGGAGTCTGGGGGAGGCTTGGTGCAGGCTGGGGGCTCT
CTGAGACTCTCCTGTGCAGTCTCTGGAACCTCCGTCAGTAGCAATGCCATG
GGTTGGTACCGCCAGGCTCCAGGGAAGCAGCGCGAGTGGGTCGGATTTATT
GATCGTATTGCTACCACGACGATTGCAACCTCCGTGAAGGGCCGATTCGCC
ATCACCAGAGACAACGCCAAGAACACGGTGTATCTCCAAATGAGCGGCCTG
AAACCTGAGGACACAGCCGTCTATTACTGTAATCATCCATTGACCGCTCGG
TGGGGCCAGGGGACCCAGGTCACCGTCTCCTCA,
(SEQ ID NO: 137)
CAGGTGCAGCTGGTGGAGTCTGGGGGAGGCTTGGTGCAGGCTGGGGGCTCT
CTGAGACTCTCCTGTGCAGCCTCTGGACGCACCTTCAGTAGCTATGCCATG
GGCTGGTTCCGCCAGGCTCCAGGGAAGGAGCGTGAGTTTGTAGCAGCTATT
ACCTGGAATGGTGGTACCACATACTATGCAGACTCCGTGAAGGGCCGATTC
ACCATCTCCAGAGACAACGCCAAGAACACGGTGTATCTGCAAATGAACAGC
CTGAAACCTGAGGACACGGCCGTTTATTACTGTGCAGCAGACCCATTCAAC
CAAGGCTACTGGGGCCAGGGGACCCAGGTCACCGTCTCCTCA,
(SEQ ID NO: 138)
GAGGTGCAGCTCGTGGAGTCTGGAGGAGGCTTGGTGCAGGCTGGGGGGTCT
CTGAGACTCTCCTGTGCAGTCTCTGGAAGCTCCGTCAGTAGCGATGCCATG
GGTTGGTACCGCCAGGCTCCAGGGAATCAGCGCGCGTGGGTCGCATTTATT
TCTGGTGGTGGTACCACAACCTATGCAGACTCCGTTAAGGGCCGATTCACC
ATCTCCAGAGACAACACCAAGAACACGGTGTATCTCCACATGAACAGCCTG
AAACCTGAAGACACAGCCGTCTATTACTGTAATCATCCATTGACGTCTCGG
TGGGGCCAGGGGACCCAGGTCACCGTCTCCTCA,
(SEQ ID NO: 139)
GAGGTGCAGGTGGTGGAGTCTGGGGGAGGATTGGTGCAGGCTGGGGGGTCT
CTGAGACTCGCCTGTGTAGCCTCTAGAAGCATCGGCAGTATCAATGTTATG
GGCTGGTACCGCCAGGCTCCAGGGAAGCAGCGCGACTTGGTCGCACGTATT
ACTGGAGGTGGCAGTACACACTACGCAGAGTCCGTGAAGGGCCGATTCACC
ATCTCCAGAGACAACGCCAAGAACACGGTGTATCTGCAAATGAACAGCCTG
GAACCTGAGGACACGGCCGTTTATTACTGTGCGTCAATGGTAAACCCTATC
ATTACGGCTTGGGGTACGATTGGTGTGCGCGAGATTCCCGACTATGACTAC
TGGGGCCAGGGGACCCAGGTCACCGTCTCCTCA,
(SEQ ID NO: 140)
GAGGTGCAGGTGGTGGAGTCTGGGGGAGGCTTGGTGCAGGCTGGGGGGTCT
CTGAGACTCTCCTGTGCAGCCTCTGGATTCACCTTCACCCGCTATGCCATG
GGCTGGTTCCGCCAGGCTCCAGGGAAGGAGCGATCGTTTGTAGCAGCTATT
AGCTGGAGTGGTAGTAGCGCAGGCTATGGAGACTCCGTGAAGGGCCGATTC
ACCATCTCCAGAGACAACGCCAAGAACACGCTGTATCTGCAAATGAACAGT
CTAAAACCTGAGGACACGGCCGTTTATTACTGTGCAGCAGACCCATTCAAC
CAAGGCTACTGGGGCCAGGGGACCCAGGTCACCGTCTCCTCA,
(SEQ ID NO: 141)
GAGGTGCAGGTGGTGGAGTCTGGGGGAGGATTGGTGCAGGCTGGGGGCTCT
CTGAGACTCTCCTGTGCAGCCTCTGGACGCACCTTCACTACCTATCGCATG
GGCTGGTTCCGCCAGGCTCCAGGGAAGGAGCGAGAGTTTGTAGCAGCTATT
CGCTGGAGTGGTGGTCGCACATTGTATGCAGACTCCGTGAAGGGCCGATTC
ACCATCTCCAGAGACAACGCCAAGAACACAGCGTATCTGCAAATGAACAAC
CTGAGACCTGAGGACACGGCCGTTTATTACTGTGCAGCAGATCTAGCCGAG
TATAGTGGTACTTACTCCAGCCCTGCGGACTCCCCCGCTGGGTATGACTAC
TGGGGCCAGGGGACCCAGGTCACCGTCTCCTCA,
or
(SEQ ID NO: 142)
CAGGTGCAGCTGGTCGAAACTGGGGGAGGATTGGTGCAGGCTGGGGACTCT
CTGAGACTCTCCTGTGCAGCCTCTGGACGCACCCTCAGCTTCAACACCTAT
GCCATGGGCTGGTTCCGCCAGGCTCCAGGGAAGGAGCGTGAATTTGTAGCC
TCTATTACCTGGAATGGTGGAAGCACAAGCTACGCAGACTCCGTGAAGGGC
CGATTCACCATCACCAGAGACAACGCCAAGAACACGGCTACTCTGCGAATG
AATAGCCTGCAGCCCGACGACACGGCCGTGTATTACTGTGCAGCAGCCCGA
TACTATGTGAGTGGTACTTACTTCCCCGCGAATTACTGGGGCCAGGGGACC
CAGGTCACCGTCTCCTCA.

Also provided are vectors comprising the nucleic acid molecules described herein. In an embodiment, the nucleic acid molecules can be incorporated into a recombinant expression vector. The present disclosure provides recombinant expression vectors comprising any of the nucleic acids of the disclosure. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

In an embodiment, the recombinant expression vector of the disclosure can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Examples of plant expression vectors include pBI01, pBI01.2, pBI121, pBI101.3, and pBIN19 (Clontech). Examples of animal expression vectors include pEUK-Cl, pMAM, and pMAMneo (Clontech). The recombinant expression vector may be a viral vector, e.g., a retroviral vector, e.g., a gamma retroviral vector.

In an embodiment, the recombinant expression vectors are prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vector may comprise regulatory sequences, such as transcription and translation initiation and termination codons, which are specific to the type of host (e.g., bacterium, plant, fungus, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the described expression vectors include, for instance, neomycin/G418 resistance genes, histidinol x resistance genes, histidinol resistance genes, tetracycline resistance genes, and ampicillin resistance genes.

The recombinant expression vector can comprise a native or normative promoter operably linked to the nucleotide sequence of the disclosure. The selection of promoters, e.g., strong, weak, tissue-specific, inducible and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an RSV promoter, an SV40 promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

The present disclosure further relates to variants of the polynucleotides described herein, wherein the variant encodes, for example, fragments, analogs, and/or derivatives of the single domain antibody that binds pIgR of the disclosure. In certain embodiments, the present disclosure provides a polynucleotide comprising a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding the single domain antibody that binds pIgR of the disclosure.

As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In certain embodiments, a polynucleotide is isolated. In certain embodiments, a polynucleotide is substantially pure.

Also provided are host cells comprising the nucleic acid molecules described herein. The host cell may be any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5a, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, Saos, PC12, SP2/0 (American Type Culture Collection (ATCC), Manassas, Va., CRL-1581), NSO (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATCC CRL-TIB-196). Other useful cell lines include those derived from Chinese Hamster Ovary (CHO) cells such as CHO-K1SV (Lonza Biologics, Walkersville, Md.), CHO-K1 (ATCC CRL-61) or DG44.

5.5. Pharmaceutical Compositions

In one aspect, the present disclosure further provides pharmaceutical compositions comprising a single domain antibody or a therapeutic molecule of the present disclosure. In some embodiments, a pharmaceutical composition comprises therapeutically effective amount of the antibody or therapeutic molecule provided herein and a pharmaceutically acceptable excipient.

In a specific embodiment, the term “excipient” can also refer to a diluent, adjuvant (e.g., Freunds' adjuvant (complete or incomplete) or vehicle. Pharmaceutical excipients can be sterile liquids, such as water and oils, including those of petroleum, animal, vegetable or synthetic origin, such as peanut oil, soybean oil, mineral oil, sesame oil and the like. Water is an exemplary excipient. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, Pa. Such compositions will contain a prophylactically or therapeutically effective amount of the antibodies or therapeutic molecules provided herein, such as in purified form, together with a suitable amount of excipient so as to provide the form for proper administration to the patient. The formulation should suit the mode of administration.

The single domain antibody or therapeutic molecule provided herein may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

The single domain antibody or therapeutic molecule provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with the single domain antibody or therapeutic molecule provided herein, including, but not limited to, encapsulation in liposomes, microparticles, microcapsules, recombinant cells capable of expressing the single domain antibody or therapeutic molecule provided herein, construction of a nucleic acid as part of a retroviral or other vector, etc.

In some embodiments, the antibody or therapeutic molecule provide herein is formulated in a pharmaceutic composition suitable for less-invasive or non-invasive administration. In a specific embodiment, the antibody or therapeutic molecule provide herein is formulated in a pharmaceutic composition suitable for oral administration. In a specific embodiment, the antibody or therapeutic molecule provide herein is formulated in a pharmaceutic composition suitable for buccal administration. In a specific embodiment, the antibody or therapeutic molecule provide herein is formulated in a pharmaceutic composition suitable for inhalation administration. In a specific embodiment, the antibody or therapeutic molecule provide herein is formulated in a pharmaceutic composition suitable for nasal administration. Non-limiting exemplary dosage forms are described in more detail in the following sections.

5.5.1. Oral Dosage Forms

In certain embodiments, the antibodies or therapeutic molecules provided herein are formulated in pharmaceutical compositions suitable for oral administration. Oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions provided herein is typically present in from about 50 to about 99 weight percent of the pharmaceutical composition or dosage form.

Disintegrants are used in compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

5.5.2. Topical and Mucosal Dosage Forms

Topical and mucosal dosage forms provided herein include, but are not limited to, sprays, aerosols, solutions, emulsions, suspensions, eye drops or other ophthalmic preparations, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4th ed., Lea & Febiger, Philadelphia (1985). In some embodiments, the mucosal dosage forms provided herein are suitable for administration to oral mucosal surface (buccal) or to nasal mucosal surface of a subject.

Suitable excipients (e.g., carriers and diluents) and other materials that can be used to provide topical and mucosal dosage forms are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th} and 18^{th} eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.5.3. Delayed Release Dosage Forms

In another embodiment, a pharmaceutical composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., a fusion protein as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 5.6. Methods and Uses

As demonstrated by the present disclosure, the single domain antibodies (e.g., VHH domains) provided herein are useful for transporting an agent from an apical surface of a pIgR-expressing cell to a basolateral surface of the pIgR-expressing cell, and can deliver the agent, e.g., to systemic circulation or lamina propria or gastrointestinal tract of a subject, via methods such as oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

Thus, in some embodiments, provided herein is a method for delivering from an apical surface of a pIgR-expressing cell to a basolateral surface of the pIgR-expressing cell comprising contacting the pIgR-expressing cell with (i) a single domain antibody that binds to pIgR provided herein, or (ii) a therapeutic molecule comprising an agent and the single domain antibody. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In some embodiments, provide herein is a single domain antibody that binds to pIgR provided herein for use in delivering an agent from an apical surface of a pIgR-expressing cell to a basolateral surface of the pIgR-expressing cell, wherein the agent is conjugated to the single domain antibody. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In some embodiments, provided herein is a use of a single domain antibody that binds to pIgR provided herein for delivering an agent from an apical surface of a pIgR-expressing cell to a basolateral surface of the pIgR-expressing cell, wherein the agent is conjugated to the single domain antibody. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In other embodiments, provided herein is a method for transporting a therapeutic molecule to a basolateral surface of the pIgR-expressing cell of a subject, comprising administering to the subject the therapeutic molecule comprising an agent and a VHH domain. In some embodiments, the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In other embodiments, provided herein is a single domain antibody for use in transporting a therapeutic molecule to a basolateral surface of the pIgR-expressing cell of a subject, wherein the therapeutic molecule comprises an agent and the single domain antibody. In some embodiments, the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In other embodiments, provided herein is a use of a single domain antibody for transporting a therapeutic molecule to a basolateral surface of the pIgR-expressing cell of a subject, wherein the therapeutic molecule comprises an agent and the single domain antibody. In some embodiments, the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a method for transporting a therapeutic molecule to systemic circulation of a subject, comprising administering to the subject the therapeutic molecule comprising an agent and a single domain antibody, wherein the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a single domain antibody for use in transporting a therapeutic molecule to systemic circulation of a subject, wherein the therapeutic molecule comprises the single domain antibody and an agent, and wherein the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a use of VHH for transporting a therapeutic molecule to systemic circulation of a subject, wherein the therapeutic molecule comprises the single domain antibody and an agent, and wherein the therapeutic molecule is administered to the subject via oral delivery, buccal delivery, nasal delivery or inhalation delivery.

In yet other embodiments, provided herein is a method for transporting a therapeutic molecule to lamina propria or gastrointestinal tract of a subject, comprising administering to the subject the therapeutic molecule comprising an agent and a single domain antibody, wherein the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a single domain antibody for use in transporting a therapeutic molecule to lamina propria or gastrointestinal tract of a subject, wherein the therapeutic molecule comprises an agent and the single domain antibody, and wherein the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a use of a single domain antibody for transporting a therapeutic molecule to lamina propria or gastrointestinal tract of a subject, wherein the therapeutic molecule comprises an agent and the single domain antibody, and wherein the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In some embodiments of the various methods and uses provided herein, the therapeutic agent is transported from an apical surface of a pIgR-expressing cell to a basolateral surface of the pIgR-expressing cell in the subject.

In some embodiments, the single domain antibody or the therapeutic molecule comprising an agent and the single domain antibody is also capable of being transported from the basolateral surface of the pIgR-expressing cell to the apical surface of the pIgR-expressing cell.

In yet other embodiments, provided herein is a method of treating a disease or disorder comprising administering a therapeutic molecule comprising an agent and the single domain antibody provided herein to a subject, wherein optionally the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a therapeutic molecule comprising an agent and a single domain antibody provided herein for use in treating a disease or disorder in subject, wherein optionally the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a use of a therapeutic molecule comprising an agent and a single domain antibody provided herein for treating a disease or disorder in subject, wherein optionally the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In some embodiments, the disease or disorder is a metabolic disease or disorder. In some embodiments, the disease or disorder is diabetes. In some embodiments, the disease or disorder is cancer. In other embodiments, the disease or disorder is an immune disease or disorder. In some embodiments, the disease or disorder is a gastrointestinal disease. In some embodiments, the disease or disorder is gastrointestinal inflammation. In some embodiments, the disease or disorder is inflammatory bowel disease (IBD). In some embodiments, the disease or disorder is Crohn's disease (CD). In some embodiments, the disease or disorder is ulcerative colitis (UC). In some embodiments, the disease or disorder is ankylosing spondylitis (AS). In some embodiments, the disease or disorder is colitis.

For example, the single domain antibodies of the disclosure may be conjugated to any agent that can be used to treat or ameliorate symptoms of intestinal inflammation, IBD, UC or AS, including agents which are inhibitors of pro-inflammatory cytokines, inhibitors of Th17 cell activation and/or differentiation, molecules inhibiting lymphocyte trafficking or adhesion, modulators of innate immune system, modulators of macrophages, dendritic cells, regulatory T cells (Treg) or effector CD8⁺ or CD4⁺ T cells. Such exemplary agents include inhibitors of TNF-α IL-12, IL-6, IL-13, IL-17A, IL17A/F, IL-18, IL-21, modulators of TLR3 or TLR4 pathway, TNF-α inhibitors infliximab, adalimumab, certolizumab, golimumab, etanercept and biosimilars thereof, IL-23 inhibitors ustekinumab, risankizumab, brazikumab and mirikizumab, IL-23 receptor inhibitors, IL-17 inhibitor secukinumab, IL-6 inhibitors tocilizumab and PF-04236921, PDE4 inhibitor apermilast, JAK inhibitors tocacifinib, filgotinib, upadacitinib or peficiting, inhibitors of cell adhesion such as natalizumab, vedolizumab, etrolizumab, abrilumab, PF-00547659, integrin antagonists or sphingosine 1 phosphate receptor modulators, or agents enhancing production of IL-10. In some embodiments, the agent is an inhibitor of IL-23 receptor. The agent targeting pathogenic pathways in intestinal inflammation herein may be a known molecule, a variant or a fragment of the known molecule, or generated de novo and genetically fused or chemically conjugated to the single domain antibody of the disclosure using known methods and those described herein.

In some embodiments, the methods or uses provided here are for delivering a vaccine for preventing an infection, such as Vibrio, Cholera, Typhoid, Rotavirus, Tuberculosis, HIV, Flu, Ebola, and Sendai.

In some embodiments of the various methods and uses provided herein, the agent in the therapeutic molecule comprises a peptide. In some embodiments of the various methods and uses provided herein, the agent in the therapeutic molecule comprises an antibody or a fragment thereof. In some embodiments of the various methods and uses provided herein, the agent in the therapeutic molecule comprises a peptide conjugated to a small molecule compound (e.g., antibody drug conjugate). In some embodiments of the various methods and uses provided herein, the agent in the therapeutic molecule comprises a nucleic acid. In some embodiments of the various methods and uses provided herein, the agent in the therapeutic molecule comprises a vaccine.

The amount of a prophylactic or therapeutic agent (e.g., an antibody or therapeutic molecule), or a composition provided herein that will be effective in the prevention and/or treatment of a disease or condition can be determined by standard clinical techniques. In addition, in vitro assays may optionally be employed to help identify optimal dosage ranges. The precise dose to be employed in the formulation will also depend on the route of administration, and the seriousness of a disease or condition, and should be decided according to the judgment of the practitioner and each patient's circumstances.

Effective doses may be extrapolated from dose-response curves derived from in vitro or animal model test systems. In certain embodiments, the route of administration for a dose of an antibody or therapeutic molecule provided herein to a patient is oral delivery, buccal delivery, nasal delivery, inhalation delivery, or a combination thereof, but other routes may be also acceptable. Each dose may or may not be administered by an identical route of administration. In some embodiments, an antibody or therapeutic molecule provided herein may be administered via multiple routes of administration simultaneously or subsequently to other doses of the same or a different agent provided herein.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### 6. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 6.1. Example 1: Immunization, Recovery and Screening of pIgR Binders

To generate a panel of single-domain antibodies that bind to pIgR, llamas were immunized with recombinant human pIgR (hpIgR) and/or mouse pIgR (mpIgR) for about 90 days. The whole blood and PBMCs was isolated from llamas, and RNA was prepared. After first-strand cDNA synthesis, llama-specific primers annealing to (i) the VH (heavy-chain variable region), (ii) VHH leader sequence genes, and (iii) the CH2 gene were used to PCR amplify the VH and VHH gene repertoires.

VHH repertoires were separated from VH repertoires by running the PCR fragments on a gel and excising the smaller band. The VHH gene repertoire was reamplified and cloned into a CMV-based mammalian vector. The VHH-gene was formatted as Ig-fusion. The library was transformed in E. coli. Single colonies were picked in a 96-well format for Sanger sequencing. From approximately 300 unique sequences, a select number of VHH sequences were selected for miniprep DNA, and then scaled-up for future recombinant expression and screening. 39 clones were chosen for miniprep DNA from the mo_pIgR_llama_Sort1 campaign and 35 chosen from hu_pIgR_llama_Sort1 campaign. Clone Selection was based on sequence uniqueness (weighted heavily on CDR3) and a Framework 2 signature indicative of VHH or Heavy-Chain only derived sequence.

B-cells that were positive for VHH and antigen binding were isolated and recovered, cloned and the VHH variable domain were sequenced using established protocols. Following VHH-region sequencing, a panel of 73 VHH molecules were expressed and purified as fusions to the human IgG1 mono-Fc protein. The sequence of the human IgG1 mono-Fc protein is as follows:

(SEQ ID NO: 146)
SPAPELLGG PSVFLFPP KPKDTLMISRTP EVTCVVVDVS
HEDPEVKFNW YVDGVEVHNA KTKPREEQYN STYRVVSVLT
VLHQDWLNGK EYKCKVSNKA LPAPIEKTIS KAKGQPREPQ
VYTKPPSREE MTKNQVSLSC LVKGFYPSDI AVEWESNGQP
ENNYKTTVPV LDSDGSFRLA SYLTVDKSRW QQGNVFSCSV
MHEALHNHYT QKSLSLSPGK

This VHH panel was screened for binding to hpIgR and mpIgR ectodomain by enzyme-linked immunosorbent assays (ELISAs) resulted in 40 positive hits.

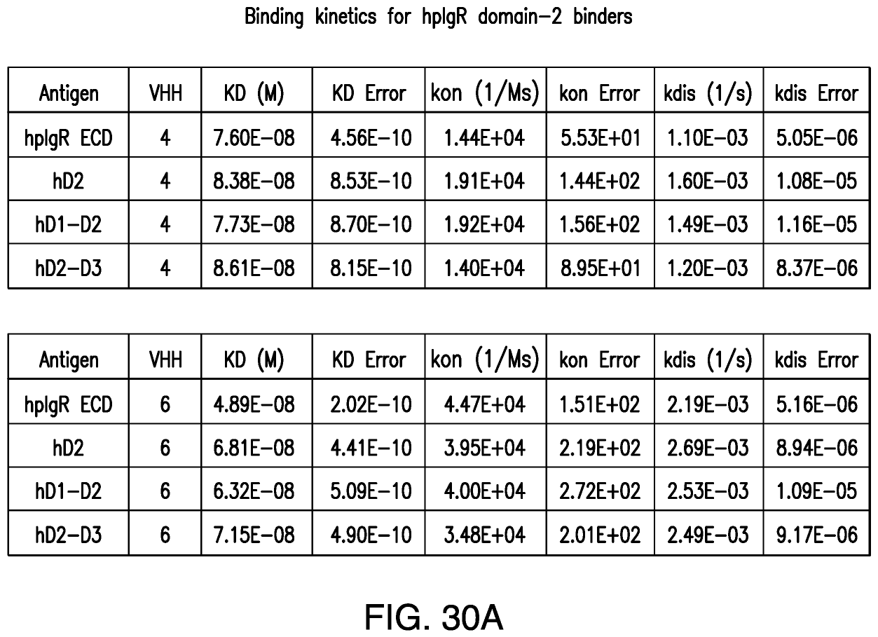

Bio-layer Interferometry was performed as follows. The ForteBioOctet RED384 system (Pall Corporation) was used to measure binding kinetics between VHH-mono-Fc molecules and pIgR proteins, and between IgA and pIgR proteins (in the absence and presence of VHH-mono-Fc molecules). Data were collected with Octet Data Acquisition version 7.1.0.87 (ForteBio) and analyzed using Octet Data Analysis version 7.1 (ForteBio). To measure binding kinetics between VHH-mono-Fc molecules and HIS-tagged pIgR proteins, VHH-mono-Fc was immobilized on amine-reactive generation-2 (ARG2) biosensors according to manufacturer's instructions and increasing concentrations of pIgR proteins were exposed to sensor-immobilized VHH. In some cases, HIS-tagged pIgR proteins were immobilized on anti-HIS biosensors and exposed to increasing concentrations of VHH-mono-Fc molecules. Association and dissociation rates were measured by the shift in wavelength (nm). For each sensor-immobilized protein, at least three different ligand concentrations were used, and K_{D} (equilibrium dissociation constant) was obtained by fitting the data to 1:1 binding model. All reactions were performed at 25° C. in PBS. The results are shown in FIGS. 30A-30B.

To measure binding kinetics between IgA and pIgR proteins, IgA was immobilized on ARG2 biosensors according to manufacturer's instructions, and immobilized IgA was exposed to increasing concentrations of pIgR ECD. To test the effect of VHH on pIgR-IgA binding, K_{D} values were measured for pIgR ECD binding to IgA in presence of VHH. IgA immobilized on ARG2 biosensors was exposed to increasing concentrations of pIgR-VHH complex, and association and dissociation rates were measured by the shift in wavelength (nm). For each sensor-immobilized IgA, at least three different pIgR or pIgR-VHH concentrations were used, and K_{D} (equilibrium dissociation constant) was obtained by fitting the data to 2:1 binding model. All reactions were performed at 25 C in PBS.

Bio-layer interferometry showed that 14 binders from this panel had K_{D} values of <100 nM for binding to the mouse or human pIgR ectodomain (5 anti-mpIgR, 6 anti-hpIgR and 3 cross-reactive).

Expression and purification of VHH in CHO cells was performed as follows. DNA constructs for VHH were sub-cloned into mammalian expression vectors using the In-Fusion® HD Cloning Kit. ExpiCHO™ cells were transfected with the appropriate expression vectors. Supernatants were harvested after 6-7 days by centrifugation (4,000 g, 15 min), passed through a 0.45-um filter, and purified at 4° C. by MabSelect™ SuRe™ chromatography on an AKTA express system (both GE Healthcare) using DPBS (Sigma) as running buffer and 0.1 M sodium acetate, pH 3.5 as elution buffer. Elutions were immediately neutralized using 25% (v/v) 2 M Tris-HCl pH 7.0, dialyzed to DPBS, sterilized by 0.22-um filtration and stored at 4° C. Concentrations were determined by absorbance at 280 nm on a Nanodrop ND-1000 spectrophotometer (ThermoFisher Scientific). The results are shown in FIG. 14.

Cloning, expression and purification of pIgR constructs in HEK293 cells was performed as follows. Gene blocks-encoding desired hpIgR domain sequences were obtained from IDT and sub-cloned into mammalian expression vectors using the InFusion® HD Cloning Kit. HEK Expi293™ cells were transfected with pIgR-domain expression vectors using ExpiFectamine™ 293 transfection kit. Supernatants were harvested after 6-7 days by centrifugation (4,000 g, 15 min), passed through a 0.45-um filter and purified by immobilized metal ion chromatography using HisPur™ Cobalt resin (Thermo scientific). Buffer NPI-20 (Teknova) was used as running buffer and Buffer NPI-300 (Teknova) containing 300 mM Imidazole was used as elution buffer. Elutions were buffer exchanged to DPBS using PD10 desalting columns (GE health care) following manufacturer's instructions and purified pIgR domains were stored at 4° C. Concentrations were determined by absorbance at 280 nm on a Nanodrop ND-1000 spectrophotometer (ThermoFisher Scientific).

Analytical-SEC was performed as follows. All purified VHH-mono-Fc molecules were analyzed by analytical high-pressure liquid chromatography on an Agilent 1200 infinity system using an Agilent AdvanceBio Size exclusion column (300 Å, 2.7 um, 4.6×150 mm). Column was equilibrated with 0.2M sodium phosphate pH 6.8 and 20 ul of samples were injected at a concentration of 0.5 mg/ml and at a flow rate of 0.35 mL/min. Monomeric VHH-mono-Fc elutes were detected at the expected retention time of ˜4 min at these settings. Data analysis was performed in OpenLab Chemstation to calculate % monomer content.

SEC-MALS was performed as follows. The molecular weight for purified VHH-mono-Fc molecules was measured by size-exclusion chromatography combined with multi-angle light scattering. The experiment was performed on a Waters high-pressure liquid chromatography instrument connected in series to Wyatt uDAWN light scattering/uTrEX instrument. An Acquity UPLC Protein BEH size-exclusion column (200 Å, 1.7 μm, 4.6×150 mm) was equilibrated with 1×DPBS pH 7.4 and 10 ul of samples were injected at a concentration of 0.5 mg/ml and at a flow rate of 0.3 mL/min. Molecular weight of the primary species (monomeric VHH-Fc) was calculated using the Astra software package (Wyatt).

### 6.2. Example 2: Biophysical Characterization of hpIgR-Specific Binders

10 pIgR binders (8 hpIgR specific and 2 human/mouse cross-reactive) from Example 1 were selected for further biophysical and functional assays. The 10 pIgR binders were expressed and purified from CHO cells using Protein-A affinity chromatography. Size-exclusion chromatography combined with multi-angle light scattering showed that molecular weight of 10 VHH-mono-Fc binders (VHH2, VHH3, VHH4, VHH5, VHH6, VHH7, VHH9, VHH10, VHH11, and VHH12) ranged from 41.3 kDa to 48.7 kDa.

Thermal stability of a sample was determined by differential scanning fluorimetry, specifically the NanoDSF method, using an automated Prometheus instrument. Measurements were made by loading a sample into a 24-well capillary from a 384-well sample plate. Duplicate runs were performed for each sample. A Prometheus NanoDSF user interface (Melting Scan tab) was used to set up the experimental parameters for the run. The thermal scans for a typical IgG sample spanned from 20° C. to 95° C. at a rate of 1.0° C./minute. Dual-UV technology monitoring of intrinsic tryptophan and tyrosine fluorescence at the emission wavelengths of 330 nm and 350 nm was undertaken. The F350 nm/F330 nm ratio was plotted against temperature to generate an unfolding curve.

The back reflection optics of the instrument was also used for the detection of sample aggregation. Such optics emitted near-UV light at a wavelength that is not absorbed by proteins. This light passed through the sample and was reflected to the detector. Protein aggregates scatter this light, and thus only non-scattered light reaches the detector. The reduction in back reflected light was a direct measure for aggregation in the sample and is plotted as mAU (Attenuation Units) against temperature. Nano DSF was used for measuring thermal unfolding parameters (Tm and Tagg) of VHH binders at 0.5 mg/mL concentration in Phosphate Buffered Saline, pH 7.4.

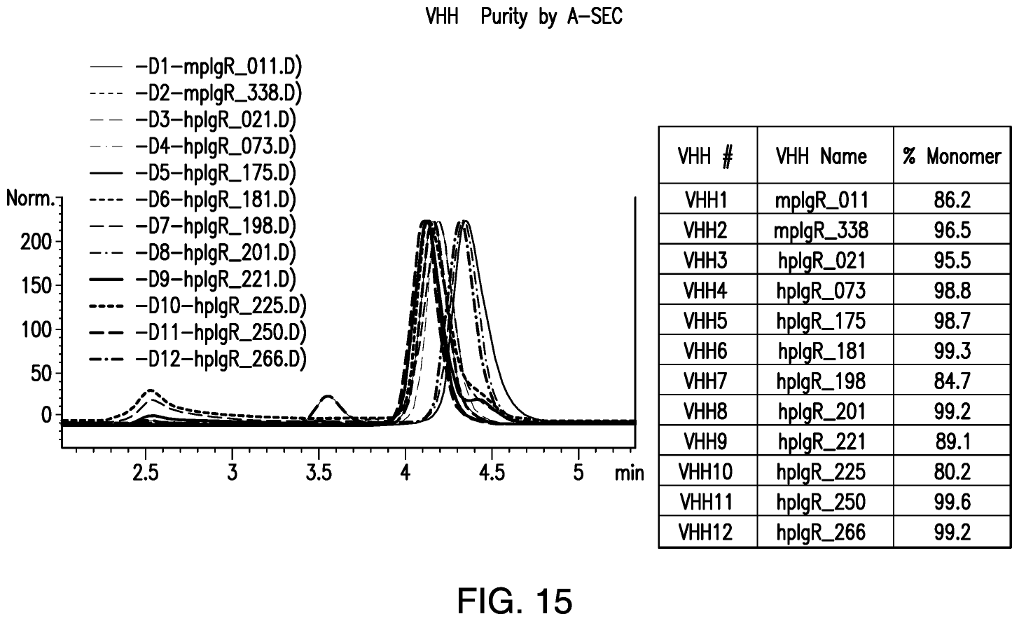

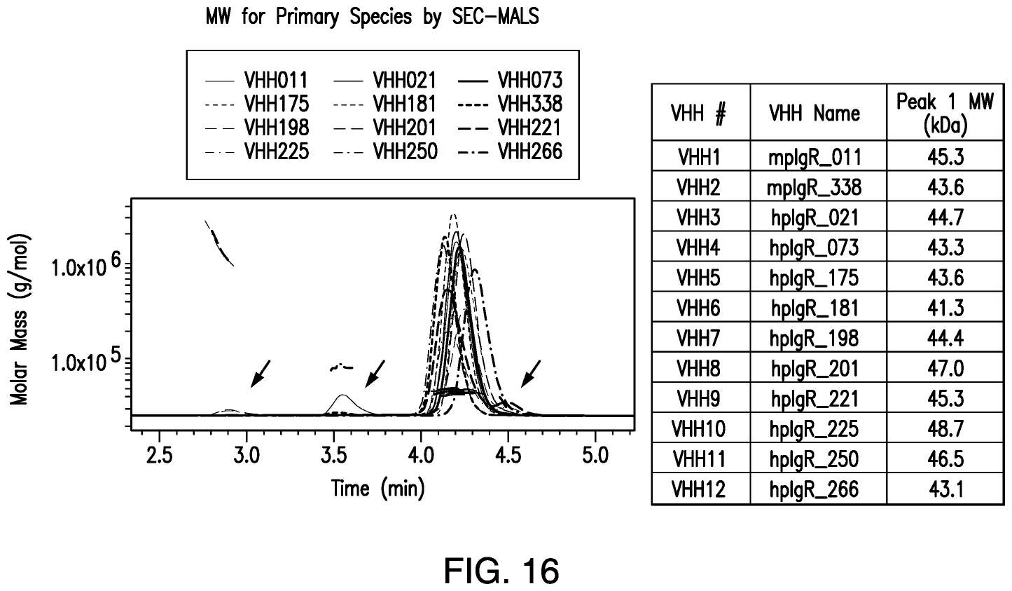

VHH-mono-Fc molecules were expressed in CHO cells and purified using Protein-A affinity chromatography. Homogeneity and molecular weight of the purified proteins were verified by analytical size-exclusion chromatography (A-SEC) and size-exclusion chromatography combined with multiple-angle light scattering (SEC-MALS), respectively. The results for A-SEC are shown in FIG. 15. The results for SEC-MALS are shown in FIG. 16.

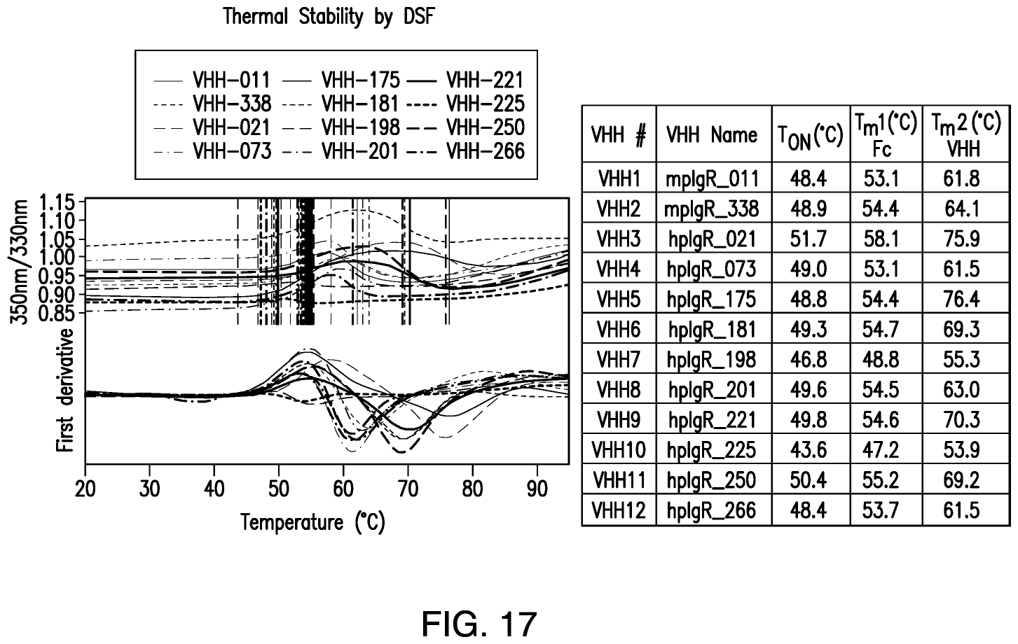

Thermal stability was assessed by differential scanning fluorimetry (DSF), with results shown in FIG. 17. The Tm for VHH molecules is reported below. K_{D} values for VHH-hpIgR ectodomain interactions were measured by bio-layer interferometry. EC₅₀ values for VHH molecules binding to MDCK-hpIgR cells were measured by flow cytometry.

Flow Cytometry was performed as follows. To test whether VHH-mono-Fc molecules recognize cell-surface hpIgR, Madin-Darby canine kidney (MDCK) cells engineered to express full-length hpIgR were used. Cells were cultured in Dulbecco's modified Eagle's medium containing 10% fetal calf serum at 37° C. with 5% CO₂. Cells were split into equal fractions (≈70,000 cells) and incubated with increasing concentrations of VHH-mono-Fc molecules for 30 min at 4 C. Cells were washed twice with cold PBS (pH 7.4) and incubated with a fluorescently-labelled anti-Fc antibody (Alexa Fluor® 647 AffiniPure F(ab′)2 fragment Goat Anti-Human IgG Fey Fragment Specific) for 30 min in staining buffer (2 μg/ml Ab) at 4 C. Cells were washed twice with cold staining buffer, resuspended in running buffer and analyzed with an iQue Screener (IntelliCyt Corporation). Binding was assessed by RL1 (A647) Geomeans from the live cell population and EC50 was calculated by fitting log VHH concentration versus MFI in Prism (Graphpad). The data are shown in Table 1 below.

TABLE 1

| | Tₘ | K_{D} | EC₅₀ | mpIgR |
|---|---|---|---|---|
| VHH | (° C.) | (nM) | (nM) | binding? |
| VHH2 | 64.1 | 21 | 6.3 | Yes |
| VHH3 | 75.9 | 5 | 6.4 | Yes |
| VHH4 | 61.5 | 22 | 32.9 | No |
| VHH5 | 76.4 | 11 | 4.3 | No |
| VHH6 | 69.3 | 27 | 11.5 | No |
| VHH7 | 55.3 | 521 | 36.4 | No |
| VHH9 | 70.3 | 4 | 1.5 | No |
| VHH10 | 53.9 | 256 | 20.4 | No |
| VHH11 | 69.2 | 19 | 1.5 | No |
| VHH12 | 61.5 | 34 | 4.6 | No |

In Table 1, differential scanning fluorimetry showed that Tm values of 10 VHH molecules ranged from 53.9° C. to 76.4° C. Differential scanning fluorimetry showed that Tm values of five potent VHH binders ranged from 61° C. to 70° C. Bio-layer interferometry showed that 8 binders from this panel had K_{D} values of <50 nM for binding to the human pIgR ectodomain, as shown in Table 1. Also, flow cytometry showed that 6 binders had EC₅₀ values of <10 nM for binding to MDCK-hpIgR cells.

### 6.3. Example 3: Cell Binding and Transcytosis Assay

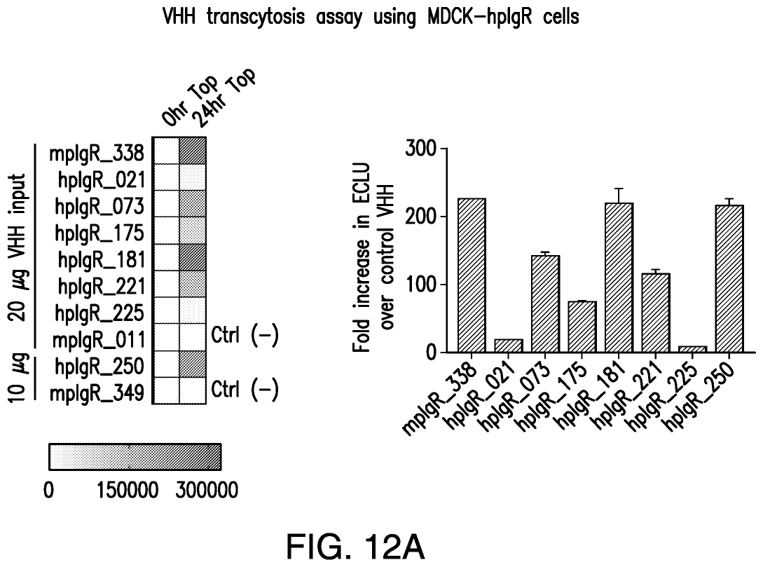

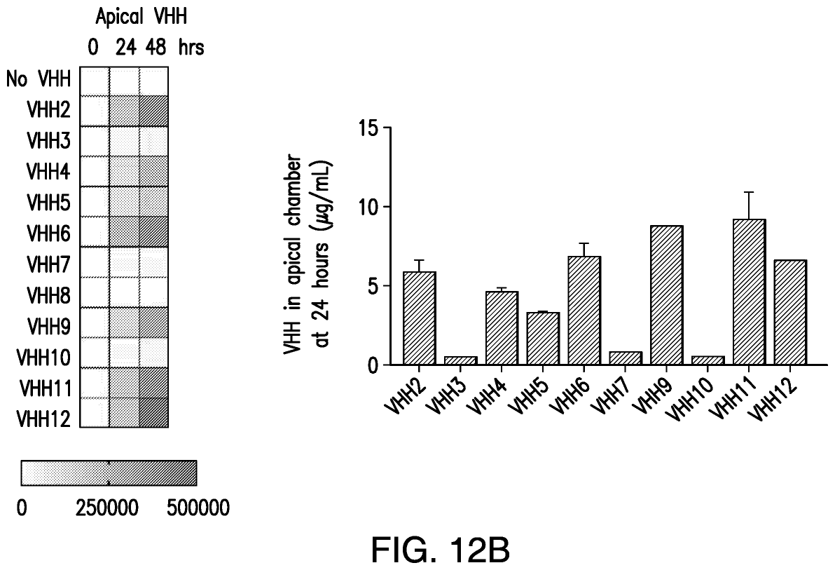

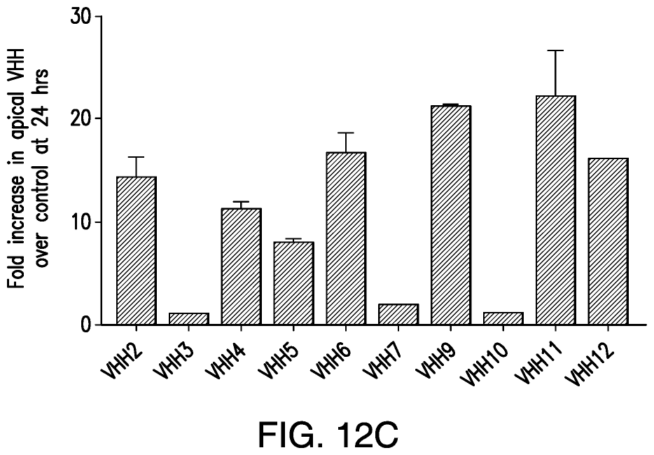

A transcytosis assay was performed as follows. Madin-Darby canine kidney (MDCK) cells, a commonly used epithelia model system, were used to investigate if VHH binders could be transported across epithelia by pIgR mediated transcytosis. MDCK cells, un-transfected or stably transfected with human pIgR were used to study transcytosis (See Natvig, I. B., Johansen, F. E., Nordeng, T. W., Haraldsen, G. & Brandtzaeg, P. Mechanism for enhanced external transfer of dimeric IgA over pentameric IgM: studies of diffusion, binding to the human polymeric Ig receptor, and epithelial transcytosis. J. Immunol. 159, 4330-4340 (1997)). Expression of hpIgR in MDCK cells and monolayer formation were confirmed by confocal laser microscopy. Approximately 5.0×10⁵ cells were seeded on 1-cm², 3.0-μm collagen-coated PTFE filters (Transwell-COL 3494; Costar). The cells were incubated for 3 days at 37° C. with 5% CO₂ in Dulbecco's modified Eagle's medium containing 10% fetal calf serum, 50 μg/ml gentamicin, and 1 mM L-glutamine. 20 μg of test VHH-mono-Fc molecules were added to the basolateral chamber, and the filters were incubated for 24 or 48 hours at 37° C. in fresh medium. A VHH-mono-Fc that did not bind to pIgR (irrelevant VHH) was used as a control together with 100 nM (15 μg/mL) human IgG (to control for unspecific transport and leakage). The apical medium was harvested, and the amount of VHHmono-Fc, transported by pIgR, was calculated by standard titration studies. IgG leakage to the apical medium was detected by MSD. The results of the transcytosis assay are shown in FIGS. 12A-12B.

Additionally, a biotinylated anti-VHH antibody was used to capture VHH-mono-Fc on streptavidin plates and a ruthenylated anti-Fc antibody to detect VHH-mono-Fc by the MSD platform. The results of this assay are shown in FIG. 12C. Six VHHs (2, 4, 6, 9, 11 and 12) showed >10-fold increase in their apical concentration relative to control VHH.

### 6.4. Example 4: Transcytosis Assays Using Primary Human Lung Tissue Model

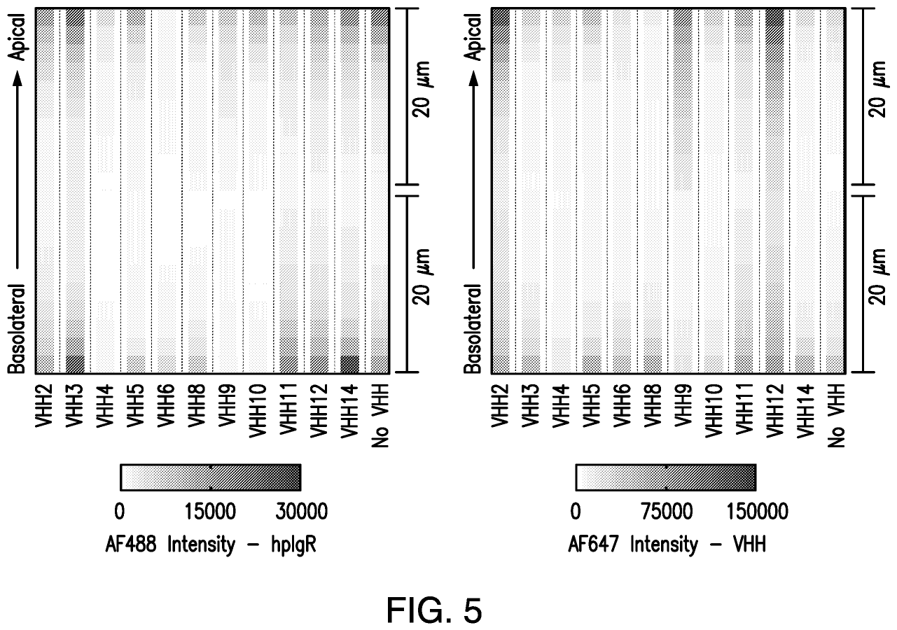

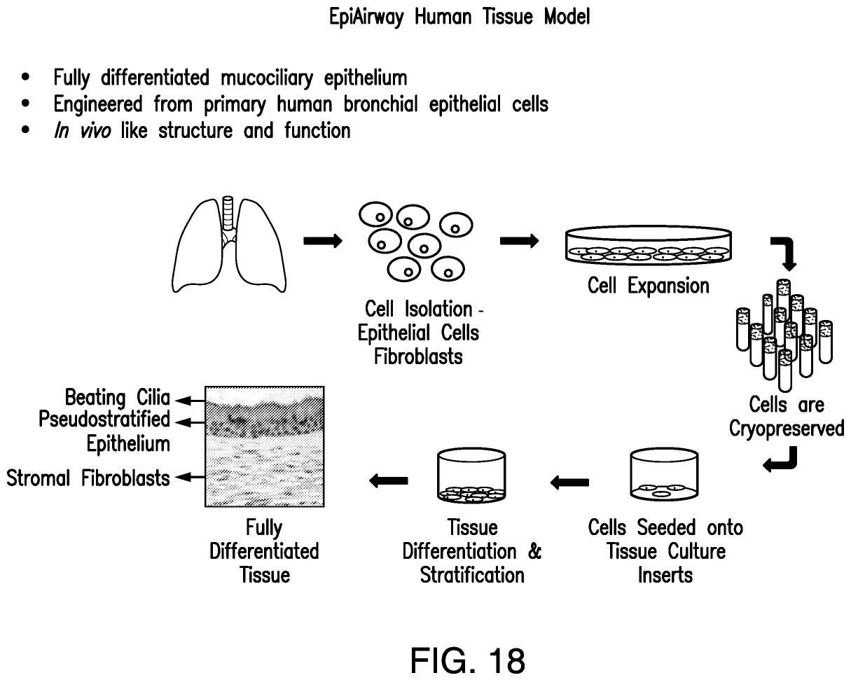

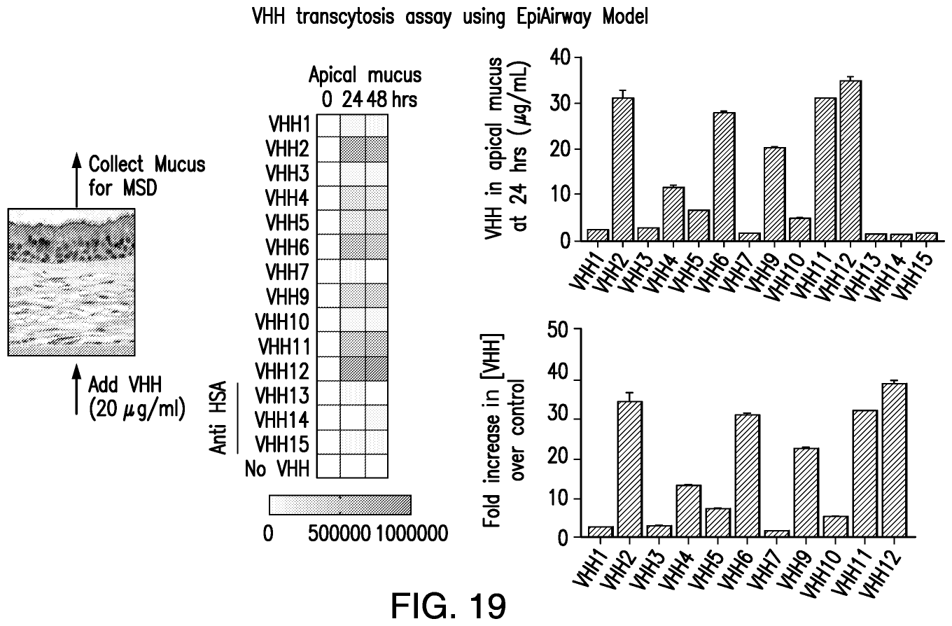

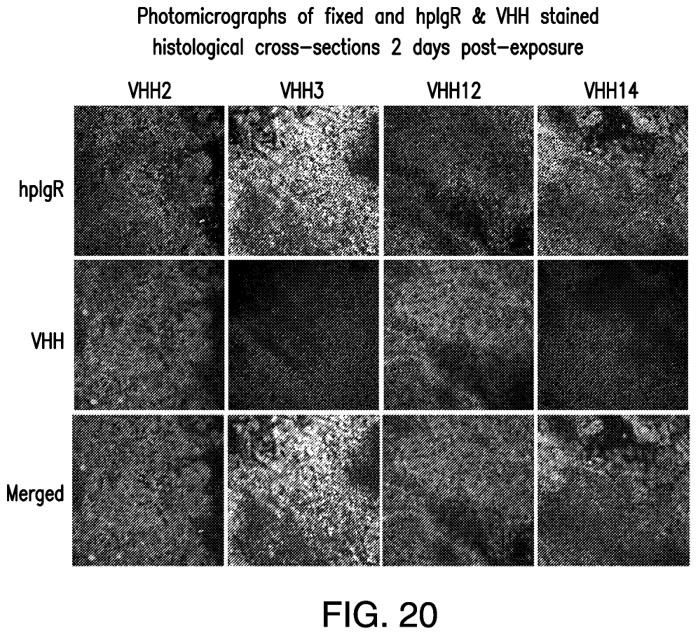

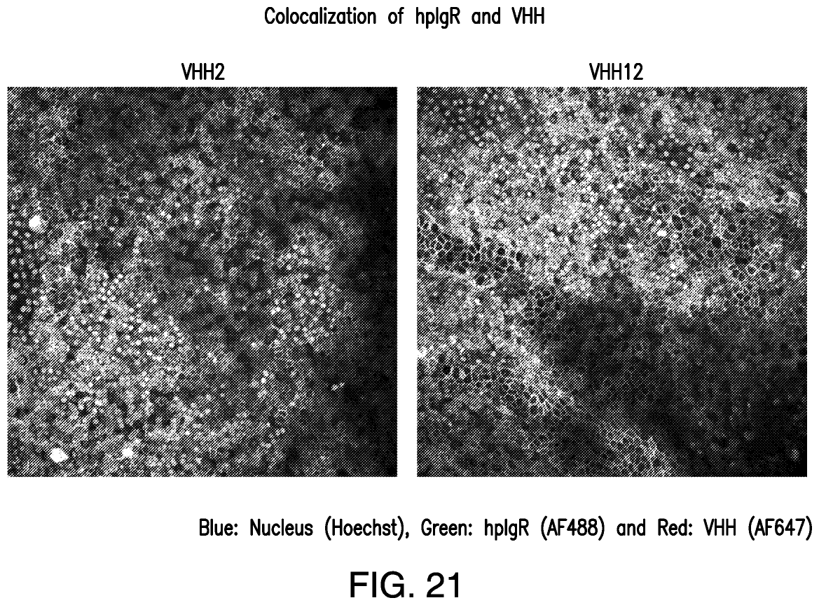

The EpiAirway human lung tissue model was also used to test the transcytosis activity of 10 VHH molecules from the basolateral to the apical epithelium and their delivery to the mucosal lumen. The EpiAirway model is depicted in FIG. 18. The EpiAirway model is an established lung tissue model engineered from primary human tracheal bronchial cells. Tissue models were obtained from Mattek Corporation and maintained according to manufacturer's instructions. 20 μg of test and control VHH-mono-Fc molecules were added to 1 ml of EpiAirway media in the basolateral chamber and 100 ul of samples were collected from the basolateral and apical chambers at 0, 24 and 48 hours. EpiAirway TEER buffer was used to collect the mucus from the apical chambers. The amount of VHH-mono-Fc present in basolateral media and apical mucus was quantified by electrochemiluminescence method. In this method, streptavidin MSD plates were coated with a biotinylated anti-VHH antibody (2 μg/ml in PBS) for 1 hour at RT with 1000 rpm, washed 3× with PBT, incubated with blocking buffer for 1 hour at RT, incubated with VHH-mono-Fc containing media/mucus (at different dilutions) for 2 hours at RT with 1000 rpm, washed 3× with PBT, incubated with ruthenylated-anti-human-Fc antibody (2 μg/ml in PBS) for 1 hour at RT with 1000 rpm, washed 3× with PBT and read plates in 40 ul reading buffer using the MSD imager. The amount of VHH-mono-Fc in basolateral and apical chambers was calculated by plotting ECLU values against VHH-mono-Fc standard curves in Prism (Graphpad). The data is shown in FIG. 19. A similar experiment in which IgG and IgA were transcytosed is shown in FIG. 20. Each photomicrograph in FIG. 20 is a representative image of one of the squares in the heat map in FIG. 5.

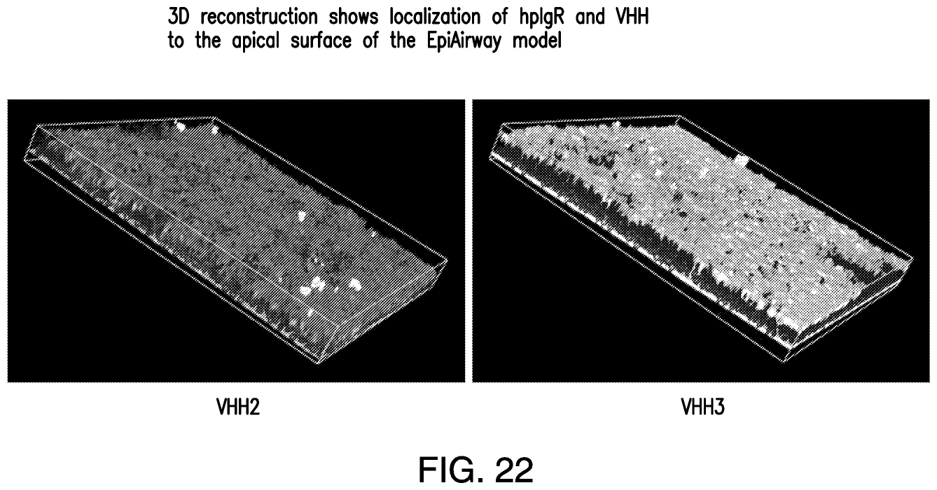

FIG. 22 shows 3D reconstruction shows localization of hpIgR and VHH to the apical surface of the EpiAirway model.

The amount of VHH present in the apical mucus 0, 24 and 48 hours post treatment was quantified by the electrochemiluminescence.

The Electrochemiluminescence assay was performed as follows. A meso-scale discovery (MSD) platform was used for conducting epitope mapping and epitope burial studies. To test the binding of VHH-mono-Fc molecules to purified pIgR protein constructs, Streptavidin MSD plates were coated with a biotinylated anti-HIS antibody (2 μg/ml in PBS) for 1 hour at RT with 1000 rpm, washed 3× with PBT (PBS+0.1% Tween-20), incubated with blocking buffer for 1 hour at RT, incubated with His-tagged pIgR proteins (10 μg/ml in PBS) for 2 hours at RT with 1000 rpm, washed 3× with PBT, incubated with VHH-mono-Fc molecules (100 μg/ml in PBS) for 2 hours at RT with 1000 rpm, washed 3× with PBT, incubated with ruthenylated-anti-human-Fc antibody (2 μg/ml in PBS) for 1 hour at RT with 1000 rpm, washed 3× with PBT and read plates in 40 ul reading buffer using the MSD imager. ECLU values were plotted as a heatmap.

To check whether VHH recognizes a buried epitope on pIgR, EC₅₀ values were measured for VHH-mono-Fc molecules binding to hpIgR-ECD protein by electrochemiluminescence using two different detection antibodies, an anti-Fc antibody and an anti-VHH antibody. pIgR ECD (10 μg/ml in PBS) was coated on high-bind MSD plates for 2 hours at RT with 1000 rpm, incubated with blocking buffer for 1 hour at RT, incubated with VHH-mono-Fc molecules (increasing concentrations in PBS) for 2 hours at RT with 1000 rpm, washed 3× with PBT, incubated with ruthenylated secondary antibody (2 μg/ml in PBS) for 1 hour at RT with 1000 rpm, washed 3× with PBT and read plates in 40 ul reading buffer using the MSD imager. EC50 was calculated by fitting log VHH concentration versus log ECLU in Prism (Graphpad). The increase in EC₅₀ (>50-fold) due to anti-VHH detection was used as a measure to determine whether VHH recognized buried epitope on pIgR.

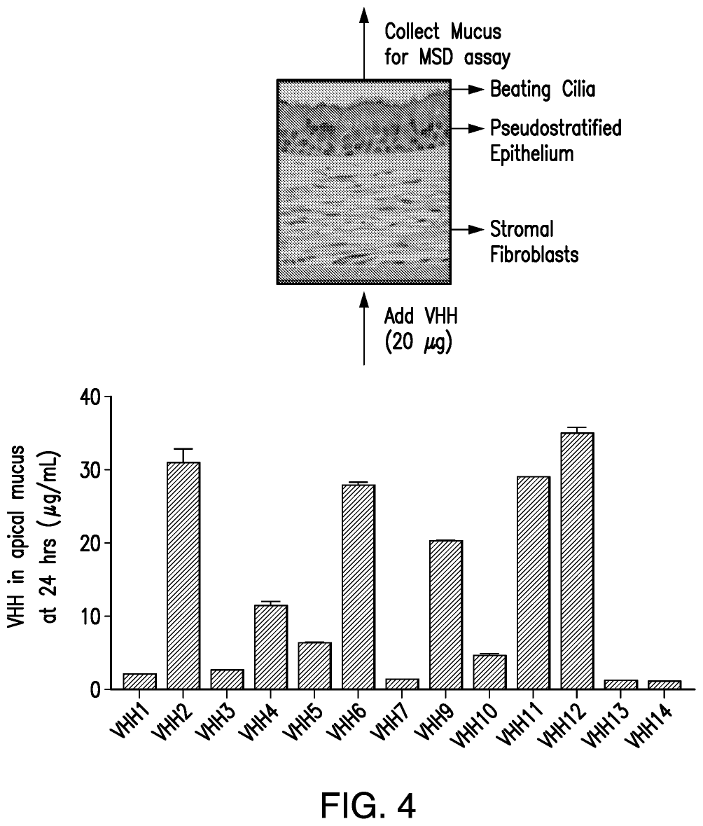

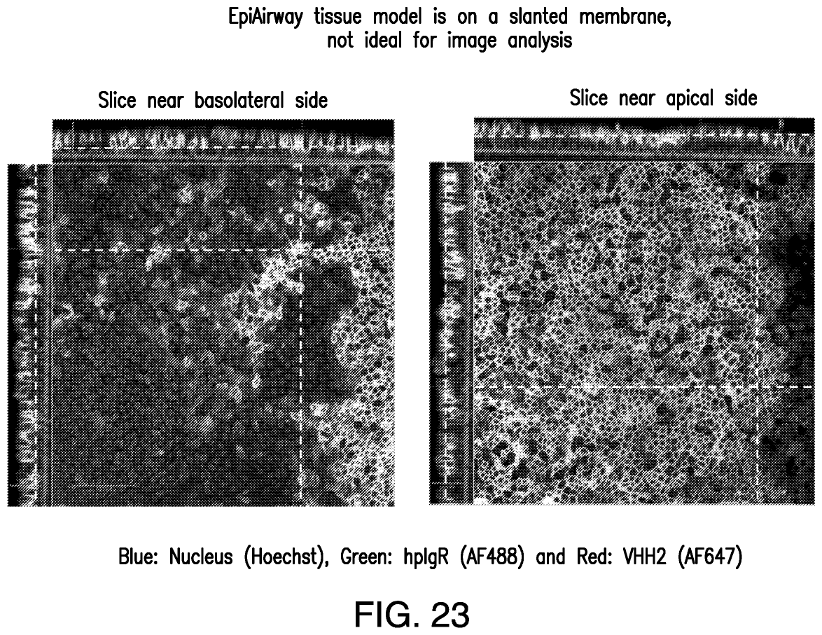

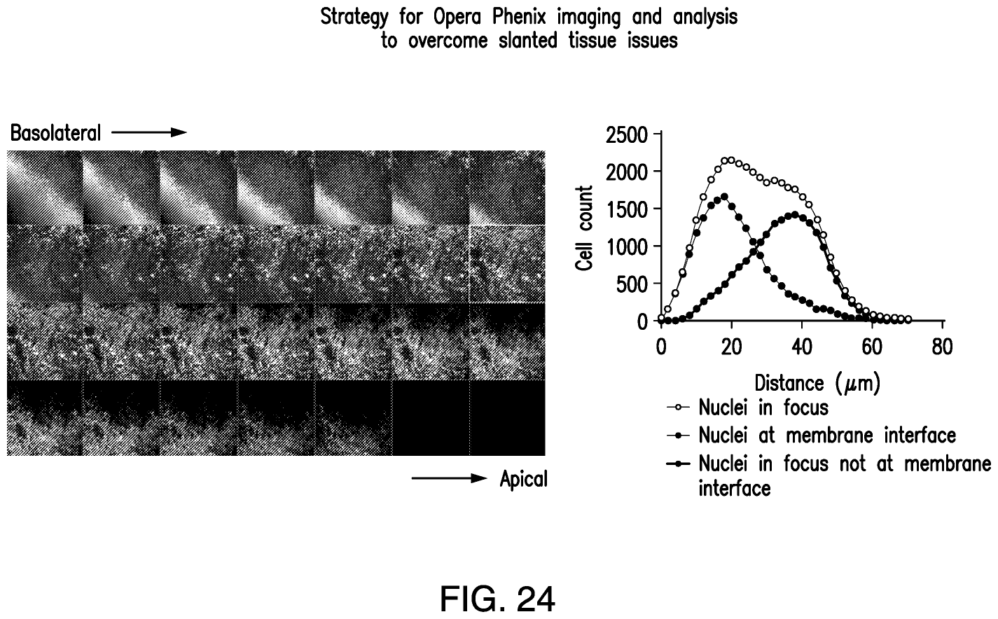

At 48 hours post-treatment, tissue samples were fixed, permeabilized and stained for tracking hpIgR and VHH across the EpiAirway model. The data is shown in FIG. 4. Five VHH molecules (VHH2, VHH6, VHH9, VHH11 and VHH12) showed greater than 20-fold increase in their mucosal amount relative to control VHH molecules. For the best pIgR agonist (VHH12), 17.5% of basolateral VHH input was secreted into mucus every 24 hours. FIG. 23 shows that the EpiAirway tissue model is on a slanted membrane, which is not ideal for image analysis. FIG. 24 illustrates a strategy for Opera Phenix imaging and analysis to overcome slanted tissue issues with EpiAirway tissue model.

Following transcytosis, indirect immunofluorescence was used to trace the location and amount of hpIgR and VHH across the EpiAirway tissue model by Opera Phenix confocal laser microscopy. Indirect immunofluorescence was used to track the amount of pIgR and VHH-mono-Fc retained across the EpiAirway model two-days post-treatment. Tissue samples were rinsed in PBS, tissues were fixed with 2 ml of 10% Formalin at RT for 20 minutes, washed three times with 2 ml PBST (1% Triton-X100 in PBS) at RT for 10 minutes each (with gentle agitation), incubated with primary antibodies (500 ul apical, 500 ul basolateral) diluted in PBTG (PBST with 10% goat serum) for 2 hours at RT (with gentle agitation), washed two times with 2 ml PBTG at RT for 10 minutes each (with gentle agitation), incubated with secondary antibodies (100 ul apical, 100 ul basolateral) diluted in PBTG for 1 hour at RT (with gentle agitation) and washed two times with 2 ml PBTG at RT for 10 minutes each (with gentle agitation). The primary antibody mix contained mouse antibody and biotinylated anti IgA antibody both at 5 μg/ml. The secondary antibody mix contained Alexa-Flour 488-labelled anti-mouse antibody (1:100 dilution), Alexa-Flour 647-labelled streptavidin (1:100 dilution) and Hoechst (1:1000 dilution). Fixed, permeabilized and stained tissues were imaged at 20× resolution (30-40 planes, 2 um distance) using Opera Phenix confocal laser microscopy. Image analysis was performed using the Harmony suite, fluorescence readouts were corrected for membrane auto-fluorescence, normalized for number of cells and plotted as heat maps in Prism (Graphpad).

The data is shown in FIG. 5. Imaging studies corroborated transcytosis results and showed colocalization of hpIgR and VHH, especially closer to the apical epithelium. Since pIgR is proteolytically cleaved and released into mucus upon transcytosis, the amount of tissue-retained pIgR inversely correlated with VHH function.

In the EpiAirway model, the presence of IgA did not affect the transcytosis of VHH9, however the presence of IgA had a negative effect on the four other VHH binders VHH2, VHH6, VHH11 and VHH12.

### 6.5. Example 5: Domain-Level Epitope Mapping

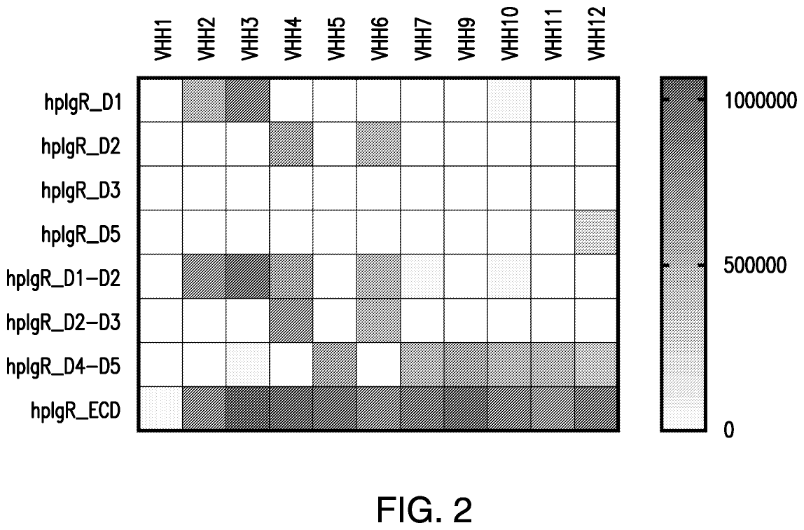

To conduct domain-level epitope mapping of VHHs, seven HIS-tagged hpIgR constructs (D1, D2, D3, D5, D1-D2, D2-D3 and D4-D5) were expressed and purified each encoding one or two domains of hpIgR ECD from HEK293 cells using immobilized metal ion affinity chromatography. Two constructs, D4 and D3-D4, showed poor expression and purification and were not used for epitope mapping assays. Binding of VHH-mFc molecules were tested to immobilized pIgR constructs by the electrochemiluminescence method. Results from the binding assay are shown as a heat map in FIG. 2.

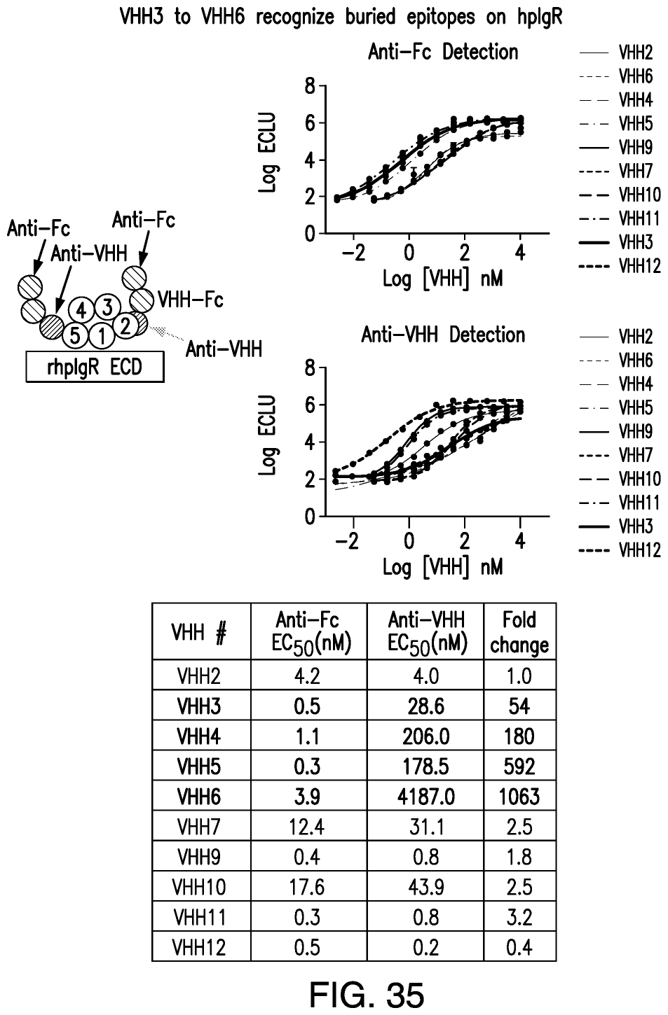

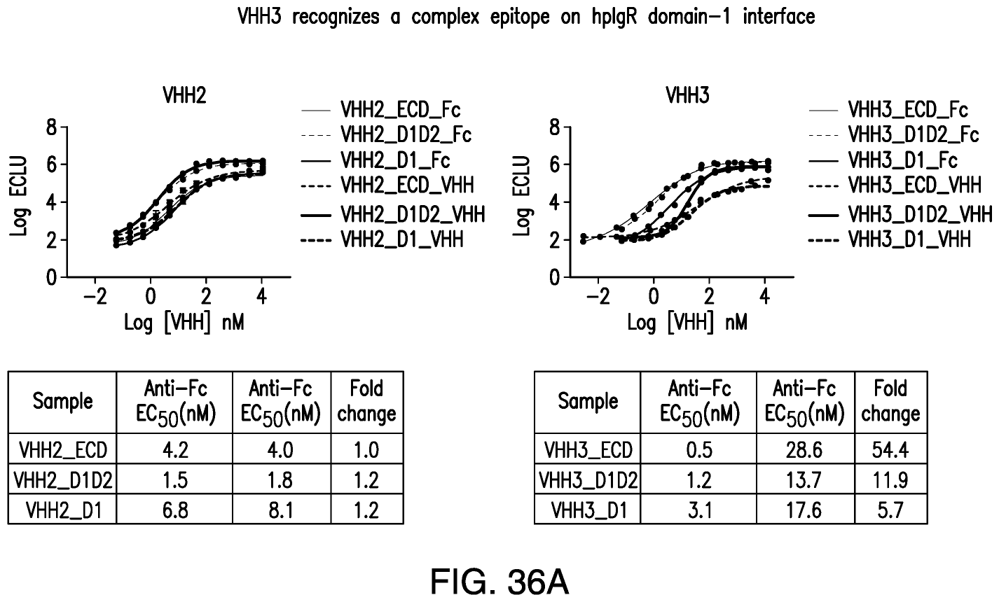

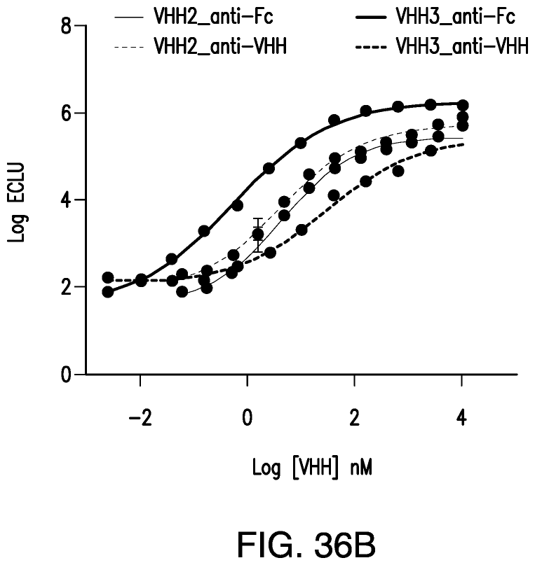

Recognition of buried epitopes by pIgR binders was performed as follows. The EC₅₀ for VHH-mono-Fc molecules binding to hpIgR-ECD protein was measured by electrochemiluminescence using two different detection antibodies, an anti-Fc antibody and an anti-VHH antibody. The increase in EC₅₀ (>50-fold) due to anti-VHH detection was used as a measure to determine whether VHH recognized buried epitope on pIgR. Four molecules (VHH3, VHH4, VHH5 and VHH6) recognized buried epitopes on pIgR, as shown in FIG. 35. As shown in FIGS. 36A-36B, VHH3 recognizes a complex epitope on the hpIgR domain-1 interface, and in particular, while no differences in EC₅₀ were observed for VHH2 (4 nM for both detection antibodies), VHH3 showed a 54-fold increase in EC₅₀ due to anti-VHH detection. Together these experiments indicated that VHH2 and VHH3 recognize domain-1 in a different fashion, that could have attributed to their differences in function.

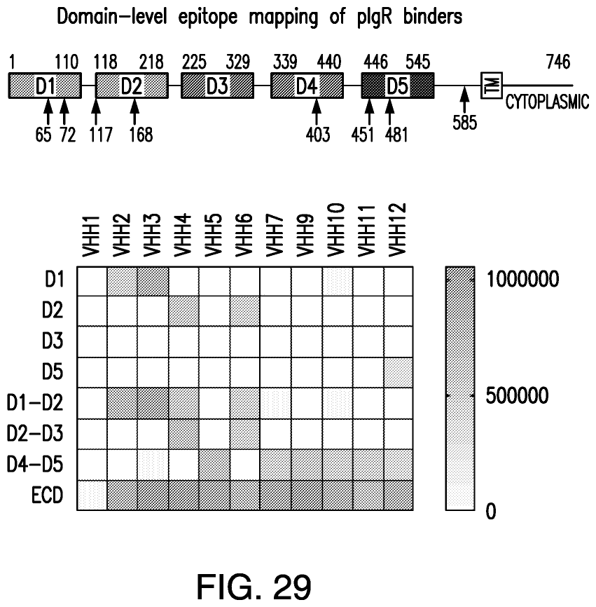

Epitope mapping showed that VHH2, VHH6 and VHH12 binds hpIgR domain 1, 2 and 5, respectively, whereas VHH9 and VHH11 binds to hpIgR domains 4-5. To test whether the VHH binding region recognizes buried epitopes on hpIgR, an electrochemiluminescence method using two different detection antibodies, an anti-Fc antibody and an anti-VHH antibody were used to generate EC₅₀ values that reflect VHH-mono-Fc molecules binding to hpIgR-ECD protein. An increase in EC₅₀ (>50-fold) due to anti-VHH detection was used as a measure to determine whether VHH recognized buried epitope on pIgR. The results are shown in Table 2 and FIG. 29.

TABLE 2

| | Anti-Fc | Anti-VHH | Fold change | Buried |
|---|---|---|---|---|
| VHH # | EC₅₀ (nM) | EC₅₀ (nM) | in EC₅₀ | epitope? |
| VHH2 | 4.2 | 4.0 | 1.0 | No |
| VHH3 | 0.5 | 28.6 | 54 | Yes |
| VHH4 | 1.1 | 206.0 | 180 | Yes |
| VHH5 | 0.3 | 178.5 | 592 | Yes |
| VHH6 | 3.9 | 4187.0 | 1063 | Yes |
| VHH7 | 12.4 | 31.1 | 2.5 | No |
| VHH9 | 0.4 | 0.8 | 1.8 | No |
| VHH10 | 17.6 | 43.9 | 2.5 | No |
| VHH11 | 0.3 | 0.8 | 3.2 | No |
| VHH12 | 0.5 | 0.2 | 0.4 | No |

The results of Table 2 indicate that four molecules (VHH3, VHH4, VHH5 and VHH6) recognized buried epitopes on pIgR. To conduct domain-level epitope mapping, seven HIS-tagged pIgR ectodomain constructs (D1, D2, D3, D5, D1-D2, D2-D3 and D4-D5) were successfully expressed and purified from HEK293 cells using immobilized metal ion affinity chromatography. The sequences of D1, D2, D3, D5, D1-D2, D2-D3, and D4-D5 comprise those of SEQ ID NOS: 216-222.

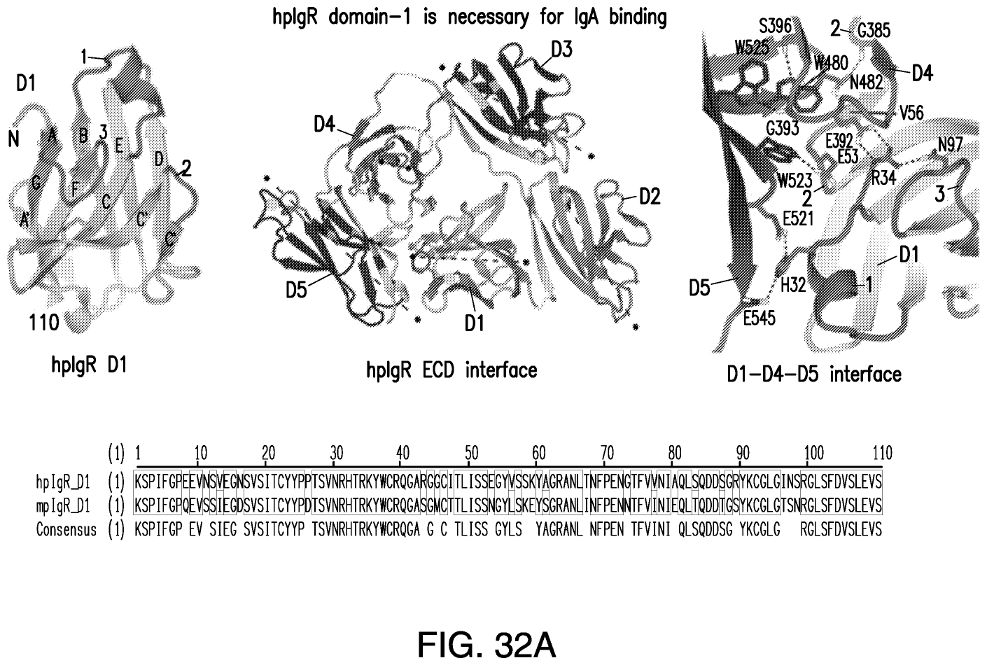

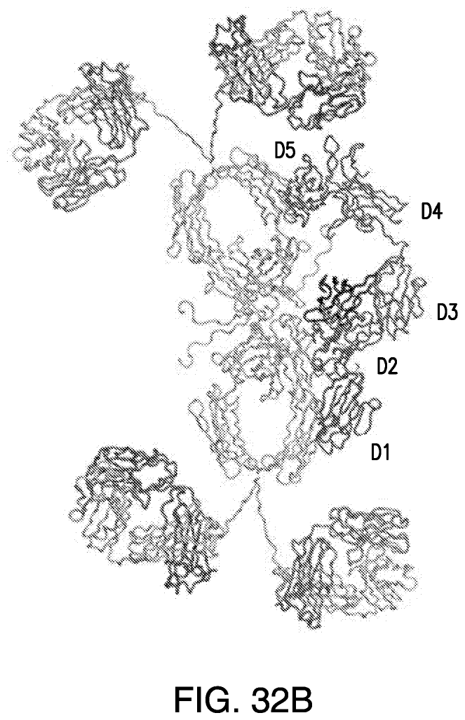

The binding of VHH-mono-Fc molecules to immobilized pIgR constructs is summarized as a heat map in FIG. 2. In brief, the epitopes of VHH2 and VHH3 are primarily contained within hpIgR domain 1 (D1), and the epitopes of VHH4 and VHH6 are primarily contained within hpIgR domain 2 (D2). As shown in FIG. 32A, D1 is necessary for IgA binding to hpIgR. The epitopes of other six VHH molecules are primarily contained within hpIgR domains 4-5 (D4-D5). Additionally, solution x-ray scattering studies conducted by Bonner et al., Mucosal Immunol., 2:74-84 (2009) suggest that upon interaction with dIgA, pIgR takes on an extended conformation, with domain-1 interacting with the Ca2 domain of one Fca subunit and domain-5 binding the Ca2 subunit on the same side of the opposite Fca subunit (FIG. 32B).

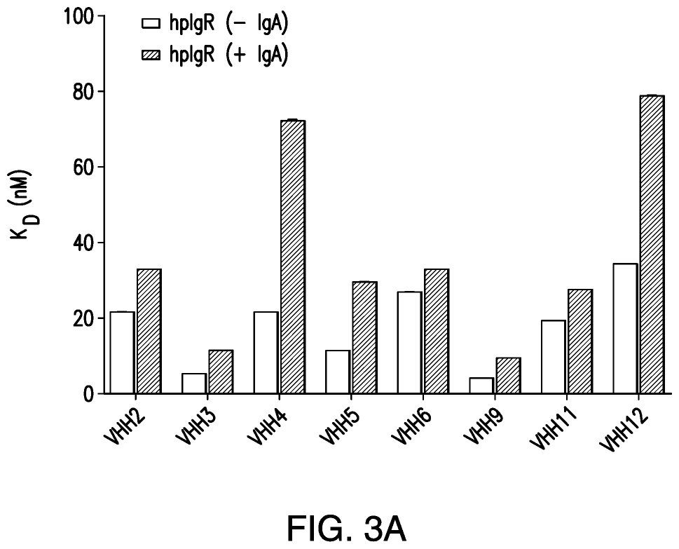

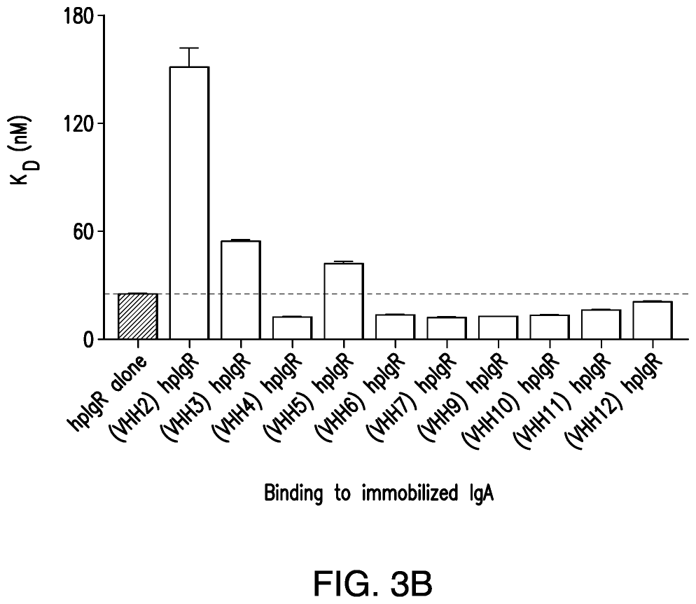

Next, competition binding assays were conducted for eight VHH-mono-Fc molecules that displayed KD values of <100 nM for binding to hpIgR. First, to test the influence of IgA on hpIgR-VHH binding, KD values were measured for full-length hpIgR ECD binding to immobilized VHH-mono-Fc molecules in the absence and presence of dIgA2 by bio-layer interferometry (FIG. 3A). In total, VHHs showed a 1.3 to 3.3-fold decrease in affinity for binding to hpIgR ECD due to the presence of dIgA. Pre-bound IgA had a small negative effect on binding of VHHs to pIgR, possibly due to steric hindrance arising from bound dIgA or conformational rearrangement of hpIgR ECD. Second, to test the effect of VHH on dIgA2 binding to hpIgR, K_{D} values for a recombinant dimeric IgA2 construct binding to the hpIgR ectodomain were measured with and without the presence of VHH-mono-Fc molecules. Three molecules (VHH2, VHH3 and VHH5) had a negative effect on IgA binding to pIgR, while other VHH molecules displayed a small positive effect on IgA binding to pIgR, as shown in FIG. 3B.

### 6.6. Example 6: VHH/IgA Competition Studies (Binding and Transcytosis)

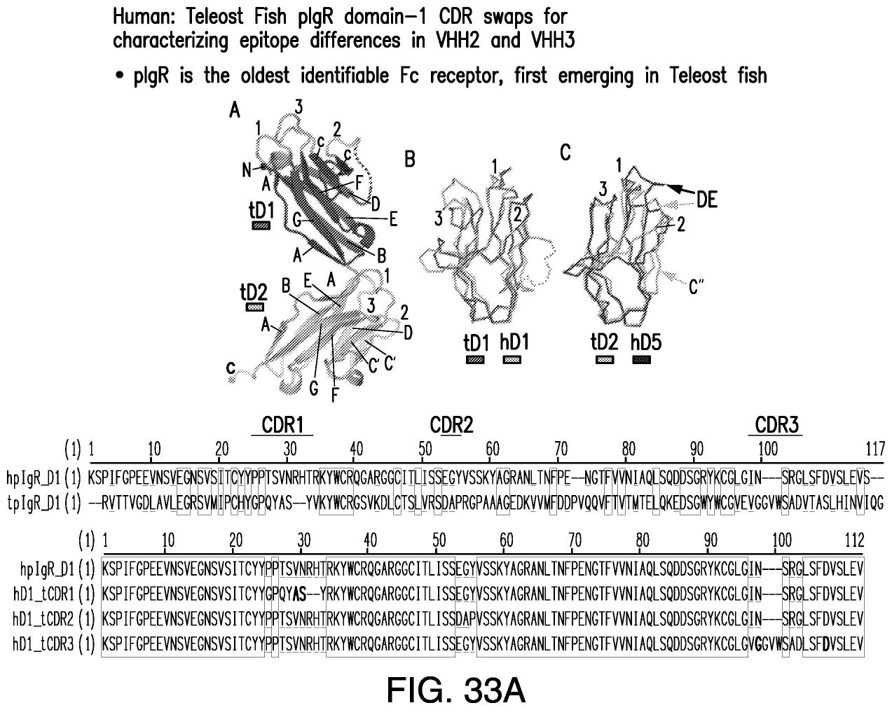

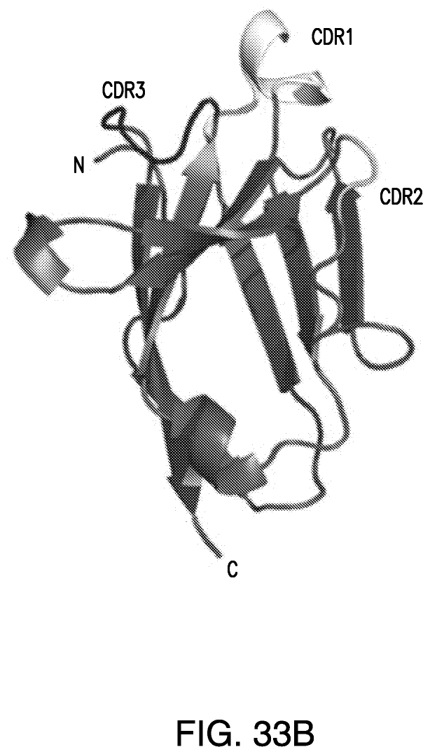

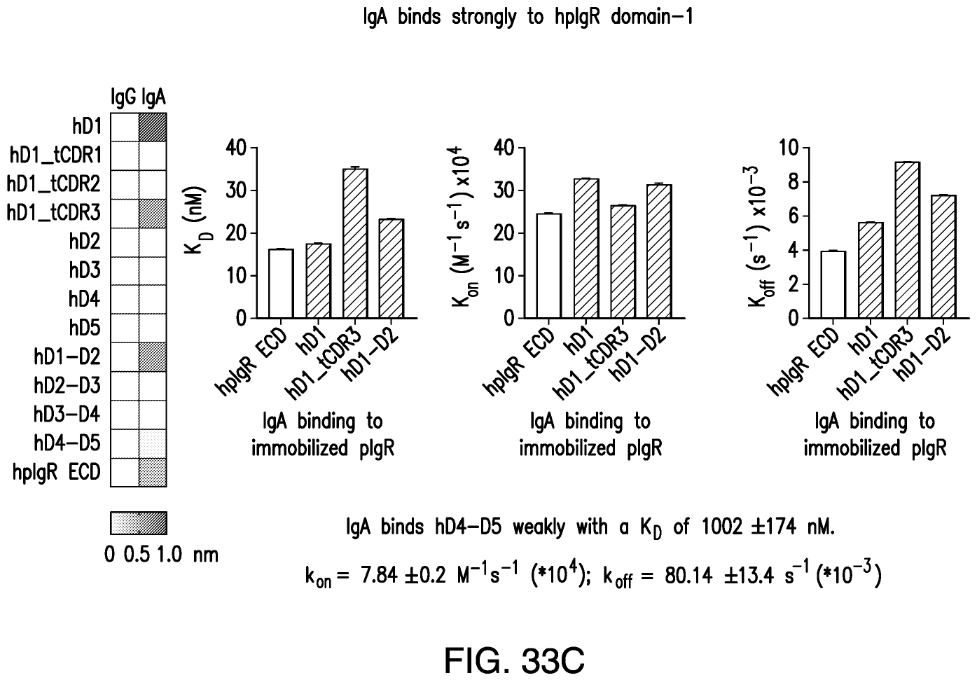

The differences in binding between VHH2, the transcytosis-positive domain-1 binder described above, and VHH3, a transcytosis-negative domain-1 binder, were compared. VHH3 binds stronger than VHH2. To test the importance of hpIgR domain-1 CDRs on VHH2 and VHH3 binding, each domain-1 CDR of human pIgR was swapped with the respective domain-1 CDR of teleost fish pIgR to make three new CDR-swapped hpIgR domain-1 constructs for use in binding studies. (Full-length hpIgR ECD was purchased from R&D Systems.) The five constructs (D1-D2, D1, D1_tCDR1, D1_tCDR2, D1_tCDR3) were expressed and purified from HEK293 cells using immobilized metal ion affinity chromatography. Three hpIgR domain-1 CDR mutants (D1_tCDR1, D1_tCDR2, D1_tCDR3) contain respective teleost fish CDR on a hpIgR domain-1 framework. His-tagged pIgR constructs were immobilized on anti-HIS biosensors and binding of VHH-mono-Fc molecules to pIgR constructs were measured by bio-layer interferometry. The data is shown in Table 3 and FIGS. 33A-33D.

TABLE 3

| | VHH2 | Fold change | VHH3 | Fold change |
|---|---|---|---|---|
| pIgR | K_{D} (nM) | in K_{D} | K_{D} (nM) | in K_{D} |
| hpIgR_ECD | 22 | 1 | 10 | 1 |
| hpIgR_D1-D2 | 23 | 1 | 13.3 | 1.3 |
| hpIgR_D1 | 15.5 | 0.7 | 7.7 | 0.8 |
| hpIgR_D1_tCDR1 | 21.3 | 1 | 77 | 7.7 |
| hpIgR_D1_tCDR2 | No binding | No binding | No binding | No binding |
| hpIgR_D1_tCDR3 | 14.9 | 0.7 | 7.5 | 0.8 |

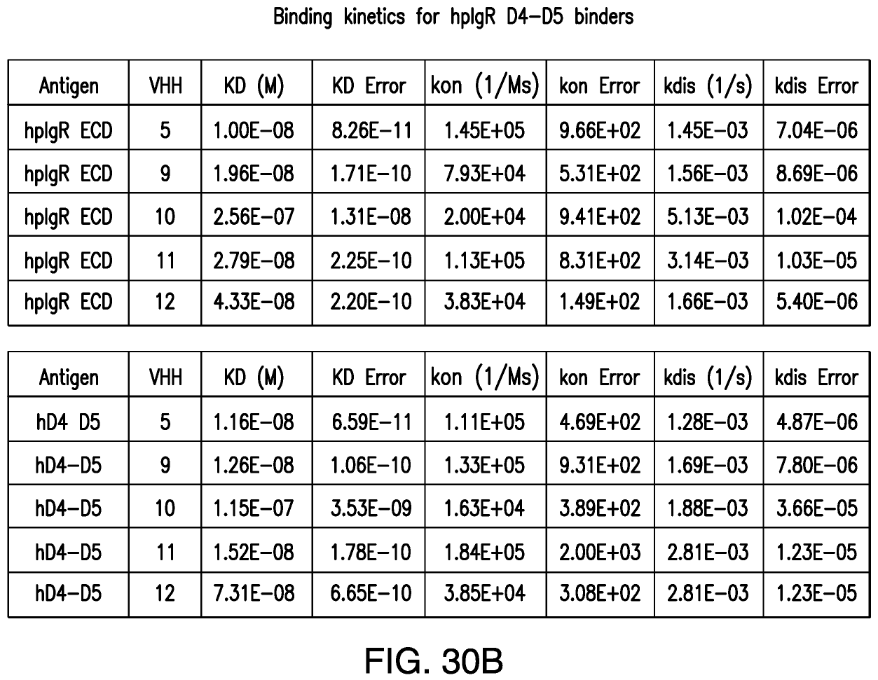

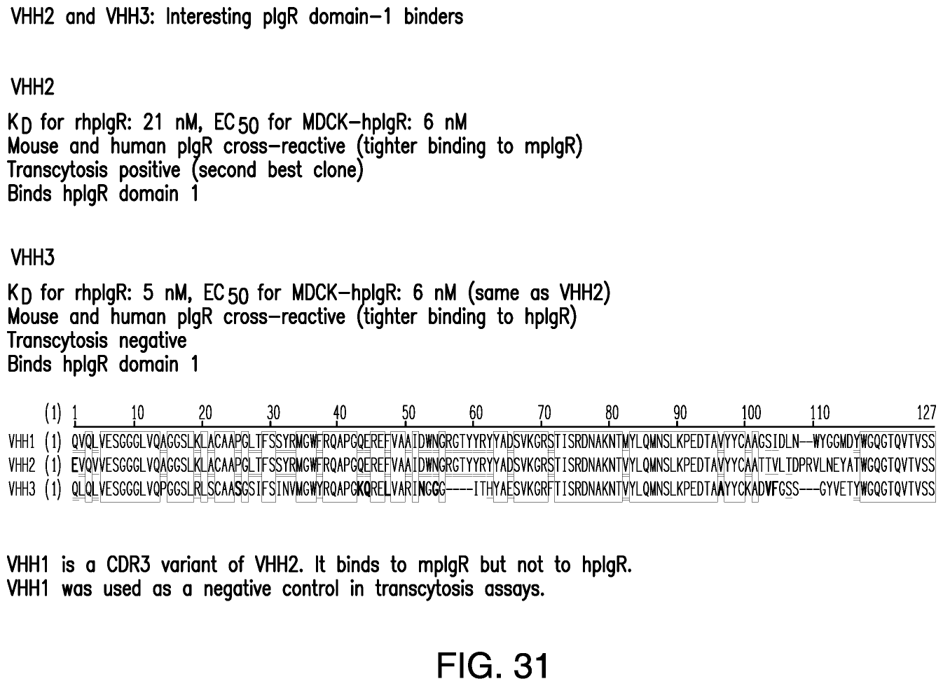

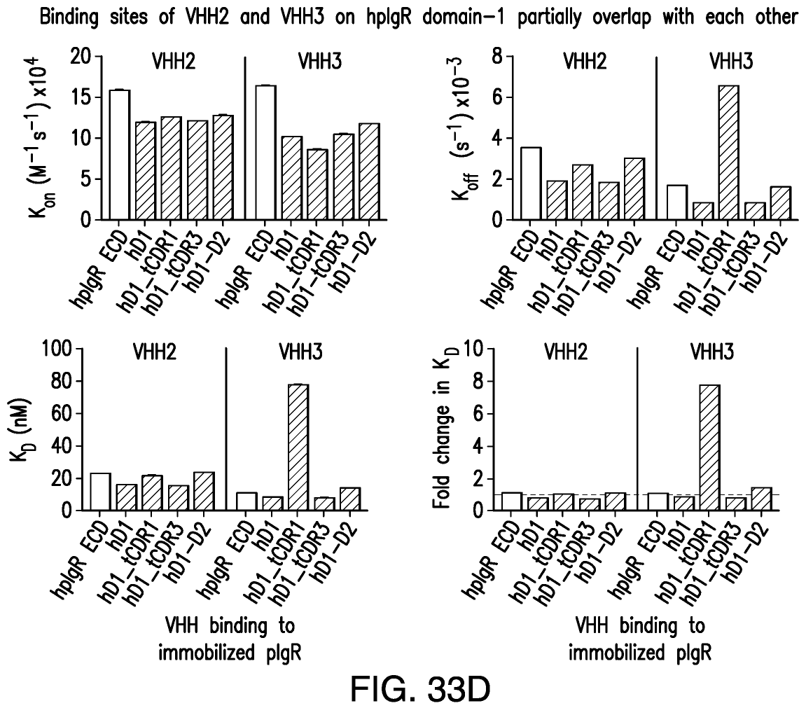

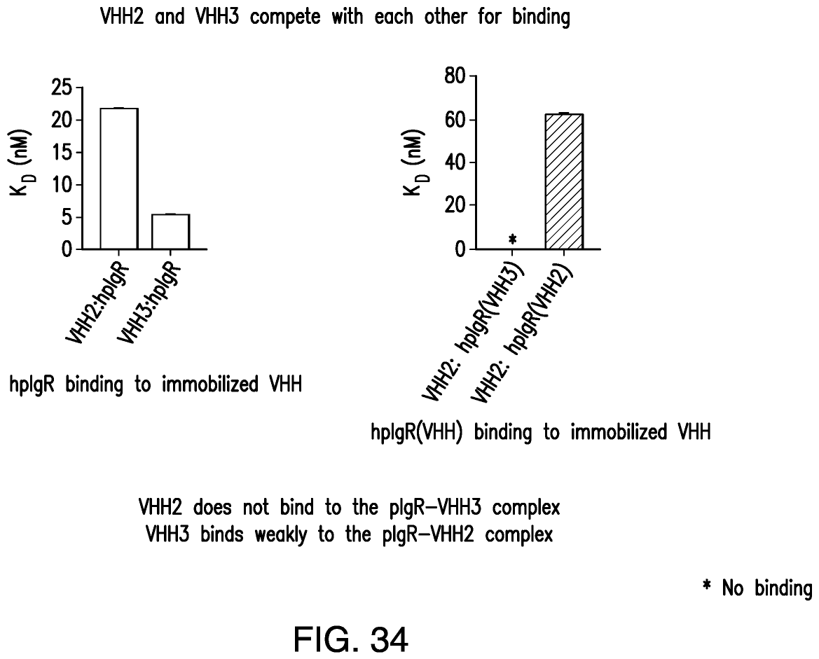

In Table 3, the K_{D} values for two VHH-mono-Fc molecules (VHH2 and VHH3) binding to six HIS-tagged pIgR constructs. VHH2 and VHH3 showed similar binding profiles towards CDR2 and CDR3 of hpIgR domain-1, while having different binding profiles towards CDR1 of hpIgR domain-1. The properties of VHH2 and VHH3 are summarized in FIG. 31. The data of FIG. 34 show that VHH2 and VHH3 compete with each other for binding to hpIgR.

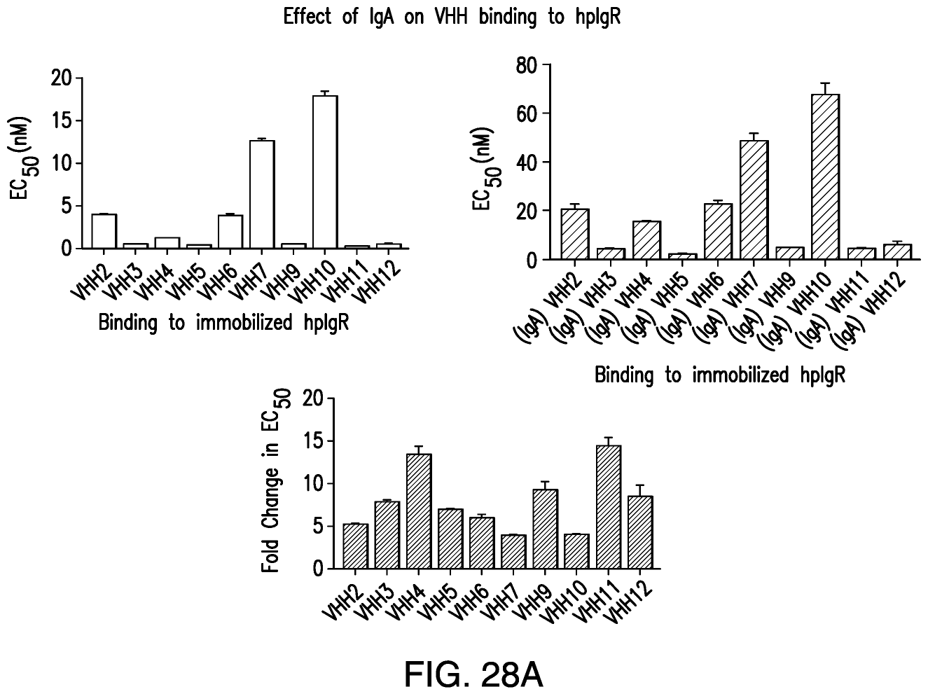

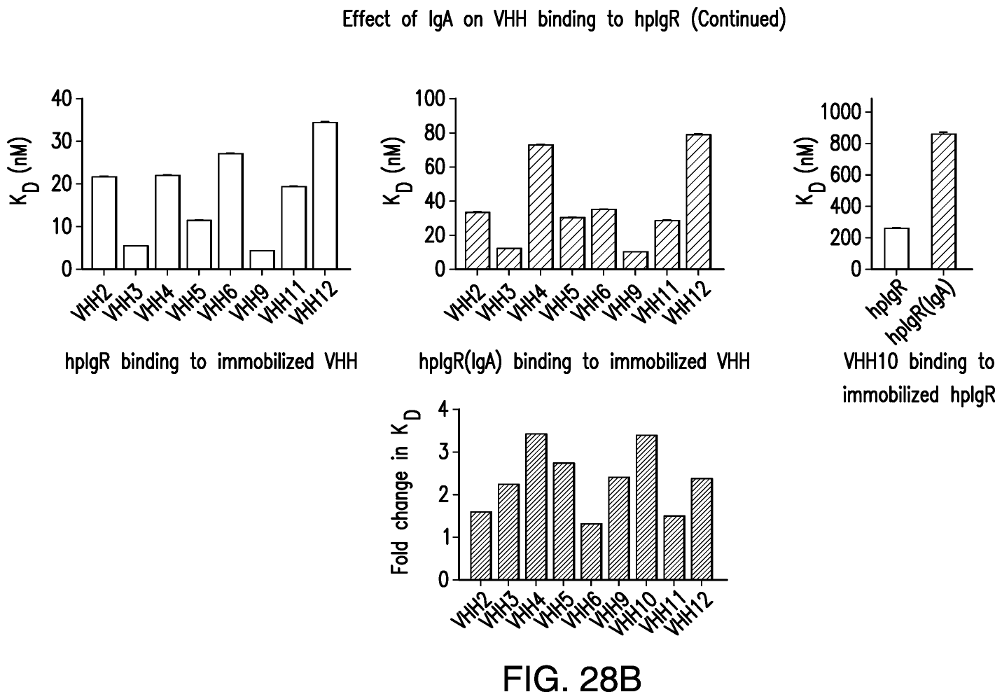

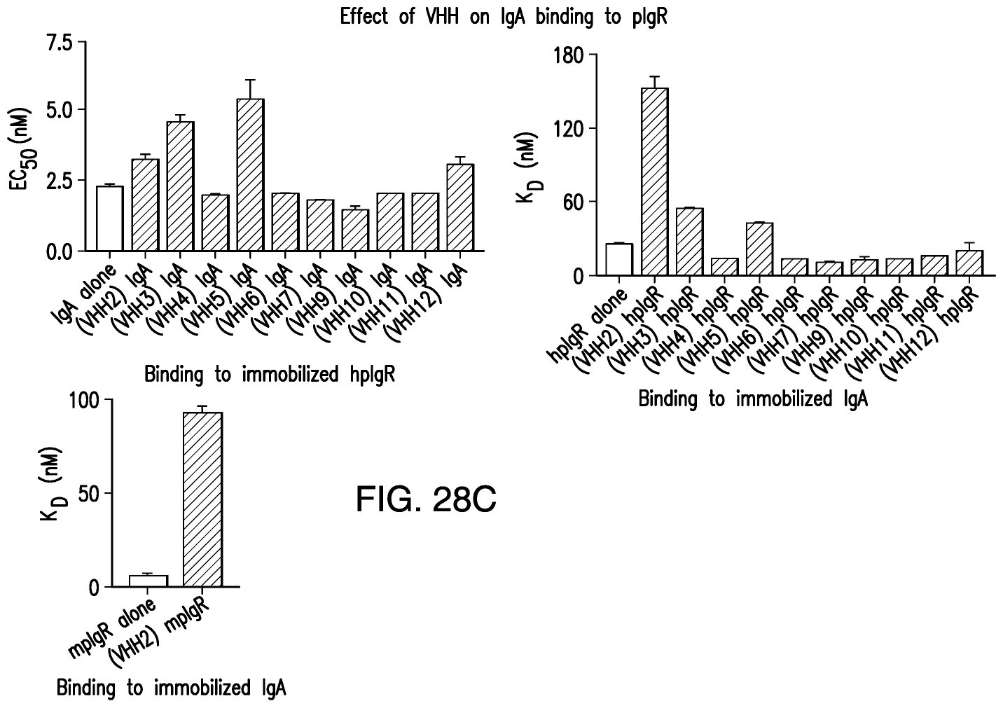

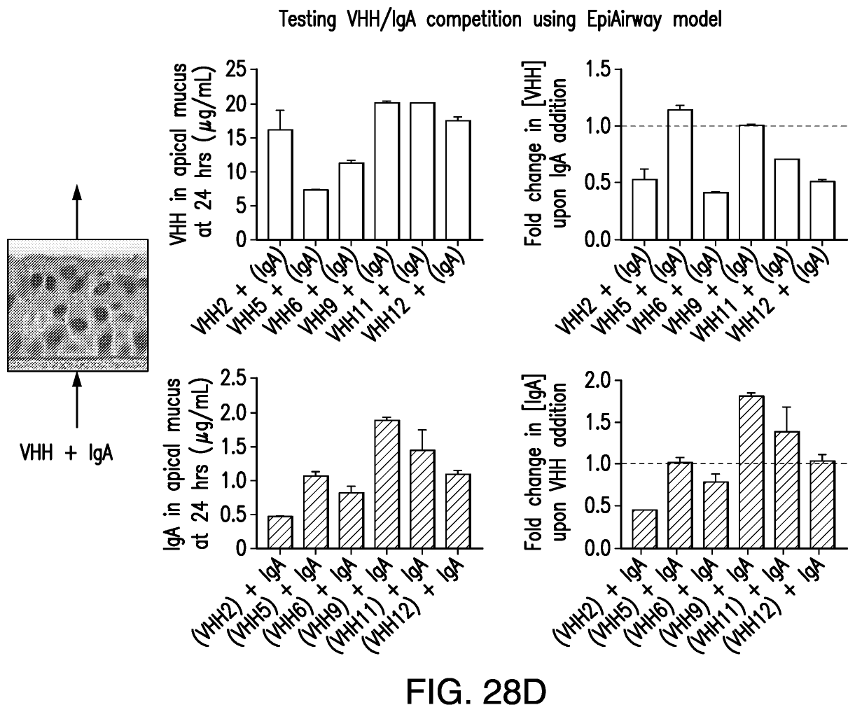

Competition binding assays showed that IgA had a negative effect on binding of VHH molecules to pIgR, possibly due to steric hindrance arising from the size difference between dimeric IgA and VHH. The data is shown in FIGS. 28A-28D. Given that hpIgR domain-1 is necessary for IgA binding, only VHH2 (domain-1 binder) had a negative effect on IgA binding to hpIgR and showed direct competition with IgA.

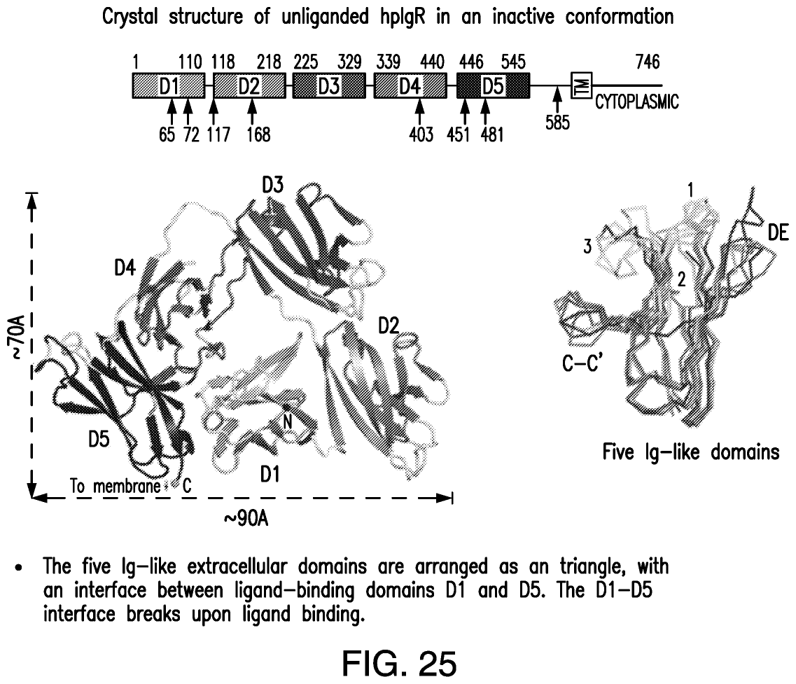

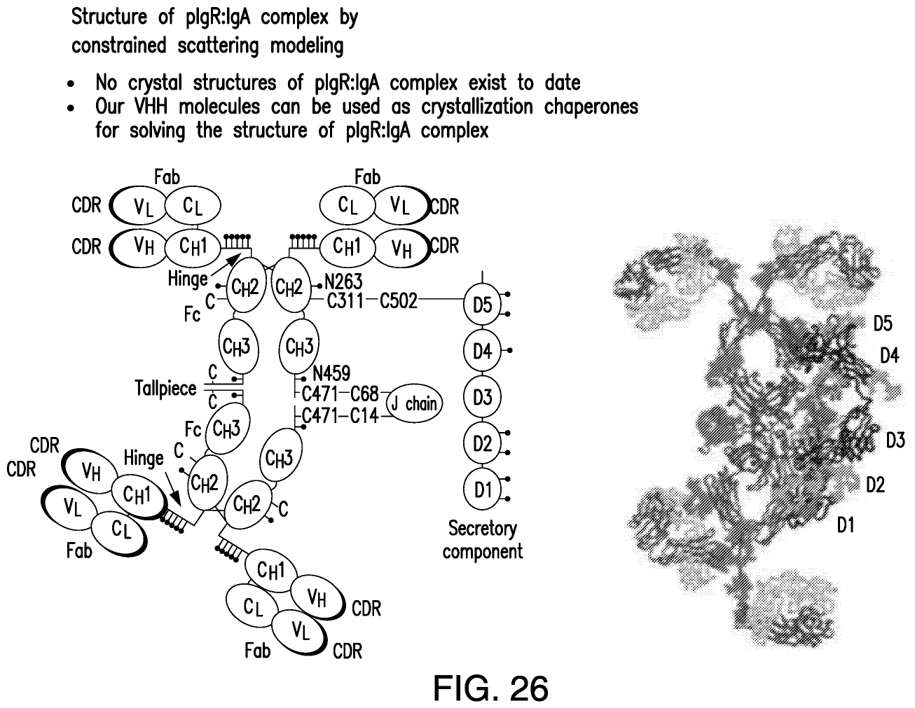

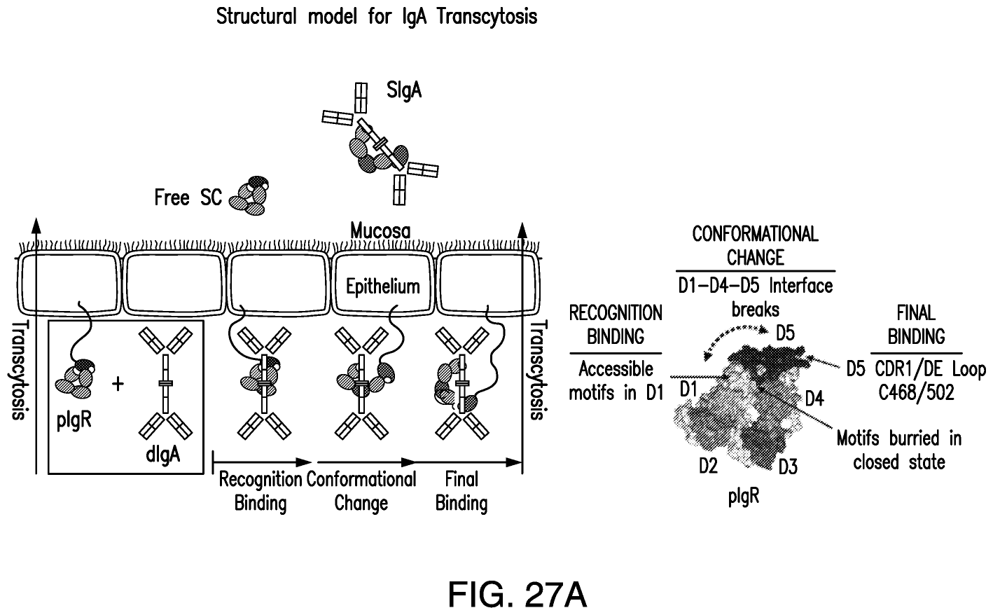

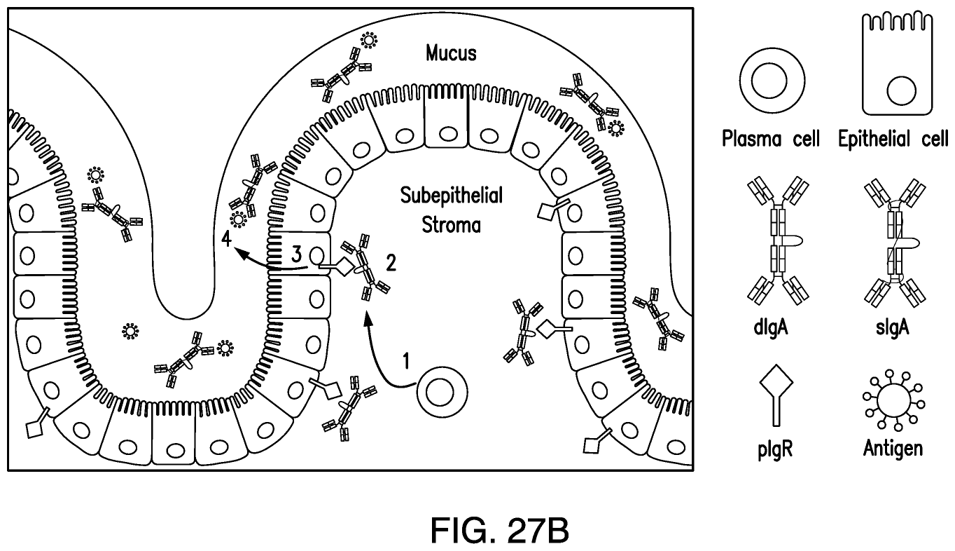

VHH2 and VHH3 showed similar binding profiles towards CDR2 and CDR3 of hpIgR domain-1, whereas showed different binding profiles towards CDR1 of hpIgR domain-1. This indicated that VHH2 and VHH3 overlap partial epitopes on domain-1 and thus competed with one another for binding to hpIgR. Further, binding assays suggested that VHH3 binds to a more hidden epitope on domain-1 relative to VHH2 (Table 2). Interestingly, VHH3-treated EpiAirway tissue model retained more pIgR in the basolateral epithelium relative to VHH2 or no VHH (FIG. 5). Given that the domain-1 plays a crucial interface and role in the inactive to active transitioning of hpIgR, these results suggest that VHH3 binding could shift the pIgR equilibrium towards an inactive conformation. As shown in FIG. 25, the five Ig-like extracellular domains are arranged as a triangle, with an interface between ligand-binding domains D1 and D5. The D1-D5 interface breaks upon ligand binding. FIG. 26 shows structure of pIgR:IgA complex by constrained scattering modeling.

A summary of the properties of the tested VHH molecules is shown in Table 4 below.

TABLE 4

| | Tₘ | K_{D} | EC₅₀ | mpIgR | Buried | pIgR Epitope | IgA Dir. | Transcytosis |
|---|---|---|---|---|---|---|---|---|
| VHH | (° C.) | (nM) | (nM) | binding? | epitope? | Domain | Comp? | (Fold Incr.) |
| VHH2 | 64.1 | 21 | 6.3 | Yes | No | 1 | Yes | 34.2 |
| VHH3 | 75.9 | 5 | 6.4 | Yes | Yes | 1 | Yes | 2.6 |
| VHH4 | 61.5 | 22 | 32.9 | No | Yes | 2 | No | 12.6 |
| VHH5 | 76.4 | 11 | 4.3 | No | Yes | 4-5 | Yes | 6.9 |
| VHH6 | 69.3 | 27 | 11.5 | No | Yes | 2 | No | 30.7 |
| VHH7 | 55.3 | 521 | 36.4 | No | No | 4-5 | No | 1.3 |
| VHH9 | 70.3 | 4 | 1.5 | No | No | 4-5 | No | 22.4 |
| VHH10 | 53.9 | 256 | 20.4 | No | No | 4-5 | No | 5.0 |
| VHH11 | 69.2 | 19 | 1.5 | No | No | 4-5 | No | 32.0 |
| VHH12 | 61.5 | 34 | 4.6 | No | No | 5 | No | 38.6 |

The above examples show the generation, screening and characterization of hpIgR-binding VHH molecules by biophysical and functional assays. VHH molecules showed varying degrees of affinity, species cross-reactivity, biophysical characteristics, epitope diversity, IgA competition profiles and transcytosis activity in a human lung tissue model.

### 6.7. Example 7: Additional Transcytosis Assays

MDCK cells expressing hpIgR as described in Example 3, are a relevant epithelia model system and were used to assay forward and reverse transcytosis activities of VHH-mono-Fc molecules.

MDCK cells expressing hpIgR were cultured in DMEM containing 10% FBS at 37° C. with 5% CO₂. To prepare monolayers of such cells (MDCK-hpIgR monolayers), 5×10⁵ cells were seeded on fibronectin- and collagen-treated Transwell™ permeable supports (Costar) containing 0.4 μm polyester membrane filter. The cells were then incubated for 3 days, serum starved for 2 hours and supplemented with DMEM containing 1% FBS (assay media). Basolateral and apical chambers contained 1.5 ml and 0.5 ml of assay media, respectively.

To test the forward transcytosis activity of VHH-mono-Fc molecules across the MDCK-hpIgR monolayers, 20 μg of test or control VHH-mono-Fc molecules were added to the basolateral chamber and 100 μl of media was collected from the basolateral and apical chambers at different time points following the addition of VHH-mono-Fc molecules (0, 4, 8, 12, 24, 36 and 48 hours).

To test the reverse transcytosis activity of VHH-mono-Fc molecules across the MDCK-hpIgR monolayers, 20 μg of test or control VHH-mono-Fc molecules were added to the apical chamber and 100 μl of media was collected from the basolateral and apical chambers at different time points following the addition of VHH-mono-Fc (0, 4, 8, 12, 24, 36 and 48 hours).

The amount of VHH-mono-Fc present in basolateral and apical media was quantified by electrochemiluminescence method. Streptavidin MSD plates were coated with a biotinylated anti-VHH antibody (2 μg/ml in PBS) for 1 hour at RT with 1000 rpm, washed 3× with PBT, incubated with blocking buffer for 1 hour at RT, incubated with VHH-mono-Fc containing media/mucus (at different dilutions) for 1 hour at RT with 1000 rpm, washed 3× with PBT, incubated with ruthenylated-anti-human-Fc antibody (2 μg/ml in PBS) for 1 hour at RT with 1000 rpm, washed 3× with PBT and read plates in 40 μl reading buffer using the MSD imager. The amount of VHH in basolateral and apical chambers were calculated by plotting ECLU values against VHH-mono-Fc standard curves in Prism (Graphpad).

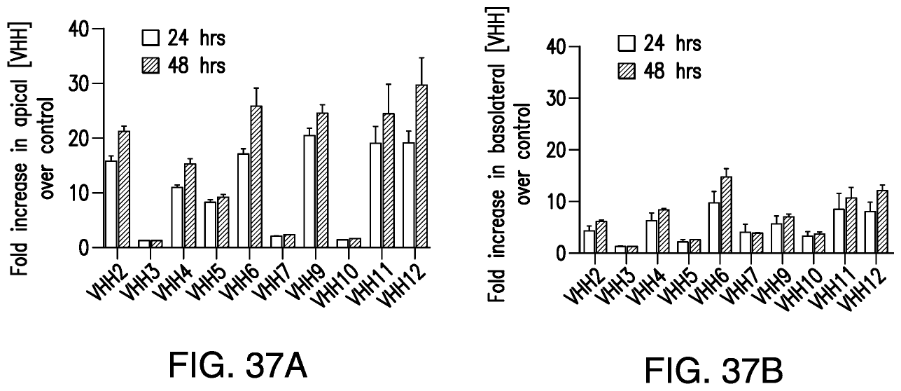

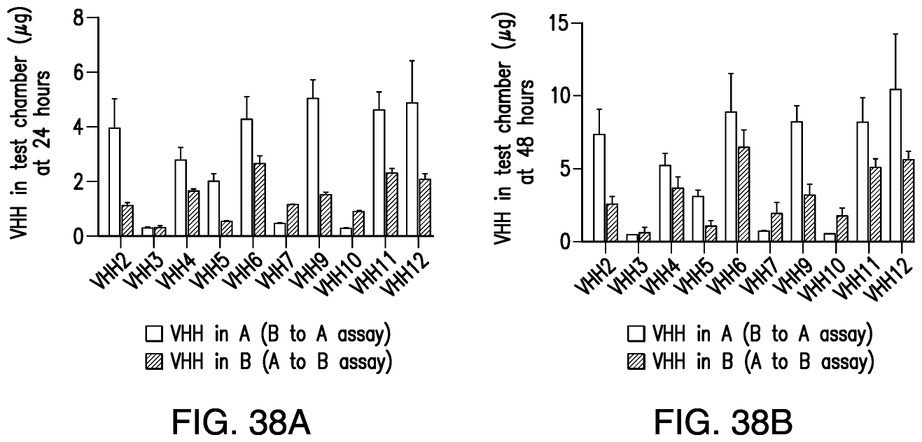

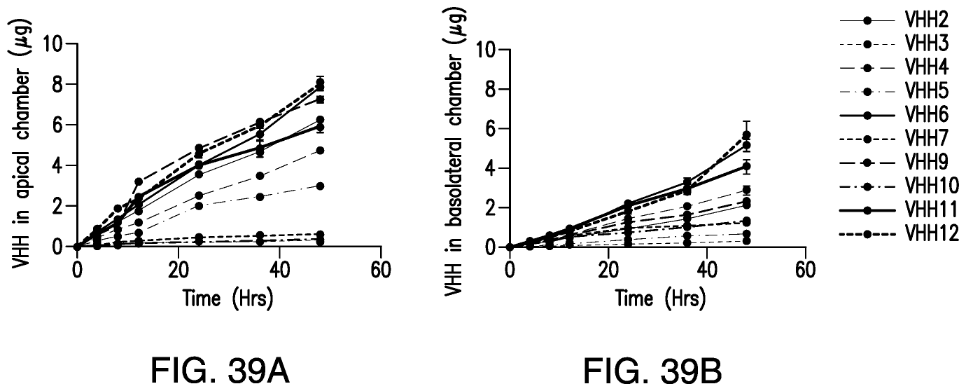

The results of the forward and reverse transcytosis assays are shown in FIGS. 37A, 37B, 38A, 38B, 39A and 39B.

A summary of the properties of the tested VHH molecules is shown in Table 5 below.

TABLE 5

| | | | | | | | For. | Rev. |
|---|---|---|---|---|---|---|---|---|
| | | | | | pIgR | IgA | Trans. | Trans |
| | Tₘ | K_{D} | EC₅₀ | mpIgR | Epitope | Dir. | (Fold | (Fold |
| VHH | (° C.) | (nM) | (nM) | binding? | Domain | Comp? | Incr.) | Incr.) |
| VHH2 | 64.1 | 21 | 6.3 | Yes | 1 | Yes | 21.0 | 5.7 |
| VHH3 | 75.9 | 5 | 6.4 | Yes | 1 | Yes | 1.0 | 1.0 |
| VHH4 | 61.5 | 22 | 32.9 | No | 2 | No | 15.1 | 8.0 |
| VHH5 | 76.4 | 11 | 4.3 | No | 4-5 | Yes | 8.9 | 2.1 |
| VHH6 | 69.3 | 27 | 11.5 | No | 2 | No | 25.6 | 14.6 |
| VHH7 | 55.3 | 521 | 36.4 | No | 4-5 | No | 1.9 | 3.3 |
| VHH9 | 70.3 | 4 | 1.5 | No | 4-5 | No | 24.2 | 6.6 |
| VHH10 | 53.9 | 256 | 20.4 | No | 4-5 | No | 1.3 | 3.4 |
| VHH11 | 69.2 | 19 | 1.5 | No | 4-5 | No | 24.2 | 10.3 |
| VHH12 | 61.5 | 34 | 4.6 | No | 5 | No | 29.5 | 11.7 |

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

SEQUENCE LISTING

SEQ ID NO: 1 - VHH1 and VHH2 CDR1 (Kabat)
SYRMG
SEQ ID NO: 2 - VHH3 CDR1 (Kabat)
INVMG
SEQ ID NO: 3 - VHH4 CDR1 (Kabat)
SNAMG
SEQ ID NO: 4 - VHH5 CDR1 (Kabat)
SYAMG
SEQ ID NO: 5 - VHH6 CDR1 (Kabat)
SDAMG
SEQ ID NO: 6 - VHH7 CDR1 (Kabat)
INVMG
SEQ ID NO: 7 - VHH9 CDR1 (Kabat)
TYRMG
SEQ ID NO: 8 - VHH10 CDR1 (Kabat)
RYAMG
SEQ ID NO: 9 - VHH12 CDR1 (Kabat)
FNTYAMG
SEQ ID NO: 10 - VHH1 and VHH2 CDR1 (Chothia)
GLTFSSY
SEQ ID NO: 11 - VHH3 CDR1 (Chothia)
GSIFSIN
SEQ ID NO: 12 - VHH4 CDR1 (Chothia)
GTSVSSN
SEQ ID NO: 13 - VHH5 CDR1 (Chothia)
GRTFSSY
SEQ ID NO: 14 - VHH6 CDR1 (Chothia)
GSSVSSD
SEQ ID NO: 15 - VHH7 CDR1 (Chothia)
RSIGSIN
SEQ ID NO: 16 - VHH9 CDR1 (Chothia)
GRTFSTY
SEQ ID NO: 17 - VHH10 CDR1 (Chothia)
GFTFTRY
SEQ ID NO: 18 - VHH11 CDR1 (Chothia)
GRTFTTY
SEQ ID NO: 19 - VHH12 CDR1 (Chothia)
GRTLSFNTY
SEQ ID NO: 20 - VHH1 and VHH2 CDR1 (IMGT)
GLTFSSYR
SEQ ID NO: 21 - VHH3 CDR1 (IMGT)
GSIFSINV
SEQ ID NO: 22 - VHH4 CDR1 (IMGT)
GTSVSSNA
SEQ ID NO: 23 - VHH5 CDR1 (IMGT)
GRTFSSYA
SEQ ID NO: 24 - VHH6 CDR1 (IMGT)
GSSVSSDA
SEQ ID NO: 25 - VHH7 CDR1 (IMGT)
RSIGSINV
SEQ ID NO: 26 - VHH9 CDR1 (IMGT)
GRTFSTYR
SEQ ID NO: 27 - VHH10 CDR1 (IMGT)
GFTFTRYA
SEQ ID NO: 28 - VHH11 CDR1 (IMGT)
GRTFTTYR
SEQ ID NO: 29 - VHH12 CDR1 (IMGT)
GRTLSFNTYA
SEQ ID NO: 30 - VHH1 and VHH2 CDR2 (Kabat)
AIDWNGRGTYYRYYADSVKG
SEQ ID NO: 31 - VHH3 CDR2 (Kabat)
RINGGGITHYAESVKG
SEQ ID NO: 32 - VHH4 CDR2 (Kabat)
FIDRIATTTIATSVKG
SEQ ID NO: 33 - VHH5 CDR2 (Kabat)
AITWNGGTTYYADSVKG
SEQ ID NO: 34 - VHH6 CDR2 (Kabat)
FISGGGTTTYADSVKG
SEQ ID NO: 35 - VHH7 CDR2 (Kabat)
RITGGGSTHYAESVKG
SEQ ID NO: 36 - VHH9 CDR2 (Kabat)
AISWSGGSTTYADPVKG
SEQ ID NO: 37 - VHH10 CDR2 (Kabat)
AISWSGSSAGYGDSVKG
SEQ ID NO: 38 - VHH11 CDR2 (Kabat)
AIRWSGGRTLYADSVKG
SEQ ID NO: 39 - VHH12 CDR2 (Kabat)
SITWNGGSTSYADSVKG
SEQ ID NO: 40 - VHH1 and VHH2 CDR2 (Chothia)
DWNGRGTYY
SEQ ID NO: 41 - VHH3 CDR2 (Chothia)
NGGGI
SEQ ID NO: 42 - VHH4 CDR2 (Chothia)
DRIAT
SEQ ID NO: 43 - VHH5 CDR2 (Chothia)
TWNGGT
SEQ ID NO: 44 - VHH6 CDR2 (Chothia)
SGGGT
SEQ ID NO: 45 - VHH7 CDR2 (Chothia)
TGGGS
SEQ ID NO: 46 - VHH9 CDR2 (Chothia)
SWSGGS
SEQ ID NO: 47 - VHH10 CDR2 (Chothia)
SWSGSS
SEQ ID NO: 48 - VHH11 CDR2 (Chothia)
RWSGGR
SEQ ID NO: 49 - VHH12 CDR2 (Chothia)
TWNGGS
SEQ ID NO: 50 - VHH1 and VHH2 CDR2 (IMGT)
IDWNGRGTYY
SEQ ID NO: 51 - VHH3 CDR2 (IMGT)
INGGGIT
SEQ ID NO: 52 - VHH4 CDR2 (IMGT)
IDRIATT
SEQ ID NO: 53 - VHH5 CDR2 (IMGT)
ITWNGGTT
SEQ ID NO: 54 - VHH6 CDR2 (IMGT)
ISGGGTT
SEQ ID NO: 55 - VHH7 CDR2 (IMGT)
ITGGGST
SEQ ID NO: 56 - VHH9 CDR2 (IMGT)
ISWSGGST
SEQ ID NO: 57 - VHH10 CDR2 (IMGT)
ISWSGSSA
SEQ ID NO: 58 - VHH11 CDR2 (IMGT)
IRWSGGRT
SEQ ID NO: 59 - VHH12 CDR2 (IMGT)
ITWNGGST
SEQ ID NO: 60 - VHH1 CDR3 (Kabat)
GSIDLNWYGGMDY
SEQ ID NO: 61 - VHH2 CDR3 (Kabat)
TTVLTDPRVLNEYAT
SEQ ID NO: 62 - VHH3 CDR3 (Kabat)
DVFGSSGYVETY
SEQ ID NO: 63 - VHH4 CDR3 (Kabat)
PLTAR
SEQ ID NO: 64 - VHH5 CDR3 (Kabat)
DPFNQGY
SEQ ID NO: 65 - VHH6 CDR3 (Kabat)
PLTSR
SEQ ID NO: 66 - VHH7 CDR3 (Kabat)
MVNPIITAWGTIGVREIPDYDY
SEQ ID NO: 67 - VHH9 CDR3 (Kabat)
DQRGY
SEQ ID NO: 68 - VHH10 CDR3 (Kabat)
DPFNQGY
SEQ ID NO: 69 - VHH11 CDR3 (Kabat)
DLAEYSGTYSSPADSPAGYDY
SEQ ID NO: 70 - VHH12 CDR3 (Kabat)
ARYYVSGTYFPANY
SEQ ID NO: 71 - VHH1 CDR3 (Chothia)
GSIDLNWYGGMDY
SEQ ID NO: 72 - VHH2 CDR3 (Chothia)
TTVLTDPRVLNEYAT
SEQ ID NO: 73 - VHH3 CDR3 (Chothia)
DVFGSSGYVETY
SEQ ID NO: 74 - VHH4 CDR3 (Chothia)
PLTAR
SEQ ID NO: 75 - VHH5 CDR3 (Chothia)
DPFNQGY
SEQ ID NO: 76 - VHH6 CDR3 (Chothia)
PLTSR
SEQ ID NO: 77 - VHH7 CDR3 (Chothia)
MVNPIITAWGTIGVREIPDYDY
SEQ ID NO: 78 - VHH9 CDR3 (Chothia)
DQRGY
SEQ ID NO: 79 - VHH10 CDR3 (Chothia)
DPFNQGY
SEQ ID NO: 80 - VHH11 CDR3 (Chothia)
DLAEYSGTYSSPADSPAGYDY
SEQ ID NO: 81 - VHH12 CDR3 (Chothia)
ARYYVSGTYFPANY
SEQ ID NO: 82 - VHH1 CDR3 (IMGT)
CAAGSIDLNWYGGMDY
SEQ ID NO: 83 - VHH2 CDR3 (IMGT)
CAATTVLTDPRVLNEYAT
SEQ ID NO: 84 - VHH3 CDR3 (IMGT)
KADVFGSSGYVETY
SEQ ID NO: 85 - VHH4 CDR3 (IMGT)
NHPLTAR
SEQ ID NO: 86 - VHH5 CDR3 (IMGT)
AADPFNQGY
SEQ ID NO: 87 - VHH6 CDR3 (IMGT)
NHPLTSR
SEQ ID NO: 88 - VHH7 CDR3 (IMGT)
ASMVNPIITAWGTIGVREIPDYDY
SEQ ID NO: 89 - VHH9 CDR3 (IMGT)
NDQRGY
SEQ ID NO: 90 - VHH10 CDR3 (IMGT)
AADPFNQGY
SEQ ID NO: 91 - VHH11 CDR3 (IMGT)
AADLAEYSGTYSSPADSPAGYDY
SEQ ID NO: 92 - VHH12 CDR3 (IMGT)
AAARYYVSGTYFPANY
SEQ ID NO: 93 - VHH1 - VH amino acid sequence
QVQLVESGGGLVQAGGSLKLACAAPGLTFSSYRMGWFRQAPGQEREFVAAIDWNGRG
TYYRYYADSVKGRSTISRDNAKNTMYLQMNSLKPEDTAVYYCAAGSIDLNWYGGMD
YWGQGTQVTVSS
SEQ ID NO: 94 - VHH2 - VH amino acid sequence
EVQVVESGGGLVQAGGSLKLACAAPGLTFSSYRMGWFRQAPGQEREFVAAIDWNGRG
TYYRYYADSVKGRSTISRDNAKNTVYLQMNSLKPEDTAVYYCAATTVLTDPRVLNEYA
TWGQGTQVTVSS
SEQ ID NO: 95 - VHH3 - VH amino acid sequence
QLQLVESGGGLVQPGGSLRLSCAASGSIFSINVMGWYRQAPGKQRELVARINGGGITHY
AESVKGRFTISRDNAKNTVYLQMNSLKPEDTAAYYCKADVFGSSGYVETYWGQGTQV
TVSS
SEQ ID NO: 96 - VHH4 - VH amino acid sequence
EVQVVESGGGLVQAGGSLRLSCAVSGTSVSSNAMGWYRQAPGKQREWVGFIDRIATTT
IATSVKGRFAITRDNAKNTVYLQMSGLKPEDTAVYYCNHPLTARWGQGTQVTVSS
SEQ ID NO: 97 - VHH5 - VH amino acid sequence
QVQLVESGGGLVQAGGSLRLSCAASGRTFSSYAMGWFRQAPGKEREFVAAITWNGGTT
YYADSVKGRFTISRDNAKNTVYLQMNSLKPEDTAVYYCAADPFNQGYWGQGTQVTVS
S
SEQ ID NO: 98 - VHH6 - VH amino acid sequence
EVQLVESGGGLVQAGGSLRLSCAVSGSSVSSDAMGWYRQAPGNQRAWVAFISGGGTT
TYADSVKGRFTISRDNTKNTVYLHMNSLKPEDTAVYYCNHPLTSRWGQGTQVTVSS
SEQ ID NO: 99 - VHH7 - VH amino acid sequence
EVQVVESGGGLVQAGGSLRLACVASRSIGSINVMGWYRQAPGKQRDLVARITGGGSTH
YAESVKGRFTISRDNAKNTVYLQMNSLEPEDTAVYYCASMVNPIITAWGTIGVREIPDY
DYWGQGTQVTVSS
SEQ ID NO: 100 - VHH9 - VH amino acid sequence
QVQLVESGGGLVQAGGSLRLSCAVSGRTFSTYRMGWFRQAPGKERSFVAAISWSGGST
TYADPVKGRFTISRDNAKNTVYLRMNSLKPEDTAVYYCNDQRGYWGQGTLVTVSS
SEQ ID NO: 101 - VHH10 - VH amino acid sequence
EVQVVESGGGLVQAGGSLRLSCAASGFTFTRYAMGWFRQAPGKERSFVAAISWSGSSA
GYGDSVKGRFTISRDNAKNTLYLQMNSLKPEDTAVYYCAADPFNQGYWGQGTQVTVS
S
SEQ ID NO: 102 - VHH11 - VH amino acid sequence
EVQVVESGGGLVQAGGSLRLSCAASGRTFTTYRMGWFRQAPGKEREFVAAIRWSGGRT
LYADSVKGRFTISRDNAKNTAYLQMNNLRPEDTAVYYCAADLAEYSGTYSSPADSPAG
YDYWGQGTQVTVSS
SEQ ID NO: 103 - VHH12 - VH amino acid sequence
QVQLVETGGGLVQAGDSLRLSCAASGRTLSFNTYAMGWFRQAPGKEREFVASITWNG
GSTSYADSVKGRFTITRDNAKNTATLRMNSLQPDDTAVYYCAAARYYVSGTYFPANY
WGQGTQVTVSS
SEQ ID NO: 104 - VHH1-linker- mono-Fc protein
QVQLVESGGGLVQAGGSLKLACAAPGLTFSSYRMGWFRQAPGQEREFVAAIDWNGRG
TYYRYYADSVKGRSTISRDNAKNTMYLQMNSLKPEDTAVYYCAAGSIDLNWYGGMD
YWGQGTQVTVSSEPKTPKPQPQPQLQPQPNPTTESKSPKSPAPELLGGPSVFLFPPKPKD
TLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTKPPSREEMTKNQVSLS
CLVKGFYPSDIAVEWESNGQPENNYKTTVPVLDSDGSFRLASYLTVDKSRWQQGNVFS
CSVMHEALHNHYTQKSLSLSPGK
SEQ ID NO: 105 - VHH2-linker- mono-Fc protein
EVQVVESGGGLVQAGGSLKLACAAPGLTFSSYRMGWFRQAPGQEREFVAAIDWNGRG
TYYRYYADSVKGRSTISRDNAKNTVYLQMNSLKPEDTAVYYCAATTVLTDPRVLNEYA
TWGQGTQVTVSSEPKTPKPQPQPQLQPQPNPTTESKSPKSPAPELLGGPSVFLFPPKPKDT
LMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTKPPSREEMTKNQVSLSC
LVKGFYPSDIAVEWESNGQPENNYKTTVPVLDSDGSFRLASYLTVDKSRWQQGNVFSC
SVMHEALHNHYTQKSLSLSPGK
SEQ ID NO: 106 - VHH3-linker- mono-Fc protein
QLQLVESGGGLVQPGGSLRLSCAASGSIFSINVMGWYRQAPGKQRELVARINGGGITHY
AESVKGRFTISRDNAKNTVYLQMNSLKPEDTAAYYCKADVFGSSGYVETYWGQGTQV
TVSSEPKTPKPQPQPQLQPQPNPTTESKSPKSPAPELLGGPSVFLFPPKPKDTLMISRTPEV
TCVVVDVSHEDPEVKFNWYVDGVEVHNAKTKPREEQYNSTYRVVSVLTVLHQDWLN
GKEYKCKVSNKALPAPIEKTISKAKGQPREPQVYTKPPSREEMTKNQVSLSCLVKGFYPS
DIAVEWESNGQPENNYKTTVPVLDSDGSFRLASYLTVDKSRWQQGNVFSCSVMHEALH
NHYTQKSLSLSPGK
SEQ ID NO: 107 - VHH4-linker- mono-Fc protein
EVQVVESGGGLVQAGGSLRLSCAVSGTSVSSNAMGWYRQAPGKQREWVGFIDRIATTT
IATSVKGRFAITRDNAKNTVYLQMSGLKPEDTAVYYCXHPXTARWGQGTQVTVSSEPK
TPKPQPQPQLQPQPNPTTESKSPKSPAPELLGGPSVFLFPPKPKDTLMISRTPEVTCVVVD
VSHEDPEVKFNWYVDGVEVHNAKTKPREEQYNSTYRVVSVLTVLHQDWLNGKEYKC
KVSNKALPAPIEKTISKAKGQPREPQVYTKPPSREEMTKNQVSLSCLVKGFYPSDIAVEW
ESNGQPENNYKTTVPVLDSDGSFRLASYLTVDKSRWQQGNVFSCSVMHEALHNHYTQ
KSLSLSPGK
SEQ ID NO: 108 - VHH5-linker- mono-Fc protein
QVQLVESGGGLVQAGGSLRLSCAASGRTFSSYAMGWFRQAPGKEREFVAAITWNGGTT
YYADSVKGRFTISRDNAKNTVYLQMNSLKPEDTAVYYCAADPFNQGYWGQGTQVTVS
SEPKTPKPQPQPQLQPQPNPTTESKSPKSPAPELLGGPSVFLFPPKPKDTLMISRTPEVTCV
VVDVSHEDPEVKFNWYVDGVEVHNAKTKPREEQYNSTYRVVSVLTVLHQDWLNGKE
YKCKVSNKALPAPIEKTISKAKGQPREPQVYTKPPSREEMTKNQVSLSCLVKGFYPSDIA
VEWESNGQPENNYKTTVPVLDSDGSFRLASYLTVDKSRWQQGNVFSCSVMHEALHNH
YTQKSLSLSPGK
SEQ ID NO: 109 - VHH6-linker- mono-Fc protein
EVQLVESGGGLVQAGGSLRLSCAVSGSSVSSDAMGWYRQAPGNQRAWVAFISGGGTT
TYADSVKGRFTISRDNTKNTVYLHMNSLKPEDTAVYYCNHPLTSRWGQGTQVTVSSEP
KTPKPQPQPQLQPQPNPTTESKSPKSPAPELLGGPSVFLFPPKPKDTLMISRTPEVTCVVV
DVSHEDPEVKFNWYVDGVEVHNAKTKPREEQYNSTYRVVSVLTVLHQDWLNGKEYK
CKVSNKALPAPIEKTISKAKGQPREPQVYTKPPSREEMTKNQVSLSCLVKGFYPSDIAVE
WESNGQPENNYKTTVPVLDSDGSFRLASYLTVDKSRWQQGNVFSCSVMHEALHNHYT
QKSLSLSPGK
SEQ ID NO: 110 - VHH7-linker- mono-Fc protein
EVQVVESGGGLVQAGGSLRLACVASRSIGSINVMGWYRQAPGKQRDLVARITGGGSTH
YAESVKGRFTISRDNAKNTVYLQMNSLEPEDTAVYYCASMVNPIITAWGTIGVREIPDY
DYWGQGTQVTVSSEPKTPKPQPQPQLQPQPNPTTESKSPKSPAPELLGGPSVFLFPPKPK
DTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTKPPSREEMTKNQVSL
SCLVKGFYPSDIAVEWESNGQPENNYKTTVPVLDSDGSFRLASYLTVDKSRWQQGNVF
SCSVMHEALHNHYTQKSLSLSPGK
SEQ ID NO: 111 - VHH9-linker- mono-Fc protein
QVQLVESGGGLVQAGGSLRLSCAVSGRTFSTYRMGWFRQAPGKERSFVAAISWSGGST
TYADPVKGRFTISRDNAKNTVYLRMNSLKPEDTAVYYCNDQRGYWGQGTLVTVSSEP
KTPKPQPQPQLQPQPNPTTESKSPKSPAPELLGGPSVFLFPPKPKDTLMISRTPEVTCVVV
DVSHEDPEVKFNWYVDGVEVHNAKTKPREEQYNSTYRVVSVLTVLHQDWLNGKEYK
CKVSNKALPAPIEKTISKAKGQPREPQVYTKPPSREEMTKNQVSLSCLVKGFYPSDIAVE
WESNGQPENNYKTTVPVLDSDGSFRLASYLTVDKSRWQQGNVFSCSVMHEALHNHYT
QKSLSLSPGK
SEQ ID NO: 112 - VHH10-linker- mono-Fc protein
EVQVVESGGGLVQAGGSLRLSCAASGFTFTRYAMGWFRQAPGKERSFVAAISWSGSSA
GYGDSVKGRFTISRDNAKNTLYLQMNSLKPEDTAVYYCAADPFNQGYWGQGTQVTVS
SEPKTPKPQPQPQLQPQPNPTTESKSPKSPAPELLGGPSVFLFPPKPKDTLMISRTPEVTCV
VVDVSHEDPEVKFNWYVDGVEVHNAKTKPREEQYNSTYRVVSVLTVLHQDWLNGKE
YKCKVSNKALPAPIEKTISKAKGQPREPQVYTKPPSREEMTKNQVSLSCLVKGFYPSDIA
VEWESNGQPENNYKTTVPVLDSDGSFRLASYLTVDKSRWQQGNVFSCSVMHEALHNH
YTQKSLSLSPGK
SEQ ID NO: 113 - VHH11-linker- mono-Fc protein
EVQVVESGGGLVQAGGSLRLSCAASGRTFTTYRMGWFRQAPGKEREFVAAIRWSGGRT
LYADSVKGRFTISRDNAKNTAYLQMNNLRPEDTAVYYCAADLAEYSGTYSSPADSPAG
YDYWGQGTQVTVSSEPKTPKPQPQPQLQPQPNPTTESKSPKSPAPELLGGPSVFLFPPKP
KDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTKPPSREEMTKNQ
VSLSCLVKGFYPSDIAVEWESNGQPENNYKTTVPVLDSDGSFRLASYLTVDKSRWQQG
NVFSCSVMHEALHNHYTQKSLSLSPGK
SEQ ID NO: 114 - VHH12-linker- mono-Fc protein
QVQLVETGGGLVQAGDSLRLSCAASGRTLSFNTYAMGWFRQAPGKEREFVASITWNG
GSTSYADSVKGRFTITRDNAKNTATLRMNSLQPDDTAVYYCAAARYYVSGTYFPANY
WGQGTQVTVSSEPKTPKPQPQPQLQPQPNPTTESKSPKSPAPELLGGPSVFLFPPKPKDTL
MISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTKPPSREEMTKNQVSLSCL
VKGFYPSDIAVEWESNGQPENNYKTTVPVLDSDGSFRLASYLTVDKSRWQQGNVFSCS
VMHEALHNHYTQKSLSLSPGK
SEQ ID NO: 115 - IgA Heavy Chain
QVQLVQSGAEVKKPGSSVKVSCKSSGGTSNNYAISWVRQAPGQGLDWMGGISPIFGST
AYAQKFQGRVTISADIFSNTAYMELNSLTSEDTAVYFCARHGNYYYYSGMDVWGQGT
TVTVSSASPTSPKVFPLSLDSTPQDGNVVVACLVQGFFPQEPLSVTWSESGQNVTARNFP
PSQDASGDLYTTSSQLTLPATQCPDGKSVTCHVKHYTNPSQDVTVPCPVPPPPPCCHPRL
SLHRPALEDLLLGSEANLTCTLTGLRDASGATFTWTPSSGKSAVQGPPERDLCGCYSVSS
VLPGCAQPWNHGETFTCTAAHPELKTPLTANITKSGNTFRPEVEILLPPPSEELALNELVT
LTCLARGFSPKDVLVRWLQGSQELPREKYLTWASRQEPSQGTTTFAVTSILRVAAEDWK
KGDTFSCMVGHEALPLAFTQKTIDRLAGKPTHVNVSVVMAEVDGTCY
SEQ ID NO: 116 - IgA Light Chain
QSALTQPPAVSGTPGQRVTISCSGSDSNIGRRSVNWYQQFPGTAPKLLIYSNDQRPSVVP
DRFSGSKSGTSASLAISGLQSEDEAEYYCAAWDDSLKGAVFGGGTQLTVLGQPKAAPSV
TLFPPSSEELQANKATLVCLISDFYPGAVTVAWKADSSPVKAGVETTTPSKQSNNKYAA
SSYLSLTPEQWKSHRSYSCQVTHEGSTVEKTVAPTECS
SEQ ID NO: 117 - IgA J Chain
SRDSSASASRVAGITAQEDERIVLVDNKCKCARITSRIIRSSEDPNEDIVERNIRIIVPLNNR
ENISDPTSPLRTRFVYHLSDLCKKCDPTEVELDNQIVTATQSNICDEDSATETCYTYDRN
KCYTAVVPLVYGGETKMVETALTPDACYPD
SEQ ID NO: 118 - human pIgR extracellular domain (ECD)
KSPIFGPEEVNSVEGNSVSITCYYPPTSVNRHTRKYWCRQGARGGCITLISSEGYVSSKY
AGRANLTNFPENGTFVVNIAQLSQDDSGRYKCGLGINSRGLSFDVSLEVSQGPGLLNDT
KVYTVDLGRTVTINCPFKTENAQKRKSLYKQIGLYPVLVIDSSGYVNPNYTGRIRLDIQG
TGQLLFSVVINQLRLSDAGQYLCQAGDDSNSNKKNADLQVLKPEPELVYEDLRGSVTF
HCALGPEVANVAKFLCRQSSGENCDVVVNTLGKRAPAFEGRILLNPQDKDGSFSVVITG
LRKEDAGRYLCGAHSDGQLQEGSPIQAWQLFVNEESTIPRSPTVVKGVAGSSVAVLCPY
NRKESKSIKYWCLWEGAQNGRCPLLVDSEGWVKAQYEGRLSLLEEPGNGTFTVILNQL
TSRDAGFYWCLTNGDTLWRTTVEIKIIEGEPNLKVPGNVTAVLGETLKVPCHFPCKFSSY
EKYWCKWNNTGCQALPSQDEGPSKAFVNCDENSRLVSLTLNLVTRADEGWYWCGVK
QGHFYGETAAVYVAVEERKAAGSRDVSLAKADAAPDEKVLDSGFREIENKAIQDPRLF
AEEKAVADTRDQADGSRASVDSGSSEEQGGSSRHHHHHH
SEQ ID NO: 119 - human pIgR extracellular domain 1 (D1)
KSPIFGPEEVNSVEGNSVSITCYYPPTSVNRHTRKYWCRQGARGGCITLISSEGYVSSKY
AGRANLTNFPENGTFVVNIAQLSQDDSGRYKCGLGINSRGLSFDVSLEVGSHHHHHH
SEQ ID NO: 120 - human pIgR extracellular domain 2 (D2)
SQGPGLLNDTKVYTVDLGRTVTINCPFKTENAQKRKSLYKQIGLYPVLVIDSSGYVNPN
YTGRIRLDIQGTGQLLFSVVINQLRLSDAGQYLCQAGDDSNSNKKNADLQVLKPEPGSH
HHHHH
SEQ ID NO: 121 - human pIgR extracellular domain 3 (D3)
KPEPELVYEDLRGSVTFHCALGPEVANVAKFLCRQSSGENCDVVVNTLGKRAPAFEGRI
LLNPQDKDGSFSVVITGLRKEDAGRYLCGAHSDGQLQEGSPIQAWQLFVNEESTGSHHH
HHH
SEQ ID NO: 122 - human pIgR extracellular domain 5 (D5)
GEPNLKVPGNVTAVLGETLKVPCHFPCKFSSYEKYWCKWNNTGCQALPSQDEGPSKAF
VNCDENSRLVSLTLNLVTRADEGWYWCGVKQGHFYGETAAVYVAVEERGSHHHHHH
SEQ ID NO: 123 - human pIgR extracellular domain 1-domain 2 (D1-D2)
KSPIFGPEEVNSVEGNSVSITCYYPPTSVNRHTRKYWCRQGARGGCITLISSEGYVSSKY
AGRANLTNFPENGTFVVNIAQLSQDDSGRYKCGLGINSRGLSFDVSLEVSQGPGLLNDT
KVYTVDLGRTVTINCPFKTENAQKRKSLYKQIGLYPVLVIDSSGYVNPNYTGRIRLDIQG
TGQLLFSVVINQLRLSDAGQYLCQAGDDSNSNKKNADLQVLKPGSHHHHHH
SEQ ID NO: 124 - human pIgR extracellular domain 2-domain 3 (D2-D3)
SQGPGLLNDTKVYTVDLGRTVTINCPFKTENAQKRKSLYKQIGLYPVLVIDSSGYVNPN
YTGRIRLDIQGTGQLLFSVVINQLRLSDAGQYLCQAGDDSNSNKKNADLQVLKPEPELV
YEDLRGSVTFHCALGPEVANVAKFLCRQSSGENCDVVVNTLGKRAPAFEGRILLNPQD
KDGSFSVVITGLRKEDAGRYLCGAHSDGQLQEGSPIQAWQLFVNGSHHHHHH
SEQ ID NO: 125 - human pIgR extracellular domain 4-domain 5 (D4-D5)
STIPRSPTVVKGVAGSSVAVLCPYNRKESKSIKYWCLWEGAQNGRCPLLVDSEGWVKA
QYEGRLSLLEEPGNGTFTVILNQLTSRDAGFYWCLTNGDTLWRTTVEIKIIEGEPNLKVP
GNVTAVLGETLKVPCHFPCKFSSYEKYWCKWNNTGCQALPSQDEGPSKAFVNCDENSR
LVSLTLNLVTRADEGWYWCGVKQGHFYGETAAVYVAVEERGSHHHHHH
SEQ ID NO: 126 - pIgR CDR1 of D1
GPQYASY
SEQ ID NO: 127 - pIgR CDR2 of D1
DAP
SEQ ID NO: 128 - pIgR CDR3 of D1
VGGVWSAD
SEQ ID NO: 129 - mouse pIgR extracellular domain (ECD)
KSPIFGPQEVSSIEGDSVSITCYYPDTSVNRHTRKYWCRQGASGMCTTLISSNGYLSKEYS
GRANLINFPENNTFVINIEQLTQDDTGSYKCGLGTSNRGLSFDVSLEVSQVPELPSDTHV
YTKDIGRNVTIECPFKRENAPSKKSLCKKTNQSCELVIDSTEKVNPSYIGRAKLFMKGTD
LTVFYVNISHLTHNDAGLYICQAGEGPSADKKNVDLQVLAPEPELLYKDLRSSVTFECD
LGREVANEAKYLCRMNKETCDVIINTLGKRDPDFEGRILITPKDDNGRFSVLITGLRKED
AGHYQCGAHSSGLPQEGWPIQTWQLFVNEESTIPNRRSVVKGVTGGSVAIACPYNPKES
SSLKYWCRWEGDGNGHCPVLVGTQAQVQEEYEGRLALFDQPGNGTYTVILNQLTTED
AGFYWCLTNGDSRWRTTIELQVAEATREPNLEVTPQNATAVLGETFTVSCHYPCKFYSQ
EKYWCKWSNKGCHILPSHDEGARQSSVSCDQSSQLVSMTLNPVSKEDEGWYWCGVKQ
GQTYGETTAIYIAVEERTRGSSHVNPTDANARAKVALEEEVVDSSISEKENKAIPNPGPF
ANEREIQNVGDQAQENRASGDAGSADGQSRSSSSKHHHHHH
SEQ ID NO: 130 - hinge region (AA)
EPKTPKPQPQPQLQPQPNPTTESKSPK
SEQ ID NO: 131 - hinge region (DNA)
GAACCCAAGACACCAAAACCACAACCACAACCACAACTACAACCACAACCCAATCC
TACAACAGAATCCAAGAGCCCCAAAA
SEQ ID NO: 132 - Human IgG1 Mono-Fc DNA sequence
AGCCCAGCACCTGAACTCCTGGGGGGACCGTCAGTCTTCCTCTTCCCCCCAAAACCC
AAGGACACCCTCATGATCTCCCGGACCCCTGAGGTCACATGCGTGGTGGTGGACGT
GAGCCACGAAGACCCTGAGGTCAAGTTCAACTGGTACGTGGACGGCGTGGAGGTGC
ATAATGCCAAGACAAAGCCGCGGGAGGAGCAGTACAACAGCACGTACCGTGTGGTC
AGCGTCCTCACCGTCCTGCACCAGGACTGGCTGAATGGCAAGGAGTACAAGTGCAA
GGTCTCCAACAAAGCCCTCCCAGCCCCCATCGAGAAAACCATCTCCAAAGCCAAAG
GGCAGCCCCGAGAACCACAGGTGTACACCAAGCCCCCATCCCGGGAGGAGATGACC
AAGAACCAGGTCAGCCTGAGCTGCCTGGTCAAAGGCTTCTATCCCAGCGACATCGC
CGTGGAGTGGGAGAGCAATGGGCAGCCGGAGAACAACTACAAGACCACGGTGCCC
GTGCTGGACTCCGACGGCTCCTTCAGACTCGCAAGCTATCTCACCGTGGACAAGAGC
AGATGGCAGCAGGGGAACGTCTTCTCATGCTCCGTGATGCATGAGGCTCTGCACAAC
CACTACACGCAGAAGAGCCTCTCCCTGTCTCCGGGTAAA
SEQ ID NO: 146 - Human IgG1 Mono-Fc AA sequence
SPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTKPPSREEMTKNQVSLSCLVKGFYPSDIAVEWESNGQPENNYKTTVPVLDSDGSFR
LASYLTVDKSRWQQGNVFSCSVMHEALHNHYTQKSLSLSPGK
SEQ ID NO: 133 - DNA sequence coding for VHH1
CAGGTGCAGCTGGTGGAGTCTGGGGGAGGATTGGTGCAGGCTGGGGGCTCTCTGAA
ACTCGCCTGTGCAGCACCTGGACTTACCTTCAGTTCGTATCGCATGGGCTGGTTCCG
CCAGGCTCCAGGGCAGGAGCGTGAGTTTGTAGCAGCTATTGATTGGAATGGTCGTG
GCACATATTATCGATACTATGCAGACTCCGTGAAGGGCCGATCCACCATTTCCAGAG
ACAACGCCAAGAACACGATGTATCTGCAAATGAACAGCCTGAAACCTGAGGACACG
GCCGTTTATTACTGTGCAGCAGGTTCGATCGACCTTAACTGGTACGGCGGCATGGAC
TACTGGGGCNANGGGACCCAGGTCACCGTCTCCTCA
SEQ ID NO: 134 - DNA sequence coding for VHH2
GAGGTGCAGGTGGTGGAGTCTGGGGGAGGATTGGTGCAGGCTGGGGGCTCTCTGAA
ACTCGCCTGTGCAGCACCTGGACTTACCTTCAGTTCGTATCGCATGGGCTGGTTCCG
CCAGGCTCCAGGGCAGGAGCGTGAGTTTGTAGCAGCTATTGATTGGAATGGTCGTG
GCACATATTATCGATACTATGCAGACTCCGTGAAGGGCCGATCCACCATTTCCAGAG
ACAACGCCAAGAACACGGTGTATCTGCAAATGAACAGCCTGAAACCTGAGGACACG
GCCGTTTATTACTGTGCAGCTACTACGGTATTAACTGACCCTAGGGTTCTTAATGAGT
ATGCCACATGGGGCCAGGGGACCCAGGTCACCGTCTCCTCA
SEQ ID NO: 135 - DNA sequence coding for VHH3
CAGTTGCAGCTCGTGGAGTCTGGGGGAGGCTTGGTGCAGCCTGGGGGGTCTCTGAG
ACTCTCCTGTGCAGCCTCTGGAAGCATCTTCAGTATCAATGTTATGGGCTGGTACCG
CCAGGCTCCAGGGAAGCAGCGCGAGTTGGTCGCACGTATTAATGGAGGTGGCATTA
CACACTATGCAGAGTCCGTGAAGGGCCGATTCACCATCTCCAGAGACAACGCCAAG
AACACGGTGTATCTGCAAATGAACAGCCTGAAACCTGAGGACACAGCCGCATATTA
CTGTAAGGCAGATGTGTTCGGTAGTAGCGGGTACGTAGAAACCTACTGGGGCCAGG
GGACCCAGGTCACCGTCTCCTCA
SEQ ID NO: 136 - DNA sequence coding for VHH4
GAGGTGCAGGTGGTGGAGTCTGGGGGAGGCTTGGTGCAGGCTGGGGGCTCTCTGAG
ACTCTCCTGTGCAGTCTCTGGAACCTCCGTCAGTAGCAATGCCATGGGTTGGTACCG
CCAGGCTCCAGGGAAGCAGCGCGAGTGGGTCGGATTTATTGATCGTATTGCTACCAC
GACGATTGCAACCTCCGTGAAGGGCCGATTCGCCATCACCAGAGACAACGCCAAGA
ACACGGTGTATCTCCAAATGAGCGGCCTGAAACCTGAGGACACAGCCGTCTATTACT
GTAATCATCCATTGACCGCTCGGTGGGGCCAGGGGACCCAGGTCACCGTCTCCTCA
SEQ ID NO: 137 - DNA sequence coding for VHH5
CAGGTGCAGCTGGTGGAGTCTGGGGGAGGCTTGGTGCAGGCTGGGGGCTCTCTGAG
ACTCTCCTGTGCAGCCTCTGGACGCACCTTCAGTAGCTATGCCATGGGCTGGTTCCG
CCAGGCTCCAGGGAAGGAGCGTGAGTTTGTAGCAGCTATTACCTGGAATGGTGGTA
CCACATACTATGCAGACTCCGTGAAGGGCCGATTCACCATCTCCAGAGACAACGCC
AAGAACACGGTGTATCTGCAAATGAACAGCCTGAAACCTGAGGACACGGCCGTTTA
TTACTGTGCAGCAGACCCATTCAACCAAGGCTACTGGGGCCAGGGGACCCAGGTCA
CCGTCTCCTCA
SEQ ID NO: 138 - DNA sequence coding for VHH6
GAGGTGCAGCTCGTGGAGTCTGGAGGAGGCTTGGTGCAGGCTGGGGGGTCTCTGAG
ACTCTCCTGTGCAGTCTCTGGAAGCTCCGTCAGTAGCGATGCCATGGGTTGGTACCG
CCAGGCTCCAGGGAATCAGCGCGCGTGGGTCGCATTTATTTCTGGTGGTGGTACCAC
AACCTATGCAGACTCCGTTAAGGGCCGATTCACCATCTCCAGAGACAACACCAAGA
ACACGGTGTATCTCCACATGAACAGCCTGAAACCTGAAGACACAGCCGTCTATTACT
GTAATCATCCATTGACGTCTCGGTGGGGCCAGGGGACCCAGGTCACCGTCTCCTCA
SEQ ID NO: 139 - DNA sequence coding for VHH7
GAGGTGCAGGTGGTGGAGTCTGGGGGAGGATTGGTGCAGGCTGGGGGGTCTCTGAG
ACTCGCCTGTGTAGCCTCTAGAAGCATCGGCAGTATCAATGTTATGGGCTGGTACCG
CCAGGCTCCAGGGAAGCAGCGCGACTTGGTCGCACGTATTACTGGAGGTGGCAGTA
CACACTACGCAGAGTCCGTGAAGGGCCGATTCACCATCTCCAGAGACAACGCCAAG
AACACGGTGTATCTGCAAATGAACAGCCTGGAACCTGAGGACACGGCCGTTTATTA
CTGTGCGTCAATGGTAAACCCTATCATTACGGCTTGGGGTACGATTGGTGTGCGCGA
GATTCCCGACTATGACTACTGGGGCCAGGGGACCCAGGTCACCGTCTCCTCA
SEQ ID NO: 140 - DNA sequence coding for VHH10
GAGGTGCAGGTGGTGGAGTCTGGGGGAGGCTTGGTGCAGGCTGGGGGGTCTCTGAG
ACTCTCCTGTGCAGCCTCTGGATTCACCTTCACCCGCTATGCCATGGGCTGGTTCCGC
CAGGCTCCAGGGAAGGAGCGATCGTTTGTAGCAGCTATTAGCTGGAGTGGTAGTAG
CGCAGGCTATGGAGACTCCGTGAAGGGCCGATTCACCATCTCCAGAGACAACGCCA
AGAACACGCTGTATCTGCAAATGAACAGTCTAAAACCTGAGGACACGGCCGTTTATT
ACTGTGCAGCAGACCCATTCAACCAAGGCTACTGGGGCCAGGGGACCCAGGTCACC
GTCTCCTCA
SEQ ID NO: 141 - DNA sequence coding for VHH11
GAGGTGCAGGTGGTGGAGTCTGGGGGAGGATTGGTGCAGGCTGGGGGCTCTCTGAG
ACTCTCCTGTGCAGCCTCTGGACGCACCTTCACTACCTATCGCATGGGCTGGTTCCG
CCAGGCTCCAGGGAAGGAGCGAGAGTTTGTAGCAGCTATTCGCTGGAGTGGTGGTC
GCACATTGTATGCAGACTCCGTGAAGGGCCGATTCACCATCTCCAGAGACAACGCC
AAGAACACAGCGTATCTGCAAATGAACAACCTGAGACCTGAGGACACGGCCGTTTA
TTACTGTGCAGCAGATCTAGCCGAGTATAGTGGTACTTACTCCAGCCCTGCGGACTC
CCCCGCTGGGTATGACTACTGGGGCCAGGGGACCCAGGTCACCGTCTCCTCA
SEQ ID NO: 142 - DNA sequence coding for VHH12
CAGGTGCAGCTGGTCGAAACTGGGGGAGGATTGGTGCAGGCTGGGGACTCTCTGAG
ACTCTCCTGTGCAGCCTCTGGACGCACCCTCAGCTTCAACACCTATGCCATGGGCTG
GTTCCGCCAGGCTCCAGGGAAGGAGCGTGAATTTGTAGCCTCTATTACCTGGAATGG
TGGAAGCACAAGCTACGCAGACTCCGTGAAGGGCCGATTCACCATCACCAGAGACA
ACGCCAAGAACACGGCTACTCTGCGAATGAATAGCCTGCAGCCCGACGACACGGCC
GTGTATTACTGTGCAGCAGCCCGATACTATGTGAGTGGTACTTACTTCCCCGCGAAT
TACTGGGGCCAGGGGACCCAGGTCACCGTCTCCTCA
SEQ ID NO: 143 - Exemplary stalk sequence of human pIgR
EKAVADTRDQADGSRASVDSGSSEEQGGSSR
SEQ ID NO: 144 - Exemplary stalk sequence of mounse pIgR
EREIQNVGDQAQENRASGDAGSADGQSRSSSSK
SEQ ID NO: 145 - Exemplary stalk sequence of mouse pIgR
EREIQNVRDQAQENRASGDAGSADGQSRSSSSK
SEQ ID NO: 147 - Exemplary flexible linker 1
(EAAAK)n, wherein n is an integer from 1 to 20
SEQ ID NO: 148 - Exemplary flexible linker 2
(GGGGS)n, wherein n is an integer from 1 to 20
SEQ ID NO: 149 - Exemplary flexible linker 3
(GGGS)n, wherein n is an integer from 1 to 20
SEQ ID NO: 150 - Exemplary hinge region 1
EPKSCDKTHTCPPCP
SEQ ID NO: 151 - Exemplary hinge region 2
ERKCCVECPPCP
SEQ ID NO: 152 - Exemplary hinge region 3
ELKTPLGDTTHTCPRCP(EPKSCDTPPPCPRCP)₃
SEQ ID NO: 153 - Exemplary hinge region 4
ESKYGPPCPSCP
SEQ ID NO: 154 - VHH1 and VHH2 CDR1 (Exemplary)
GLTFSSYRMG
SEQ ID NO: 155 - VHH3 CDR1 (Exemplary)
GSIFSINVMG
SEQ ID NO: 156 - VHH4 CDR1 (Exemplary)
GTSVSSNAMG
SEQ ID NO: 157 - VHH5 CDR1 (Exemplary)
GRTFSSYAMG
SEQ ID NO: 158 - VHH6 CDR1 (Exemplary)
GSSVSSDAMG
SEQ ID NO: 159 - VHH7 CDR1 (Exemplary)
RSIGSINVMG
SEQ ID NO: 160 - VHH9 CDR1 (Exemplary)
GRTFSTYRMG
SEQ ID NO: 161 - VHH10 CDR1 (Exemplary)
GFTFTRYAMG
SEQ ID NO: 162 - VHH11 CDR1 (Exemplary)
GRTFTTYRMG
SEQ ID NO: 163 - VHH12 CDR1 (Exemplary)
GRTLSFNTYAMG
SEQ ID NO: 164 - VHH1 and VHH2 CDR1 (Contact)
SSYRMG
SEQ ID NO: 165 - VHH3 CDR1 (Contact)
SINVMG
SEQ ID NO: 166 - VHH4 CDR1 (Contact)
SSNAMG
SEQ ID NO: 167 - VHH5 CDR1 (Contact)
SSYAMG
SEQ ID NO: 168 - VHH6 CDR1 (Contact)
SSDAMG
SEQ ID NO: 169 - VHH7 CDR1 (Contact)
SINVMG
SEQ ID NO: 170 - VHH9 CDR1 (Contact)
STYRMG
SEQ ID NO: 171 - VHH10 CDR1 (Contact)
TRYAMG
SEQ ID NO: 172 - VHH11 CDR1 (Contact)
TTYRMG
SEQ ID NO: 173 - VHH12 CDR1 (Contact)
SFNTYAMG
SEQ ID NO: 174 - VHH1 and VHH2 CDR1 (AbM)
GLTFSSYRMG
SEQ ID NO: 175 - VHH3 CDR1 (AbM)
GSIFSINVMG
SEQ ID NO: 176 - VHH4 CDR1 (AbM)
GTSVSSNAMG
SEQ ID NO: 177 - VHH5 CDR1 (AbM)
GRTFSSYAMG
SEQ ID NO: 178 - VHH6 CDR1 (AbM)
GSSVSSDAMG
SEQ ID NO: 179 - VHH7 CDR1 (AbM)
RSIGSINVMG
SEQ ID NO: 180 - VHH9 CDR1 (AbM)
GRTFSTYRMG
SEQ ID NO: 181 - VHH10 CDR1 (AbM)
GFTFTRYAMG
SEQ ID NO: 182 - VHH11 CDR1 (AbM)
GRTFTTYRMG
SEQ ID NO: 183 - VHH12 CDR1 (AbM)
GRTLSFNTYAMG
SEQ ID NO: 184 - VHH1 and VHH2 CDR2 (Exemplary)
AIDWNGRGTYYRYYADSVKG
SEQ ID NO: 185 - VHH3 CDR2 (Exemplary)
RINGGGITHYAESVKG
SEQ ID NO: 186 - VHH4 CDR2 (Exemplary)
FIDRIATTTIATSVKG
SEQ ID NO: 187 - VHH5 CDR2 (Exemplary)
AITWNGGTTYYADSVKG
SEQ ID NO: 188 - VHH6 CDR2 (Exemplary)
FISGGGTTTYADSVKG
SEQ ID NO: 189 - VHH7 CDR2 (Exemplary)
RITGGGSTHYAESVKG
SEQ ID NO: 190 - VHH9 CDR2 (Exemplary)
AISWSGGSTTYADPVKG
SEQ ID NO: 191 - VHH10 CDR2 (Exemplary)
AISWSGSSAGYGDSVKG
SEQ ID NO: 192 - VHH11 CDR2 (Exemplary)
AIRWSGGRTLYADSVKG
SEQ ID NO: 193 - VHH12 CDR2 (Exemplary)
SITWNGGSTSYADSVKG
SEQ ID NO: 194 - VHH1 and VHH2 CDR2 (Contact)
FVAAIDWNGRGTYYRY
SEQ ID NO: 195 - VHH3 CDR2 (Contact)
LVARINGGGITH
SEQ ID NO: 196 - VHH4 CDR2 (Contact)
WVGFIDRIATTT
SEQ ID NO: 197 - VHH5 CDR2 (Contact)
FVAAITWNGGTTY
SEQ ID NO: 198 - VHH6 CDR2 (Contact)
WVAFISGGGTTT
SEQ ID NO: 199 - VHH7 CDR2 (Contact)
LVARITGGGSTH
SEQ ID NO: 200 - VHH9 CDR2 (Contact)
FVAAISWSGGSTT
SEQ ID NO: 201 - VHH10 CDR2 (Contact)
FVAAISWSGSSAG
SEQ ID NO: 202 - VHH11 CDR2 (Contact)
FVAAIRWSGGRTL
SEQ ID NO: 203 - VHH12 CDR2 (Contact)
FVASITWNGGSTS
SEQ ID NO: 204 - VHH1 and VHH2 CDR2 (AbM)
AIDWNGRGTYYRY
SEQ ID NO: 205 - VHH3 CDR2 (AbM)
RINGGGITH
SEQ ID NO: 206 - VHH4 CDR2 (AbM)
FIDRIATTT
SEQ ID NO: 207 - VHH5 CDR2 (AbM)
AITWNGGTTY
SEQ ID NO: 208 - VHH6 CDR2 (AbM)
FISGGGTTT
SEQ ID NO: 209 - VHH7 CDR2 (AbM)
RITGGGSTH
SEQ ID NO: 210 - VHH9 CDR2 (AbM)
AISWSGGSTT
SEQ ID NO: 211 - VHH10 CDR2 (AbM)
AISWSGSSAG
SEQ ID NO: 212 - VHH11 CDR2 (AbM)
AIRWSGGRTL
SEQ ID NO: 213 - VHH12 CDR2 (AbM)
SITWNGGSTS
SEQ ID NO: 214 - VHH1 CDR3 (Exemplary)
GSIDLNWYGGMDY
SEQ ID NO: 215 - VHH2 CDR3 (Exemplary)
TTVLTDPRVLNEYAT
SEQ ID NO: 216 - VHH3 CDR3 (Exemplary)
DVFGSSGYVETY
SEQ ID NO: 217 - VHH4 CDR3 (Exemplary)
PLTAR
SEQ ID NO: 218 - VHH5 CDR3 (Exemplary)
DPFNQGY
SEQ ID NO: 219 - VHH6 CDR3 (Exemplary)
PLTSR
SEQ ID NO: 220 - VHH7 CDR3 (Exemplary)
MVNPIITAWGTIGVREIPDYDY
SEQ ID NO: 221 - VHH9 CDR3 (Exemplary)
QRGY
SEQ ID NO: 222 - VHH10 CDR3 (Exemplary)
DPFNQGY
SEQ ID NO: 223 - VHH11 CDR3 (Exemplary)
DLAEYSGTYSSPADSPAGYDY
SEQ ID NO: 224 - VHH12 CDR3 (Exemplary)
ARYYVSGTYFPANY
SEQ ID NO: 225 - VHH1 CDR3 (Contact)
AAGSIDLNWYGGMD
SEQ ID NO: 226 - VHH2 CDR3 (Contact)
AATTVLTDPRVLNEYA
SEQ ID NO: 227 - VHH3 CDR3 (Contact)
KADVFGSSGYVET
SEQ ID NO: 228 - VHH4 CDR3 (Contact)
NHPLTA
SEQ ID NO: 229 - VHH5 CDR3 (Contact)
AADPFNQG
SEQ ID NO: 230 - VHH6 CDR3 (Contact)
NHPLTS
SEQ ID NO: 231 - VHH7 CDR3 (Contact)
ASMVNPIITAWGTIGVREIPDYD
SEQ ID NO: 232 - VHH9 CDR3 (Contact)
NDQRG
SEQ ID NO: 233 - VHH10 CDR3 (Contact)
AADPFNQG
SEQ ID NO: 234 - VHH11 CDR3 (Contact)
AADLAEYSGTYSSPADSPAGYD
SEQ ID NO: 235 - VHH12 CDR3 (Contact)
AAARYYVSGTYFPAN
SEQ ID NO: 236 - VHH1 CDR3 (AbM)
GSIDLNWYGGMDY
SEQ ID NO: 237 - VHH2 CDR3 (AbM)
TTVLTDPRVLNEYAT
SEQ ID NO: 238 - VHH3 CDR3 (AbM)
DVFGSSGYVETY
SEQ ID NO: 239 - VHH4 CDR3 (AbM)
PLTAR
SEQ ID NO: 240 - VHH5 CDR3 (AbM)
DPFNQGY
SEQ ID NO: 241 - VHH6 CDR3 (AbM)
PLTSR
SEQ ID NO: 242 - VHH7 CDR3 (AbM)
MVNPIITAWGTIGVREIPDYDY
SEQ ID NO: 243 - VHH9 CDR3 (AbM)
QRGY
SEQ ID NO: 244 - VHH10 CDR3 (AbM)
DPFNQGY
SEQ ID NO: 245 - VHH11 CDR3 (AbM)
DLAEYSGTYSSPADSPAGYDY
SEQ ID NO: 246 - VHH12 CDR3 (AbM)
ARYYVSGTYFPANY
SEQ ID NO: 247 - hpIgR 073
EVQVVESGGGLVQAGGSLRLSCAVSGTSVSSNAMGWYRQAPGKQREWVGFIDRIATTT
IATSVKGRFAITRDNAKNTVYLQMSGLKPEDTAVYYCXHPXTARWGQGTQVTVSS
SEQ ID NO: 248 - hpIgR 201
QVQLVESGGGLVQPGGSLRLSCAASGRPNSKYAMAWFRRAPGKEREFQAAINWSGAN
TYYGDSVKGRFTISRDNAKNTVTLQMNNLNPEDTAVYYCAADNRAYTYHTFDYYQTD
ASYVYWGQGTQVTVSS
SEQ ID NO: 249 - mpIgR 349
QLQLVESGGGLVQAGGSLRLSCAASGRTFSTSTMGWFRQAPGKEREFVAAIQWSSASA
STYYYYADSVKGRFTISRDNARNTVSLQMNSLKPEDTAVYYCANLVFRVGALKERDDY
WGQGTQVTVSS
SEQ ID NO: 250 - hpIgR D1
KSPIFGPEEVNSVEGNSVSITCYYPPTSVNRHTRKYWCRQGARGGCITLISSEGYVSSKY
AGRANLTNFPENGTFVVNIAQLSQDDSGRYKCGLGINSRGLSFDVSLEVS
SEQ ID NO: 251 - mpIgR D1
KSPIFGPQEVSSIEGDSVSITCYYPDTSVNRHTRKYWCRQGASGMCTTLISSNGYLSKEYS
GRANLINFPENNTFVINIEQLTQDDTGSYKCGLGTSNRGLSFDVSLEVS
SEQ ID NO: 252 - Consensus
KSPIFGPX₁EVX₂SIEGX₃SVSITCYYPX₄TSVNRHTRKYWCRQGAX₅GX₆CX₇TLISSX₈GYL
SX₉X₁₀YAGRANLX₁₁NFPENX₁₂TFVINIX₁₃QLSQDDSGX₁₄YKCGLGX₁₅X₁₆X₁₇RGLSFDVS
LEVS
X₁ is E or Q, X₂ is N or S, X₃ is N or D, X₄ is P or D, X₅ is R or S, X₆ is G or M, X₇ is I or T, X₈
is E or N, X₉ is S or K, X₁₀ is K or E, X₁₁ is T or I, X₁₂ is G or N, X₁₃ is A or E, X₁₄ is R or S,
X₁₅ is I or T, X₁₆ is N or S, X₁₇ is S or N
SEQ ID NO: 253 - tpIgR D1
RVTTVGDLAVLEGRSVMIPCHYGPQYASYVKYWCRGSVKDLCTSLVRSDAPRGPAAA
GEDKVVMFDDPVQQVFTVTMTELQKEDSGWYWCGVEVGGVWSADVTASLHINVIQG
SEQ ID NO: 254 - hpIgR D1
KSPIFGPEEVNSVEGNSVSITCYYPPTSVNRHTRKYWCRQGARGGCITLISSEGYVSSKY
AGRANLTNFPENGTFVVNIAQLSQDDSGRYKCGLGINSRGLSFDVSLEV
SEQ ID NO: 255 - hD1 tCDR1
KSPIFGPEEVNSVEGNSVSITCYYGPQYASYRKYWCRQGARGGCITLISSEGYVSSKYAG
RANLTNFPENGTFVVNIAQLSQDDSGRYKCGLGINSRGLSFDVSLEV
SEQ ID NO: 256 - hD1 tCDR2
KSPIFGPEEVNSVEGNSVSITCYYPPTSVNRHTRKYWCRQGARGGCITLISSDAPVSSKYA
GRANLTNFPENGTFVVNIAQLSQDDSGRYKCGLGINSRGLSFDVSLEV
SEQ ID NO: 257 - hD1 tCDR3
KSPIFGPEEVNSVEGNSVSITCYYPPTSVNRHTRKYWCRQGARGGCITLISSEGYVSSKY
AGRANLTNFPENGTFVVNIAQLSQDDSGRYKCGLGVGGVWSADLSFDVSLEV
SEQ ID NO: 258 - VHH9 CDR1 (Kabat)
FTTYRMG
SEQ ID NO: 259 - VHH10 CDR1 (Kabat) - alternative
TYRMG
SEQ ID NO: 260 - VHH1 and VHH2 CDR2 (Chothia) - alternative
WNGRGTY
SEQ ID NO: 261 - VHH3 CDR2 (Chothia) - alternative
GGG
SEQ ID NO: 262 - VHH4 CDR2 (Chothia) - alternative
RIA
SEQ ID NO: 263 - VHH5 CDR2 (Chothia) - alternative
WNGG
SEQ ID NO: 264 - VHH6 CDR2 (Chothia) - alternative
GGG
SEQ ID NO: 265 - VHH7 CDR2 (Chothia) - alternative
GGG
SEQ ID NO: 266 - VHH9 CDR2 (Chothia) - alternative
WSGG
SEQ ID NO: 267 - VHH10 CDR2 (Chothia) - alternative
WSGS
SEQ ID NO: 268 - VHH11 CDR2 (Chothia) - alternative
WSGG
SEQ ID NO: 269 - VHH12 CDR2 (Chothia) - alternative
WNGG
SEQ ID NO: 270 - VHH1 and VHH2 CDR2 (IMGT) - alternative
IDWNGRGTYYR
SEQ ID NO: 271 - VHH9 CDR3 (Kabat) - alternative
QRGY
SEQ ID NO: 272 - VHH1 CDR3 (Chothia) - alternative
SIDLNWYGGMD
SEQ ID NO: 273 - VHH2 CDR3 (Chothia) - alternative
TVLTDPRVLNEYA
SEQ ID NO: 274 - VHH3 CDR3 (Chothia) - alternative
VFGSSGYVET
SEQ ID NO: 275 - VHH4 CDR3 (Chothia) - alternative
LTA
SEQ ID NO: 276 - VHH5 CDR3 (Chothia) - alternative
PFNQG
SEQ ID NO: 277 - VHH6 CDR3 (Chothia) - alternative
LTS
SEQ ID NO: 278 - VHH7 CDR3 (Chothia) - alternative
VNPIITAWGTIGVREIPDYD
SEQ ID NO: 279 - VHH9 CDR3 (Chothia) - alternative
RG
SEQ ID NO: 280 - VHH10 CDR3 (Chothia) - alternative
PFNQG
SEQ ID NO: 281 - VHH11 CDR3 (Chothia) - alternative
LAEYSGTYSSPADSPAGYD
SEQ ID NO: 282 - VHH12 CDR3 (Chothia) - alternative
RYYVSGTYFPAN
SEQ ID NO: 283 - VHH1 CDR3 (IMGT) - alternative
AAGSIDLNWYGGMDY
SEQ ID NO: 284 - VHH2 CDR3 (IMGT) - alternative
AATTVLTDPRVLNEYAT

What is claimed:

1. A method for delivering a single domain antibody or a therapeutic molecule from an apical surface of a polymeric immunoglobulin receptor (pIgR)-expressing cell to a basolateral surface of the pIgR-expressing cell comprising contacting the pIgR-expressing cell with the single domain antibody or the therapeutic molecule, wherein the single domain antibody binds to pIgR and the therapeutic molecule comprises an agent and the single domain antibody.

2. A method for transporting a therapeutic molecule to a basolateral surface of the pIgR-expressing cell of a subject, comprising administering to the subject the therapeutic molecule comprising an agent and a single domain antibody that binds to pIgR.

3. The method of claim 2, wherein the therapeutic molecule is administered to the subject via oral delivery, buccal delivery, nasal delivery or inhalation delivery.

4. A method for transporting a therapeutic molecule to systemic circulation of a subject, comprising administering to the subject a therapeutic molecule comprising an agent and a single domain antibody that binds to pIgR, wherein the therapeutic molecule is administered to the subject via oral delivery, buccal delivery, nasal delivery or inhalation delivery.

5. A method for transporting a therapeutic molecule to lamina propria or gastrointestinal tract of a subject, comprising administering to the subject a therapeutic molecule comprising an agent and a single domain antibody that binds to pIgR, wherein the therapeutic molecule is administered to the subject via oral delivery, buccal delivery, nasal delivery or inhalation delivery.

6. The method of any one of claims 2 to 5, wherein the therapeutic agent is transported from an apical surface of a pIgR-expressing cell to a basolateral surface of the pIgR-expressing cell in the subject.

7. The method of claim 1 or claim 6, wherein the single domain antibody or the therapeutic molecule comprising the agent and the single domain antibody is capable of being transported from the basolateral surface of the pIgR-expressing cell to the apical surface of the pIgR-expressing cell.

8. The method of any one of claims 1 to 7, wherein the pIgR-expressing cell is an epithelial cell.

9. The method of claim 8, wherein the epithelia cell is an intestinal lumen cell or an airway epithelial cell.

10. The method of any one of claims 1 to 9, wherein the agent is a diabetes medication.

11. The method of claim 10, wherein the diabetes medication is selected from a group consisting of insulin, glucagon-like-peptide-1, insulin-mimic peptides, and glucagon-like-peptide-1-mimic peptides.

12. The method of any one of claims 1 to 9, wherein the agent is a peptide or an antibody or a fragment thereof.

13. The method of claim 12, wherein the antibody or fragment thereof is selected from a group consisting of an anti-TNF-alpha antibody or a fragment thereof, an anti-IL23 antibody or a fragment thereof, an antibody or a fragment thereof that binds to a receptor of IL23, or an inhibitor of the receptor of IL23.

14. The method of any one of claims 1 to 9, wherein the agent is a vaccine.

15. The method of claim 14, wherein the vaccine is for preventing an infection selected from a group consisting of Vibrio, Cholera, Typhoid, Rotavirus, Tuberculosis, HIV, Flu, Ebola, and Sendai.

16. The method of any one of claims 1 to 15, wherein the single domain antibody binds to an extracellular domain 1, an extracellular domain 2, an extracellular domain 1-2, an extracellular domain 3, an extracellular domain 2-3, an extracellular domain 4-5, or an extracellular domain 5 of pIgR.

17. The method of any one of claims 1 to 15, wherein the single domain antibody binds to an extracellular domain 1 of pIgR.

18. The method of any one of claims 1 to 15, wherein the single domain antibody binds to an extracellular domain 2 of pIgR.

19. The method of any one of claims 1 to 15, wherein the single domain antibody binds to an extracellular domain 1-2 of pIgR.

20. The method of any one of claims 1 to 15, wherein the single domain antibody binds to an extracellular domain 3 of pIgR.

21. The method of any one of claims 1 to 15, wherein the single domain antibody binds to an extracellular domain 2-3 of pIgR.

22. The method of any one of claims 1 to 15, wherein the single domain antibody binds to an extracellular domain 4-5 of pIgR.

23. The method of any one of claims 1 to 15, wherein the single domain antibody binds to an extracellular domain 5 of pIgR.

24. The method of any one of claims 1 to 23, wherein the single domain antibody competes with IgA binding to the pIgR.

25. The method of any one of claims 1 to 23, wherein the single domain antibody promotes IgA binding to the pIgR.

26. The method of any one of claims 1 to 25, wherein the K_{D} of the binding of the single domain antibody to pIgR is from about 4 to about 525 nM.

27. The method of any one of claims 1 to 25, wherein the K_{D} of the binding of the single domain antibody to pIgR is less than about 50 nM.

28. The method of any one of claims 1 to 25, wherein the K_{D} of the binding of the single domain antibody to pIgR is from about 4 to about 34 nM.

29. The method of any one of claims 1 to 28, wherein the Tₘ of the single domain antibody is from about 53 to about 77° C.

30. The method of any one of claims 1 to 28, wherein the Tₘ of the single domain antibody is from 53.9 to 76.4° C.

31. The method of any one of claims 1 to 30, wherein pIgR is human pIgR.

32. The method of any one of claims 1 to 30, wherein pIgR is mouse pIgR.

33. The method of any one of claims 1 to 30, wherein the single domain antibody does not bind to a stalk sequence of human pIgR and/or a stalk sequence of mouse pIgR.

34. The method of any one of claims 1 to 33, wherein the single domain antibody comprises a CDR3 sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

35. The method of any one of claims 1 to 34, wherein the single domain antibody comprises a CDR2 sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

36. The method of any one of claims 1 to 35, wherein the single domain antibody comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

37. The method of any one of claims 1 to 36, wherein the single domain antibody comprises a CDR1 sequence, a CDR2 sequence, and a CDR3 sequence of the single domain antibody selected from the group consisting of:
a) VHH1:
i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
ii) the CDR1 sequence of GSIF SIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

38. The method of any one of claims 1 to 37, wherein the single domain antibody comprises a framework derived from the framework of any of the single domain antibodies comprising the sequences of
(SEQ ID NO: 93)
QVQLVESGGGLVQAGGSLKLACAAPGLTFSSYRMGWFRQAPGQEREFVAAI
DWNGRGTYYRYYADSVKGRSTISRDNAKNTMYLQMNSLKPEDTAVYYCAAG
SIDLNWYGGMDYWGQGTQVTVSS,
(SEQ ID NO: 94)
EVQVVESGGGLVQAGGSLKLACAAPGLTFSSYRMGWFRQAPGQEREFVAAI
DWNGRGTYYRYYADSVKGRSTISRDNAKNTVYLQMNSLKPEDTAVYYCAAT
TVLTDPRVLNEYATWGQGTQVTVSS,
(SEQ ID NO: 95)
QLQLVESGGGLVQPGGSLRLSCAASGSIFSINVMGWYRQAPGKQRELVARI
NGGGITHYAESVKGRFTISRDNAKNTVYLQMNSLKPEDTAAYYCKADVFGS
SGYVETYWGQGTQVTVSS,
(SEQ ID NO: 96)
EVQVVESGGGLVQAGGSLRLSCAVSGTSVSSNAMGWYRQAPGKQREWVGFI
DRIATTTIATSVKGRFAITRDNAKNTVYLQMSGLKPEDTAVYYCNHPLTAR
WGQGTQVTVSS,
(SEQ ID NO: 97)
QVQLVESGGGLVQAGGSLRLSCAASGRTFSSYAMGWFRQAPGKEREFVAAI
TWNGGTTYYADSVKGRFTISRDNAKNTVYLQMNSLKPEDTAVYYCAADPFN
QGYWGQGTQVTVSS,
(SEQ ID NO: 98)
EVQLVESGGGLVQAGGSLRLSCAVSGSSVSSDAMGWYRQAPGNQRAWVAFI
SGGGTTTYADSVKGRFTISRDNTKNTVYLHMNSLKPEDTAVYYCNHPLTSR
WGQGTQVTVSS,
(SEQ ID NO: 99)
EVQVVESGGGLVQAGGSLRLACVASRSIGSINVMGWYRQAPGKQRDLVARI
TGGGSTHYAESVKGRFTISRDNAKNTVYLQMNSLEPEDTAVYYCASMVNPI
ITAWGTIGVREIPDYDYWGQGTQVTVSS,
(SEQ ID NO: 100)
QVQLVESGGGLVQAGGSLRLSCAVSGRTFSTYRMGWFRQAPGKERSFVAAI
SWSGGSTTYADPVKGRFTISRDNAKNTVYLRMNSLKPEDTAVYYCNDQRGY
WGQGTLVTVSS,
(SEQ ID NO: 101)
EVQVVESGGGLVQAGGSLRLSCAASGFTFTRYAMGWFRQAPGKERSFVAAI
SWSGSSAGYGDSVKGRFTISRDNAKNTLYLQMNSLKPEDTAVYYCAADPFN
QGYWGQGTQVTVSS,
(SEQ ID NO: 102)
EVQVVESGGGLVQAGGSLRLSCAASGRTFTTYRMGWFRQAPGKEREFVAAI
RWSGGRTLYADSVKGRFTISRDNAKNTAYLQMNNLRPEDTAVYYCAADLAE
YSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFVA
SITWNGGSTSYADSVKGRFTITRDNAKNTATLRMNSLQPDDTAVYYCAAAR
YYVSGTYFPANYWGQGTQVTVSS.

39. The method of any one of claims 1 to 37, wherein the single domain antibody comprises a framework comprising sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVAAI
DWNGRGTYYRYYADSVKGRSTISRDNAKNTMYLQMNSLKPEDTAVYYCAAG
SIDLNWYGGMDYWGQGTQVTVSS,
(SEQ ID NO: 94)
EVQVVESGGGLVQAGGSLKLACAAPGLTFSSYRMGWFRQAPGQEREFVAAI
DWNGRGTYYRYYADSVKGRSTISRDNAKNTVYLQMNSLKPEDTAVYYCAAT
TVLTDPRVLNEYATWGQGTQVTVSS,
(SEQ ID NO: 95)
QLQLVESGGGLVQPGGSLRLSCAASGSIFSINVMGWYRQAPGKQRELVARI
NGGGITHYAESVKGRFTISRDNAKNTVYLQMNSLKPEDTAAYYCKADVFGS
SGYVETYWGQGTQVTVSS,
(SEQ ID NO: 96)
EVQVVESGGGLVQAGGSLRLSCAVSGTSVSSNAMGWYRQAPGKQREWVGFI
DRIATTTIATSVKGRFAITRDNAKNTVYLQMSGLKPEDTAVYYCNHPLTAR
WGQGTQVTVSS,
(SEQ ID NO: 97)
QVQLVESGGGLVQAGGSLRLSCAASGRTFSSYAMGWFRQAPGKEREFVAAI
TWNGGTTYYADSVKGRFTISRDNAKNTVYLQMNSLKPEDTAVYYCAADPFN
QGYWGQGTQVTVSS,
(SEQ ID NO: 98)
EVQLVESGGGLVQAGGSLRLSCAVSGSSVSSDAMGWYRQAPGNQRAWVAFI
SGGGTTTYADSVKGRFTISRDNTKNTVYLHMNSLKPEDTAVYYCNHPLTSR
WGQGTQVTVSS,
(SEQ ID NO: 99)
EVQVVESGGGLVQAGGSLRLACVASRSIGSINVMGWYRQAPGKQRDLVARI
TGGGSTHYAESVKGRFTISRDNAKNTVYLQMNSLEPEDTAVYYCASMVNPI
ITAWGTIGVREIPDYDYWGQGTQVTVSS,
(SEQ ID NO: 100)
QVQLVESGGGLVQAGGSLRLSCAVSGRTFSTYRMGWFRQAPGKERSFVAAI
SWSGGSTTYADPVKGRFTISRDNAKNTVYLRMNSLKPEDTAVYYCNDQRGY
WGQGTLVTVSS,
(SEQ ID NO: 101)
EVQVVESGGGLVQAGGSLRLSCAASGFTFTRYAMGWFRQAPGKERSFVAAI
SWSGSSAGYGDSVKGRFTISRDNAKNTLYLQMNSLKPEDTAVYYCAADPFN
QGYWGQGTQVTVSS,
(SEQ ID NO: 102)
EVQVVESGGGLVQAGGSLRLSCAASGRTFTTYRMGWFRQAPGKEREFVAAI
RWSGGRTLYADSVKGRFTISRDNAKNTAYLQMNNLRPEDTAVYYCAADLAE
YSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFVA
SITWNGGSTSYADSVKGRFTITRDNAKNTATLRMNSLQPDDTAVYYCAAAR
YYVSGTYFPANYWGQGTQVTVSS.

40. The method of any one of claims 1 to 39, wherein the single domain antibody is comprised of a sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVAAI
DWNGRGTYYRYYADSVKGRSTISRDNAKNTMYLQMNSLKPEDTAVYYCAAG
SIDLNWYGGMDYWGQGTQVTVSS,
(SEQ ID NO: 94)
EVQVVESGGGLVQAGGSLKLACAAPGLTFSSYRMGWFRQAPGQEREFVAAI
DWNGRGTYYRYYADSVKGRSTISRDNAKNTVYLQMNSLKPEDTAVYYCAAT
TVLTDPRVLNEYATWGQGTQVTVSS,
(SEQ ID NO: 95)
QLQLVESGGGLVQPGGSLRLSCAASGSIFSINVMGWYRQAPGKQRELVARI
NGGGITHYAESVKGRFTISRDNAKNTVYLQMNSLKPEDTAAYYCKADVFGS
SGYVETYWGQGTQVTVSS,
(SEQ ID NO: 96)
EVQVVESGGGLVQAGGSLRLSCAVSGTSVSSNAMGWYRQAPGKQREWVGFI
DRIATTTIATSVKGRFAITRDNAKNTVYLQMSGLKPEDTAVYYCNHPLTAR
WGQGTQVTVSS,
(SEQ ID NO: 97)
QVQLVESGGGLVQAGGSLRLSCAASGRTFSSYAMGWFRQAPGKEREFVAAI
TWNGGTTYYADSVKGRFTISRDNAKNTVYLQMNSLKPEDTAVYYCAADPFN
QGYWGQGTQVTVSS,
(SEQ ID NO: 98)
EVQLVESGGGLVQAGGSLRLSCAVSGSSVSSDAMGWYRQAPGNQRAWVAFI
SGGGTTTYADSVKGRFTISRDNTKNTVYLHMNSLKPEDTAVYYCNHPLTSR
WGQGTQVTVSS,
(SEQ ID NO: 99)
EVQVVESGGGLVQAGGSLRLACVASRSIGSINVMGWYRQAPGKQRDLVARI
TGGGSTHYAESVKGRFTISRDNAKNTVYLQMNSLEPEDTAVYYCASMVNPI
ITAWGTIGVREIPDYDYWGQGTQVTVSS,
(SEQ ID NO: 100)
QVQLVESGGGLVQAGGSLRLSCAVSGRTFSTYRMGWFRQAPGKERSFVAAI
SWSGGSTTYADPVKGRFTISRDNAKNTVYLRMNSLKPEDTAVYYCNDQRGY
WGQGTLVTVSS,
(SEQ ID NO: 101)
EVQVVESGGGLVQAGGSLRLSCAASGFTFTRYAMGWFRQAPGKERSFVAAI
SWSGSSAGYGDSVKGRFTISRDNAKNTLYLQMNSLKPEDTAVYYCAADPFN
QGYWGQGTQVTVSS,
(SEQ ID NO: 102)
EVQVVESGGGLVQAGGSLRLSCAASGRTFTTYRMGWFRQAPGKEREFVAAI
RWSGGRTLYADSVKGRFTISRDNAKNTAYLQMNNLRPEDTAVYYCAADLAE
YSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFVA
SITWNGGSTSYADSVKGRFTITRDNAKNTATLRMNSLQPDDTAVYYCAAAR
YYVSGTYFPANYWGQGTQVTVSS.

41. The method of any one of claims 1 to 40, wherein the single domain antibody is genetically fused or chemically conjugated to the agent.

42. The method of claim 41, further comprising a linker between the single domain antibody and the agent.

43. The method of claim 42, wherein the linker is a polypeptide.

44. The method of claim 43, wherein the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

45. The method of any one of claims 41 to 44, wherein the single domain antibody is chemically-conjugated to the agent.

46. The method of any one of claims 41 to 44, wherein the single domain antibody is non-covalently bound to the agent.

47. The method of any one of claims 1 to 46, wherein the method does not inhibit pIgR-mediated transcytosis of IgA.

48. The method of claim 47, wherein the single domain antibody comprises a CDR1 sequence of SNAMG (SEQ ID NO: 3), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GTSVSSN (SEQ ID NO: 12), GRTFSSY (SEQ ID NO: 13), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GTSVSSNA (SEQ ID NO: 22), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GTSVSSNAMG (SEQ ID NO: 156), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSNAMG (SEQ ID NO: 166), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GTSVSSNAMG (SEQ ID NO: 176), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

49. The method of claim 47 or claim 48, wherein the single domain antibody comprises a CDR2 sequence of FIDRIATTTIATSVKG (SEQ ID NO: 32), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DRIAT (SEQ ID NO: 42), MA (SEQ ID NO: 262), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDRIATT (SEQ ID NO: 52), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), FIDRIATTTIATSVKG (SEQ ID NO: 186), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), WVGFIDRIATTT (SEQ ID NO: 196), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), FIDRIATTT (SEQ ID NO: 206), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213)

50. The method of any one of claims 47 to 49, wherein the single domain antibody comprises a CDR3 sequence of PLTAR (SEQ ID NO: 63), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), PLTAR (SEQ ID NO: 74), LTA (SEQ ID NO: 275), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), NHPLTAR (SEQ ID NO: 85), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), PLTAR (SEQ ID NO: 217), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), NHPLTA (SEQ ID NO: 228), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), PLTAR (SEQ ID NO: 239), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

51. A process for providing a molecule to a subject, comprising administering to the subject the molecule comprising an agent and a single domain antibody that binds to polymeric immunoglobulin receptor (pIgR), wherein the molecule is administered to the subject via oral delivery, buccal delivery, nasal delivery or inhalation delivery.

52. The process of claim 51, wherein the molecule is capable of being provided to a basolateral surface of an pIgR-expressing cell from an apical surface of the pIgR-expressing cell in the subject.

53. The process of claim 51 or claim 52, wherein the molecule is capable of being provided to an apical surface of the pIgR-expressing cell from a basolateral surface of an pIgR-expressing cell in the subject.

54. The process of any one of claims 51 to 53, wherein the pIgR-expressing cell is an epithelial cell.

55. The process of claim 54, wherein the epithelia cell is an intestinal lumen cell or an airway epithelial cell.

56. The process of any one of claims 51 to 55, wherein the agent is a diabetes medication.

57. The process of claim 56, wherein the diabetes medication is selected from a group consisting of insulin, glucagon-like-peptide-1, insulin-mimic peptides, and glucagon-like-peptide-1-mimic peptides.

58. The process of any one of claims 51 to 55, wherein the agent is a peptide or an antibody or a fragment thereof.

59. The process of claim 58, wherein the antibody or fragment thereof is selected from a group consisting of an anti-TNF-alpha antibody or a fragment thereof, an anti-IL23 antibody or a fragment thereof, and an antibody that binds to a receptor of IL23 or a fragment thereof.

60. The process of any one of claims 51 to 55, wherein the agent is a vaccine.

61. The process of claim 60, wherein the vaccine is for preventing an infection selected from a group consisting of Vibrio, Cholera, Typhoid, Rotavirus, Tuberculosis, HIV, Flu, Ebola, and Sendai.

62. The process of any one of claims 51 to 61, wherein the single domain antibody binds to an extracellular domain 1, an extracellular domain 2, an extracellular domain 1-2, an extracellular domain 3, an extracellular domain 2-3, an extracellular domain 4-5, or an extracellular domain 5 of pIgR.

63. The process of any one of claims 51 to 61, wherein the single domain antibody binds to an extracellular domain 1 of pIgR.

64. The process of any one of claims 51 to 61, wherein the single domain antibody binds to an extracellular domain 2 of pIgR.

65. The process of any one of claims 51 to 61, wherein the single domain antibody binds to an extracellular domain 1-2 of pIgR.

66. The process of any one of claims 51 to 61, wherein the single domain antibody binds to an extracellular domain 3 of pIgR.

67. The process of any one of claims 51 to 61, wherein the single domain antibody binds to an extracellular domain 2-3 of pIgR.

68. The process of any one of claims 51 to 61, wherein the single domain antibody binds to an extracellular domain 4-5 of pIgR.

69. The process of any one of claims 51 to 61, wherein the single domain antibody binds to an extracellular domain 5 of pIgR.

70. The process of any one of claims 51 to 69, wherein the single domain antibody competes with IgA binding to the pIgR.

71. The process of any one of claims 51 to 69, wherein the single domain antibody promotes IgA binding to the pIgR.

72. The process of any one of claims 51 to 71, wherein the K_{D} of the binding of the single domain antibody to pIgR is from about 4 to about 525 nM.

73. The process of any one of claims 51 to 71, wherein the K_{D} of the binding of the single domain antibody to pIgR is less than about 50 nM.

74. The process of any one of claims 51 to 71, wherein the K_{D} of the binding of the single domain antibody to pIgR is from about 4 to about 34 nM.

75. The process of any one of claims 51 to 74, wherein the Tₘ of the single domain antibody is from about 53 to about 77° C.

76. The process of any one of claims 51 to 74, wherein the Tₘ of the single domain antibody is from 53.9 to 76.4° C.

77. The process of any one of claims 51 to 76, wherein pIgR is human pIgR.

78. The process of any one of claims 51 to 76, wherein pIgR is mouse pIgR.

79. The process of any one of claims 51 to 76, wherein the single domain antibody does not bind to a stalk sequence of human pIgR and/or a stalk sequence of mouse pIgR.

80. The process of any one of claims 51 to 79, wherein the single domain antibody comprises a CDR3 sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

81. The process of any one of claims 51 to 80, wherein the single domain antibody comprises a CDR2 sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

82. The process of any one of claims 51 to 81, wherein the single domain antibody comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

83. The process of any one of claims 51 to 82, wherein the single domain antibody comprises a CDR1 sequence, a CDR2 sequence, and a CDR3 sequence of the single domain antibody selected from the group consisting of:
a) VHH1:
i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
ii) the CDR1 sequence of GTSVSSN (SEQ ID NO: 12), the CDR2 sequence of DRIAT (SEQ ID NO: 42) or MA (SEQ ID NO: 262), and the CDR3 sequence of PLTAR (SEQ ID NO: 74) or LTA (SEQ ID NO: 275);
iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

84. The process of any one of claims 51 to 83, wherein the single domain antibody comprises a framework derived from the framework of any of the single domain antibodys comprising the sequences of
(SEQ ID NO: 93)
QVQLVESGGGLVQAGGSLKLACAAPGLTFSSYRMGWFRQAPGQEREFVAAI
DWNGRGTYYRYYADSVKGRSTISRDNAKNTMYLQMNSLKPEDTAVYYCAAG
SIDLNWYGGMDYWGQGTQVTVSS,
(SEQ ID NO: 94)
EVQVVESGGGLVQAGGSLKLACAAPGLTFSSYRMGWFRQAPGQEREFVAAI
DWNGRGTYYRYYADSVKGRSTISRDNAKNTVYLQMNSLKPEDTAVYYCAAT
TVLTDPRVLNEYATWGQGTQVTVSS,
(SEQ ID NO: 95)
QLQLVESGGGLVQPGGSLRLSCAASGSIFSINVMGWYRQAPGKQRELVARI
NGGGITHYAESVKGRFTISRDNAKNTVYLQMNSLKPEDTAAYYCKADVFGS
SGYVETYWGQGTQVTVSS,
(SEQ ID NO: 96)
EVQVVESGGGLVQAGGSLRLSCAVSGTSVSSNAMGWYRQAPGKQREWVGFI
DRIATTTIATSVKGRFAITRDNAKNTVYLQMSGLKPEDTAVYYCNHPLTAR
WGQGTQVTVSS,
(SEQ ID NO: 97)
QVQLVESGGGLVQAGGSLRLSCAASGRTFSSYAMGWFRQAPGKEREFVAAI
TWNGGTTYYADSVKGRFTISRDNAKNTVYLQMNSLKPEDTAVYYCAADPFN
QGYWGQGTQVTVSS,
(SEQ ID NO: 98)
EVQLVESGGGLVQAGGSLRLSCAVSGSSVSSDAMGWYRQAPGNQRAWVAFI
SGGGTTTYADSVKGRFTISRDNTKNTVYLHMNSLKPEDTAVYYCNHPLTSR
WGQGTQVTVSS,
(SEQ ID NO: 99)
EVQVVESGGGLVQAGGSLRLACVASRSIGSINVMGWYRQAPGKQRDLVARI
TGGGSTHYAESVKGRFTISRDNAKNTVYLQMNSLEPEDTAVYYCASMVNPI
ITAWGTIGVREIPDYDYWGQGTQVTVSS,
(SEQ ID NO: 100)
QVQLVESGGGLVQAGGSLRLSCAVSGRTFSTYRMGWFRQAPGKERSFVAAI
SWSGGSTTYADPVKGRFTISRDNAKNTVYLRMNSLKPEDTAVYYCNDQRGY
WGQGTLVTVSS,
(SEQ ID NO: 101)
EVQVVESGGGLVQAGGSLRLSCAASGFTFTRYAMGWFRQAPGKERSFVAAI
SWSGSSAGYGDSVKGRFTISRDNAKNTLYLQMNSLKPEDTAVYYCAADPFN
QGYWGQGTQVTVSS,
(SEQ ID NO: 102)
EVQVVESGGGLVQAGGSLRLSCAASGRTFTTYRMGWFRQAPGKEREFVAAI
RWSGGRTLYADSVKGRFTISRDNAKNTAYLQMNNLRPEDTAVYYCAADLAE
YSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFVA
SITWNGGSTSYADSVKGRFTITRDNAKNTATLRMNSLQPDDTAVYYCAAAR
YYVSGTYFPANYWGQGTQVTVSS.

85. The process of any one of claims 51 to 83, wherein the single domain antibody comprises a framework comprising sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVAAI
DWNGRGTYYRYYADSVKGRSTISRDNAKNTMYLQMNSLKPEDTAVYYCAAG
SIDLNWYGGMDYWGQGTQVTVSS,
(SEQ ID NO: 94)
EVQVVESGGGLVQAGGSLKLACAAPGLTFSSYRMGWFRQAPGQEREFVAAI
DWNGRGTYYRYYADSVKGRSTISRDNAKNTVYLQMNSLKPEDTAVYYCAAT
TVLTDPRVLNEYATWGQGTQVTVSS,
(SEQ ID NO: 95)
QLQLVESGGGLVQPGGSLRLSCAASGSIFSINVMGWYRQAPGKQRELVARI
NGGGITHYAESVKGRFTISRDNAKNTVYLQMNSLKPEDTAAYYCKADVFGS
SGYVETYWGQGTQVTVSS,
(SEQ ID NO: 96)
EVQVVESGGGLVQAGGSLRLSCAVSGTSVSSNAMGWYRQAPGKQREWVGFI
DRIATTTIATSVKGRFAITRDNAKNTVYLQMSGLKPEDTAVYYCNHPLTAR
WGQGTQVTVSS,
(SEQ ID NO: 97)
QVQLVESGGGLVQAGGSLRLSCAASGRTFSSYAMGWFRQAPGKEREFVAAI
TWNGGTTYYADSVKGRFTISRDNAKNTVYLQMNSLKPEDTAVYYCAADPFN
QGYWGQGTQVTVSS,
(SEQ ID NO: 98)
EVQLVESGGGLVQAGGSLRLSCAVSGSSVSSDAMGWYRQAPGNQRAWVAFI
SGGGTTTYADSVKGRFTISRDNTKNTVYLHMNSLKPEDTAVYYCNHPLTSR
WGQGTQVTVSS,
(SEQ ID NO: 99)
EVQVVESGGGLVQAGGSLRLACVASRSIGSINVMGWYRQAPGKQRDLVARI
TGGGSTHYAESVKGRFTISRDNAKNTVYLQMNSLEPEDTAVYYCASMVNPI
ITAWGTIGVREIPDYDYWGQGTQVTVSS,
(SEQ ID NO: 100)
QVQLVESGGGLVQAGGSLRLSCAVSGRTFSTYRMGWFRQAPGKERSFVAAI
SWSGGSTTYADPVKGRFTISRDNAKNTVYLRMNSLKPEDTAVYYCNDQRGY
WGQGTLVTVSS,
(SEQ ID NO: 101)
EVQVVESGGGLVQAGGSLRLSCAASGFTFTRYAMGWFRQAPGKERSFVAAI
SWSGSSAGYGDSVKGRFTISRDNAKNTLYLQMNSLKPEDTAVYYCAADPFN
QGYWGQGTQVTVSS,
(SEQ ID NO: 102)
EVQVVESGGGLVQAGGSLRLSCAASGRTFTTYRMGWFRQAPGKEREFVAAI
RWSGGRTLYADSVKGRFTISRDNAKNTAYLQMNNLRPEDTAVYYCAADLAE
YSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFVA
SITWNGGSTSYADSVKGRFTITRDNAKNTATLRMNSLQPDDTAVYYCAAAR
YYVSGTYFPANYWGQGTQVTVSS.

86. The process of any one of claims 51 to 85, wherein the single domain antibody is comprised of a sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVAAI
DWNGRGTYYRYYADSVKGRSTISRDNAKNTMYLQMNSLKPEDTAVYYCAAG
SIDLNWYGGMDYWGQGTQVTVSS,
(SEQ ID NO: 94)
EVQVVESGGGLVQAGGSLKLACAAPGLTFSSYRMGWFRQAPGQEREFVAAI
DWNGRGTYYRYYADSVKGRSTISRDNAKNTVYLQMNSLKPEDTAVYYCAAT
TVLTDPRVLNEYATWGQGTQVTVSS,
(SEQ ID NO: 95)
QLQLVESGGGLVQPGGSLRLSCAASGSIFSINVMGWYRQAPGKQRELVARI
NGGGITHYAESVKGRFTISRDNAKNTVYLQMNSLKPEDTAAYYCKADVFGS
SGYVETYWGQGTQVTVSS,
(SEQ ID NO: 96)
EVQVVESGGGLVQAGGSLRLSCAVSGTSVSSNAMGWYRQAPGKQREWVGFI
DRIATTTIATSVKGRFAITRDNAKNTVYLQMSGLKPEDTAVYYCNHPLTAR
WGQGTQVTVSS,
(SEQ ID NO: 97)
QVQLVESGGGLVQAGGSLRLSCAASGRTFSSYAMGWFRQAPGKEREFVAAI
TWNGGTTYYADSVKGRFTISRDNAKNTVYLQMNSLKPEDTAVYYCAADPFN
QGYWGQGTQVTVSS,
(SEQ ID NO: 98)
EVQLVESGGGLVQAGGSLRLSCAVSGSSVSSDAMGWYRQAPGNQRAWVAFI
SGGGTTTYADSVKGRFTISRDNTKNTVYLHMNSLKPEDTAVYYCNHPLTSR
WGQGTQVTVSS,
(SEQ ID NO: 99)
EVQVVESGGGLVQAGGSLRLACVASRSIGSINVMGWYRQAPGKQRDLVARI
TGGGSTHYAESVKGRFTISRDNAKNTVYLQMNSLEPEDTAVYYCASMVNPI
ITAWGTIGVREIPDYDYWGQGTQVTVSS,
(SEQ ID NO: 100)
QVQLVESGGGLVQAGGSLRLSCAVSGRTFSTYRMGWFRQAPGKERSFVAAI
SWSGGSTTYADPVKGRFTISRDNAKNTVYLRMNSLKPEDTAVYYCNDQRGY
WGQGTLVTVSS,
(SEQ ID NO: 101)
EVQVVESGGGLVQAGGSLRLSCAASGFTFTRYAMGWFRQAPGKERSFVAAI
SWSGSSAGYGDSVKGRFTISRDNAKNTLYLQMNSLKPEDTAVYYCAADPFN
QGYWGQGTQVTVSS,
(SEQ ID NO: 102)
EVQVVESGGGLVQAGGSLRLSCAASGRTFTTYRMGWFRQAPGKEREFVAAI
RWSGGRTLYADSVKGRFTISRDNAKNTAYLQMNNLRPEDTAVYYCAADLAE
YSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFVA
SITWNGGSTSYADSVKGRFTITRDNAKNTATLRMNSLQPDDTAVYYCAAAR
YYVSGTYFPANYWGQGTQVTVSS.

87. The process of any one of claims 51 to 86, wherein the single domain antibody is genetically fused or chemically conjugated to the agent.

88. The process of claim 87, further comprising a linker between the single domain antibody and the agent.

89. The process of claim 88, wherein the linker is a polypeptide.

90. The process of claim 89, wherein the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

91. The process of any one of claims 87 to 90, wherein the single domain antibody is chemically-conjugated to the agent.

92. The process of any one of claims 87 to 90, wherein the single domain antibody is non-covalently bound to the agent.

93. The process of any one of claims 51 to 92, wherein the process does not inhibit pIgR-mediated transcytosis of IgA.

94. The process of claim 93, wherein the single domain antibody comprises a CDR1 sequence of SNAMG (SEQ ID NO: 3), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GTSVSSN (SEQ ID NO: 12), GRTFSSY (SEQ ID NO: 13), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GTSVSSNA (SEQ ID NO: 22), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GTSVSSNAMG (SEQ ID NO: 156), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSNAMG (SEQ ID NO: 166), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GTSVSSNAMG (SEQ ID NO: 176), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182) or GRTLSFNTYAMG (SEQ ID NO: 183).

95. The process of claim 93 or claim 94, wherein the single domain antibody comprises a CDR2 sequence of FIDRIATTTIATSVKG (SEQ ID NO: 32), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DRIAT (SEQ ID NO: 42), MA (SEQ ID NO: 262), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDRIATT (SEQ ID NO: 52), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), FIDRIATTTIATSVKG (SEQ ID NO: 186), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), WVGFIDRIATTT (SEQ ID NO: 196), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), FIDRIATTT (SEQ ID NO: 206), RITGGGSTH (SEQ ID NO: 209), AISWSGGSTT (SEQ ID NO: 210), AISWSGSSAG (SEQ ID NO: 211), AIRWSGGRTL (SEQ ID NO: 212), SITWNGGSTS (SEQ ID NO: 213).

96. The process of any one of claims 93 to 95, wherein the single domain antibody comprises a CDR3 sequence of PLTAR (SEQ ID NO: 63), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), PLTAR (SEQ ID NO: 74), LTA (SEQ ID NO: 275), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), NHPLTAR (SEQ ID NO: 85), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), PLTAR (SEQ ID NO: 217), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), NHPLTA (SEQ ID NO: 228), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), PLTAR (SEQ ID NO: 239), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

97. A process comprising steps for providing a molecule to a subject.

98. The process of claim 97, wherein the molecule comprises an agent and a single domain antibody that binds to pIgR.

99. The process of claim 98, wherein the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an antibiotic, or an antibody-antibiotic conjugate.

100. The process of any one of claims 97 to 99, wherein the agent is an antibody or fragment thereof, a peptide, or a vaccine.

101. The process of any one of claims 98 to 100, wherein the single domain antibody is genetically fused or chemically conjugated to the agent.

102. A system for providing a molecule to lamina propria of a subject, comprising a molecule suitable for administering to the subject, the molecule comprising an agent and a single domain antibody that binds to pIgR, wherein the molecule is administered to the subject via oral delivery, buccal delivery, nasal delivery or inhalation delivery, or a combination thereof.

103. The system of claim 102, wherein the agent is a diabetes medication.

104. The system of claim 103, wherein the diabetes medication is selected from a group consisting of insulin, glucagon-like-peptide-1, insulin-mimic peptides, and glucagon-like-peptide-1-mimic peptides.

105. The system of claim 102, wherein the agent is a peptide or an antibody or a fragment thereof.

106. The system of claim 105, wherein the antibody or fragment thereof is selected from a group consisting of an anti-TNF-alpha antibody or a fragment thereof, an anti-IL23 antibody or a fragment thereof, and an antibody that binds to a receptor of IL23 or a fragment thereof.

107. The system of claim 102, wherein the agent is a vaccine.

108. The system of claim 107, wherein the vaccine is for preventing an infection selected from a group consisting of Vibrio, Cholera, Typhoid, Rotavirus, Tuberculosis, HIV, Flu, Ebola, and Sendai.

109. The system of any one of claims 102 to 108, wherein the single domain antibody binds to an extracellular domain 1, an extracellular domain 2, an extracellular domain 1-2, an extracellular domain 3, an extracellular domain 2-3, an extracellular domain 4-5, or an extracellular domain 5 of pIgR.

110. The system of any one of claims 102 to 108, wherein the single domain antibody binds to an extracellular domain 1 of pIgR.

111. The system of any one of claims 102 to 108, wherein the single domain antibody binds to an extracellular domain 2 of pIgR.

112. The system of any one of claims 102 to 108, wherein the single domain antibody binds to an extracellular domain 1-2 of pIgR.

113. The system of any one of claims 102 to 108, wherein the single domain antibody binds to an extracellular domain 3 of pIgR.

114. The system of any one of claims 102 to 108, wherein the single domain antibody binds to an extracellular domain 2-3 of pIgR.

115. The system of any one of claims 102 to 108, wherein the single domain antibody binds to an extracellular domain 4-5 of pIgR.

116. The system of any one of claims 102 to 108, wherein the single domain antibody binds to an extracellular domain 5 of pIgR.

117. The system of any one of claims 102 to 116, wherein the single domain antibody competes with IgA binding to the pIgR.

118. The system of any one of claims 102 to 116, wherein the single domain antibody promotes IgA binding to the pIgR.

119. The system of any one of claims 102 to 118, wherein the K_{D} of the binding of the single domain antibody to pIgR is from about 4 to about 525 nM.

120. The system of any one of claims 102 to 118, wherein the K_{D} of the binding of the single domain antibody to pIgR is less than about 50 nM.

121. The system of any one of claims 102 to 118, wherein the K_{D} of the binding of the single domain antibody to pIgR is from about 4 to about 34 nM.

122. The system of any one of claims 102 to 121, wherein the Tₘ of the single domain antibody is from about 53 to about 77° C.

123. The system of any one of claims 102 to 121, wherein the Tₘ of the single domain antibody is from 53.9 to 76.4° C.

124. The system of any one of claims 102 to 123, wherein pIgR is human pIgR.

125. The system of any one of claims 102 to 123, wherein pIgR is mouse pIgR.

126. The system of any one of claims 102 to 123, wherein the single domain antibody does not bind to a stalk sequence of human pIgR and/or a stalk sequence of mouse pIgR.

127. The system of any one of claims 102 to 126, wherein the single domain antibody comprises a CDR3 sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

128. The system of any one of claims 102 to 127, wherein the single domain antibody comprises a CDR2 sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

129. The system of any one of claims 102 to 128, wherein the single domain antibody comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

130. The system of any one of claims 102 to 129, wherein the single domain antibody comprises a CDR1 sequence, a CDR2 sequence, and a CDR3 sequence of the single domain antibody selected from the group consisting of:
a) VHH1:
i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
ii) the CDR1 sequence of GSIF SIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
ii) the CDR1 sequence of GTSVSSN (SEQ ID NO: 12), the CDR2 sequence of DRIAT (SEQ ID NO: 42) or MA (SEQ ID NO: 262), and the CDR3 sequence of PLTAR (SEQ ID NO: 74) or LTA (SEQ ID NO: 275);
iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

131. The system of any one of claims 102 to 130, wherein the single domain antibody comprises a framework derived from the framework of any of the single domain antibodys comprising the sequences of
(SEQ ID NO: 93)
QVQLVESGGGLVQAGGSLKLACAAPGLTFSSYRMGWFRQAPGQEREFVAAI
DWNGRGTYYRYYADSVKGRSTISRDNAKNTMYLQMNSLKPEDTAVYYCAAG
SIDLNWYGGMDYWGQGTQVTVSS,
(SEQ ID NO: 94)
EVQVVESGGGLVQAGGSLKLACAAPGLTFSSYRMGWFRQAPGQEREFVAAI
DWNGRGTYYRYYADSVKGRSTISRDNAKNTVYLQMNSLKPEDTAVYYCAAT
TVLTDPRVLNEYATWGQGTQVTVSS,
(SEQ ID NO: 95)
QLQLVESGGGLVQPGGSLRLSCAASGSIFSINVMGWYRQAPGKQRELVARI
NGGGITHYAESVKGRFTISRDNAKNTVYLQMNSLKPEDTAAYYCKADVFGS
SGYVETYWGQGTQVTVSS,
(SEQ ID NO: 96)
EVQVVESGGGLVQAGGSLRLSCAVSGTSVSSNAMGWYRQAPGKQREWVGFI
DRIATTTIATSVKGRFAITRDNAKNTVYLQMSGLKPEDTAVYYCNHPLTAR
WGQGTQVTVSS,
(SEQ ID NO: 97)
QVQLVESGGGLVQAGGSLRLSCAASGRTFSSYAMGWFRQAPGKEREFVAAI
TWNGGTTYYADSVKGRFTISRDNAKNTVYLQMNSLKPEDTAVYYCAADPFN
QGYWGQGTQVTVSS,
(SEQ ID NO: 98)
EVQLVESGGGLVQAGGSLRLSCAVSGSSVSSDAMGWYRQAPGNQRAWVAFI
SGGGTTTYADSVKGRFTISRDNTKNTVYLHMNSLKPEDTAVYYCNHPLTSR
WGQGTQVTVSS,
(SEQ ID NO: 99)
EVQVVESGGGLVQAGGSLRLACVASRSIGSINVMGWYRQAPGKQRDLVARI
TGGGSTHYAESVKGRFTISRDNAKNTVYLQMNSLEPEDTAVYYCASMVNPI
ITAWGTIGVREIPDYDYWGQGTQVTVSS,
(SEQ ID NO: 100)
QVQLVESGGGLVQAGGSLRLSCAVSGRTFSTYRMGWFRQAPGKERSFVAAI
SWSGGSTTYADPVKGRFTISRDNAKNTVYLRMNSLKPEDTAVYYCNDQRGY
WGQGTLVTVSS,
(SEQ ID NO: 101)
EVQVVESGGGLVQAGGSLRLSCAASGFTFTRYAMGWFRQAPGKERSFVAAI
SWSGSSAGYGDSVKGRFTISRDNAKNTLYLQMNSLKPEDTAVYYCAADPFN
QGYWGQGTQVTVSS,
(SEQ ID NO: 102)
EVQVVESGGGLVQAGGSLRLSCAASGRTFTTYRMGWFRQAPGKEREFVAAI
RWSGGRTLYADSVKGRFTISRDNAKNTAYLQMNNLRPEDTAVYYCAADLAE
YSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFVA
SITWNGGSTSYADSVKGRFTITRDNAKNTATLRIVINSLQPDDTAVYYCAA
ARYYVSGTYFPANYWGQGTQVTVSS.

132. The system of any one of claims 102 to 130, wherein the single domain antibody comprises a framework comprising sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVAAI
DWNGRGTYYRYYADSVKGRSTISRDNAKNTMYLQMNSLKPEDTAVYYCAAG
SIDLNWYGGMDYWGQGTQVTVSS,
(SEQ ID NO: 94)
EVQVVESGGGLVQAGGSLKLACAAPGLTFSSYRMGWFRQAPGQEREFVAAI
DWNGRGTYYRYYADSVKGRSTISRDNAKNTVYLQMNSLKPEDTAVYYCAAT
TVLTDPRVLNEYATWGQGTQVTVSS,
(SEQ ID NO: 95)
QLQLVESGGGLVQPGGSLRLSCAASGSIFSINVMGWYRQAPGKQRELVARI
NGGGITHYAESVKGRFTISRDNAKNTVYLQMNSLKPEDTAAYYCKADVFGS
SGYVETYWGQGTQVTVSS,
(SEQ ID NO: 96)
EVQVVESGGGLVQAGGSLRLSCAVSGTSVSSNAMGWYRQAPGKQREWVGFI
DRIATTTIATSVKGRFAITRDNAKNTVYLQMSGLKPEDTAVYYCNHPLTAR
WGQGTQVTVSS,
(SEQ ID NO: 97)
QVQLVESGGGLVQAGGSLRLSCAASGRTFSSYAMGWFRQAPGKEREFVAAI
TWNGGTTYYADSVKGRFTISRDNAKNTVYLQMNSLKPEDTAVYYCAADPFN
QGYWGQGTQVTVSS,
(SEQ ID NO: 98)
EVQLVESGGGLVQAGGSLRLSCAVSGSSVSSDAMGWYRQAPGNQRAWVAFI
SGGGTTTYADSVKGRFTISRDNTKNTVYLHMNSLKPEDTAVYYCNHPLTSR
WGQGTQVTVSS,
(SEQ ID NO: 99)
EVQVVESGGGLVQAGGSLRLACVASRSIGSINVMGWYRQAPGKQRDLVARI
TGGGSTHYAESVKGRFTISRDNAKNTVYLQMNSLEPEDTAVYYCASMVNPI
ITAWGTIGVREIPDYDYWGQGTQVTVSS,
(SEQ ID NO: 100)
QVQLVESGGGLVQAGGSLRLSCAVSGRTFSTYRMGWFRQAPGKERSFVAAI
SWSGGSTTYADPVKGRFTISRDNAKNTVYLRMNSLKPEDTAVYYCNDQRGY
WGQGTLVTVSS,
(SEQ ID NO: 101)
EVQVVESGGGLVQAGGSLRLSCAASGFTFTRYAMGWFRQAPGKERSFVAAI
SWSGSSAGYGDSVKGRFTISRDNAKNTLYLQMNSLKPEDTAVYYCAADPFN
QGYWGQGTQVTVSS,
(SEQ ID NO: 102)
EVQVVESGGGLVQAGGSLRLSCAASGRTFTTYRMGWFRQAPGKEREFVAAI
RWSGGRTLYADSVKGRFTISRDNAKNTAYLQMNNLRPEDTAVYYCAADLAE
YSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFVA
SITWNGGSTSYADSVKGRFTITRDNAKNTATLRMNSLQPDDTAVYYCAAAR
YYVSGTYFPANYWGQGTQVTVSS.

133. The system of any one of claims 102 to 132, wherein the single domain antibody is comprised of a sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVAAI
DWNGRGTYYRYYADSVKGRSTISRDNAKNTMYLQMNSLKPEDTAVYYCAAG
SIDLNWYGGMDYWGQGTQVTVSS,
(SEQ ID NO: 94)
EVQVVESGGGLVQAGGSLKLACAAPGLTFSSYRMGWFRQAPGQEREFVAAI
DWNGRGTYYRYYADSVKGRSTISRDNAKNTVYLQMNSLKPEDTAVYYCAAT
TVLTDPRVLNEYATWGQGTQVTVSS,
(SEQ ID NO: 95)
QLQLVESGGGLVQPGGSLRLSCAASGSIFSINVMGWYRQAPGKQRELVARI
NGGGITHYAESVKGRFTISRDNAKNTVYLQMNSLKPEDTAAYYCKADVFGS
SGYVETYWGQGTQVTVSS,
(SEQ ID NO: 96)
EVQVVESGGGLVQAGGSLRLSCAVSGTSVSSNAMGWYRQAPGKQREWVGFI
DRIATTTIATSVKGRFAITRDNAKNTVYLQMSGLKPEDTAVYYCNHPLTAR
WGQGTQVTVSS,
(SEQ ID NO: 97)
QVQLVESGGGLVQAGGSLRLSCAASGRTFSSYAMGWFRQAPGKEREFVAAI
TWNGGTTYYADSVKGRFTISRDNAKNTVYLQMNSLKPEDTAVYYCAADPFN
QGYWGQGTQVTVSS,
(SEQ ID NO: 98)
EVQLVESGGGLVQAGGSLRLSCAVSGSSVSSDAMGWYRQAPGNQRAWVAFI
SGGGTTTYADSVKGRFTISRDNTKNTVYLHMNSLKPEDTAVYYCNHPLTSR
WGQGTQVTVSS,
(SEQ ID NO: 99)
EVQVVESGGGLVQAGGSLRLACVASRSIGSINVMGWYRQAPGKQRDLVARI
TGGGSTHYAESVKGRFTISRDNAKNTVYLQMNSLEPEDTAVYYCASMVNPI
ITAWGTIGVREIPDYDYWGQGTQVTVSS,
(SEQ ID NO: 100)
QVQLVESGGGLVQAGGSLRLSCAVSGRTFSTYRMGWFRQAPGKERSFVAAI
SWSGGSTTYADPVKGRFTISRDNAKNTVYLRMNSLKPEDTAVYYCNDQRGY
WGQGTLVTVSS,
(SEQ ID NO: 101)
EVQVVESGGGLVQAGGSLRLSCAASGFTFTRYAMGWFRQAPGKERSFVAAI
SWSGSSAGYGDSVKGRFTISRDNAKNTLYLQMNSLKPEDTAVYYCAADPFN
QGYWGQGTQVTVSS,
(SEQ ID NO: 102)
EVQVVESGGGLVQAGGSLRLSCAASGRTFTTYRMGWFRQAPGKEREFVAAI
RWSGGRTLYADSVKGRFTISRDNAKNTAYLQMNNLRPEDTAVYYCAADLAE
YSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFVA
SITWNGGSTSYADSVKGRFTITRDNAKNTATLRMNSLQPDDTAVYYCAAAR
YYVSGTYFPANYWGQGTQVTVSS.

134. The system of any one of claims 102 to 133, wherein the single domain antibody is genetically fused or chemically conjugated to the agent.

135. The system of claim 134, further comprising a linker between the single domain antibody and the agent.

136. The system of claim 135, wherein the linker is a polypeptide.

137. The system of claim 136, wherein the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 147) and (GGGS)n (SEQ ID NO: 149), wherein n is an integer from 1 to 20.

138. The system of any one of claims 134 to 137, wherein the single domain antibody is chemically-conjugated to the agent.

139. The system of any one of claims 134 to 137, wherein the single domain antibody is non-covalently bound to the agent.

140. The system of any one of claims 102 to 139, wherein the system does not inhibit pIgR-mediated transcytosis of IgA.

141. The system of claim 140, wherein the single domain antibody comprises a CDR1 sequence of SNAMG (SEQ ID NO: 3), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GTSVSSN (SEQ ID NO: 12), GRTFSSY (SEQ ID NO: 13), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GTSVSSNA (SEQ ID NO: 22), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GTSVSSNAMG (SEQ ID NO: 156), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSNAMG (SEQ ID NO: 166), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GTSVSSNAMG (SEQ ID NO: 176), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182) or GRTLSFNTYAMG (SEQ ID NO: 183).

142. The system of claim 140 or claim 141, wherein the single domain antibody comprises a CDR2 sequence of FIDRIATTTIATSVKG (SEQ ID NO: 32), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DRIAT (SEQ ID NO: 42), MA (SEQ ID NO: 262), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDRIATT (SEQ ID NO: 52), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), FIDRIATTTIATSVKG (SEQ ID NO: 186), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), WVGFIDRIATTT (SEQ ID NO: 196), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), FIDRIATTT (SEQ ID NO: 206), RITGGGSTH (SEQ ID NO: 209), AISWSGGSTT (SEQ ID NO: 210), AISWSGSSAG (SEQ ID NO: 211), AIRWSGGRTL (SEQ ID NO: 212), SITWNGGSTS (SEQ ID NO: 213).

143. The system of any one of claims 140 to 142, wherein the single domain antibody comprises a CDR3 sequence of PLTAR (SEQ ID NO: 63), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), PLTAR (SEQ ID NO: 74), LTA (SEQ ID NO: 275), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), NHPLTAR (SEQ ID NO: 85), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), PLTAR (SEQ ID NO: 217), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), NHPLTA (SEQ ID NO: 228), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), PLTAR (SEQ ID NO: 239), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

144. A system comprising a means for providing a molecule to lamina propria of a subject.

145. The system of claim 144, wherein the molecule comprises an agent and a single domain antibody that binds to pIgR.

146. The system of claim 145, wherein the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an antibiotic, or an antibody-antibiotic conjugate.

147. The system of any one of claims 144 to 146, wherein the agent is an antibody or fragment thereof, a peptide, or a vaccine.

148. The system of any one of claims 145 to 147, wherein the single domain antibody is genetically fused or chemically conjugated to the agent.
